(19) United States
(12) Patent Application Publication
(10) Pub. No.: US 2009/0068223 A1
(43) Pub. Date: Mar. 12, 2009

(54) COMBINATION VACCINE COMPRISING AN ATTENUATED BOVINE VIRAL DIARRHEA VIRUS

(75) Inventors: Gregor Meyers, Walddorfhaeslach (DE); Jeffrey Knittel, Parkville, MO (US); Knut Elbers, Gau Algesheim (DE); Rob Tremblay, Burlington (CA); Craig Jones, Kansas City, MO (US)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: BOEHRINGER INGELHEIM VETMEDICA, INC., St. Joseph, MO (US)

(21) Appl. No.: 11/559,316

(22) Filed: Nov. 13, 2006

Related U.S. Application Data
(60) Provisional application No. 60/736,705, filed on Nov. 15, 2005.

(51) Int. Cl. A61K 39/00; A61P 31/00

(52) U.S. Cl. 424/201.1; 424/202.1

(57) ABSTRACT

The present invention relates to combination vaccines for the prophylaxis and treatment of microbiological infections in cattle which comprise an attenuated bovine viral diarrhea virus (BVDV) for the prophylaxis and treatment of BVDV caused infections, and a further immunological active component for the prophylaxis and treatment of microbiological infections other than BVDV.

### RELATED APPLICATIONS

This application claims the priority benefit of application Ser. No. 60/736,705, filed Nov. 15, 2005, the leaching and contents of which are hereby incorporated by reference.

### SEQUENCE LISTING

This application contains a sequence listing, submitted in electronic format in accordance with the EFS-Web electronic filing system. This sequence listing is hereby incorporated by reference in its entirety.

### BACKGROUND OF THE INVENTION

1. Technical Field

The present invention relates to the field of animal health and in particular to combination vaccines which comprise an attenuated bovine viral diarrhea virus (BVDV) and at least one further immunological active component for treating or preventing diseases or disorders in cattle caused by infectious agents.

2. Background Information

Bovine viral diarrhea virus (BVDV) type 1 (BVDV-1) and type 2 (BVDV-2) cause bovine viral diarrhea (BVD) and mucosal disease (MD) in cattle (Baker, 1987; Moennig and Plagemann, 1992; Thiel et al., 1996). The division of BVDV into 2 species is based on significant differences at the level of genomic sequences (summarized in Heinz et al., 2000) which are also obvious from limited cross neutralizing antibody reactions (Ridpath et al. 1994). The viral proteins of BVDV, and any other virus of the pestivirus family, are arranged in the poly protein in the order NH₂—N^{pro}—C-E^{rns}-E1-E2-p7-NS2-NS3-NS4A-NS4B-NS5A-NS5B-COOH (Lindenbach and Rice, 2001). Protein C (=core- or capsidprotein) and the glycoproteins E^{rns}, E1 and E2 represent structural components of the BVDV. E^{rns} and E2 were found to be targets for antibody neutralization (Donis et al., 1988; Paton et al., 1992; van Rijn et al., 1993; Wetland et al. 1990, 1992). E^{rns} lacks a typical membrane anchor and is secreted in considerable amounts from the infected cells; this protein has been reported to exhibit RNase activity (Hulst et al., 1994; Schneider et al., 1993; Windisch et al., 1996). The function of this enzymatic activity for the viral life cycle is presently unknown. The enzymatic activity depends on the presence of two stretches of amino acids conserved between the pestivirus E^{rns} and different known RNases of plant and fungal origin. Both of these conserved sequences contain a histidine residue (Schneider et al., 1993). Exchange of each of these residues against lysine in the E^{rns} protein of a Classical Swine Fever Virus (CSFV) vaccine strain resulted in the destruction of RNase activity (Hulst et al., 1998). Introduction of these mutations into the genome of the CSFV vaccine strain did not influence viral viability or growth properties but led to a virus exhibiting a cytopathogenic phenotype (Hulst et al. 1998). Similarly, Meyers et al. showed that an RNase negative variant of the virulent CSFV strain Alfort/Tübingen was fully viable. However, the respective virus mutant showed no cytopathogenic phenotype (Meyers et al., 1999).

N^{pro} represents the first protein encoded by the long open reading frame in the pestivirus RNA. N^{pro} represents a nonstructural protein that has protease activity and cleaves itself of the nascent polyprotein (Stark et al., 1993; Wiskerchen et al., 1991) presumably already during translation. N^{pro} is a cysteine protease (Rümenapf et al., 1998) that is not essential for virus replication (Tratschin et al., 1998). Recently, it was shown that N^{pro} somehow interferes with the cellular antiviral defense so that it can be hypothesized to modulate the immune system within an infected host (Rüggli et al., 2003). Mayer and coworkers presented indications for an attenuation of CSFV in consequence of a deletion of the N^{pro} gene (Mayer et al., 2004).

Present BVDV vaccines for the prevention and treatment of BVDV infections still have drawbacks (Oirschot et al. 1999). Vaccines against the classical BVDV-1 provide only partial protection from BVDV-2 infection, and vaccinated dams may produce calves that are persistently infected with virulent BVDV-2 (Bolin et al., 1991, Ridpath et al., 1994). This problem is probably due to the great antigenic diversity between type 1 and type 2 strains which is most pronounced in the glycoprotein E2, the major antigen for virus neutralization. (Tijssen et al., 1996). Most monoclonal antibodies against type 1 strains fail to hind to type 2 viruses (Ridpath et al., 1994).

Currently, licensed BVDV MLV vaccines are produced using attenuated viruses obtained via repeated passage in bovine or porcine cells (Coggins et al., Cornell Vet. 51: 539-, 1961; Philips et al., Am. J. Vet. Res. 36: 135-, 1975), or using chemically modified viruses which exhibit a temperature-sensitive phenotype (Lobmann et al., Am. J. Vet. Res. 45: 2498-, 1984; 47: 557-561, 1986). A single dose of MLV vaccine is sufficient for immunization, and duration of the immunity can last for years in vaccinated cattle. However, as these vaccines have been developed using type I BVDV virus strains, the protection is against type I virus only. Moreover, these vaccines, although attenuated, are most often associated with safety problems. The vaccine viruses may cross the placenta of pregnant animals, e.g. cows and lead to clinical manifestations in the fetus and/or the induction of persistently infected calves. Therefore, they cannot be applied to breeding herds that contain pregnant cows. Pregnant cows have to be kept separate from vaccinated cattle to protect fetuses and must not be vaccinated themselves.

Parainfluenza-3 virus (PI-3) is an RNA virus classified in the paramyxovirus family. Infections caused by PI-3 are common in cattle. Although PI-3 is capable of causing disease, it is usually associated with mild to subclinical infections. The most important role of PI-3 is to serve as an initiator that can lead to the development of secondary bacterial pneumonia. Clinical signs include pyrexia, cough, serous nasal and lacrimal discharge, increased respiratory rate, and increased breath sounds. The severity of signs worsen with the onset of bacterial pneumonia. Fatalities from uncomplicated PI-3 pneumonia are rare. Lesions include cranioventral lung consolidation, bronchiolitis, and alveolitis with marked congestion and hemorrhage. Inclusion bodies may be identified. Most fatal cases will also have a concurrent bacterial bronchopneumonia.

Bovine Respiratory Syncytial Firm (BRSV) is an RNA virus classified as a pneumovirus in the paramyxovirus family. In addition to cattle, sheep and goats can also be infected by respiratory syncytial viruses. This virus was named for its characteristic cytopathic effect—the formation of syncytial cells. Antigenic subtypes are known to exist for BRSV, and preliminary evidence suggests that there may be antigenic subtypes of BRSV. BRSV is distributed worldwide, and the virus is indigenous in the cattle population, BRSV infections associated with respiratory disease occur predominantly in young beef and dairy cattle. Passively derived immunity does not appear to prevent BRSV infections but will reduce the severity of disease. Initial exposures to the virus are associated with severe respiratory disease; subsequent exposures result in mild to subclinical disease. BRSV appears to be an important virus in the bovine respiratory disease complex because of its frequency of occurrence, predilection for the lower respiratory tract, and its ability to predispose the respiratory tract to secondary bacterial infection. In outbreaks, morbidity tends to be high, and case fatality can be 0-20%. Signs include increased rectal temperature 40-42° C., depression, decreased feed intake, increased respiratory rate, cough, and nasal and lacrimal discharge. Generally, respiratory signs predominate. Dyspnea may become pronounced in the later stages of the disease. Subcutaneous emphysema, is sometimes reported. Secondary bacterial pneumonia is a frequent occurrence. A biphasic disease pattern has been described but is not consistent Gross lesions include a diffuse interstitial pneumonia with subpleural and interstitial emphysema along with interstitial edema. These lesions are similar to and must be differentiated from other causes of interstitial pneumonia. See also atypical interstitial pneumonia. Histologic examination reveals syncytial cells in bronchiolar epithelium and lung parenchyma, intracytoplasmic inclusion bodies, proliferation and/or degeneration of bronchiolar epithelium, alveolar epithelialization, edema, and hyaline membrane formation.

Bovine Herpesvirus (BHV-1) is associated with several diseases and symptoms in cattle: Infectious bovine rhinotracheitis (IBR), infectious pustular vulvovaginitis (IPV), balanoposthitis, conjunctivitis, abortion, encephalomyelitis, and mastitis. Only a single serotype of BHV-1 is recognized; however, three subtypes of BHV-1 have been described on the basis of endonuclease cleavage patterns of viral DNA. These types are referred to as BHV-1.1 (respiratory subtype), BHV-1.2 (genital subtype), and BHV-1.3 (encephalitic subtype). Recently, BHV-1.3 has been reclassified as a distinct herpesvirus designated BHV-5. BHV-1 infections are widespread in the cattle population. In feedlot cattle, the respiratory form is most common. The viral infection alone is not life-threatening but predisposes cattle to secondary bacterial pneumonia, which may result in death. In breeding cattle, abortion or genital infections are more common. Genital infections can occur in bulls (infectious pustular balanoposthitis) and cows (IPV) within 1-3 days of mating or close contact with an infected animal. Transmission can occur in the absence of visible lesions and through artificial insemination with semen from subclinically infected bulls. Cattle with latent BHV-1 infections generally show no clinical signs when the virus is reactivated, but they do serve as a source of infection for other susceptible animals and thus perpetuate the disease. The incubation period for the respiratory and genital forms is 2-6 days. In the respiratory form, clinical signs range from mild to severe, depending on the presence of secondary bacterial pneumonia. Clinical signs include pyrexia, anorexia, coughing, excessive salivation, nasal discharge that progresses from serous to mucopurulent, conjunctivitis with lacrimal discharge, inflamed nares (hence the common name “red nose”), and dyspnea if the larynx becomes occluded with purulent material. Pustules may develop on the nasal mucosa and later form diphtheritic plaques. Conjunctivitis with corneal opacity may develop as the only manifestation of BHV-1 infection. In the absence of bacterial pneumonia, recovery generally occurs 4-5 days after the onset of clinical signs. Abortions may occur concurrently with respiratory disease but can also occur up to 100 days after infection. Abortions can occur regardless of the severity of disease in the dam. Abortions generally occur during the second half of pregnancy, but early embryonic death may also occur. The first signs of genital infections in cows are frequent urination, elevation of the tailhead, and a mild vaginal discharge. The vulva is swollen, and small papules, then erosions and ulcers, are present on the mucosal surface. If secondary bacterial infections do not occur, animals recover in 10-14 days. If bacterial infection, occurs, there may be inflammation of the uterus and transient infertility, with purulent vaginal discharge for several weeks. In bulls, similar lesions occur on the penis and prepuce. BHV-1 infection can be severe in young calves and cause a generalized disease. Pyrexia, ocular and nasal discharges, respiratory distress, diarrhea, incoordination, and eventually convulsions and death may occur in a short period after generalized viral infection. IBR is rarely fatal in cattle unless complicated by bacterial pneumonia, in uncomplicated IBR infections, most lesions are restricted to the upper respiratory tract and trachea. Petechial to ecchymotic hemorrhages may be found in the mucous membranes of the nasal cavity and the paranasal sinuses. Focal areas of necrosis develop in the nose, pharynx, larynx, and trachea. The lesions may coalesce to form plaques. The sinuses are often filled with a serous or serofibrinous exudate. As the disease progresses, the pharynx becomes covered with a serofibrinous exudate, and blood-tinged fluid may be found in the trachea. The pharyngeal and pulmonary lymph nodes may be acutely swollen and hemorrhagic. The tracheitis may extend into the bronchi and bronchioles; when this occurs, epithelium is sloughed in the airways. The viral lesions are often masked by secondary bacterial infections. In young animals with generalized BHV-1 infection, erosions and ulcers overlaid with debris may be found in the nose, esophagus, and forestomachs. In addition, white foci may be found in the liver, kidney, spleen, and lymph nodes. Aborted fetuses may have pale, focal, necrotic lesions in all tissues, but which are especially visible in the liver.

A number of other Bovine Respiratory Viruses have been identified as being involved in BRD. Bovine herpesvirus-4 has been implicated in several diseases, including BRD. Bovine adenovirus has been associated with a wide spectrum of diseases, with bovine adenovirus type 3 being the serotype most often associated with BRD. Two serotypes of bovine rhinovirus have been recognized to cause respiratory tract infections in cattle. Other viruses reported to be associated with BRD include bovine reovirus, enterovirus, and coronavirus. These viruses have a role similar to the other viruses previously discussed in that, in combination with other stressors, they can serve as initiators of bacterial pneumonia. Bovine coronavirus is also commonly associated with diarrhea in calves. It replicates in the epithelium of the upper respiratory tract and in the enterocytes of the intestine, where it produces similar lesions to rotavirus but also infects the epithelial cells of the large intestine to produce atrophy of the colonic ridges. Vaccines are not available for prevention of these viral respiratory diseases.

Bovine rotavirus is the most common viral cause of diarrhea in calves. Group A and B rotavirus are involved, but group A is the most prevalent and clinically important and contains several serotypes of differing virulence. Rotavirus replicates in the mature absorptive and enzyme-producing enterocytes on the villi of the small intestine, leading to rupture and sloughing of the enterocytes with release of virus to infect adjacent cells. Rotavirus does not infect the immature cells of the crypts. With virulent strains of rotavirus, the loss of enterocytes exceeds the ability of the intestinal crypts to replace them; hence, villous height is reduced, with a consequent decrease in intestinal absorptive surface area and intestinal digestive enzyme activity.

Other viruses, including Breda virus, a calici-like virus. Adenovirus, Astrovirus and Parvovirus, have been demonstrated in the feces of calves with diarrhea and can produce diarrhea in calves experimentally. However, these agents can also be demonstrated in the feces of healthy calves. The importance of these agents in the syndrome of neonatal diarrhea has yet to be determined. Manheimia haemolytica (formerly Pasteurella haemolytica) biotype A, serotype I is the bacterium most frequently isolated from the lungs of cattle with BRD. Although less frequently cultured than M. haemolytica, Pasteurella multocida is also an important cause of bacterial pneumonia. When pulmonary abscessation occurs, generally in association with chronic pneumonia, Actinomyces pyogenes is frequently isolated. Under normal conditions, M. haemolytica remains confined to the upper respiratory tract, in particular the tonsillar crypts, and is difficult to culture from healthy cattle. After stress or viral infection, the replication rate of M. haemolytica in the upper respiratory tract increases rapidly, as does the likelihood of culturing the bacterium. The increased bacterial growth rate and colonization of the lungs may be due to suppression of the host's defense mechanism related to environmental stressors or viral infections. It is during this log phase of growth that virulence factors are elaborated by M. haemolytica, such as an exotoxin that has been referred to as leukotoxin. The interaction between the virulence factors of the bacteria and host defenses results in tissue damage and development of pneumonia. Clinical signs of bacterial pneumonia are often preceded by signs of viral infection of the respiratory tract. With the onset of bacterial pneumonia, the severity of clinical signs increases and are characterized by depression and toxemia. There will be pyrexia (40-41° C.); serous to mucopurulent nasal discharge; moist cough; and a rapid, shallow respiratory rate. Auscultation of the cranioventral lung field reveals increased bronchial sounds, crackles, and wheezes. In severe cases, pleurisy may develop, which is characterized by an irregular breathing pattern and grunting on expiration. The animal will become unthrifty in appearance if the pneumonia becomes chronic, which is usually associated with the formation of pulmonary abscesses. M. haemolytica causes a severe, acute fibrinous pneumonia or fibrinonecrotic pneumonia. The pneumonia has a bronchopneumonia pattern. Grossly, there is extensive reddish black to grayish brown cranioventral regions of consolidation with gelatinous thickening of interlobular septa and fibrinous pleuritis. There are extensive thromboses, foci of lung necrosis, and limited evidence of bronchitis and bronchiolitis, P. multocida is associated with a less fulminating fibrinous to fibrinopurulent bronchopneumonia. In contrast to M. haemolytica, P. multocida is associated with only small amounts of fibrin exudation, some thromboses, limited lung necrosis, and suppurative bronchitis and bronchiolitis.

Haemophilus somnus is being increasingly recognized as an important pathogen in BRD; these bacteria are normal inhabitants of the nasopharynx of cattle. H. somnus infection of the lungs results in purulent bronchopneumonia that may be followed by septicemia and infection of multiple organs. Occasionally, H. somnus is associated with extensive pleuritis. H. somnus can cause an acute, usually fatal, septicemic disease that can involve the nervous, musculoskeletal, circulatory, and respiratory systems, either singly or together. The reproductive system is often affected but usually without the other systems being clinically involved. The disease may be characterized by fever, severe depression, ataxia, weakness, blindness, coma, and death within several hours to several days, it occurs sporadically in individual beef and dairy cattle and is found nearly worldwide. H. somnus is a gram-negative, nonmotile, nonsporeforming, pleomorphic coccobacillus that requires an enriched medium and a microaerophilic atmosphere for culture. It appears to be identical to Histophilus ovis and Haemophilus agni, etiologic agents of ovine septicemia, mastitis, and epididymitis; however, transmission of H. somnus between sheep and cattle has not been demonstrated. Pathogenic and nonpathogenic strains have been differentiated by intracisternal inoculation of young calves with organisms from various sources. Pathogenic and nonpathogenic strains of H. somnus are carried in the sheath and prepuce of males, the vagina of female cattle, and in the nasal passages of both sexes. The organism may colonize the respiratory tract, presumably after inhalation, and is frequently found in urine. Prevalence of the organism in cattle is probably high because high titers of specific antibodies are found in a large proportion of tested cattle. Several disease syndromes caused by H. somnus have been recognized, including thrombomeningoencephalitis, fibrinopurulent bronchopneumonia, fibrinous pleuritis, and polyarthritis. Myocardial and skeletal muscle necrosis occur. Suppurative vaginitis, cervicitis, and endometritis have been documented in cows infected experimentally and naturally after breeding, and the organism is a cause of sporadic abortion. Strains of H. somnus that cause disease adhere to the endothelium of vessels, resulting in contraction, exposure of collagen, platelet adhesion, and thrombosis. TME results when this occurs in the brain and associated membranes, after invasion of the organism into the bloodstream of susceptible cattle. Strains may adhere to endothelium in vessels of the pleura, myocardium, synovium, or a variety of other tissues and produce inflammation, in those sites (e.g., infections of the larynx and middle ear have been recorded). The susceptibility of individual animals and variations in the preference of strains of the organism for vessels in different tissues may be important in the development of the form of disease, but the mechanisms involved are incompletely understood. Reproductive problems may not necessarily be preceded by bacteremia, but the pathogenesis is poorly defined. A fever as high as 42° C. is often the first sign of disease; however, this usually falls to normal or subnormal within hours. Other findings are determined by the system(s) involved and may include rapid respiration, stiffness, knuckling at the fetlocks, severe depression, ataxia, paralysis, and opisthotonos, followed by coma and death within several hours. Affected animals may be blind, and retinal hemorrhages with gray foci of retinal necrosis are sometimes seen. Signs such as hypersensitivity, convulsions, excitement, nystagmus, and circling occur inconsistently and may be related to the regions of the CNS affected in the course of disease development. Occasionally, animals are found, dead, indicating a rapidly fatal course. A marked change in the total and differential WBC count is common; leukopenia and neutropenia occur in severe, usually acute, fatal disease, while neutrophilia may be present in less severe disease. In TME, the total cell count of the CSF is markedly increased, and neutrophils predominate. During septicemia, the organism can be recovered from blood, synovial fluid, CSF, brain, kidneys, urine, and a variety of other organs. The lesions are characterized by vascular thrombosis and infarction of the surrounding tissue. Randomly distributed red to brown foci of necrosis with hemorrhage on the surface and cut sections of the brain and spinal cord, retina, skeletal muscle, myocardium, kidney, intestine, and spleen are characteristic. A fibrinopurulent meningitis with cloudy CSF may sometimes be seen on the surface of the brain and spinal cord, and a polyserositis, especially of joints and pleura, may occur. An acute fibrinous bronchopneumonia with tissue necrosis may develop after airborne infections.

The exact role of mycoplasmas and ureaplasmas in BRD requires better definition. Mycoplasmas can be recovered from the respiratory tract of nonpneumonic calves, but the frequency of isolation is greater in those with respiratory tract disease. The mycoplasmas commonly recovered from the lungs of pneumonic calves include Mycoplasma dispar, Mycoplasma bovis, and Ureaplasma spp. Experimental infections usually result in inapparent to mild signs of respiratory disease. Tins does not preclude a synergistic role for mycoplasmas in conjunction with viruses and bacteria in BRD. Lesions described include peribronchial and peribronchiolar lymphoid cuffing and alveolitis. Culture of these organisms requires special media and conditions and may take up to a week for growth of the organisms.

Chlamydiae have been identified in various parts of the world as a cause of enzootic pneumonia in calves. The causative agent is Chlamydia psittaci. Some respiratory isolates from calves have properties of immunotypes 1 and 6 and are similar to strains recovered from intestinal infections and abortions of cattle and sheep. Immunotype 6 has been recovered from pneumonic lungs of calves and pigs. Thus, the GI tract must be considered as an important site in the pathogenesis of chlamydial infections and as a natural reservoir and source of the organisms. Chlamydial pneumonia has affected calves under a whole range of conditions as well as on dairy farms. A synergism between Chlamydia and P. haemolytica has been demonstrated experimentally. Calves with chlamydial pneumonia are usually febrile, lethargic, and dyspneic, and have a serous and later mucopurulent nasal discharge and a dry hacking cough. Calves of weanling age are affected most frequently, but older cattle may also show signs. The acute pulmonary lesion is a bronchointerstitial pneumonia. The anteroventral parts of the lungs are affected but, in severe cases, entire lobes can be involved. The dry cough is attributed to tracheitis. Microscopic changes in the lungs include suppurative bronchitis and alveolitis progressing to type II pneumocyte hyperplasia and interstitial thickening.

Bovine genital campylobacteriosis is a venereal disease of cattle characterized primarily by early embryonic death, infertility, a protracted calving season, and occasionally, abortion. Distribution is probably worldwide. The cause is the motile, gram-negative, curved or spiral, polar flagellated bacterium Campylobacter fetus venerealis or Campylobacter fetus fetus. For many years, it was thought that C. fetus fetus (formerly C. fetus intestinalis) was generally an intestinal organism, only occasionally caused abortion in cattle, and was not a cause of infertility. However, it has been shown that C. fetus fetus can also be a significant cause of the classic infertility syndrome usually attributed to Campylobacter fetus venerealis. There are several strains of C. fetus fetus, and the only way to determine if a strain is a cause of infertility is to test that possibility in a group of heifers. Campylobacter spp are very labile and are destroyed quickly by heating, drying, and exposure to the atmosphere. Unless cultured quickly after collection from the animal and grown under microaerophilic or anaerobic conditions, campylobacters will not grow. Campylobacter fetus is transmitted venereally and also by contaminated instruments, betiding, or by artificial insemination using contaminated semen. Individual bulls vary in their susceptibility to infection because some become permanent carriers, while others appear to be resistant to infection. Bulls can also transmit the infection mechanically for several hours after copulating with an infected cow. In cows, the duration of the carrier state is also variable; some clear the infection rapidly, while others can carry C. fetus for ≧2 yr. IgA antibodies are shed in cervical mucus in significant amounts in ˜50% of cows for several months after infection and are useful diagnostically. Although most, of the genital tract may be free of infection when a cow eventually conceives, the vagina may remain chronically infected, even through pregnancy. Cows are systemically normal, but there is a variable degree of mucopurulent endometritis that causes early embryonic death, prolonged luteal phases, irregular estrous cycles, repeat breeding and, as a result, protracted calving periods. Observed abortions are not common. In herds not managed intensively, disease may be noticed only when pregnancy examinations reveal low or marginally low pregnancy rates but, more importantly, great variations in gestation lengths, especially when the disease has recently been introduced to the herd. In subsequent years, infertility is usually confined to replacement heifers and a few susceptible cows. Bulls are asymptomatic and produce normal semen.

Leptospirosis is a contagious disease of animals, including man, caused by various immunologically distinct leptospiral serovars, most of which are regarded as subgroups of Leptospira interrogans. Infections may be asymptomatic or cause various signs, including fever, icterus, hemoglobinuria, renal failure, infertility, abortion, and death. After acute infection, leptospires frequently localize in the kidneys or reproductive organs and are shed in the urine, sometimes in large numbers for months or years. Because the organisms survive in surface waters for extended periods, the disease is often waterborne. In the USA, the disease is primarily due to the serovars Leptospira hardjo, Leptospira pomona, and Leptospira grippotyphosa. However, Leptospira canicola and Leptospira icterohaemorrhagiae serovars also have been isolated. Calves may have fever, anorexia, and dyspnea, and in Leptospira pomona infections, icterus, hemoglobinuria, and anemia. Body temperature may rise suddenly to 40.5-41° C. Hemoglobinuria rarely lasts longer than 48-72 hrs. Icterus clears rapidly and is followed by anemia. The RBC's begin to increase in number by 4-5 days and return, to normal 7-10 days later. However, Leptospira hardjo infections usually do not cause hemolytic anemia, which makes diagnosis more difficult. Morbidity and mortality are higher in calves than in adult cattle. In older cattle, signs vary greatly and diagnosis is more difficult. Enzootic Leptospira hardjo infections, which usually result in abnormal milk, are more obvious in daily than in beef cattle. Signs usually are restricted to lowered milk and calf production; a hemolytic crisis does not occur. The milk is thick, yellow, and blood-tinged; it may contain clots, although there is little evidence of mammary inflammation. Milk production returns to normal in 10-14 days, even in the absence of treatment. Abortion and stillbirths, which are common in Leptospira pomona infections and sporadic in Leptospira hardjo infections, generally occur 3-10 weeks after initial infection. The abortions are more common during the third trimester. An abortion storm in a breeding herd is often the first indication that leptospirosis exists, because the mild initial signs often pass unnoticed. In endemically infected herds, abortions occur mostly in younger animals and are sporadic, rather than being manifested as abortion storms. Calves reared by previously infected cows are protected by colostral antibodies for up to 6 mos. The calves generally have an antibody titer similar to that of their dams. In the acute form, anemia, icterus, hemoglobinuria, and submucosal hemorrhages are prominent. The kidneys are swollen, with multifocal petechial and ecchymotic hemorrhages that become pale with time. The liver may be swollen, with minute areas of focal necrosis. Petechiae in other organs are seen in fulminating cases; however, in the more prevalent Leptospira hardjo infections, the lesions are primarily restricted to the kidneys.

Brucellosis is caused by bacteria, of the genus Brucella and is characterized by abortion, retained placenta, and to a lesser extent, orchitis and infection of the accessory sex glands in males. The disease in cattle, water buffalo, and bison is caused almost exclusively by Brucella abortus; however, Brucella suis or Brucella melitensis is occasionally implicated in some cattle herds. Brucella suis does not appear to be contagious from cow to cow. Infection spreads rapidly and causes many abortions in unvaccinated herds. Typically, in a herd in which disease is endemic, an infected cow aborts only once after exposure; subsequent gestations and lactations appear normal. After exposure, many cattle become bacteremic for a short period and develop agglutinins and other antibodies; others resist infection, and a small percentage of infected cows recover. A positive serum agglutination test usually precedes abortion or a normal parturition, but may be delayed in ˜15% of animals. The incubation period may be variable and is related to the stage of gestation at time of exposure. Organisms are shed in milk and uterine discharges, and the cow may become temporarily sterile. Bacteria may be found in the uterus during pregnancy, uterine involution, and infrequently, for a prolonged time in the nongravid uterus. Shedding from the vagina largely disappears with reduction of the fluids after parturition. Some infected cows that aborted previously shed brucellae from the uterus at subsequent normal parturitions. Organisms are shed in milk for a variable length of time—in most cattle for life. Natural transmission occurs by ingestion of organisms, which are present in large numbers in aborted fetuses, fetal membranes, and uterine discharges. Cattle may ingest contaminated feed and water, or lick contaminated genitals of other animals. Venereal transmission by infected bulls to susceptible cows appears to be rare. Transmission may occur by artificial insemination when Brucella-contaminated semen is deposited in the uterus but, reportedly, not when deposited in the midcervix. Brucellae may enter the body through mucous membranes, conjunctivae, wounds, or even intact skin. Mechanical vectors (eg, other animals, including man) may spread infection. Brucellae have been recovered from fetuses and from manure that has remained in a cool environment for >2 mo. Exposure to direct sunlight kills the organisms within a few hours. Abortion is the most obvious manifestation. Infections may also cause stillborn or weak calves, retained placentas, and reduced milk yield. Usually, general health is not impaired in uncomplicated abortions. Seminal vesicles, ampullae, testicles, and epididymides may be infected in bulls; therefore, organisms are in the semen. Agglutinins may be demonstrated in seminal plasma from infected bulls. Testicular abscesses may occur. Long-standing infections may result in arthritic joints in some cattle.

Actinomyces (Corynebacterium) pyogenes causes sporadic abortion in the last trimester. Rarely, the incidence in a herd may reach enzootic (64%) levels. The bacteria are present, on mucous membranes of many normal cows, as well as in uterine and abscess discharges. They gain entry to the bloodstream and cause an endometritis and placentitis, which is diffuse with a reddish brown to brown color. The fetus is usually autolyzed, with fibrinous pericarditis, pleuritis, or peritonitis possible.

Clostridia, are relatively large, anaerobic, spore-forming, rod-shaped organisms. The spores are oval, sometimes spherical, and are central, subterminal, or terminal in position. The vegetative forms of clostridia in tissue fluids of infected animals occur singly, in pairs, or rarely in chains. Differentiation of the various pathogenic and related species is based on cultural characteristics, spore shape and position, biochemical reactions, and the antigenic specificity of toxins or surface antigens. The natural habitats of the organisms are the soil and intestinal tract of animals, including man. Pathogenic strains may be acquired by susceptible animals either by wound contamination or by ingestion. Diseases thus produced are a constant threat to successful livestock production in many parts of the world.

Clostridium haemolyticum is a soil-borne organism that may be found naturally in the GI tract of cattle. It can survive for long periods in contaminated soil or in bones from carcasses of animals that had been infected. After ingestion, latent spores ultimately become lodged in the liver. The incubation period is extremely variable, and the onset depends on the presence of a locus of anaerobiosis in the liver. Such a nidus for germination is most often caused by fluke infection, much less often by high nitrate content of the diet, accidental liver puncture, liver biopsy, or any other cause of localized necrosis. When conditions for anaerobiosis are favorable, the spores germinate, and the resulting vegetative cells multiply and produce β toxin (phospholipase C), which causes intravascular hemolysis and its sequelae, including hemolytic anemia and hemoglobinuria. Cattle may be found dead without premonitory signs. Usually, there is a sudden onset of severe depression, fever, abdominal pain, dyspnea, dysentery, and hemoglobinuria Anemia and jaundice are present in varying degrees. Edema of the brisket may occur. Hgb and RBC levels are quite low. The duration of clinical signs varies from ˜12 hr in pregnant cows to ˜3-4 days in other cattle. The mortality in untreated animals is ˜95%. Some cattle suffer from subclinical attacks of the disease and thereafter act as immune carriers. Dehydration, anemia, and sometimes subcutaneous edema are present. There is bloody fluid in the abdominal and thoracic cavities. The lungs are not grossly affected, and the trachea contains bloody froth with hemorrhages in the mucosa. The small intestine and occasionally the large intestine are hemorrhagic; their contents often contain free or clotted blood. An anemic infarct in the liver is virtually pathognomonic; it is slightly elevated, lighter in color than the surrounding tissue, and outlined by a bluish red zone of congestion. The kidneys are dark, friable, and usually studded with petechiae. The bladder contains purplish red urine. After death, rigor mortis sets in more rapidly than usual.

Clostridium chauvoei occurs naturally in the intestinal tract of animals. It probably can remain viable in the soil for many years, although it does not actively grow there. Contaminated pasture appears to be a source of organisms. Outbreaks of blackleg have occurred in cattle on farms in which recent excavations have occurred, which suggests that disturbance of soil may activate latent spores. The organisms probably are ingested, pass through the wall of the GI tract, and after gaining access to the bloodstream, are deposited in muscle and other tissues. In cattle, blackleg infection is endogenous, in contrast to malignant edema. Lesions develop without any history of wounds, although bruising or excessive exercise may precipitate some cases. Commonly, the animals that contract blackleg are of the beef breeds, in excellent health, gaining weight, and usually the best animals of their group. Outbreaks occur in which a few new cases are found each day for several days. Most cases occur in cattle from 6 months to 2 years old, but thrifty calves as young as 6 weeks and cattle as old as 10-12 years may be affected. The disease usually occurs in summer and fall and is uncommon during the winter. In sheep, the disease is not restricted to the young, and most cases follow some form of injury such as shearing cuts, docking, crutching, or castration. Usually, onset is sudden and a few cattle may be found dead without premonitory signs. Acute lameness and marked depression are common. Initially, there is a fever but, by the time clinical signs are obvious, the temperature may be normal or subnormal. Characteristic edematous and crepitant swellings develop in the hip, shoulder, chest, back, neck, or elsewhere. At first the swelling is small, hot, and painful. As the disease rapidly progresses, the swelling enlarges, there is crepitation on palpation, and the skin becomes cold and insensitive as the blood supply to the area diminishes. General signs include prostration and tremors. Death occurs in 12-48 hrs. In some cattle, the lesions are restricted to the myocardium and the diaphragm, with no reliable ante mortem evidence of the localized lesion.

Clostridium novyi has been suspected but not yet confirmed as a cause of sudden death in cattle and pigs fed high-level grain diets, and in which pre-existing lesions of the liver were not detectable. The lethal and necrotizing toxins (primarily a toxin) damage hepatic parenchyma, thereby permitting the bacteria to multiply and produce a lethal amount of toxin. Usually, death is sudden with no well-defined signs. Affected animals tend to lag behind the flock, assume sternal recumbency, and die within a few hours. Most cases occur in the summer and early fall when liver fluke infection is at its height. The disease is most prevalent in 1- to 4-year-old sheep and is limited to animals infected with liver flukes. Differentiation from acute faseioliasis may be difficult, but peracute deaths of animals that show typical lesions on necropsy should arouse suspicion of infectious necrotic hepatitis. The most characteristic lesions are the grayish yellow necrotic foci in the liver that often follow the migratory tracks of the young flukes. Other common findings are an enlarged pericardial sac filled with straw-colored fluid, and excess fluid in the peritoneal and thoracic cavities. Usually, there is extensive rupture of the capillaries in the subcutaneous tissue, which causes the adjacent skin to turn black (hence the common name, black disease).

Clostridium septicum is found in soil and intestinal contents of animals (including man) throughout the world. Infection ordinarily occurs through contamination of wounds containing devitalized tissue, soil, or some other tissue-debilitant. Wounds caused by accident, castration, docking, insanitary vaccination, and parturition may become infected. General signs, such as anorexia, intoxication, and high fever, as well as local lesions, develop within a few hours to a few days after predisposing injury. The local lesions are soft swellings that pit on pressure and extend rapidly because of the formation of large quantities of exudate that infiltrates the subcutaneous and intramuscular connective tissue of the affected areas. The muscle in such areas is dark brown to black. Accumulations of gas are uncommon. Severe edema of the head of rams occurs after infection of wounds inflicted by fighting. Malignant edema associated with lacerations of the vulva at parturition is characterized by marked edema of the vulva, severe toxemia, and death in 24-48 hours. Similarity to blackleg is marked, and differentiation made on necropsy is unreliable; laboratory confirmation is the only certain procedure. Horses and pigs are susceptible to malignant edema but not to blackleg.

Infectious disease caused by Clostridium sordellii are also characterized by a nongaseous, nonhemorrhagic, edematous swelling of the head, face, and neck of young rams. This infection is initiated in young rams by their continual butting of one another. The bruised and battered subcutaneous tissues provide conditions suitable for growth of pathogenic clostridia, and the breaks in the skin offer an opportunity for their entrance

Infection with C. perfringens types A, B and C causes severe enteritis, dysentery, toxemia, and high mortality in young calves. Types B and C both produce the highly necrotizing and lethal β toxin that is responsible for the severe intestinal damage. This toxin is sensitive to proteolytic enzymes, and disease is associated with inhibition of proteolysis in the intestine. Sow colostrum, which contains a trypsin inhibitor, has been suggested as a factor in the susceptibility of young piglets. Type C also causes enterotoxemia in adult cattle. In calves, there is acute diarrhea dysentery, abdominal pain, convulsions, and opisthotonos. Death may occur in a few hours, but less severe cases survive for a few days, and recovery over a period of several days is possible. Hemorrhagic enteritis with ulceration of the mucosa is the major lesion in all species. Grossly, the affected portion of the intestine is deep blue-purple and appears at first glance to be an infarction associated with mesenteric torsion. Smears of intestinal contents can be examined for large numbers of gram-positive, rod-shaped bacteria, and filtrates made for detection of toxin and subsequent identification by neutralization with specific antiserum.

This classic enterotoxemia caused by C. perfringens type D rarely occurs in cattle. It is worldwide in distribution and may occur in animals of any age. The disease has been suspected in well-nourished beef calves nursing high-producing cows grazing lush pasture and in sudden death syndrome in feedlot cattle; however, supportive laboratory evidence in the latter is lacking. Acutely affected calves not found dead show mania, convulsions, blindness, and death in a few hours. Subacutely affected calves are stuporous for a few days and may recover.

Tetanus toxemia is caused by a specific neurotoxin produced by Clostridium tetani in necrotic tissue. Almost all mammals are susceptible to this disease. Although tetanus is worldwide in distribution, there are some areas, such as the northern Rocky Mountain section of the USA, where the organism is rarely found in the soil and where tetanus is almost unknown. In general, the occurrence of C tetani in the soil and the incidence of tetanus in man and horses is higher in the warmer parts of the various continents. Clostridium tetani, an anaerobe with terminal, spherical spores, is found in soil and intestinal tracts. In most cases, it is introduced into the tissues through wounds, particularly deep puncture wounds, that provide a suitable anaerobic environment.

Infection with Salmonella spp can produce diarrhea in animals of all ages, especially those that are stressed, closely stocked, or exposed to a heavily contaminated feed or water supply. Salmonellosis is caused by many species of salmonellae and characterized clinically by one or more of three major syndromes—septicemia, acute enteritis, and chronic enteritis. The incidence has increased with the intensification of livestock production. Young calves usually develop the septicemic form. Adult cattle, develop acute enteritis. Conic enteritis may develop occasionally in cattle. Pregnant animals may abort. In older animals, the disease is manifested by dysentery and toxemia, and mortality can be significant. While many other Salmonella spp may cause disease, the more relevant in cattle are S. typhimurium, S. dublin, and S. newport. Although their resulting clinical patterns are not distinct, different species of salmonellae tend to differ in their epidemiology. Plasmid profile and drug-resistance patterns are sometimes useful markers for epidemiologic studies, feces of infected animals can contaminate feed and water, milk, fresh and processed meats from abattoirs, plant and animal products used as fertilizers or feeds tuffs, pasture and rangeland, and many inert materials. The organisms may survive for months in wet, warm areas such as in feeder pig barns or in water dugouts but survive less than 1 week in composted cattle manure. Rodents and wild birds also are sources of infection. The prevalence of infection varies among species and countries and is much higher than the incidence of clinical disease, which is commonly precipitated by stressful situations such as sudden deprivation of feed, transportation, drought, crowding, parturition, and the administration of some drugs.

Further relevant gastro-intestinal pathogens are Cryptosporidium parvum and Mycobacterium avium paratuberculosis. Paratuberculosis is a chronic, contagious enteritis characterized by persistent and progressive diarrhea, weight loss, debilitation, and eventually death. It affects cattle, sheep, goats, llamas, camels, farmed deer, and other domestic, exotic, and wild ruminants. It has also been recognized in wild rabbits; horses and pigs can be infected experimentally. Distribution is worldwide. There are conflicting data on the involvement of the organism in Crohn's disease, a chronic enteritis in people. Animals with paratuberculosis should be considered as potential zoonotic risks until the situation is clarified. The causative organism is Mycobacterium avium paratuberculosis, formerly known as M. paratuberculosis or M. johnei. Occasionally, other M. avium subspecies are isolated from cases. The organism is quite resistant and can survive on pasture for more than 1 year, but sunlight, alkaline soils, and drying reduce its survival rate. It is shed in large numbers in feces of infected animals, and infection is acquired by ingestion of contaminated feed and water. Introduction of the disease into a clean herd is usually by subclinically infected carriers. Infection is acquired early in life, but clinical signs rarely develop in cattle <2 yrs old. Resistance increases with age, and cattle first exposed as adults are unlikely to become infected. Most calves are infected soon after birth either by nursing udders contaminated with feces from infected animals or by being housed in contaminated pens. The organism can also be present in colostrum and milk of infected cows, and intrauterine infections have also been described. After ingestion, the bacteria infect macrophages in the mucosa of the lower small intestine and in associated lymph nodes. Most animals will eliminate infection by an early cell-mediated immune response that encourages microbicidal activity in macrophages. In susceptible animals, the organisms multiply and provoke a chronic enteritis that leads to clinical disease. This may take months to years to develop and is usually paralleled by a decline in cell-mediated immunity and a rise in ineffective serum antibody. However, fecal shedding begins before clinical signs are apparent. Mycobacterium avium paratuberculosis can be isolated from feces, mesenteric and ileocecal lymph nodes, thickened intestinal walls, and less frequently the udder and the reproductive tracts of both sexes.

Cryptosporidiosis is an enterocolitis of cosmopolitan distribution caused by the coccidian parasite Cryptosporidium parvum. It is not host-specific and is common in young ruminants, particularly calves; it is also found in man and pigs and is rare in dogs, cats, and horses. Other cryptosporidia cause disease in reptiles and birds. The disease in calves, characterized by weight loss and watery diarrhea, is clinically indistinguishable from many other causes of calf diarrhea. Cryptosporidium parvum is a minute protozoan that is transmitted by the fecal-oral route. Oocysts are sporulated (four sporozoites) when shed in the feces and, therefore, are immediately infective. The mean incubation period is ˜4 days. Calves 1-3 weeks old seem to be most susceptible. Signs such as anorexia, weight loss, diarrhea, and tenesmus, resemble those caused by several other intestinal pathogens; however, infections without signs do occur. Uncomplicated cryptosporidiosis is seldom fatal. Disease can be severe in immunocompromised individuals. If severe disease in calves is seen, other disease agents or concurrent infections should be ruled out. Although C. parvum can infect virtually the entire intestinal tract, the distal small intestine usually is affected most severely. Infection, in horses is limited to the small intestine. Gross lesions may consist of hyperemic intestinal mucosa and yellowish intestinal contents. Microscopically, mild to severe villous atrophy with spherical organisms in the brush border is evident. Unlike Eimeria and Isospora spp, which are intracellular parasites. C. parvum is intramembranous and resides within the brush border of the intestinal epithelial cells.

Chlamydia psittaci causes sporadic abortion after the fourth month of gestation but usually in the last trimester. The chlamydia cause placentitis, fetal pneumonia, and hepatitis. Stained smears of cotyledons may reveal the organisms; if not, tissues may be cultured in embryonating chicken eggs. Abortion is usually sporadic in cows, but an ovine chlamydial vaccine has been used in cattle.

Inflammation of the mammary gland (mastitis) is almost always due to the effects of infection by bacterial or mycotic pathogens. Mastitis may be associated with infection by many other organisms, including Streptococcus uberis, Streptococcus dysgalactiae, Klebsiella spp. Pseudomonas aeruginosa, Actinomyces pyogenes, Mycoplasma spp, Nocardia asteroides, Serratia, Mycobacterium spp, Clostridium perfringens, Pasteurella spp, yeasts, and Prototheca spp.

Dermatomycoses (Dermatophytosis) in animals are anthropozoonotic diseases of the skin and to related tissue. Clinical symptoms are characterized by loss of hair in the affected area hyperemia, scaling and asbestos-like scabs. Inflammation is often accompanied by suppuration, Dermatomycoses are often also characterized by localized infection of the skin. Dermatomycoses in animals carry a substantial socioeconomic impact. Diseased animals required prolonged treatment and can spread infection to both animals and humans. Dermatophytosis are caused by mycosis infections of Trichophyton spp. or Microsporum spp. Most relevant causes for cattle are Trichophyton verrucosum, Trichophyton mentagrophytes or Trichophyton sarkisovii.

An infection of the lower respiratory tract, usually resulting in bronchitis or pneumonia, can be caused by any of several parasitic nematodes, including Dictyocaulus viviparus in cattle. This lungworm belongs to the superfamily Trichostrongyloidea and has direct life cycles. The cattle lungworm is common in northwest Europe and is the cause of severe outbreaks of “husk” or “hoose” in young grazing cattle. Because D. viviparus infection in cattle is the most economically important, it has been most investigated and many of the observations from it are applicable to the other species. Clinical disease usually develops on first exposure to sufficient infective larvae. In cattle, this usually occurs during their first season at pasture; however, an increase in the number of older cattle affected has been reported. Signs of lungworm infection range from moderate coughing with slightly increased respiratory rates to severe persistent coughing and respiratory distress and even failure. Reduced weight-gains, reduced milk yields, and weight loss accompany many infections in cattle. Patent subclinical infections can occur in all species. The most consistent signs in cattle are tachypnea and coughing.

Trichomoniasis is a venereal protozoal disease of cattle characterized primarily by early fetal death and infertility, resulting in extended calving intervals. Distribution is probably worldwide. The causative protozoan. Trichomonas (Tritrichomonas) foetus, is pyriform and ordinarily 10-15×5-10 μm, but there is considerable pleomorphism. It may become spherical when cultured in artificial media. At its anterior end, there are three flagella about the same length as the body of the parasite. An undulating membrane extends the length of the body and is bordered by a marginal filament that continues beyond the membrane as a posterior flagellum. Although T foetus can survive the process used for freezing semen, it is killed by drying or high temperatures. Trichomonas foetus is found in the genital tracts of cattle. When cows are bred naturally by an infected bull, 30-90% become infected, suggesting that strain differences exist. Variation in breed susceptibility to trichomoniasis may also exist. Bulls of all ages can remain infected indefinitely but this is less likely in younger males. By contrast, most cows are free of infection within 3 months after breeding. However, immunity is not long lasting and reinfection does occur. Transmission can also occur when the semen from infected bulls is used for artificial insemination. The most common sign is infertility caused by embryonic death. This results in repeat breeding and a prolonged calving season. Fetal death and abortions can also occur but are not as common as losses earlier in gestation. Trichomonas foetus has been found in vaginal cultures taken, as late as 8 months of gestation and, apparently, live calves can be born to infected dams. Pyometra occasionally develops after breeding.

Neospora caninum is an obligate intracellular protozoan parasite that has been confused previously with Toxoplasma gondii. Only asexual stages are known, and they resemble T gondii. The complete life cycle of N caninum is unknown, but it can be transmitted transplacentally in dogs, cattle, goats, sheep, and cats, and subsequent offspring may be affected. Tachyzoites are 5-7×1-5 μm, depending on the stage of division. They divide by endodyogeny. Tachyzoites are found in myocytes, neural cells, dermal cells, macrophages, and other cells. Tissue cysts up to 100 μm in diameter are found in neural cells; the cyst wall is amorphous and up to 4 μm thick. Cysts have no septa and enclose slender 7×1.5 μm bradyzoites. In dairy cattle, N caninum is a major cause of abortion in many countries, particularly in the USA. Calves may be aborted, stillborn, born underweight, weak, or paralyzed, or they may become paralyzed within 4 weeks of birth. Non-suppurative encephalitis is the main lesion in aborted fetal tissues. Abortion can occur throughout gestation, and some cows may abort again; dams of these calves are clinically normal.

Babesiosis is caused by intraerythrocytic protozoan parasites of the genus Babesia. A wide range of domestic and wild animals and occasionally man is affected by the disease, which is transmitted by ticks and has a worldwide distribution. Two important species in cattle—Babesia bigemina and Babesia bovis—are widespread in tropical and subtropical areas and are the focus of this discussion. In endemic areas, two features are important in determining the risk of clinical disease: 1) calves have a degree of immunity (related both to colostral-derived antibodies and to age) that persists for ˜6 months, and 2) animals that recover from Babesia infections are immune for life. Thus, at high levels of tick transmission, all newborn calves will become infected with Babesia by 6 mos. of age, show few if any clinical signs, and subsequently be immune. This situation of endemic stability can be upset by either a natural (eg, climatic) or artificial (eg, acaricide treatment) reduction in tick numbers to levels where tick transmission of Babesia to calves is insufficient to ensure all are infected during this critical early period. Other circumstances that can lead to clinical outbreaks include the introduction of susceptible cattle to endemic areas and the incursion of Babesia-infected ticks into previously tick-free areas. Strain variation in immunity has been demonstrated but is probably not of significance in the field. The acute disease generally runs a course of ˜1 week. The first sign is fever (frequently 41° C. or higher), which persists throughout, and is accompanied later by inappetence, increased respiratory rate, muscle tremors, anemia, jaundice, and loss of weight with hemoglobinemia and hemoglobinuria in the final stages. CNS involvement due to sludging of parasitized erythrocytes in brain capillaries occurs frequently with B. bovis infection. Either constipation or diarrhea may be present. Pregnant cows often abort. With virulent strains of B. bovis, a hypotensive shock syndrome, combined with generalized nonspecific inflammation, coagulation disturbances, and erythrocytic stasis in capillaries, contribute to the pathogenesis. With most strains of B. bigemina, the pathogenic effects relate more directly to erythrocyte destruction. Animals that recover from the acute disease remain infected for a number of years with B. bovis and for a few months in the case of B. bigemina. No signs are apparent during this carrier state. Lesions include an enlarged, and friable spleen; a swollen liver with an enlarged gallbladder containing thick granular bile; congested, dark-colored kidneys; and generalized anemia and jaundice. The urine is often, but not invariably, red. Other organs, including the brain and heart, may show congestion or petechial hemorrhages. The susceptibility of cattle breeds to Babesia infections varies; for example. Brahman cattle are more resistant to B. bovis infection than are British breeds.

### BRIEF DESCRIPTION OF THE DRAWINGS

FIG. 1 Serum neutralisation against NY93/C (BVDV type II)

FIG. 2 Serum neutralisation assay against KE9 (BVDV type I)

FIG. 3 Serum neutralisation assay against NY93/C (BVDV type II)

All subsequent sequences show the deleted regions indicated with dashes (-), which are also numbered, whereas the sequences in the sequence listing attached hereto are continuously numbered without the deleted regions or amino acid codons.

SEQ ID NO:1 XIKE-A-cDNA sequence
SEQ ID NO:2 XIKE-A-NdN-cDNA sequence
SEQ ID NO:3 XIKE-B-cDNA sequence

### SEQ ID NO:4 XIKE-B-NdN-cDNA

SEQ ID NO:5 XIKE-A amino acid sequence
SEQ ID NO:6 XIKE-A-NdN amino acid sequence
SEQ ID NO:7 XIKE-B amino acid sequence
SEQ ID NO:8 XIKE-B-NdN amino acid sequence
SEQ ID NO:9 XIKE-C-NdN amino acid sequence
SEQ ID NO:10 XIKE-C-NdN-cDNA sequence
SEQ ID NO:11 XIKE-C-cDNA sequence
SEQ ID NO:12 XIKE-C amino acid sequence

### BRIEF SUMMARY OF THE INVENTION

The present invention relates to combination vaccines for the treatment and/or prophylaxis of cattle against microbiological infections, wherein one of the infections is caused by BVDV. The combination vaccine as described herein comprises at least one attenuated BVDV, wherein said attenuated BVDV comprises at least one mutation in the coding sequence for glycoprotein E^{rns} and at least another mutation in the coding sequence for N^{pro} which preferably leads to combined inactivation of the RNase activity residing in glycoprotein E^{rns} in addition to the inactivation of the (hypothesized) immunomodulating activity residing in N^{pro}. The invention also relates to methods for producing such combination vaccines.

According to a preferred embodiment, the present invention relates to a combination vaccine for the treatment and/or prophylaxis of cattle against infections of the respiratory and reproductive system in cattle, wherein the combination vaccine comprises an attenuated BVDV as described herein and at least one further immunological active component effective for the treatment and/or prophylaxis of infections caused by Bovine Herpes virus (BHV), Bovine Respiratory Syncytial Virus (BRSV), Parainfluenza Virus (PI-3), Campylobacter fetus, Leptospira canicola, Leptospira grippotyphosa, Leptospira borgpetersenii, Leptospira prajitno, Leptospira icterohaemmorrhagiae, Leptospira hardjo, Leptospira bovis, Leptospira interrogans and/or Leptospira ponoma. According to a more preferred embodiment, the combination vaccine comprises an attenuated BVDV as described herein and at least one antigen of BHV, BRSV, PI-3, Campylobacter fetus, Leptospira canicola, Leptospira grippotyphosa, Leptospira borgpetersenii Leptospira prajitno, Leptospira hardjo (Leptospira hardjo prajitno and Leptospira hardjo-bovis), Leptospira icterohaemmorrhagiae, Leptospira bovis, Leptospira interrogans and/or Leptospira ponoma.

According to a more preferred embodiment, the present invention relates to a combination vaccine for the treatment and/or prophylaxis of cattle against viral infections of the respiratory and reproductive system in cattle caused by BVDV, PI-3, BRSV, IBR and/or BHV, wherein said vaccine comprises at least an attenuated BVDV as described herein and at least one further further immunological active component effective for the treatment and/or prophylaxis of infections caused by PI-3, BRSV IBR, and BHV. According to a further more preferred embodiment, the present invention relates to a combination vaccine for the treatment and/or prophylaxis of cattle against viral infections of the respiratory and reproductive system in cattle caused by BVDV, PI-3, IBR, BRSV and/or BHV, wherein said vaccine comprises at least an attenuated BVDV as described herein and at least one antigen of PI-3, IBR, BRSV and/or BHV.

### DETAILED DESCRIPTION OF THE INVENTION

### Definitions of Terms Used in the Description

Before the embodiments of the present invention it must be noted that as used herein and in the appended claims, the singular forms “a”, “an”, and “the” include plural reference unless the context clearly dictates otherwise. Thus, for example, reference to “a BVDV” includes a plurality of such BVDV, reference to the “cell” is a reference to one or more cells and equivalents thereof known to those skilled in the art, and so forth. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference in their entireties including for the purpose of describing and disclosing the cell lines, vectors, and methodologies as reported in the publications, which might be used in connection with the invention. Nothing herein is to be construed as an admission that the invention is not entitled to antedate such disclosure by virtue of prior invention.

The term “BVDV” as used herein refers to all viruses belonging to species bovine viral diarrhea virus (BVDV) type 1 (BVDV-1) and BVDV type 2 (BVDV-2), including any sub-species such as 1a, 1b, 2a, 2,b, and the like in the genus Pestvirus within the family Flaviviridae (Heinz et al., 2000). The more classical BVDV type 1 strains and the more recently recognized BVDV type 2 strains display some limited but distinctive differences in nucleotide and amino acid sequences.

“Protein C” or “C protein” or “C-protein” as used herein relates to a structural component of the pestivirus virion (Thiel et al., 1991). “Protein C” is the capsid or core protein of pestiviruses. Said term, depending on the context, may also relate to the “Protein C” with one or several amino acids exchanges resulting from mutation of the encoding nucleotide sequence.

The term “N^{pro}” as understood herein relates to the first protein encoded by the viral open reading frame that cleaves itself from the rest of the synthesized polyprotein (Stark, et al., J. Virol. 67:7088-7093 (1993); Wiskerchen, et al., Virol. 65:4508-4514 (1991)). Said term, depending on the context, may also relate to the remaining “N^{pro}” amino acids after mutation of the encoding nucleotide sequence or to the coding nucleotide sequence for said protein itself. “Protease activity residing in N^{pro}” relates to the polypeptide cleavage activity of said “N^{pro}”.

Inactivation of N^{pro} as used herein means the prevention or considerable reduction of the probable immunemodulating activity of N^{pro} by mutation. In a preferred embodiment this mutation prevents or considerably reduces the interference of N^{pro} with the induction of an interferon response by the infected cells as described by Rüggli et al., (2003). In this case, the inactivation of N^{pro} would allow the cell to mount a normal interferon response.

“Processing signal” as used herein relates to a substance that ensures the generation of a functional N-terminal of the C protein of the pestivirus, preferably of BVDV, in particular a substance selected from the group of ubiquitin, LC3, SUMO-1, NEDD8, GATE-16 and GABA(A)RAP. Also proteases selected from the group of Intern, picornavirus 3C, caridovirus 2A and p15 of rabbit hemorrhagic disease virus are understood as “processing signals” as used herein. Any other similar processing signal known to the skilled person that ensures the generation of a functional N-terminal of the C protein shall also be comprised in the term “processing signal”.

“E^{rns}” as used herein relates to the glycoprotein E^{rns} which represents a structural component of the pestivirus virion (Thiel et al., 1991). E^{rns} lacks a typical membrane anchor and is secreted in considerable amounts from the infected cells; this protein has been reported to exhibit RNase activity (Hulst et al., 1994; Schneider et al., 1993; Windisch et al., 1996). It should be noted that the term glycoprotein E0 is often used synonymously with glycoprotein E^{rns} in publications. Said term, depending on the context, may also relate to the mutated “E^{rns}” protein after mutation of the encoding nucleotide sequence or to the coding nucleotide sequence for said protein itself. “RNase activity residing in glycoprotein E^{rns}” relates to the RNA cleavage activity of said glycoprotein, i.e. the ability of the glycoprotein E^{rns} to hydrolyze RNA. The term “inactivation of the RNase activity residing in said glycoprotein” refers to the inability or reduced capability of a modified glycoprotein E^{rns} to hydrolyze RNA as compared to the unmodified wild type of said glycoprotein E^{rns}.

Inactivation of E^{rns} as used herein means RNase activity not significantly above the level measured for noninfected control cells in an RNase assay as described in Meyers et al., 1999. “Not significantly above the level measured for noninfected control cells in an RNase assay as described in Meyers et al., 1999”, means for example, that the RNase activity is less than 150% compared to the noninfected control cells.

Attenuation: “An attenuated pestivirus or BVDV particle” as used herein means that there is a statistically significant difference between the virulence of attenuated pestivirus or BVDV particles of the present invention, wherein said attenuated viral particles being attenuated by a method described herein, and wild-type pestivirus or BVDV isolates from which said attenuated pestivirus or BVDV particles have been derived, for the predominant clinical parameters, in case of BVDV for diarrhea, pyrexia and lethality in animals infected with the same dose, preferably 6×10⁶ TCID₅₀. Thus, said attenuated BVDV particles do not cause diarrhea, pyrexia and lethality and thus may be used in a vaccine.

“Bovine pathogen” as used herein means a microorganism that has an impact on the healthiness of cattle.

“Immunological active component” or “immunologically active component” as used herein means a component that induces or stimulates the immune response in an animal to which said component is administered. According to a preferred embodiment, said immune response is directed to said component or to an microorganism comprising said component. According to a further preferred embodiment, the immunological active component is an attenuated microorganism, including modified live virus (MLV), a killed-microorganism or at least an immunological active part of a microorganism.

“immunological active part of a microorganism” as used herein means a protein-, sugar-, and or glycoprotein containing fraction of a microorganism that comprises at least one antigen that induces or stimulates the immune response in an animal to which said component is administered. According to a preferred embodiment, said immune response is directed to said immunological active part of a microorganism or to a microorganism comprising said immunological active part.

The term “vaccine” as used herein refers to a pharmaceutical composition comprising at least one immunologically active component that induces an immunological response in an animal and possibly but not necessarily one or more additional components that enhance the immunological activity of said active component. A vaccine may additionally comprise further components typical to pharmaceutical compositions. The immunologically active component of a vaccine may comprise complete virus particles in either their original form or as attenuated particles in a so-called modified live vaccine (MLV) or particles inactivated by appropriate methods in a so-called killed vaccine (KV). In another form, the immunologically active component of a vaccine may comprise appropriate elements of said organisms (subunit vaccines) whereby these elements are generated either by destroying the whole particle or the growth cultures containing such particles and optionally, subsequent purification steps yielding the desired structure(s), or by synthetic processes including an appropriate manipulation by use of a suitable system based on, for example, bacteria, insects, mammalian or other species, plus optionally subsequent isolation and purification procedures, or by induction of said synthetic processes in the animal needing a vaccine by direct incorporation, of genetic material using suitable pharmaceutical compositions (polynucleotide vaccination). A vaccine may comprise one or simultaneously more than one of the elements described above. The term “vaccine” as understood herein is a vaccine for veterinary use comprising antigenic substances and is administered for the purpose of inducing a specific and active immunity against a disease provoked by a microbiological infection, preferably by a BVDV infection. The BVDV as described herein, confer active immunity that may be transferred passively via maternal antibodies against the immunogens it contains and sometimes also against antigenically related organisms. A vaccine of the invention refers to a vaccine as defined above, wherein one immunologically active component is a BVDV or derived from a nucleotide sequence that is more than 70% homologous to any known BVDV sequence (sense or antisense).

The term “live vaccine” refers to a vaccine comprising a replication competent, in particular, a replication compentent viral active component.

“Combination vaccine” as used herein means a vaccine that comprises attenuated BVDV as described herein together with a monovalent, bivalent or multivalent combination of immunological active components).

“Microbiological infection” as used herein means an infection as caused by a microorganism that is to pathogenic for cattle. Such microorganisms include but are not limited to bacteria, viruses, yeasts or fungi, mycoplasms, and parasites.

A fragment” according to the invention is any subunit of a polynucleotide molecule according to the invention, i.e. any subset. For DNA, said fragment is characterized in that it is shorter than the DNA covering the full-length viral genome.

A functional variant” of the nucleotide molecule as used herein is a nucleotide molecule which possesses a biological activity (either functional or structural) that is substantially similar to the nucleotide molecule according to the invention. The term “functional variant” also includes “a fragment”, “a functional variant”, “a variant based on the degenerative nucleic acid code” or “a chemical derivative”. Such “a functional variant” e.g. may carry one or several nucleotide exchanges, deletions or insertions. Said functional variant at least partially retains its biological activity, e.g. functions as an infectious clone or a vaccine strain, or even exhibits improved biological activity.

“Possess a biological activity that is substantially similar” means with respect to the pestiviruses provided herewith, for example, that said pestivirus is attenuated in a manner described herein and results in an non-pathogenic virus suitable for the production of live attenuated virus, which loses ability to pass the placenta but mediates an immune response after vaccination.

A “variant based on the degenerative nature of the genetic code” is a variant resulting from the fact that a certain amino acid may be encoded by several different nucleotide triplets. Said variant at least partially retains its biological activity, or even exhibits improved biological activity.

A molecule is “substantially similar” to another molecule if both molecules have substantially similar nucleotide sequences or biological activity. Thus, provided that two molecules possess a similar activity, they are considered variants as that term is used herein if the nucleotide sequence is not identical, and two molecules which have a similar nucleotide sequence are considered variants as that term is used herein even if their biological activity is not identical.

A “mutation” as used herein relates to modifications in the nucleic acid molecules encoding the proteins/amino acids according to the invention. Said mutations relate to, but are not limited to, substitutions (replacement of one or several nucleotides/base pairs), deletions (removal of one or to several nucleotides/base pairs), and/or insertions (addition of one or several nucleotides/base pairs). As used herein, mutation may refer to a single mutation or several mutations, therefore, often the term “mutation(s)” is used and relates to both a single mutation and several mutations. Said mutations include, but are not limited to point mutations (single nucleotide mutations) or larger mutations wherein e.g. parts of the encoding nucleic acid molecules are deleted, substituted and/or additional coding nucleic acids are inserted. Said mutations may result in a modified expressed polypeptide due to the change in the coding sequence. Such modified polypeptides are desired, as set out in the disclosure of the invention as set out below.

Additional components to enhance the immune response are constituents commonly referred to as “adjuvants”, like e.g. aluminiumhydroxide, mineral or other oils or ancillary molecules added to the vaccine or generated by the body after the respective induction, by such additional components, like but not restricted to interferons, interleukins or growth factors.

A “pharmaceutical composition” essentially consists of one or more ingredients capable of modifying physiological e.g. immunological functions of the organism it is administered to, or of organisms living in or on the organism. The term includes, but is not restricted to, antibiotics or antiparasitics, as well as other constituents commonly used to achieve certain other objectives like, but not limited to, processing traits, sterility, stability, feasibility to administer the composition via enteral or parenteral routes such as oral, intranasal, intravenous, intramuscular, subcutaneous, intradermal or other suitable route, tolerance after administration, and controlled release properties. One non-limiting example of such a pharmaceutical composition, solely given for demonstration purposes, could be prepared as follows: Cell culture supernatant of an infected cell culture is mixed with a stabilizer (e.g. spermidine and/or BSA (bovine serum albumin)) and the mixture is subsequently lyophilized or dehydrated by other methods. Prior to vaccination, said mixture is then rehydrated in aqueous (e.g. saline, PBS (phosphate buffered saline)) or non-aqueous solutions (e.g. oil emulsion, aluminum-based adjuvant).

### DISCLOSURE OF THE INVENTION

The solution to the above technical problem is achieved by the description and the embodiments characterized in the claims.

The present invention relates to a combination vaccine for the treatment and/or prophylaxis of microbiological infection in cattle, that comprises a live attenuated BVDV as described herein and at least one further immunological active component for treating or preventing diseases or disorders in cattle caused by an infectious agents other that BVDV.

Attenuated BVDV

It was described in WO 99/64604 that BVDV can be attenuated by introducing at least one mutation in the coding sequence of glycoporiteion E^{rns}, wherein said mutation(s) result in an inactivation of the RNAse activity residing in the E^{rns} gene region. Moreover, it has also surprisingly been found that BVDV can be more effectively attenuated by introducing at least one mutation in the coding sequence for glycoprotein E^{rns} and at least another mutation in the coding sequence for N^{pro} which preferably leads to the combined inactivation of the RNase activity residing in glycoprotein E^{rns} in addition to the inactivation of the immunomodulating activity residing in N^{pro} (WO2005/111201). An immunomodulating effect in one aspect is indicated but not limited to the indicated function for one pestivirus in an exemplary manner by Rüggli et al. (2003). Thus according to one aspect, the present invention provides a combination vaccine that comprises at least an attenuated BVDV having at least one mutation in the coding sequence for glycoprotein E^{rns} and/or at least another mutation in the coding sequence for N^{pro}. Preferably, in such attenuated BVDV, said mutation in the coding sequence for glycoprotein E^{rns} leads to inactivation of the RNase activity residing in E^{rns} and/or said mutation in the coding sequence for N^{pro} leads to inactivation of said N^{pro}.

The attenuated BVDV as described herein can be advantageously used in combination vaccines for the treatment and/or prophylaxis of microbiological infections in cattle. Surprisingly, the BVDV as described herein, comprising any of the modifications in the N^{pro} and E^{rns} gene region are safe for use in pregnant animals as they do not cross the placenta. This is exemplified in a non-limiting manner for BVDV in example 3. Furthermore, the BVDV with defined mutations within the N^{pro} and E^{rns} as a basis for attenuation will allow to avoid the risk of reversion to a more pathogenic strain. A further advantage of said attenuating mutations lies in their molecular uniqueness, which allows to use them as distinctive labels for an attenuated BVDV and to distinguish them from BVDV from the field. Therefore, in a further aspect the present invention provides a combination vaccine that comprises at least an attenuated BVDV having at least one mutation in the coding sequence for glycoprotein E^{rns} and at least another mutation in the coding sequence for N^{pro}. Preferably, in such attenuated BVDV, said mutation in the coding sequence for glycoprotein E^{rns} leads to inactivation of the RNase activity residing in E^{rns} and/or said mutation in the coding sequence for N^{pro} leads to inactivation of said N^{pro}. Said inactivation may take place by any mutation known to the person skilled in the art of the E^{rns}- and the N^{pro}-coding sequence, wherein the mutations are any mutation as defined in the “definitions” section, such as deletions, insertion mutations and/or substitution mutations. Most preferably, the mutation(s) are deletions, as the likelihood for reversion to the wild type is the lowest for deletions.

Altogether, the term attenuated BVDV or attenuated pestivirus in general as used herein, means but is not limited to any attenuated BVDV or pestivirus, having at least one modification in the coding sequence for glycoprotein E^{rns} and/or at least one modification in the coding sequence for N^{pro}. In the following, specific embodiments of any of such modification in the E^{rns} and/or N^{pro} are described more in detail. According to a preferred aspect, the term attenuated BVDV or attenuated pestivirus in general as used herein, means, but is not limited to, any attenuated BVDV or pestivirus, having at least one modification in the coding sequence for glycoprotein E^{rns} and at least one modification in the coding sequence for N^{pro}. However, it is hereby understood that the present invention shall not be limited to the specific modification described herein. A person skilled in the art with the knowledge of the teaching provided herewith, is able to generate and introduce further modifications within the glycoprotein E^{rns} and/or N^{pro} having the effect of attenuation as described herein.

### Modifications of the E^{rns} of BVDV

It has been shown that the glycoprotein E^{rns} forms a disulfide-bonded homodimer of about 97 kD, wherein each monomer consists of 227 amino acids corresponding to the amino acids 268 to 494 of the CSFV poly protein as described by Rümenapf et al. (1993). The genome sequence of the Alfort/Tübingen strain of CSFV is available in the GenBank/EMBL data library under accession number J04358; alternatively, the amino acid sequence for the BVDV strain CP7 can be accessed in the GenBank/EMBL data library (accession number U63479); in the BVDV CP7 polyprotein, the E^{rns} protein corresponds to residues 271 to 497. Two regions of amino acids are highly conserved in glycoprotein E^{rns} as well as in some plant and fungal RNase-active proteins (Schneider et al., 1993). These two regions are of particular importance to the RNase enzymatic activity. The first region consists of the region at the amino acids at position 295 to 307 (298 to 310 for BVDV strain cp7) and the second region consists of the amino acids at position 338 to 357 (341 to 360 for BVDV strain cp7) of said viral poly protein as exemplified for the Alfort strain of CSFV in Meyers et al., 1999 (numbering according to the published deduced amino acid sequence of CSFV strain Alfort/Tübingen (Meyers et al., 1989). The amino acids of particular importance to the RNase activity as mentioned above are by no means limited to the exact position as defined for the Alfort/Tübingen strain of CSFV but are simply used in an exemplary manner to point out the preferred amino acids being at that position or corresponding to that position in oilier strains such as found in BVDV, BDV and pestiviruses in general since they are highly conserved. For pestiviruses other than the CSFV Alfort/Tübingen strain the numbering of the positions of the preferred amino acids can be different but an expert in the field of the molecular biology of pestiviruses will easily identify these preferred amino acids by the high degree of conservation of this ammo acid sequence and the position of these motifs in the sequence context. In one particular non-limiting example, the position of CSFV Alfort/Tübingen 346 is identical to position 349 of BVDV strain cp7.

As a consequence, the present invention preferably relates to a BVDV according to the invention, wherein said mutation(s) in the coding sequence for glycoprotein E^{rns} are located in the encoding nucleotide sequence corresponding to amino acids at position 298 to 310 and/or position 341 to 360. Preferably, such a mutation is (amino acids are given in the one letter symbols; the amino acid before the position number indicates the amino acid to be substituted, the amino acid after the position number the substituting amino acid (del indicates deletion) for example, H300L, which means that histidine at position 300 was substituted by leucine:

Suitable modification of the glycoprotein E^{rns} are for example, the single substitutions/deletions: S298G, H300K, H300L, H300R, H300del, W303G, P304del, E305A, C308G, R343G, E345del, W346G, K348A, H349K, H349L, H349del, H349Q, H349SV (mutation H349S and insertion of V), K348R, W351P, W351G, W351L, W351K, W351H; the double substitutions/deletions: H300L/H349L, K348del/H349del, H349del/G350del, E345del/H349del, W303G/E305A, H300K/H349K, H300K/H349L and the triple deletions: L299del/H300del/G301del, K348del/H349del/G350del. Numbering is according to the published amino acid sequence of BVDV CP7 for all the mutants listed above. All the above-listed mutants were at least tested as BVDV mutants without mutations in the N^{pro} region. Suitable mutants of the pestiviral such BVDV glycoprotein. E^{rns} are provided, for example, by WO 99/64604, which is incorporated herein at its whole. It should be noted, however, that according to a further preferred embodiment of the present invention, at least one additional mutation in the N^{pro} region, as disclosed in further detail below, must be present.

It was particularly found that deletion or substitution of the histidine residue at position 349 (BVDV) leads to effective inactivation of E^{rns} and therefore leads to particularly useful BVDV live vaccines. The present invention demonstrates that BVDV are viable and code for an E^{rns} protein without RNase activity when the histidine residue at position at position 349 (numbering according to the published sequence of BVDV CP7 (Meyers et al., 1996b)) is deleted. Thus, preferably, the BVDV as used in the combination vaccine bears a mutation in the coding sequence for glycoprotein E^{rns} is a deletion or substitution of the histidine residue at position 349. Even more specifically, the putative active site of the RNase is represented by the conserved E^{rns} sequences SLHGIWPEKICTG (SEQ ID NO 13) and/or LQRHEWNKHGWCNWFHIEPW (SEQ ID NO 14) (sequence of the BVDV-2 New York'93 protein is given here in an exemplary manner; minor changes can possibly be found in other BVDV sequences but the identity of the motif will always be obvious for an expert in the field). As an example, the corresponding amino acid sequences of BVDV-1 CP7 would be SLHGIWPEKICTG (SEQ ID NO 13) and/or LQRHEWNKHGWCNWYNIEPW (SEQ ID NO 15). Thus, preferably, the BVDV of the combination vaccine bears mutation(s) in the coding sequence for glycoprotein E^{rns} are located in the nucleotide sequence coding for the conserved E^{rns} sequence SLHGIWPEKICTG (SEQ ID NO 13) and/or LQRHEWNKHGWCNWFHIEPW (SEQ ID NO 14). These sequences are representing the putative active site of the RNase. The sequences SLHGIWPEKIC (SEQ ID NO 16) and RHEWNKHGWCNW (SEQ ID NO 17) of the putative E^{rns} active site are even more conserved across pestiviruses. Thus, preferably, the BVDV used for the preparation of a combination vaccine as described herein has at least one mutation in the coding sequence of the N^{pro} protein and/or the glycoprotein E^{rns}, wherein said mutation(s) in the coding sequence for glycoprotein E^{rns} are located in the nucleotide sequence coding for the conserved E^{rns} sequence SLHGIWPEKIC (SEQ ID NO 16) and/or RHEWNKHGWCNW (SEQ ID NO 15). Preferably, the mutation is located in only one of said sequences. Thus the BVDV of the combination vaccine described herein having at least one mutation in the coding sequence of the N^{pro} protein and/or the glycoprotein E^{rns}, wherein said mutation(s) in the coding sequence for glycoprotein E^{rns} are located in the nucleotide sequence coding for the conserved E^{rns} sequence SLHGIWPEKIC (SEQ ID NO 16) or RHEWNKHGWCNW (SEQ ID NO 17). Preferably, such mutations concern two different amino acids, i.e. are double mutations. Thus, said mutations may be 1 to 3 nucleotide mutations in two different triplets encoding two amino acids. Thus, the invention also relates to a combination vaccine comprising a live attenuated BVDV having at least one mutation in the coding sequence of the N^{pro} protein and/or the glycoprotein E^{rns}, wherein said mutation(s) in the coding sequence for glycoprotein E^{rns} are two mutations located in the nucleotide sequence coding for the conserved E^{rns} sequence SLHGIWPEKIC (SEQ ID NO 16) and/or RHEWNKHGWCNW (SEQ ID NO 17). Preferably, such mutations concern a single amino acid. Thus, said mutation may be 1 to 3 nucleotide mutations in one triplett encoding one amino acid. Thus, the invention also relates to combination vaccine for the treatment and/or prophylaxis of cattle against microbiological infections comprising a live attenuated BVDV having at least one mutation in the coding sequence of the N^{pro} protein and/or the glycoprotein E^{rns}, wherein a single mutation is located in the conserved E^{rns} sequence SLHGIWPEKIC (SEQ ID NO 16) and/or RHEWNKHGWCNW (SEQ ID NO 17).

### Modification of the N^{pro} Gene of BVDV

As mentioned above, the attenuated BVDV provided herein, having at least one mutation in the coding sequence of the glycoprotein E^{rns} and/or in the coding sequence of the N^{pro} protein, wherein said mutation preferably results in inactivation of the RNase activity residing in the glycoprotein E^{RNS} and/or of the immunomodulating activity residing in N^{pro}. Inactivation of N^{pro} is achieved in BVDV of the specified formula described more in detail below, wherein between 0 and all amino acids of N^{pro} are present; ubiquitin or LC3 or another sequence serves as processing signal (e.g. SUMO-1, NEDD8, GATE-16, GABA(A)RAP, or proteases like e.g. Intern, picornavirus 3C, cardovirus 2A, or p15 of rabbit hemorrhagic disease virus) are present or absent. In case a processing signal is present, the coding sequence of the processing signal is inserted at or close to the C-terminal end of the (remaining part of the) N^{pro}-protein. Only in the case that a processing signal is present, any number of amino acids coding for N^{pro} (=N^{pro} amino acids) may be present. In case no processing signal sequence is inserted, a maximum of 12 amino acids, preferably aminoterminal amino acids, of N^{pro} may be present, the remaining amino acids have to be deleted. Furthermore, other than the E^{rns} mutations as disclosed above (at least one of which has to be present in the attenuated BVDV as described herein), the remaining sequences of the attenuated BVDV may remain unchanged, i.e. are not mutated, or may also have mutations close to the N-terminal end of the C-protein. A number of more specific embodiments as disclosed below exemplify this.

Thus, the attenuated BVDV of the combination vaccine is modified in the coding regions of the E^{rns} as described above and/or in the N^{pro}, wherein said mutation(s) in the coding sequence for N^{pro} leads to an encoded poly protein as characterized by the following formula:

[N^{pro}]ₓ-[PS]_{y}-[C-term]
and wherein:
[N^{pro}] relates to the N^{pro} portion of said polyprotein, wherein “x” represents the number of amino acids of the N^{pro} present in the polyprotein;
[PS] relates to a processing signal selected from: ubiquitin, LC3, SUMO-1, NEDD8, GATE-16 or GABA(A)RAP or proteases like e.g. lutein, picornavirus 3C, cardovirus 2A, or p15 of rabbit hemorrhagic disease virus, or any processing signal known to the skilled person that ensures the generation of a functional N-terminal of the C-protein. “Y” may be =0, which means that no processing signal is present (=PS is absent), or “Y” may be =1, which means that a processing signal is present (=PS present).
[C-term] relates to the complete pestivirus, in particular the complete BVDV polyprotein except for N^{pro}, but including the capsid (C)-protein and any other protein present in the pestivirus polyprotein, in particular in the BVDV polyprotein including the carboxyterminal NS5B. Preferably, the glycoprotein E^{rns} in said [C-term] is mutated, in such that the RNase activity residing in the glycoprotein E^{rns} is inactivated. The term “any other protein present in the pestivirus polyprotein/BVDV polyprotein” relates to E^{rns}, E1, E2, p7, NS2, NS3, NS4A, NS4B and NS5A, wherein glycoprotein E^{rns} is mutated, preferably as disclosed herein (see above), in such that the RNase activity residing in the glycoprotein E^{rns} is inactivated. Preferably, the pestivirus, in particular the BVDV according to the invention has a C-protein which is not mutated except for the amino acid at position 2 which is changed from D to N. Therefore, [C-term*] is the same as [C-term] but with a mutation at position 2 of the C-protein (N instead of D);
if “y” is =0 (means no [PS] present) then “x” is 0 to 12, (means no N^{pro} specific amino acid or 1 to 12 amino acids of N^{pro}, preferably of the N-terminus of N^{pro}, are present);
if “y” is =1 (means [PS] is present) then “x” is 0 to 168; (means no N^{pro} specific amino acid or 1 to all 168 amino acids of N^{pro}, preferably of the N-terminus of N^{pro}, are present).

[N^{pro}]₁-[PS]₀-[C-term]
and wherein the definitions are as defined above.

A specific example thereof is disclosed below, wherein the N-terminal methionine is followed by the C-protein and any other protein present in the poly protein including the carboxy terminal NS5B Hence, most preferably, the attenunated BVDV as described herein is encoded by a poly protein as characterized by the following formula:

M[C-term]
and wherein the definitions are as defined above.

Also more preferably, the invention relates to a combination vaccine for the treatment and/or prophylaxis of cattle against microbiological infections, wherein said vaccine comprises an attenuated BVDV that is modified in the coding regions of the E^{rns} as described above and/or in the N^{pro}, wherein said mutation(s) in the coding sequence for N^{pro} leads to an encoded polyprotein as characterized by the following formula:

[N^{pro}]₃-[PS]₀-[C-term]
and wherein the definitions are as defined above.

A further specific example of an attenuated BVDV is disclosed below, wherein the N-terminal methionine is followed by the N^{pro} sequence EL and the C-protein and any other protein present in the polyprotein including the carboxy terminal NS5B. Hence, most preferably, the attenuated BVDV as described herein is encoded by a polyprotein as characterized by the following formula:

MEL-[C-term]
and wherein the definitions are as defined above.

Also more preferably, the invention relates to a combination vaccine for the treatment and/or prophylaxis of cattle against microbiological infections, wherein said vaccine comprises an attenuated BVDV that is modified in the coding regions of the E^{rns} as described above and/or in the N^{pro}, wherein said mutation(s) in the coding sequence for N^{pro} leads to an encoded polyprotein as characterized by the following formula:

[N^{pro}]₄-[PS]₀-[C-term]
and wherein the definitions are as defined above.

A further specific example of BVDV is disclosed below, wherein the N-terminal methionine is followed by the N^{pro} sequence ELF (SEQ ID NO 18) and the C-protein and any other protein present in the polyprotein including the carboxyterminal NS5B. Hence, most preferably, the invention refers to a BVDV, wherein said mutation(s) in the coding sequence for N^{pro} leads to an encoded polyprotein as characterized by the following formula:

MELF-[C-term].
and wherein the definitions are as defined above.

Also more preferably, the invention relates to a combination vaccine for the treatment and/or prophylaxis of cattle against microbiological infections, wherein said vaccine comprises an attenuated BVDV that is modified in the coding regions of the E^{rns} as described above and/or in the N^{pro}, wherein said mutation(s) in the coding sequence for N^{pro} leads to an encoded polyprotein as characterized by the following formula:

[N^{pro}]₆-[PS]₀-[C-term]

and wherein the definitions are as defined above.

A further specific example of an attenuated BVDV as described herein is given below, wherein the N-terminal methionine is followed by the N^{pro} sequence ELFSN (SEQ ID NO 19) and the C-protein and any other protein present in the polyprotein including the carboxyterminal NS5B. Hence, most preferably, the invention relates to a combination vaccine for the treatment and/or prophylaxis of cattle against microbiological infections, wherein said vaccine comprises an attenuated BVDV that is modified in the coding regions of the E^{rns} as described above and/or in the N^{pro}, wherein said mutation(s) in the coding sequence for N^{pro} leads to an encoded polyprotein as characterized by the following formula:

MELFSN-[C-term].
and wherein the definitions are as defined above.

A further specific example of an attenuated BVDV as described herein is given below, wherein the N-terminal methionine is followed by the N^{pro} sequence ELFSNE (SEQ ID NO 20); ELFSNEL (SEQ ID NO 21); ELFSNELL (SEQ ID NO 22); ELFSNELLY (SEQ ID NO 23); ELFSNELLYK (SEQ ID NO 24); or ELFSNELLYKT (SEQ ID NO 25) and the C-protein and any other protein present in the polyprotein including the carboxyterminal NS5B. Hence, most preferably, the invention relates to a combination vaccine for the treatment and/or prophylaxis of cattle against microbiological infections, wherein said vaccine comprises an attenuated BVDV that is modified in the coding regions of the E^{rns} as described above and/or in the N^{pro}, wherein said mutation(s) in the coding sequence for N^{pro} leads to an encoded polyprotein as characterized by the following formula:

MELFSN-[C-term].

MELFSNE-[C-term];

MELFSNEL-[C-term];

MELFSNELL-[C-term]:

MELFSNELLY-[C-term];

MELFSNELLYK-[C-TERM]

MELFSNELLYKT-[C-TERM]
and wherein the definitions are as defined above.

Also more preferably, the invention relates to a combination vaccine for the treatment and/or prophylaxis of cattle against microbiological infections, wherein said vaccine comprises an attenuated BVDV that is modified in the coding regions of the E^{rns} as described above and/or in the N^{pro}, wherein said mutation(s) in the coding sequence for N^{pro} leads to an encoded polyprotein as characterized by the following formula:

[N^{pro}]₄-[PS]₀-[C-term*]
and wherein the definitions are as defined above except for the fact that the amino terminal part of the C-protein is changed, also as described above.

A further specific example of BVDV is disclosed below, wherein the N-terminal methionine is followed by the N^{pro} sequence ELF and in the C-protein sequence, the amino acid at position 2 is changed from D to N. Therefore, the aminoterminal C-protein sequence is SNEGSK (SEQ ID NO 26), instead of SDEGSK (SEQ ID NO 27). Hence, most preferably, the invention relates to a combination vaccine for the treatment and/or prophylaxis of cattle against microbiological infections, wherein said vaccine comprises an attenuated BVDV that is modified in the coding regions of the E^{rns} as described above and/or in the N^{pro}, wherein said mutation(s) in the coding sequence for N^{pro} leads to an encoded polyprotein as characterized by the following formula:

MELF-[C-term*],
wherein in the C-protein the amino acid at position 2 is changed from D to N, and
wherein the definitions are as defined above.

Also more preferably, the invention relates to a combination vaccine for the treatment and/or prophylaxis of cattle against microbiological infections, wherein said vaccine comprises an attenuated BVDV that is modified in the coding regions of the E^{rns} as described above and/or in the N^{pro}, wherein said mutation(s) in the coding sequence for N^{pro} lead to an encoded polyprotein as characterized by the following formula:

[N^{pro}]ₓ-[PS]₁-[C-term],
wherein the definitions are as defined as above,
and wherein PS is any of the PS disclosed above, and more preferably selected from the group of ubiquitin or LC3.

A further specific example of BVDV is disclosed below, wherein the N-terminal methionine is followed by any 21 or 28 N^{pro} amino acids, ubiquitin or LC3, and the C-protein. Hence most preferably, the invention relates to a combination vaccine for the treatment and/or prophylaxis of cattle against microbiological infections, wherein said vaccine comprises an attenuated BVDV that is modified in the coding regions of the E^{rns} as described above and/or of the N^{pro}, wherein said mutation(s) in the coding sequence for N^{pro} lead to an encoded polyprotein as characterized by the following formula:

[N^{pro}]₂₂-[PS]₁-[C-term], wherein preferably, the PS is ubiquitin or LC3 or

[N^{pro}]₂₉-[PS]₁-[C-term], wherein preferably, the PS is ubiquitin or LC3,

Ubiquitin is a well known highly conserved cellular protein of 76 amino acids. Among other functions, ubiquitin is a key player in protein catabolism since conjugation with ubiquitin can mark a protein for degradation via the proteasome. Ubiquitin conjugated with or fused to other proteins via the carboxyterminal glycine can be cleaved off by cellular ubiquitin-specific proteases. Thus, fusion, of a protein to the carboxyterminus of ubiquitin will usually result in defined proteolytic cleavage of the fusion protein into its components when expressed within a cell.

LC3 (light chain 3 of microtubule associated proteins) represents a cellular protein of 125 amino acids that serves a variety of functions (length given for bovine LC3). Recently, a fundamental role of the protein in autophagy has been defined. During this process, LC3 is activated by carboxyterminal cleavage. Thereby, a new carboxyterminus is generated that consists of glycine. LC3 is then conjugated via the carboxyterminal glycine to phosphatidylethanolamine present in the to membranes of autophagic vesicles. Because of this process, a protein fused to the carboxy terminus of LC3 will be cleaved off by a cellular protease at a defined position.

Also more preferably the invention relates to a combination vaccine for the treatment and/or prophylaxis of cattle against microbiological infections, wherein said vaccine comprises an attenuated BVDV that is modified in the coding regions of the E^{rns} as described above and/or in the N^{pro}, wherein said mutation(s) in the coding sequence for N^{pro} lead to an encoded polyprotein as characterized by the following formula, selected from the group of:

[N^{pro}]₂-[PS]_{y}-[C-term] and preferably ME-[PS]_{y}-[C-term];

[N^{pro}]₅-[PS]_{y}-[C-term] and preferably MELFS-[PS]_{y}-[C-term];

[N^{pro}]₇-[PS]_{y}-[C-term] and preferably MELFSNE-[PS]_{y}-[C-term];

[N^{pro}]₈-[PS]_{y}-[C-term] and preferably MELFSNEL-[PS]_{y}-[C-term];

[N^{pro}]₉-[PS]_{y}-[C-term] and preferably MELFSNELL-[PS]_{y}-[C-term];

[N^{pro}]₁₀-[PS]_{y}-[C-term] and preferably MELFSNELLY-[PS]_{y}-[C-term];

[N^{pro}]₁₁-[PS]_{y}-[C-term] and preferably MELFSNELLYK-[PS]_{y}-[C-term]; and

[N^{pro}]₁₂-[PS]_{y}-[C-term] and preferably MELFSNELLYKT-[PS]_{y}-[C-term]
and wherein the definitions are as defined as above. The preferably disclosed embodiments refer to BVDV.
Most preferably, y is 0 (no PS present).

Suitable E^{RNS} N^{pro} double mutants of BVDV include those listed in the fable below:

TABLE
BVDV E^{Rns} N^{pro} double mutants:

| | |
|---|---|
| Modification of E^{RNS} | Modification of N^{Pro} |

| | |
|---|---|
| S298G | ME-, MEL-, MELF-, MELFSN-, MELFSNELL- |
| | MELFSNELLY-, MELFSNELLYK-, OR MELFSNELLYKT-, |
| | MESDEGSK-, MELFSSDEGSK-. MELFSNESDEGDK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| H300K | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| H300L | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| H300del | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDGSK-, MELFSNESDEGSK-, |
| | MELFSNELSNELSDEGSK-, MELFSNELLSDEGSK-, |
| | MELFSNELLYSDEGSK-, MELFSNELLYKSDEGSK-, |
| | MELFSNELLYKTSDEGSK- |
| W303G | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| P304del | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| E305A | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| C308G | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| R343G | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| E345del | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| W346G | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| K348A | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| K348R | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| H349K | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| H349L | MR-, MEL-, MELF-, MELFS-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGK-, MELFSNELLYKSDEGSK-, |
| | MELFSNELLYKTSDEGSK- |
| H349Q | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| H349del | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| H349SV | ME-, MEL-, MELF-, MELFSN-, MELFSNEL-, MELFSNELL-, |
| (mutation | MELFSNELLY-, MELFSNELLYK-, or MELFSNELLYKT-, |
| H349S | MESDEGSK-, MELFSSDEGSK-, MELFSNESDEGSK-, |
| and insertion | MELFSNELSDEGSK-, MELFSNELLSDEGSK-, |
| of V) | MELFSNELLYSDEGSK-, MELFSNELLYKSDEGSK-, |
| | MELFSNELLYKTSDEGSK- |
| W351P | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYSTSDEGSK- |
| W351K | ME-, MEL-, MELF-, MELFSN-, MELFSNEL-, MELFSNELL-, |
| | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSELLYSDEGSK-, MELFSNELLYKSDEGSK-, |
| | MELFSNELLYKTSDEGSK- |
| H300L/ | ME-, MEL-, MELF-, MELFSN-, MELFSNEL-, MELFSNELL-, |
| H349L | MELFSNELLY-, MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| H300K/ | ME-, MEL-, MELF-, MELFSN-, MELFSNEL-, MELFSNELL-, |
| H349K | MELFSNELLY-. MELFSNELLYK-, or MELFSNELLYKT-, |
| | MESDEGSK-, MELFSSDEGSK-, MELFSNESDEGSK-, |
| | MELFSNELSDEGSK-, MELFSNELLSDEGSK-, |
| | MELFSNELLYSDEGSD-, MELFSNELLYKSDEGSK-, |
| | MELFSNELLYKTSDEGSK- |
| H300K/ | ME-, MEL-, MELF-, MELFSN-, MELFSNEL-, MELFSNELL-, |
| H349L | MELFSNELLY-, MELFSNELLYK-, or MELFSNELLYKT-, |
| | MESDEGSK-, MELFSSDEGSK0, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| W303G/ | ME-, MEL-, MELF-, MELFSN-, MELFSNEL-, MELFSNELL-, |
| E305A | MELFSNELLY-, MELFSNELLYK-, or MELFSNELLYKT-, |
| | MESDEGSK-, MELFSSDEGSK0, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| K348del/ | ME-, MEL-, MELF-, MELFSN-, MELFSNEL-, MELFSNELL-, |
| H349del | MELFSNELLY-, MELFSNELLYK-, or MELFSNELLYKT-, |
| | MESDEGSK-, MELFSSDEGSK0, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| K345del/ | ME-, MEL-, MELF-, MELFSN-, MELFSNEL-, MELFSNELL-, |
| H349del | MELFSNELLY-, MELFSNELLYK-, or MELFSNELLYKT-, |
| | MESDEGSK-, MELFSSDEGSK0, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| K349del/ | ME-, MEL-, MELF-, MELFSN-, MELFSNEL-, MELFSNELL-, |
| G350del | MELFSNELLY-, MELFSNELLYK-, or MELFSNELLYKT-, |
| | MESDEGSK-, MELFSSDEGSK0, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| L299deL/ | ME-, MEL-, MELF-, MELFSN-, MELFSNEL-, MELFSNELL-, |
| H300del | MELFSNELLY-, MELFSNELLYK-, or MELFSNELLYKT-, |
| G301del | MESDEGSK-, MELFSSDEGSK0, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |
| L299del/ | ME-, MEL-, MELF-, MELFSN-, MELFSNEL-, MELFSNELL-, |
| H300del | MELFSNELLY-, MELFSNELLYK-, or MELFSNELLYKT-, |
| G301del | MESDEGSK-, MELFSSDEGSK0, MELFSNESDEGSK-, |
| | MELFSNELSDEGSK-, MELFSNELLSDEGSK-, |
| | MELFSNELLYSDEGSK-, MELFSNELLYKSDEGSK-, |
| | MELFSNELLYKTSDEGSK- |

According to one embodiment of the present invention, the attenuated BVDV of the combination vaccine as provided herein is a BVDV type 1. Preferably, the attenuated BVDV is based on one of the following BVDV type 1 strains: NADL, Osloss, SD-1, CP7 or KE9, wherein each of the strains comprises at least one of the E^{RNS} and/or N^{pro} mutations as described above, and preferably at least one of the double-mutants within the E^{RNS} and N^{pro} region as listed in the table above. According to a further embodiment, said attenuated BVDV of the combination vaccine as provided herein is a BVDV type 2. Preferably, the attenuated BVDV is based on one of the following BVDV type 2 strains: 890, C413, or New York'93C, wherein each of the strains comprises al least one of the E^{RNS} and/or N^{pro} mutations as described above, and preferably at least one of the double-mutants within the E^{RNS} and N^{pro} region as listed in the table above.

BVDV-1 and BVDV-2 are differentiated according to features of their genomic sequences (Heinz et al., 2000 and references therein). BVDV-1 as disclosed herein may be used in the manufacture of a composition for use in the prevention and/or treatment of BVDV type 1 infections in breeding stocks of cattle, in pregnant cows and in the induction of fetal protection against BVDV type 1 infection is pregnant cows. Surprisingly, a BVDV-2 as disclosed herein may be used in the manufacture of a combination vaccine for use in the prevention and/or treatment of BVDV type 1 infections in breeding stocks of cattle. In particular, the invention relates to the use of a BVDV type 2 as described herein in the manufacture of a combination vaccine for use in the prevention and/or treatment of BVDV type 1 infections in pregnant cows. Preferably, the BVDV type 2 as provided herein may be used in the manufacture of a combination vaccine for use in the induction of fetal protection against BVDV type 1 infections in pregnant cows. Surprisingly also, a BVDV-1 as disclosed herein may be used in the manufacture of a combination vaccine for use in the prevention and/or treatment of BVDV type 2 infections in breeding stocks of cattle. In particular, the invention relates to the use of a BVDV type 1 as described herein in the manufacture of a combination vaccine for use in the prevention and/or treatment of BVDV type 2 infections in pregnant cows. Preferably, the BVDV type 1 according to the invention may be used in the manufacture of a combination vaccine for use in the induction of fetal protection against BVDV type 2 infections in pregnant cows.

Most preferred is the use of BVDV type 1 and type 2 in combination for the manufacture of the combination vaccines described herein for use in the prevention and/or treatment of BVDV type 1 and or type 2 infections in breeding stocks of cattle, in pregnant cows and in the induction of fetal protection against BVDV type 1 and/or type 2 infections in pregnant cows. Thus, preferably the combination vaccines provided herewith comprise one or more attenuated BVDV type 1 and type 2 as described above. For example, the combination vaccines provided herewith comprise an attenuated BVDV of type 1 and type 2, based on the strains: NADL/890; NADL/C413; NADL/New York'93/C: CP7/890; CP7/C413; CP7/New York'93/C; KE9/890; KE9/C413; KE9/New York'93/C, wherein, each of the strains comprises at least one of the E^{RNS} and/or N^{pro} mutations as described above, and more preferably at least one of the double-mutants within the E^{RNS} and N^{pro} region as listed in the table above. More preferably, any of the combination vaccines provided herewith may include one of more sub-types of attenuated BVDV type 1 and one or more sub-types of attenuated BVDV-2, e.g one or more attenuated BVDV of sub-types 1a, 1b, 1c, 1d, 1e, 1f, 1g, 1h, and the like and one or more attenuated BVDV of sub-types 2a, 2b and the like. Most preferred is a combination vaccine comprising attenuated BVDV of sub-types 1a, 1b, and 2a. Thus, according to a preferred embodiment of the present invention, the phrase “attenuated BVDV (types 1 and/or 2)” includes but is not limited to combinations of BVD viruses comprising one or more attenuated BVDV of type 1, preferably of sub-type 1b and one or more attenuated BVDV of type 2, preferably of subtype 2a. According to further embodiment of the present invention the phrase “attenuated BVDV (types 1 and/or 2)” includes but is not limited to combinations of BVD viruses comprising one or more “attenuated BVDV of sub-type 1a, one or more attenuated BVDV of sub-type 1b, and one or more attenuated BVDV of type 2, preferably of sub-type 2a.

If more than one attenuated BVDV is used in the combination vaccines as described herein, each of die attenuated BVDV should mutated in same genomic site of the E^{RNS} and/or the N^{pro} such that the none of the attenuated BVDV can recombine with any of the others to eliminate the mutations which are essential and responsible for the attenuation of the viruses. For example if BVDV type 1a with one of the following E^{RNS} mution is used: H349K, H349L, H349del, H349Q, H349SV (mutation H349S and insertion of V), a BVDV type 1b and/or type 2 should be used, which are/is mutated in the same site of the E^{RNS}, i.e. at position 349 or at amino acid position which corresponds to position 349 of BVDV type 1 in order to avoid revertation of attenuated BVDV type 1 or type 2. This principle also applies to any mutation within the N^{pro} region. Thus, according to a preferred embodiment of the present invention, the phrase “attenuated BVDV (types 1 and/or 2)” includes but is not limited to combinations of BVD viruses comprising one or more attenuated BVDV of type 1 and one or more attenuated BVDV of type 2, wherein each of the attenuated BVDV is mutated in same genomic site of the E^{RNS} and/or the N^{pro} such that none of the attenuated BVDV cat recombine with any of the others to eliminate the mutations within the E^{RNS} and N^{pro} which are essential and responsible for the attenuation of the viruses. According to another embodiment of the present invention, the phrase “attenuated BVDV (types 1 and/or 2)” includes, but is not limited to, a combination of BVD viruses comprising one or more “attenuated BVDV of sub-type 1a, one or more attenuated BVDV of sub-type 1b, and one or more attenuated BVDV of type 2, preferably of sub-type 2a, wherein each of the attenuated BVDV is mutated in same genomic site of the E^{RNS} and/or the N^{pro} such that none of attenuated BVDV can recombine with any of the others to eliminate the mutations within the E^{RNS} and/or N^{pro} which are essential and responsible for the attenuation of the viruses.

### Preparation of the Attenuated BVDV

Another important aspect of the invention is a method for attenuating a pestivirus, characterized in that at least one mutation in the coding sequence for glycoprotein E^{rns} and/or at least another mutation in the coding sequence for N^{pro} is generated in the BVDV genome.

According to a more preferred embodiment, said method comprises the steps:
a) reverse transcription of a wild-type pestivirus nucleotide sequence into a cDNA;
b) cloning said cDNA;
c) introducing mutations selected from the group of deletions, insertion mutations and/or substitution mutations into said cDNA, wherein said mutations are located in the coding sequence encoding glycoprotein E^{rns} and/or the protease N^{pro},
d) incorporating the cDNA into a plasmid or into a DMA virus capable of directing the transcription of BVDV cDNA into RNA in vitro or upon infection of suitable cells.

Regarding the method for attenuating a BVDV according to the invention, said preferred method comprises the steps:
a) reverse transcription of a wild-type BVDV nucleotide sequence into a cDNA;
b) cloning said cDNA;
c) introducing mutations selected from the group of deletions, insertion mutations and/or substitution mutations into said cDNA, wherein said mutations are located in the coding sequence encoding glycoprotein E^{rns} and/or the protease N^{pro},
d) incorporating the cDNA into a plasmid or into a DNA virus capable of directing the transcription of BVDV cDNA into RNA in vitro or upon infection of suitable cells.

There are several nucleotide sequences blown in the art, which represents the basis for the production of a polynucleotide molecule coding for a BVDV attenuated as described herein, having at least one mutation in the coding sequence of N^{pro} and/or at least one in the coding sequence of glycoprotein E^{rns}, wherein said mutations result in a combined inactivation of the RNase activity residing in glycoprotein E^{rns} and in the inactivation of the immunomodulating activity residing in N^{pro}. Examples of nuclecic acid sequences of wild-type sequences of several BVDV strains are listed below:

Bovine viral diarrhea virus 1

| | |
|---|---|
| Strain NADL | NCBI GenBank Accession No. [M31182] |
| Strain Osloss | NCBI GenBank Accession No. [M96687] |
| Strain SD-1 | NCBI GenBank Accession No. [M96751] |
| Strain CP7 | NCBI GenBank Accession No. [U63479] |
| Strain KE9 | (SEQ ID NO: 1) |

Bovine viral diarrhea virus 2

| | |
|---|---|
| Strain 890 | NCBI GenBank Accession No. [U18059] |
| Strain C413 | NCBI GenBank Accession No. [AF002227] |
| Strain NewYork'93/C | NCBI GenBank Accession No. [AF502399] |

The mutations/modifications relating to the coding sequences of N^{pro} and E^{rns} are described above more in detail. Having this information, a person skilled in the art is able to realize the manufacture of any polynucleotide/polynucleic acid coding for an attenuated BVDV as provided herewith. Molecular methods for introducing a mutation into a polynucleotide sequence, as well as the cloning and amplification of said mutated polynucleotide are for example provided by Sambrook et al. 1989 or Ausubel et al. 1994.

Most preferably, the wild type BVDV which is to be mutated as disclosed herein corresponds to amino acid sequence SEQ ID No. 5 (termed XIKE A) or is a functional variant thereof. Most preferably also, the BVDV has a N^{pro} mutation as described herein corresponding to amino acid sequence SEQ ID No. 6 (termed XIKE-A-NdN) or is a functional variant thereof. Preferably, such a functional variant is at least 65% homologous to the amino acid sequence disclosed herein. On the amino acid level, homologies are very roughly: BVDV-1/-BVDV-1: 93%; BVDV-1/-BVDV-2: 84%; BVDV-2/-BVDV-2: 98%. Therefore, more preferably, such a functional variant is at least 70%, 71%, 72%, 73%, 74%, 75%, 76%, 77%, 78%, 79%, 80%, 81%, 82%, 83%, 84%, 85%, 86%, 87%, 88%, 89% or 90% homologous to the amino acid sequence disclosed herein. More preferably also, such functional variant is at least 91%, 92%, 93%, 94%, 95%, 96%, 97% or 98% homologous to the amino acid sequence disclosed herein. Most preferably, such functional variant is at least 99% or 99.9% homologous to the amino acid sequence disclosed herein.

Most preferably also, the attenuated BVDV as described herein has a E^{rns} mutation which has a deletion of the codon coding for histidine 349 (termed XIKE-B), or is a functional variant thereof. Most preferably also, the attenuated BVDV has both a E^{rns} mutation and/or a N^{pro} mutation as described herein, wherein the codon coding for histidine 349 of E^{rns} is deleted and also the complete N^{pro} coding region is deleted, except for codons 1 to 4, thus amino acids MELF of N^{pro} remain. Said double mutant corresponds to amino acid sequence SEQ ID No. 8 (termed XIKE-B-NdN) or is a functional variant thereof. Preferably, such a functional variant is at least 65% homologous to the amino acid sequence disclosed herein. More preferably, such a functional variant is at least 70%, 71%, 72%, 73%, 74%, 75%, 76%, 77%, 78%, 79%, 80%, 81%, 82%, 83%, 84%, 85%, 86%, 87%, 88%, 89% or 90% homologous to the amino acid sequence disclosed herein. More preferably also, such functional variant is at least 91%, 92%, 93%, 94%, 95%, 96%, 97% or 98% homologous to the amino acid sequence disclosed herein. Most preferably, such functional variant is at least 99% or 99.9% homologous to the amino acid sequence disclosed herein.

Most preferably also, the BVDV according to the invention has an E^{rns} mutation according to the invention which has a substitution of the codon coding for histidine 300 by the codon coding for leucine (termed XIKE-C), or is a functional variant thereof. Most preferably also, the BVDV according to the invention has both a E^{rns} mutation, and a N^{pro} mutation according to the invention, wherein the codon coding for histidine 300 is substituted by the codon coding for leucine and also the complete N^{pro} coding region is deleted, except for codons 1 to 4, thus amino acids MELF of N^{pro} remain. Said mutant corresponds to amino acid sequence SEQ ID No. 10 (termed XIKE-C NdN) or is a functional variant thereof. Preferably, such a functional variant is at least 65% homologous to the amino acid sequence disclosed herein. More preferably, such a functional variant is at least 70%, 71%, 72%, 73%, 74%, 75%, 76%, 77%, 78%, 79%, 80%, 81%, 82%, 83%, 84%, 85%, 86%, 87%, 88%, 89% or 90% homologous to the amino acid sequence disclosed herein. More preferably also, such functional variant is at least 91%, 92%, 93%, 94%, 95%, 96%, 97% or 98% homologous to the amino acid sequence disclosed herein. Most preferably, such functional variant is at least 99% or 99.9% homologous to the amino acid sequence disclosed herein.

### Combination Partners:

As described above, the present invention relates to a combination vaccine for the treatment and/or prophylaxis of cattle against microbiological infections, wherein said combination vaccine comprises attenuated BVDV (types 1 and/or 2) as described above, and at least one further immunological active component effective for the treatment and or prophylaxis of infections caused by a bovine pathogen other than BVDV. As mentioned above, the combination vaccine preferably comprises attenuated BVDV type 1 and BVDV type 2, both having at least one mutation in the coding sequence for glycoprotein E^{rns} and/or at least another mutation in the coding sequence for N^{pro}, wherein said mutation in the coding sequence for glycoprotein E^{rns} leads to inactivation of RNase activity residing in E^{rns} and/or said mutation in the coding sequence for N^{pro} leads to inactivation of said N^{pro}. Even more preferably, each of the attenuated BVDV is mutated in same genomic site of the E^{RNS} and/or the N^{pro} such that none of the attenuated BVDV can recombine with any of the others to eliminate the mutations within the E^{RNS} and/or N^{pro}, which are essential and responsible for the attenuation of the viruses. Even more preferably, the combination vaccine comprises attenuated BVDV type 1 and BVDV type 2, both having at least one mutation in the coding sequence for glycoprotein E^{rns} and at least another mutation in the coding sequence for N^{pro}, wherein said mutation in the coding sequence for glycoprotein E^{rns} leads to inactivation of RNase activity residing in E^{rns} and/or said mutation in the coding sequence for N^{pro} leads to inactivation of said N^{pro}. Most preferably, each of the attenuated BVDV is mutated in same genomic site of the E^{RNS} and the N^{pro} such that none of the attenuated BVDV can recombine with any of the others to eliminate the mutations within the E^{RNS} and/or N^{pro}, which are essential and responsible for the attenuation of the viruses.

Relevant bovine pathogens other titan BVDV include but are not limited to: 1) pathogens of viral origin such as Parainfluenza-3 Virus (PI-3), Infectious Bovine Rhinotracheitis virus (IBR), Bovine Respiratory Syncytial Virus (BRSV), Bovine Herpesvirus (BHV), Bovine Rotavirus (BRV), Bovine Enterovirus (BEV), Bovine Coronovirus (BCV). Bovine Rabies (BR), Bovine Parvovirus (PPV), and Adenovirus and Astrovirus; ii) pathogens of bacterial origin, such as Mannheimia haemolytica (formerly Pasteurella haemolytica), Pasteurella mullocida, Haemophilus somnus (Histophilus ovis and Haemophilus agni), Actinomyces (Corynebacterium), Actinomyces pyogenes, Chlamydia psittaci, Campylobacter fetus venerealis and Campylobacter fetus fetus (formerly C fetus intestinalis). Leptospira interrogans, Leptospira pomona, and Leptospira grippotyphosa, Leptospira canicola, Leptospira grippotyphosa, Leptospira hardjo (Leptospira hardjoprajitno and Leptospira hardjo-bovis), Brucella abortus, Brucella suis and Brucella melitensis, Listeria monocytogenes, Chlamydia psittaci, Clostridium chauvoei, Clostridium septicum, Clostridium haemolyticum, Clostridium novyi, Clostridium sordellii, Clostridium perfringens, Clostridium tetani, Moraxella bovis, Klebsiella spp. Klebsiella, pneumoniae, Salmonella typhimurium; Salmonella newport, Mycobacterium avium paratuberculosis, Staphylococcus aureus, Streptococcus dysgalactiae, and Streptococcus uberus iii) pathogens of other origin, such as Mycoplasma dispar, Mycoplasma bovis, and Ureaplasma spp., Tritrichomonas foetus, Trichophyton verrucosum, Trichophyton mentagrophytes, Trichophyton, sarkisovii, Neospora caninum (formerly Toxoplasma gondii), Cryptsporidium parvum, Cryptsporidium hominis, Babesia bigemina and Babesia bovis, and Dictyocaulus viviparous (Lungworm disease).

Thus, the present invention relates to a combination vaccine for the treatment and/or prophylaxis of cattle against microbiological infections in cattle, wherein said vaccine comprises attenuated BVDV (types 1 and/or 2) as described herein and a further immunological active component effective for the treatment and/or prophylaxis of infections caused by Parainfluenza-3 Virus (PI-3), Infectious Bovine Rhinotracheitis virus (IBR), Bovine Respiratory Syncytial Virus (BRSV), Bovine Herpesvirus (BHV), Bovine Rotavirus (BRV), Bovine Enterovirus (BEV), Bovine Coronovirus (BCV), Bovine Rabies (BR), Bovie Parvovirus (PPV), Adenovirus Astrovirus, Mannheimia haemolytica (formerly Pasteurella haemolytica), Pasteurella multocida, Haemophilus somnus (Histophilus ovis and Haemophilus agni), Actinomyces (Corynebacterium), Actinomyces pyogenes, Chlamydia psittaci, Campylobacter fetus venerealis and Campylobacter fetus fetus (formerly C fetus inlestinalis), Leptospira interrogans, Leptospira pomona, and Leptospira grippotyphosa, Leptospira canicola, Leptospira grippotyphosa, Leptospira hardjo (Leptospira hardjoprajitno and Leptospira hardjo-bovis), Brucella abortus, Brucella suis and Brucella melitensis, Listeria monocytogenes, Chlamydia psittaci, Clostridium chauvoei, Clostridium septicum, Clostridium haemolyticum, Clostridium novyi, Clostridium sordellii, Clostridium perfringens, Clostridium tetani, Moraxella bovis, Klebsiella spp, Klebsiella pneumoniae, Salmonella typhimurium; Salmonella newport, Mycobacterium avium paratuberculosis, Cryptsporidium parvum, Cryptsporidium hominis, Staphylococcus aureus, Streptococcus dysgalactiae, Streptococcus uberus, Mycoplasma spp. Mycoplasma dispar, Mycoplasma bovis, and Ureaplasma spp., Tritrichomonas foetus, Trichophyton verrucosum, Trichophyton mentagrophytes, Trichophyton sarkisovii, Neospora caninum (formerly Toxoplasma gondii), Babesia bigemina and Babesia bovis, and Dictyocaulus viviparous (Lungworm disease).

According to a further embodiment, the present invention relates to a combination vaccine for the treatment and/or prophylaxis of cattle against microbiological infections in cattle, wherein said vaccine comprises attenuated BVDV (types 1 and/or 2) as described herein, and at least one antigen of Parainfluenza-3 Virus (PI-3), Infectious Bovine Rhinotracheitis virus (IBR), Bovine Respiratory Syncytial Virus (BRSV), Bovine Herpesvirus (BHV), Bovine Rotavirus (BRV), Bovine Enterovirus (BEV), Bovine Coronovirus (BCV), Bovine Rabies (BR), Bovine Parvovirus (PPV), Adenovirus Astrovirus, Mannheimia haemolytica (formerly Pasteurella haemolytica), Pasteurella multocida, Haemophilus somnus (Histophilus ovis and Haemophilus agni), Actinomyces (Corynebacterium), Actinomyces pyogenes, Chlamydia, psittaci, Campylobacter fetus venerealis and Campylobacter fetus fetus (formerly C fetus intestinalis), Leptospira interrogans, Leptospira hardjo, Leptospira pomona, and Leptospira grippotyphosa, Leptospira canicola, Leptospira grippotyphosa, Leptospira hardjo (Leptospira hardjoprajitno and Leptospira hardjo-bovis), Brucella abortus, Brucella suis and Brucella melitensis, Listeria monocytogenes, Chlamydia psittaci, Clostridium chauvoei, Clostridium septicum, Clostridium haemolyticum, Clostridium novyi, Clostridium sordellii, Clostridium perfringens, Clostridium tetani, Moraxella bovis, Klebsiella spp, Klebsiella pneumoniae, Salmonella typhimurium; Salmonella newport, Mycobacterium avium paratuberculosis, Cryptsporidium parvum, Cryptsporidium hominis, Staphylococcus aureus, Streptococcus dysgalactiae, Streptococcus uberus, Mycoplasma spp. Mycoplasma dispar, Mycoplasma bovis, and Ureaplasma spp., Tritrichomonas foetus, Trichophyton verrucosum, Trichophyton mentagrophytes, Trichophyton sarkisovii, Neospora caninum (formerly Toxoplasma gondii), Babesia bigemina and Babesia bovis, and Dictyocaulus viviparous (Lungworm disease)

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR [combo 001]. According to a preferred embodiment, the combination vaccine comprises attenuated BVDV (types 1 and/or 2) as described herein and at least one antigen of IBR [combo 002]. According to a preferred embodiment, the IBR antigen is a live modified virus [combo 003]. According to a further embodiment, the combination to vaccine of attenuated BVDV and IBR contains an antibiotic, e.g. neomycin, for preservation [combo 004].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and al least one further immunological active component effective for the treatment and/or prophylaxis of infections caused by PI-3 [combo 005]. According to a preferred embodiment, the combination vaccine comprises attenuated BVDV (types 1 and/or 2) as described herein and at least one antigen of PI-3 [combo 006]

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and al least one further immunological active component effective for the treatment and/or prophylaxis of infections caused by BRSV [combo 007]. According to a more preferred embodiment, the combination vaccine comprises attenuated BVDV (types 1 and/or 2) as described herein and at least one antigen of BRSV [combo 008].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and at least one further immunological active component effective for the treatment and/or prophylaxis of infections caused by BHV [combo 009]. According to a preferred embodiment, the combination vaccine comprises attenuated BVDV (types 1 and/or 2) as described herein and at least one antigen of BHV [combo 010].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR and PI-3 [combo 011]. According to a preferred embodiment, the combination vaccine comprises attenuated BVDV (types 1 and/or 2) as to described herein and at least one antigen of IBR and PI-3 [combo 012].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) is as described herein and at least one further immunological active component effective for the treatment and/or prophylaxis of infections caused by IBR and BRSV [combo 013]. According to a preferred embodiment, the combination vaccine comprises attenuated BVDV (types 1 and/or 2) as described herein and at least one antigen of IBR and BRSV [combo 014].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and al least one further immunological active component effective for the treatment and/or prophylaxis of infections caused by IBR and BHV [combo 015]. According to a preferred embodiment, the combination vaccine comprises attenuated BVDV (types 1 and/or 2) as described herein and at least one antigen of IBR and BHV [combo 016].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and at least one further immunological active component effective for the treatment and/or prophylaxis of infections caused by PI-3 and BRSV [combo 017]. According to a preferred embodiment, the combination vaccine comprises attenuated BVDV (types 1 and/or 2) as described herein and al least one antigen of PI-3 and BRSV [combo 018].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and at least one further immunological active component effective for the treatment and/or prophylaxis of infections caused by PI-3 and BHV [combo 019]. According to a preferred embodiment, the combination vaccine comprises attenuated BVDV (types 1 and/or 2) as described herein and at least one antigen of PI-3 and BHV [combo 020].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR, PI-3 and BRSV [combo 021]. According to a preferred embodiment, the combination vaccine comprises attenuated BVDV (types 1 and/or 2) as described herein and at least one antigen of IBR, PI-3 and BRSV [combo 022]. Preferably, all viral antigens am modified five viruses [combo 023]. According to a further embodiment of said combination vaccine, the IBR and PI-3 antigens are modified live viruses and BRSV antigen is a killed virus [combo 024]. According to a further embodiment of said combination vaccine, the IBR, PI-3 and BRSV antigens are killed viruses [combo 025].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR, BRSV and BHV [combo 026]. According to a preferred embodiment, the combination vaccine comprises attenuated BVDV (types 1 and/or 2) as described herein and at least one antigen of IBR, BRSV and BHV [combo 027].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and at least one further immunological active component effective for the treatment and/or prophylaxis of infections caused by PI-3, BRSV and BHV [combo 028]. According to a preferred embodiment, the combination vaccine comprises attenuated BVDV (types 1 and/or 2) as described herein and at least one antigen of PI-3, BRSV and BHV [combo 029].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR, PI-3 and BHV [combo 030]. According to a preferred embodiment, the combination vaccine comprises attenuated BVDV (types 1 and/or 2) as described herein and at least one antigen of IBR, PI-3 and BHV [combo 031].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR, PI-3, BRSV and BHV [combo 032]. According to a more preferred embodiment, the combination vaccine comprises attenuated BVDV (types 1 and/or 2) as described herein and at least one antigen of IBR, PI-3, BRSV and BHV [combo 033].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and at least one further immunological active component effective for the treatment and/or prophylaxis of infections caused by H. somnus [combo 034]. According to a more preferred embodiment, the combination vaccine comprises attenuated BVDV (types 1 and/or 2) as described herein and at least one antigen of H. somnus [combo 035].

According to a further embodiment the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR and M. somnus [combo 036]. According to a more preferred embodiment, the combination vaccine comprises attenuated BVDV (types 1 and/or 2) as described herein and at least one antigen of H. somnus [combo 037].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR, PI-3, and H. somnus [combo 038]. According to a more preferred embodiment, the combination vaccine comprises attenuated BVDV (types 1 and/or 2) as described herein and at least one antigen of IBR, PI-3, and H. somnus [combo 039].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and al least one further immunological active component effective for the treatment and/or prophylaxis of infections caused by IBR, PI-3, BRSV and H. somnus [combo 040]. According to a more preferred embodiment, the combination vaccine comprises attenuated BVDV (types 1 and/or 2) as described herein and at least one antigen of IBR, PI-3, BRSV and H. somnus [combo 041]. Preferably, all viral antigens are modified live viruses. According to a further embodiment of said combination vaccine, the IBR and PI-3 antigens are modified live viruses, whereas the BRSV antigen is a killed virus [combo 038]. According to a further embodiment of said combination vaccine, the IBR, PI-3 and BRSV antigens are killed viruses [combo 042]. According, to a further embodiment, any of said combination vaccines, preferably the combination vaccine that comprises killed IBR, killed PI-3 and killed BRSV as antigens, contains neomycin and thimerosal as preservatives [combo 043].

According to a further embodiment, the present invention relates to a combination vaccine for the treatment and/or prophylaxis of cattle against viral infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR, PI-3, BRSV, BHV and H. somnus [combo 044]. According to a more preferred embodiment, the combination vaccine comprises attenuated BVDV (types 1 and/or 2) as described herein and at least one antigen of IBR, PI-3, BHV and H. somnus [combo 045].

According to a further embodiment, the present invention relates to a combination vaccine for the treatment and/or prophylaxis of cattle against infections of the respiratory and reproductive systems in cattle, wherein the combination vaccine comprises attenuated BVDV (types 1 and/or 2) as described herein and at least one further immunological active component effective for the treatment and/or prophylaxis of infections caused by one or more pathogenic specie(s) of Leptospira, preferably selected from Leptospira canicola, Leptospira grippotyphosa, Leptospira borgpetersenii, Leptospira hardjo (Leptospira hardjoprajitno and Leptospira hardjo-bovis), Leptospira prajitno, Leptospira icterohaemmorrhagiae, Leptospira bovis, Leptospira interrogans and Leptospira pomona [combo 046]. According to a more preferred embodiment, the combination vaccine comprises attenuated BVDV (types 1 and/or 2) as described herein and antigen of at least one or more pathogenic specie(s) of Leptospira, preferably selected from Leptospira canicola, Leptospira grippotyphosa, Leptospira borgpetersenii, Leptospira hardjo (Leptospira hardjoprajitno and Leptospira hardjo-bovis), Leptospira prajitno, Leptospira icterohaemmorrhagiae, Leptospira bovis, Leptospira interrogans and Leptospira pomona [combo 047].

According to a further embodiment, the present invention relates to a combination vaccine for the treatment and/or prophylaxis of cattle against infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR, and one or more pathogenic specie(s) of Leptospira, preferably selected from Leptospira canicola, Leptospira grippotyphosa, Leptospira borgpetersenii, Leptospira hardjo (Leptospira hardjoprajitno and Leptospira hardjo-bovis), Leptospira prajitno, Leptospira icterohaemmorrhagiae, Leptospira bovis, Leptospira interrogans and Leptospira pomona, [combo 048]. According to a more preferred embodiment, the combination vaccine comprises attenuated BVDV (types 1 and/or 2) as described herein and at least one antigen of IBR and one or more pathogenic specie(s) of Leptospira, preferably selected from Leptospira canicola, Leptospira grippotyphosa, Leptospira borgpetersenii, Leptospira hardjo (Leptospira hardjoprajitno and Leptospira hardjo-bovis), Leptospira prajitno, Leptospira icterohaemmorrhagiae, Leptospira bovis, Leptospira interrogans and Leptospira pomona [combo 049].

According to a further embodiment, the present invention relates to a combination vaccine for the treatment and/or prophylaxis of cattle against infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR and Leptospira pomona [combo 050]. According to a more preferred embodiment, the combination vaccine comprises attenuated BVDV (types 1 and/or 2) as described herein and at least one antigen of IBR, preferably a live modified virus, and Leptospira pomona bacterin [combo 51].

According to a further embodiment, the present invention relates to a combination vaccine for the treatment and/or prophylaxis of cattle against infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR, PI-3, and one or more pathogenic specie(s) of Leptospira, preferably selected from Leptospira canicola, Leptospira grippotyphosa, Leptospira borgpetersenii, Leptospira hardjo (Leptospira hardjoprajitno and Leptospira hardjo-bovis), Leptospira prajitno, Leptospira icterohaemmorrhagiae, Leptospira bovis, Leptospira interrogans and Leptospira pomona [combo 052]. According to a more preferred embodiment the combination vaccine comprises attenuated BVDV (types 1 and/or 2) as described herein and at least one antigen of IBR, PI-3 and one or more pathogenic specie(s) of Leptospira, preferably selected from Leptospira canicola, Leptospira grippotyphosa, Leptospira borgpetersenii, Leptospira hardjo (Leptospira hardjoprajitno and Leptospira hardjo-bovis), Leptospira prajitno, Leptospira icterohaemmorrhagiae, Leptospira bovis, Leptospira interrogans and Leptospira pomona [combo 053].

According to a further embodiment, the present invention relates to a combination vaccine for the treatment and/or prophylaxis of cattle against infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR, PI-3, Leptospira canicola, Leptospira grippotyphosa, Leptospira hardjo (Leptospira hardjoprajitno and/or Leptospira hardjo-bovis), Leptospira icterohaemorrhagiae, and Leptospira pomona [combo 054]. According to a more preferred embodiment, the combination vaccine comprises attenuated BVDV (types 1 and/or 2) as described herein and at least one antigen of IBR, PI-3, Leptospira canicola, Leptospira grippotyphosa, Leptospira hardjo, Leptospira icterohaemorrhagiae, and Leptospira pomona [combo 055].

According to a further embodiment, the present invention relates to a combination vaccine for the treatment and/or prophylaxis of cattle against infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR, PI-3, BRSV and one or more pathogenic specie(s) of Leptospira, preferably selected from Leptospira canicola, Leptospira grippotyphosa, Leptospira borgpetersenii, Leptospira hardjo (Leptospira hardjoprajitno and Leptospira hardjo-bovis), Leptospira prajitno, Leptospira icterohaemmorrhagiae, Leptospira bovis, Leptospira interrogans and Leptospira pomona [combo 056]. According to a more preferred embodiment, the combination vaccine composes attenuated BVDV (types 1 and/or 2) as described herein and at least one antigen of IBR, PI-3, BRSV and one or more pathogenic species of Leptospira preferably selected from Leptospira canicola, Leptospira grippotyphosa, Leptospira borgpetersenii, Leptospira prajitno, Leptospira icterohaemmorrhagiae, Leptospira bovis, Leptospira interrogans and Leptospira pomona [combo 057].

According to a further embodiment, the present invention relates to a combination vaccine for the treatment and/or prophylaxis of cattle against infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR, PI-3, BRSV, Leptospira canicola, Leptospira grippotyphosa, Leptospira hardjo (Leptospira hardjoprajitno and/or Leptospira hardjo-bovis), Leptospira icterohaemorrhagiae, and Leptospira pomona [combo 058]. According to a more preferred embodiment, the combination vaccine comprises attenuated BVDV (types 1 and/or 2) as described herein and at least one antigen of IBR, PI-3, BRSV, Leptospira canicola, Leptospira grippotyphosa, Leptospira hardjo (Leptospira hardjoprajitno and/or Leptospira hardjo-bovis), Leptospira icterohaemorrhagiae, and Leptospira pomona [combo 059]. According to a preferred embodiment, the viral antigens are killed viruses and the bacterial antigens are bacterins [combo 060]. Preferably, said combination vaccines as described in this paragraph further contain neomycin and thimerosal as preservatives [combo 061].

According to a further embodiment, the combination vaccine comprises attenuated BVDV (types 1 and/or 2) as described herein, live modified viruses of IBR, PI-3, BRSV, and bacterin of Leptospira canicola, Leptospira grippotyphosa, Leptospira hardjo (Leptospira hardjoprajitno and/or Leptospira hardjo-bovis), Leptospira icterohaemorrhagiae and Leptospira pomona [combo 062]. According to a further preferred embodiment, the combination vaccine described in this paragraph comprises neomycin as a preservative [combo 063].

According to a further embodiment, the present invention relates to a combination vaccine for the treatment and/or prophylaxis of cattle against infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR, PI-3, BRSV, BHV and one or more pathogenic specie(s) of Leptospira, preferably selected from Leptospira canicola, Leptospira grippotyphosa, Leptospira borgpetersenii, Leptospira hardjo (Leptospira hardjoprajitno and Leptospira hardjo-bovis), Leptospira prajitno, Leptospira icterohaemmorrhagiae, Leptospira bovis, Leptospira interrogans and Leptospira pomona. [combo 064]. According to a more preferred embodiment the combination vaccine comprises attenuated BVDV (types 1 and/or 2) as described herein and at least one antigen of IBR, PI-3, BRSV, BHV and one or more pathogenic specie(s) of Leptospira, preferably selected from Leptospira canicola, Leptospira grippotyphosa, Leptospira borgpetersenii, Leptospira hardjo (Leptospira hardjoprajitno and Leptospira hardjo-bovis), Leptospira prajitno, Leptospira icterohaemmorrhagiae, Leptospira bovis, Leptospira interrogans and Leptospira pomona [combo 065].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by one or more pathogenic specie(s) of Leptospira, as mentioned above, and H. somnus [combo 066]. According to a more preferred embodiment, the combination vaccine comprises attenuated BVDV (types 1 and/or 2) as described herein and at least antigen of one or more pathogenic species of Leptospira, as mentioned above, and H. somnus [combo 067].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by IBR and one or more pathogenic specie(s) of Leptospira, as mentioned above, and H. somnus [combo 068]. According to a more preferred embodiment the combination vaccine comprises attenuated BVDV (types 1 and/or 2) as described herein and at least one antigen of IBR and one or more pathogenic species of Leptospira, as mentioned above, and H. somnus [combo 069].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by IBR, PI-3 and one or more pathogenic specie(s) of Leptospira, as mentioned above, and H. somnus [combo 070]. According to a more preferred embodiment, the combination vaccine comprises attenuated BVDV (types 1 and/or 2) as described herein and at least one antigen of IBR, PI-3, and one or more pathogenic species of Leptospira, as mentioned above, and H. somnus [combo 071].

According to a further embodiment, the present invention relates to a combination vaccine for the treatment and/or prophylaxis of cattle against infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR, PI-3, Leptospira canicola, Leptospira grippotyphosa, Leptospira hardjo (Leptospira hardjoprajitno and/or Leptospira hardjo-bovis), Leptospira icterohaemorrhagiae, Leptospira pomona and H. somnus [combo 072]. According to a more preferred embodiment, the combination vaccine comprises attenuated BVDV (types 1 and/or 2) as described herein and at least one antigen of IBR, PI-3, Leptospira canicola, Leptospira grippotyphosa, Leptospira hardjo (Leptospira hardjoprajitno and/or Leptospira hardjo-bovis), Leptospira icterohaemorrhagiae, Leptospira pomona, and H. somnus [combo 073].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by IBR, PI-3, BRSV, and one or more pathogenic specie(s) of Leptospira, as mentioned above, and H. somnus [combo 074]. According to a preferred embodiment, the combination vaccine comprises attenuated BVDV (types 1 and/or 2) as described herein and at least one antigen of IBR, PI-3, BRSV, and one or more pathogenic species of Leptospira, as mentioned above, and H. somnus [combo 075].

According to a further embodiment, the present invention relates to a combination vaccine for the treatment and/or prophylaxis of cattle against infections of the respiratory and reproductive systems in cattle, wherein the combination vaccine comprises attenuated BVDV (types 1 and/or 2) as described herein and at least one further immunological active component effective for the treatment and/or prophylaxis of infections caused by IBR, PI-3, BRSV, Leptospira canicola, Leptospira grippotyphosa, Leptospira hardjo (Leptospira hardjo prajitno and/or Leptospira hardjo-bovis), Leptospira icterohaemorrhagiae, Leptospira pomona and H. somnus [combo 076]. According to a more preferred embodiment, the combination vaccine comprises attenuated BVDV (types 1 and/or 2) as described herein and at least one antigen of IBR, PI-3, BRSV, Leptospira canicola, Leptospira grippotyphosa, Leptospira hardjo, Leptospira icterohaemorrhagiae, Leptospira pomona and H. somnus [combo 077].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by IBR, PI-3, BRSV, BHV and one or more pathogenic specie(s) of Leptospira, as mentioned above, and H. somnus [combo 078]. According to a more preferred embodiment, the combination vaccine comprises attenuated BVDV (types 1 and/or 2) as described herein and at least one antigen of IBR, PI-3, BRSV, BHV and one or more pathogenic species of Leptospira, as mentioned above, and H. somnus [combo 079].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by one or more pathogenic specie(s) of Leptospira, as mentioned above, and Campylobacter fetus [combo 080]. According to a more preferred embodiment, the combination vaccine comprises attenuated BVDV (types 1 and/or 2) as described herein and at least antigen of one or more pathogenic species of Leptospira, as mentioned above, and Campylobacter fetus [combo 081].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by Leptospira canicola, Leptospira grippotyphosa, Leptospira hardjo (Leptospira hardjoprajitno and/or Leptospira hardjo-bovis), Leptospira icterohaemorrhagiae, Leptospira pomona and Campylobacter fetus [combo 082]. According to a preferred embodiment, the combination vaccine comprises attenuated BVDV (types 1 and/or 2) as described herein and at least antigen of Leptospira canicola, Leptospira grippotyphosa, Leptospira hardjo (Leptospira hardjoprajitno and Leptospira hardjo-bovis), Leptospira icterohaemorrhagiae, Leptospira pomona and Campylobacter fetus [combo 083]. According to a more preferred embodiment, the bacterial antigens are chemically inactivated, aluminum hydroxide adsorbed, whole cultures of said bacteria [combo 084]. According to a further preferred embodiment, said combination vaccine comprises gentamicin and Amphotericin B as preservatives [combo 085].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least, one further immunological active component effective for the treatment and/or prophylaxis of infections caused by IBR and one or more pathogenic specie(s) of Leptospira, as mentioned above, and Campylobacter fetus [combo 086]. According to a more preferred embodiment, the combination vaccine comprises attenuated BVDV (types 1 and/or 2) as described herein and at least one antigen of IBR and one or more pathogenic species of Leptospira, as mentioned above, and Campylobacter fetus [combo 087].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by IBR, PI-3 and one or more pathogenic specie(s) of Leptospira, as mentioned above, and Campylobacter fetus [combo 088]. According to a more preferred embodiment, the combination vaccine comprises attenuated BVDV (types 1 and/or 2) as described herein and at least one antigen of IBR, PI-3, and one or more pathogenic species of Leptospira, as mentioned above, and Campylobacter fetus [combo 089].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of to infections caused by IBR, PI-3, BRSV, and one or more pathogenic specie(s) of Leptospira, as mentioned above, and Campylobacter fetus [combo 090]. According to a more preferred embodiment, the combination vaccine comprises attenuated BVDV (types 1 and/or 2) as described herein and at least one antigen of IBR, PI-3, BRSV, and one or more pathogenic species of Leptospira, as mentioned above, and Campylobacter fetus [combo 091].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused, by IBR, PI-3, BRSV, BHV, one or more pathogenic specie(s) of Leptospira, as mentioned above, and Campylobacter fetus [combo 092]. According to a more preferred embodiment, the combination vaccine comprises attenuated BVDV (types 1 and/or 2) as described herein and at least one antigen of IBR, PI-3, BRSV, BHV and one or more pathogenic species of Leptospira, as mentioned above, and Campylobacter fetus [combo 093].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by one or more pathogenic specie(s) of Leptospira, as mentioned above, H. somnus and Campylobacter fetus [combo 094]. According to a more preferred embodiment, the combination vaccine comprises attenuated BVDV (types 1 and/or 2) as described herein and at least antigen of one or more pathogenic species of Leptospira, as mentioned above, H. somnus and Campylobacter fetus [combo 095].

According to a further embodiment, the present invention, relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by IBR, one or more pathogenic specie(s) of Leptospira, as mentioned above, H. somnus and Campylobacter fetus [combo 096]. According to a more preferred embodiment, the combination vaccine comprises attenuated BVDV (types 1 and/or 2) as described herein and at least one antigen of IBR, one or more pathogenic species of Leptospira, as mentioned above, H. somnus and Campylobacter fetus [combo 097].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by IBR, PI-3, and one or more pathogenic specie(s) of Leptospira, as mentioned above, H. somnus and Campylobacter fetus [combo 098]. According to a preferred embodiment the combination vaccine comprises attenuated BVDV (types 1 and/or 2) as described herein and at least one antigen of IBR, PI-3, and one or more pathogenic species of Leptospira, as mentioned above, H. somnus and Campylobacter fetus [combo 099].

According to a further embodiment the present invention relates to a combination vaccine for the treatment and/or prophylaxis of cattle against microbiological infections in cattle, wherein the combination vaccine comprises attenuated BVDV (types 1 and/or 2) as described herein and at least one further immunological, active component effective for the treatment and/or prophylaxis of infections caused by IBR, PI-3, Leptospira canicola, Leptospira grippotyphosa, Leptospira hardjo, Leptospira icterohaemorrhagiae, Leptospira pomona, H. somnus and Campylobacter fetus [combo 100]. According to a further embodiment, the combination vaccine comprises attenuated BVDV (types 1 and/or 2) as described herein and at least one antigen of IBR, PI-3, Leptospira canicola, Leptospira grippotyphosa, Leptospira hardjo (Leptospira hardjoprajitno and/or Leptospira hardjo-bovis), Leptospira icterohaemorrhagiae, Leptospira pomona, H. somnus and Campylobacter fetus [combo 101].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by IBR, PI-3, BRSV, and one or more pathogenic specie(s) of Leptospira, as mentioned above, H. somnus and Campylobacter fetus [combo 102]. According to a more preferred embodiment, the combination vaccine comprises attenuated BVDV (types 1 and/or 2) as described herein and at least one antigen of IBR, PI-3, BRSV, and one or more pathogenic species of Leptospira, as mentioned above, H. somnus and Campylobacter fetus [combo 100].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by IBR, PI-3, BRSV, Leptospira canicola, Leptospira grippotyphosa, Leptospira hardjo (Leptospira hardjoprajitno and/or Leptospira hardjo-bovis), Leptospira icterohaemorrhagiae, Leptospira Pomona, H. somnus and Campylobacter fetus [combo 103]. According to a further embodiment, the combination vaccine comprises attenuated BVDV (types 1 and/or 2) as described herein and at least one antigen of IBR, PI-3, BRSV, Leptospira canicola, Leptospira grippotyphosa, Leptospira hardjo (Leptospira hardjoprajitno and/or Leptospira hardjo-bovis), Leptospira icterohaemorrhagiae, Leptospira pomona, H. somnus and Campylobacter fetus [combo 104].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by IBR, PI-3, BRSV, BHV and one or more pathogenic specie(s) of Leptospira, as mentioned above, H. somnus and Campylobacter fetus [combo 105]. According to a more preferred embodiment the combination vaccine comprises attenuated BVDV (types 1 and/or 2) as described herein and at least one antigen of IBR, PI-3, BRSV, BHV and one or more pathogenic species of Leptospira, as mentioned above, H. somnus and Campylobacter fetus [combo 106].

According to a further embodiment, the present invention relates to a combination vaccine for the treatment and/or prophylaxis of cattle against infections of the respiratory and reproductive systems in cattle, wherein the combination vaccine comprises attenuated BVDV (types 1 and/or 2) as described herein and at least one further immunological active component effective for the treatment and/or prophylaxis of infections caused by BHV, BRSV, PI-3, IBR, Leptospira canicola, Leptospira grippotyphosa, Leptospira borgpetersenii Leptospira hardjo (Leptospira hardjoprajitno and/or Leptospira hardjo-bovis), Leptospira prajitno, Leptospira icterohaemmorrhagiae, Leptospira bovis, Leptospira interrogans and Campylobacter fetus [combo 107]. According to a more preferred embodiment, the combination vaccine comprises attenuated BVDV as described herein and at least one antigen of BHV, BRSV, IBR, PI-3, Leptospira canicola, Leptospira grippotyphosa, Leptospira borgpetersenii Leptospira hardjo (Leptospira hardjoprajitno and/or Leptospira hardjo-bovis), Leptospira prajitno, Leptospira icterohaemmorrhagiae, Leptospira borgpetersenii, Leptospira bovis, Leptospira interrogans and Campylobacter fetus [combo 108].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by Pasteurella haemolytica and Pasteurella multocida [combo 109]. According to a preferred embodiment, the combination vaccine comprises attenuated BVDV (types 1 and/or 2) as described herein and at least one antigen of Pasteurella haemolytica bacterin and Pasteurella multocida bacterin, [combo 110] According to a further preferred embodiment, said combination vaccine comprises neomycin and thimerosal as preservatives [combo 111].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, Pasteurella haemolytica and Pasteurella multocida [combo 112]. According to a preferred embodiment, the combination vaccine comprises attenuated BVDV (types 1 and/or 2) as described herein and at least one antigen of IBR, preferably as live modified viruses, and Pasteurella haemolytica bacterin and Pasteurella multocida bacterin [combo 113]. According to a further preferred embodiment, said combination vaccine comprises neomycin and thimerosal as preservatives [combo 114].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, PI-3, Pasteurella haemolytica and Pasteurella multocida [combo 115]. According to a preferred embodiment, the combination vaccine comprises attenuated BVDV (types 1 and/or 2) as described herein and at least one antigen of IBR, PI-3, preferably as live modified viruses, and Pasteurella haemolytica bacterin and Pasteurella multocida bacterin [combo 116]. According to a further preferred embodiment, said combination vaccine comprises neomycin and thimerosal as preservatives [combo 117].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, PI-3, BRSV, Pasteurella haemolytica and Pasteurella multocida [combo 118]. According to a preferred embodiment, the combination vaccine comprises attenuated BVDV (types 1 and/or 2) as described herein and at least one antigen of IBR, PI-3, BRSV, preferably as live modified viruses, and Pasteurella haemolytica bacterin and Pasteurella multocida bacterin [combo 119]. According to a further preferred embodiment, said combination vaccine comprises neomycin and thimerosal as preservatives [combo 120].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, PI-3, BRSV, BHV, Pasteurella haemolytica and Pasteurella multocida [combo 121]. According to a preferred embodiment, the combination vaccine comprises attenuated BVDV (types 1 and/or 2) as described herein and at least one antigen of IBR, PI-3, BRSV, BHV, preferably as live modified viruses, and Pasteurella haemolytica bacterin and Pasteurella multocida bacterin [combo 122]. According to a further preferred embodiment, said combination vaccine comprises neomycin and thimerosal as preservatives [combo 123].

According to a further embodiment the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by Mycoplasma bovis [combo 124]. According to a preferred embodiment, the combination vaccine comprises attenuated BVDV (types 1 and/or 2) as described herein and at least antigen of Mycoplasma bovis [combo 125].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, and Mycoplasma bovis [combo 126]. According to a preferred embodiment, the combination vaccine comprises attenuated BVDV (types 1 and/or 2) as described herein and at least antigen of IBR, preferably as live modified viruses, and Mycoplasma bovis [combo 127].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, PI-3, and Mycoplasma bovis [combo 128]. According to a preferred embodiment, the combination vaccine comprises attenuated BVDV (types 1 and/or 2) as described herein and at least one antigen of IBR, PI-3, preferably as live modified viruses, and Mycoplasma bovis [combo 129].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, PI-3, BRSV, and Mycoplasma bovis [combo 130]. According to a preferred embodiment, the combination vaccine comprises attenuated BVDV (types 1 and/or 2) as described herein and at least one antigen of IBR, PI-3, BRSV, preferably as live modified viruses, and Mycoplasma bovis [combo 131].

According to a further embodiment the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, PI-3, BRSV, BHV, and Mycoplasma bovis [combo 132]. According to a preferred embodiment, the combination vaccine comprises attenuated BVDV (types 1 and/or 2) as described herein and at least one antigen of IBR, PI-3, BRSV, BHV, preferably as live modified viruses, and Mycoplasma bovis [combo 133].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by Pasteurella haemolytica, Pasteurella multocida and Mycoplasma bovis [combo 134]. According to a preferred embodiment, the combination vaccine comprises attenuated BVDV (types 1 and/or 2) as described herein and at least one antigen of Pasteurella haemolytica, Pasteurella multocida and Mycoplasma bovis [combo 135].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, Pasteurella haemolytica, Pasteurella multocida and Mycoplasma bovis [combo 136]. According, to a preferred embodiment, the combination vaccine comprises attenuated BVDV (types 1 and/or 2) as described herein and at least one antigen of IBR, preferably as live modified viruses, Pasteurella haemolytica, Pasteurella multocida and Mycoplasma bovis [combo 137].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, PI-3, Pasteurella haemolytica, Pasteurella multocida and Mycoplasma bovis [combo 138]. According to a preferred embodiment, the combination vaccine comprises attenuated BVDV (types 1 and/or 2) as described herein and at least one antigen of IBR, PI-3, preferably as live modified viruses, Pasteurella haemolytica, Pasteurella multocida and Mycoplasma bovis [combo 139].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, PI-3, BRSV, Pasteurella haemolytica, Pasteurella multocida and Mycoplasma bovis [combo 140]. According to a preferred embodiment, the combination vaccine comprises attenuated BVDV (types 1 and/or 2) as described herein and at least one antigen of IBR, PI-3, BRSV, preferably as live modified viruses, Pasteurella haemolytica, Pasteurella multocida and Mycoplasma bovis [combo 141].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, PI-3, BRSV, BHV, Pasteurella haemolytica, Pasteurella multocida and Mycoplasma bovis [combo 140]. According to a preferred embodiment, the combination vaccine comprises attenuated BVDV (types 1 and/or 2) as described herein and at least one antigen of IBR, PI-3, BRSV, BHV, preferably as live modified viruses, Pasteurella haemolytica, Pasteurella multocida and Mycoplasma bovis [combo 141].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by Pasteurella haemolytica, Pasteurella multocida and H. somnus [combo 142]. According to a preferred embodiment, the combination vaccine comprises attenuated BVDV (types 1 and/or 2) as described herein and at least one antigen of Pasteurella haemolytica, Pasteurella multocida and H. somnus [combo 143].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, Pasteurella haemolytica, Pasteurella multocida and H. somnus [combo 144]. According to a preferred embodiment, the combination vaccine comprises attenuated BVDV (types 1 and/or 2) as described herein and at least one antigen of IBR, preferably as live modified virus, and Pasteurella haemolytica, Pasteurella multocida and H. somnus [combo 145].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, PI-3, Pasteurella haemolytica, Pasteurella multocida and H. somnus [combo 146]. According to a preferred embodiment, the combination vaccine comprises attenuated BVDV (types 1 and/or 2) as described herein and at least one antigen of IBR, PI-3, preferably as live modified viruses, and Pasteurella haemolytica, Pasteurella multocida and H. somnus [combo 147].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, PI-3, BRSV, Pasteurella haemolytica, Pasteurella multocida and H. somnus [combo 148]. According to a preferred embodiment, the combination vaccine comprises attenuated BVDV (types 1 and/or 2) as described herein and at least one antigen of IBR, PI-3, BRSV, preferably as live modified viruses, and Pasteurella haemolytica, Pasteurella multocida and H. somnus [combo 149].

According to a further embodiment, the present invention relates to a combination vaccine for the treatment and/or prophylaxis of cattle against infections in cattle, wherein the combination vaccine comprises attenuated BVDV (types 1 and/or 2) as described herein and at least one further immunological active component effective for the treatment and/or prophylaxis of infections caused by IBR, PI-3, BRSV, BHV, Pasteurella haemolytica, Pasteurella multocida and H. somnus [combo 150]. According to a preferred embodiment, the combination vaccine comprises attenuated BVDV (types 1 and/or 2) as described herein and at least one antigen of IBR, PI-3, BRSV, BHV, preferably as live modified viruses, and Pasteurella haemolytica, Pasteurella multocida and H. somnus [combo 151].

According to a further embodiment, the present invention relates to a combination vaccine according to any one of [combo 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 and 151], that further comprises an immunological active component effective for the treatment and/or prophylaxis of infections caused by one or more pathogenic species of Leptospira, preferably selected from Leptospira canicola, Leptospira grippotyphosa, Leptospira borgpetersenii, Leptospira hardjo, Leptospira prajitno, Leptospira icterohaemmorrhagiae, Leptospira bovis, Leptospira interrogans and Leptospira pomona [combo 152]. According to a further embodiment, the present invention relates to a combination vaccine according to any one of [combo 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 and 151], that further comprises antigen of one or more specie(s) of Leptospira, preferably selected from Leptospira canicola, Leptospira grippotyphosa, Leptospira borgpetersenii, Leptospira hardjo (Leptospira hardjoprajitno and Leptospira hardjo-bovis), Leptospira prajitno, Leptospira icterohaemmorrhagiae, Leptospira bovis, Leptospira interrogans and Leptospira pomona, [combo 153].

According to a further embodiment, the present invention, relates to a combination vaccine according to any one of [combo 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 and 151], that further comprises an immunological active component effective for the treatment and/or prophylaxis of infections caused by Campylobacter fetus [combo 154]. According to a further embodiment, the present invention relates to a combination vaccine according to any one of [combo 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 and 151], that further comprises antigen of Campylobacter fetus [combo 155].

According to a further embodiment, the present invention relates to a combination vaccine according to any one of [combo 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 and 151], that further comprises an immunological active component effective for the treatment and/or prophylaxis of infections caused by one or more pathogenic specie(s) of Leptospira, preferably selected from Leptospira canicola, Leptospira grippotyphosa, Leptospira borgpetersenii, Leptospira hardjo (Leptospira hardjoprajitno and Leptospira hardjo-bovis), Leptospira prajitno, Leptospira icterohaemmorrhagiae, Leptospira bovis, Leptospira interrogans and Leptospira pomona, and Campylobacter fetus [combo 156]. According to a further embodiment, the present invention relates to a combination vaccine according to any one of [combo 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 and 151], that further comprises antigen of Campylobacter fetus and of one or more specie(s) of Leptospira, preferably selected from Leptospira canicola, Leptospira grippotyphosa, Leptospira borgpetersenii, Leptospira hardjo (Leptospira hardjoprajitno and Leptospira hardjo-bovis), Leptospira prajitno, Leptospira icterohaemmorrhagiae, Leptospira bovis, Leptospira interrogans and Leptospira pomona, and [combo 157].

According to a further embodiment, the present invention relates to a combination vaccine for the treatment and/or prophylaxis of cattle against, microbiological infections in cattle, wherein the combination vaccine comprises attenuated BVDV (types 1 and/or 2) as described herein and at least one further immunological active component effective for the treatment and/or prophylaxis of infections caused by Clostridium perfringens, preferably Types A, C and/or D [combo 158]. According to a preferred embodiment, the combination vaccine comprises attenuated BVDV (types 1 and/or 2) as described herein and toxins of Clostridium perfringens Types C and D [combo 254]. According to a more preferred embodiment, said vaccine comprises antigens, preferably toxins, of Clostridium perfringens, preferably Types A, B, C, and/or D [combo 159].

According to a further embodiment, die present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156 and 157], that further comprises an immunological active component effective for the treatment and/or prophylaxis of infections caused by infections caused by Clostridium perfringens, preferably Types A, C and/or D [combo 160]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156 and 157], feat further comprises antigen of Clostridium perfringens, preferably, Types A, C, and/or D [combo 161]. According to a further embodiment, the present invention, relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156 and 157], that further comprises antigen of Clostridium perfringens Types, B, C, and/or D [combo 162].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Clostridium perfringens Types A, C and/or D, and Clostridium tetani [combo 163]. According to a preferred embodiment, the combination vaccine comprises attenuated BVDV (types 1 and/or 2) as described herein and toxins of Clostridium perfringens Types A, C and/or D, and Clostridium tetani [combo 164]. According to a more preferred embodiment, said vaccine comprises antigens, preferably toxins, of Clostridium perfringens Types A, B, C, and/or D, and Clostridium tetani [combo 165].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156 and 157], that further comprises an immunological active component effective for the treatment and/or prophylaxis of infections caused by infections caused by Clostridium perfringens Types A, C and/or D, and Clostridium tetani [combo 166]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156 and 157], that further comprises antigen of Clostridium perfringens Types A, C, and/or D, and Clostridium tetani [combo 167]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156 and 157], that further comprises antigen of Clostridium perfringens Types A, B, C, and/or D, and Clostridium tetani [combo 168].

According to a further embodiment the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Clostridium chauvoei, Clostridium septicum, Clostridium novyi, Clostridium sordellii, and Clostridium perfringens Types A, C and/or D [combo 169]. According to a preferred embodiment, the combination vaccine comprises attenuated BVDV (types 1 and/or 2) as described herein and antigens, preferably toxins, of Clostridium chauvoei, Clostridium septicum, Clostridium novyi, Clostridium sordellii, and Clostridium perfringens Types A, C and/or D [combo 170].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156 and 157], that further comprises an immunological active component effective for the treatment and/or prophylaxis of infections caused by infections caused by Clostridium chauvoei, Clostridium septicum, Clostridium novyi, Clostridium sordellii, and Clostridium perfringens Types A, C and/or D [combo 171]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156 and 157], that further comprises antigen of Clostridium chauvoei, Clostridium septicum, Clostridium novyi, Clostridium sordellii, and Clostridium perfringens Types A, C and/or D [combo 172]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156 and 157], that further comprises antigen of Clostridium perfringens Types, A, B, C, and/or D, Clostridium chauvoei, Clostridium septicum, Clostridium novyi, Clostridium sordellii and Clostridium tetani [combo 173].

According to more preferred embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Clostridium chauvoei, Clostridium septicum, Clostridium novyi, Clostridium sordellii, Clostridium perfringens Types A, C and/or D and Mycoplasma bovis [combo 174]. According to a preferred embodiment, the combination vaccine comprises attenuated BVDV (types 1 and/or 2) as described herein and antigens, preferably toxins, of Clostridium chauvoei, Clostridium septicum, Clostridium novyi, Clostridium sordellii, and Clostridium perfringens Types A, C and/or D and Mycoplasma bovis [combo 175].

According to more preferred embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Clostridium chauvoei, Clostridium septicum, Clostridium novyi, Clostridium sordellii, Clostridium perfringens Types A, C and/or D, and H. somnus. [combo 176]. According to a preferred embodiment, the combination vaccine comprises attenuated BVDV (types 1 and/or 2) as described herein and antigens, preferably toxins, of Clostridium chauvoei, Clostridium septicum, Clostridium novyi, Clostridium sordellii, and Clostridium perfringens Types C and D and H. somnus. [combo 177].

According to more preferred embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Clostridium chauvoei, Clostridium septicum, Clostridium novyi, Clostridium sordellii, Clostridium perfringens Types A, C and/or D, Mycoplasma bovis, and H. somnus [combo 178]. According to a preferred embodiment, the combination vaccine comprises attenuated BVDV (types 1 and/or 2) as described herein and antigens, preferably toxins, of Clostridium chauvoei, Clostridium septicum, Clostridium novyi, Clostridium sordellii, and Clostridium perfringens Types A, C and/or D, Mycoplasma bovis, and H. somnus [combo 179].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Salmonella, preferably Salmonella dublin, Salmonella newport and Salmonella typhimurium [combo 180]. According to a preferred embodiment, the combination vaccine comprises attenuated BVDV (types 1 and/or 2) as described herein and toxins of Salmonella, preferably Salmonella dublin, Salmonella newport, and Salmonella typhimurium [combo 181].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178 and 179], that further comprises an immunological active component effective for the treatment and/or prophylaxis of infections caused by infections caused by Salmonella, preferably Salmonella dublin, Salmonella newport and Salmonella typhimurium [combo 182]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178 and 179], that further comprises antigen, preferably a toxin, of Salmonella, preferably Salmonella dublin, Salmonella newport and Salmonella typhimurium [combo 183].

According to a preferred embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Pasteurella haemolytica, Pasteurella multocida, Salmonella, preferably Salmonella dublin, Salmonella newport and Salmonella typhimurium [combo 184]. According to a preferred embodiment, the combination vaccine comprises attenuated BVDV (types 1 and/or 2) as described herein and Pasteurella haemolytica, Pasteurella multocida, Salmonella, preferably Salmonella dublin, Salmonella newport, and Salmonella typhimurium Bacterin-Toxoid [combo 185]. According to more preferred embodiment, said combination vaccine comprises multiple isolates of Pasteurella haemolytica Type A1 and an associated toxoid fraction, and single isolates of P multocida, S dublin, and S typhimurium [combo 186].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Moraxella bovis and/or Klebsiella spp., preferably Klebsiella pneumoniae [combo 187]. According to a preferred embodiment, the combination vaccine comprises attenuated BVDV (types 1 and/or 2) as described herein and toxins of Moraxella bovis and/or Klebsiella spp. preferably Klebsiella pneumoniae [combo 188].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, and 186], that further comprises an immunological active component effective for the treatment and/or prophylaxis of infections caused by infections caused by Moraxella bovis and/or Klebsiella spp., preferably Klebsiella pneumoniae [combo 189]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 163, 164, 165, 169, 170, 174, 175, 176, 177, 178, 179, 180, 181, 184, 185 and 186], that further comprises antigen, preferably a toxin, of Moraxella bovis and/or Klebsiella spp., preferably, Klebsiella pneumoniae [combo 190].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Escherichia coli [combo 191]. According to a preferred embodiment, the combination vaccine comprises attenuated BVDV (types 1 and/or 2) as described herein and toxins of Escherichia coli [combo 192].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189 and 190], that further comprises an immunological active component effective for the treatment and/or prophylaxis of infections caused by infections caused by Escherichia coli [combo 193]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 305, 106, 107, 108, 109, 110, 111, 112, 313, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189 and 190], that further comprises antigen, preferably a toxin, of Escherichia coli [combo 194].

According to a preferred embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Pasteurella haemolytica, Pasteurella multocida, Salmonella dublin, Salmonella typhimurium and Eschericha coli [combo 195]. According to a preferred embodiment, the combination vaccine comprises attenuated BVDV (types 1 and/or 2) as described herein and Pasteurella haemolytica, Pasteurella multocida, Salmonella dublin, Salmonella typhimurium and Escherichia coli Bacterin-Toxoid [combo 196]. According to more preferred embodiment, said combination vaccine comprises multiple isolates of Pasteurella haemolytica Type A1 and an associated toxoid fraction, and single isolates of P multocida, S dublin, and S typhimurium [combo 197].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by bovine Rotavirus [combo 198]. According to a preferred embodiment, the combination vaccine comprises attenuated BVDV (types 1 and/or 2) as described herein and antigen of bovine Rotavirus [combo 199].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 195, 196, and 197], that further comprises an immunological active component effective for the treatment and/or prophylaxis of infections caused by infections caused by bovine Rotavirus [200]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196 and 197], that further comprises antigen of bovine Rotavirus [combo 201].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by bovine Coronavirus [combo 202]. According to a preferred embodiment, the combination vaccine comprises attenuated BVDV (types 1 and/or 2) as described herein and antigen of bovine Coronavirus [combo 203].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196 and 197], that further comprises an immunological active component effective for the treatment and/or prophylaxis of infections caused by infections caused by bovine Coronavirus [combo 204]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, and 197], that further comprises antigen of bovine Coronavirus [combo 205].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by bovine Coronavirus and bovine Rotavirus [combo 206]. According to a preferred embodiment, the combination vaccine comprises attenuated BVDV (types 1 and/or 2) as described herein and antigen of bovine Coronavirus and bovine Rotavirus [combo 207].

According to a further embodiment, the present invention, relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196 and 197], that further comprises an immunological active component effective for the treatment and/or prophylaxis of infections caused by infections caused by bovine Coronavirus and bovine Rotavirus [combo 208]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, and 197], that further comprises antigen of bovine Coronavirus and bovine Rotavirus [combo 209].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological, active component effective for the treatment and/or prophylaxis of infections caused by Cryptosporidium parvum [combo 210]. According to a preferred embodiment, the combination vaccine comprises attenuated BVDV (types 1 and/or 2) as described herein and antigen of Cryptosporidium parvum [combo 211].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184 184, 185, 186, 187, 188, 189, 190, 191, 192, 193, 194, 195, 196, 197, 198, 199, 200, 201, 202, 203, 204, 205, 206, 207, 208 and 209], that further comprises an immunological active component effective for the treatment and/or prophylaxis of infections caused by infections caused by Cryptosporidium parvum [combo 212]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 198, 199, 200, 201, 202, 203, 204, 205, 206, 207, 208 and 209], that further comprises antigen of Cryptosporidium parvum [combo 213].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Cryptosporidium hominis [combo 214]. According to a preferred embodiment, the combination vaccine comprises attenuated BVDV (types 1 and/or 2) as described herein and antigen of Cryptosporidium hominis [combo 215].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184 184, 185, 186, 187, 188, 189, 190, 191, 192, 193, 194, 195, 196, 197, 198, 199, 200, 201, 202, 203, 204, 205, 206, 207, 208 and 209], that further comprises an immunological active component effective for the treatment and/or prophylaxis of infections caused by infections caused by Cryptosporidium hominis [combo 216]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 07.1, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208 and 209], that further comprises antigen of Cryptosporidium hominis [combo 217].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Cryptosporidium parvum and Cryptosporidium hominis [combo 218]. According to a preferred embodiment, the combination vaccine comprises attenuated BVDV (types 1 and/or 2) as described herein and antigen of Cryptosporidium parvum and Cryptosporidium hominis [combo 219].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184 184, 185, 186, 187, 188, 189, 190, 191, 192, 193, 194, 195, 196, 197, 198, 199, 200, 201, 202, 203, 204, 205, 206, 207, 208 and 209], that further comprises an immunological active component effective for the treatment and/or prophylaxis of infections caused by infections caused by Cryptosporidium parvum and Cryptosporidium hominis [combo 220]. According to a further embodiment the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208 and 209], that further comprises antigen of Cryptosporidium parvum and Cryptosporidium hominis [combo 221].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Mycobacterium avium paratuberculosis [combo 222]. According to a preferred embodiment, the combination vaccine comprises attenuated BVDV (types 1 and/or 2) as described herein and antigen of Mycobacterium avium paratuberculosis [combo 223].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 21.8, 219, 220 and 221], that further comprises an immunological active component effective for the treatment and/or prophylaxis of infections caused by infections caused by Mycobacterium avium paratuberculosis [combo 224]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 198, 199, 200, 201, 202, 203, 204 205, 206, 207, 208, 209, 210, 211, 212, 213, 214, 215, 216, 217, 218, 219, 220 and 221], that further comprises antigen of Mycobacterium avium paratuberculosis [combo 225].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Adenovirus [combo 226]. According to a preferred embodiment, the combination vaccine comprises attenuated BVDV (types 1 and/or 2) as described herein and antigen of Adenovirus [combo 227].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224 and 225], that further comprises an immunological active component effective for the treatment and/or prophylaxis of infections caused by infections caused by Adenovirus [combo 228]. According to a further embodiment the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 198, 199, 200, 201, 202, 203, 204 205, 206, 207, 208, 209, 210, 211, 212, 213, 214, 215, 216, 217, 218, 219, 220, 221, 222, 223, 224, and 225], that further comprises antigen of Adenovirus [combo 229].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Astrovirus [combo 230]. According to a preferred embodiment, the combination vaccine comprises attenuated BVDV (types 1 and/or 2) as described herein and antigen of Astrovirus [combo 231].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228 and 229], that further comprises an immunological active component effective for the treatment and/or prophylaxis of infections caused by infections caused by Astrovirus [combo 232]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 198, 199, 200, 201, 202, 203, 204 205, 206, 207, 208, 209, 210, 211, 212, 213, 214, 215, 216, 217, 218, 219, 220, 221, 222, 223, 224, 225, 226, 227, 228 and 229], that further comprises antigen of Astrovirus [combo 233].

According to a further embodiment the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by bovine Parvovirus [combo 234]. According to a preferred embodiment, the combination vaccine comprises attenuated BVDV (types 1 and/or 2) as described herein and antigen of bovine Parvovirus [combo 235].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232 and 233], that further comprises an immunological active component effective for the treatment and/or prophylaxis of infections caused by infections caused by bovine Parvovirus [combo 236]. According to a further embodiment, the present invention relates to a combination vaccine according to any one of [combo 001, 002, 003, 004, 005, 006, 007, 008, 009, 010, 011, 012, 013, 014, 015, 016, 017, 038, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 198, 199, 200, 201, 202, 203, 204 205, 206, 207, 208, 209, 210, 211, 212, 213, 214, 215, 216, 217, 218, 219, 220, 221, 222, 223, 224, 225, 226, 227, 228, 229, 230, 231, 232, and 233], that further comprises antigen of bovine Parvovirus [combo 237].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Cryptosporidium parvum, Adenovirus, Astrovirus, bovine Parvovirus and Mycobacterial avium paratuberculosis [combo 238]. According to a preferred embodiment, the combination vaccine comprises attenuated BVDV (types 1 and/or 2) as described herein and antigen of Cryptosporidium parvum, Adenovirus, Astrovirus, bovine Parvovirus and Mycobacterium avium paratuberculosis [combo 239].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Escherichia coli, Salmonella spp., preferably Salmonella dublin, Salmonella typhimurium and Salmonella newport, bovine Rotavirus and bovine Coronavirus, Cryptosporidium parvum, Adenovirus, Astrovirus, bovine Parvovirus and Mycobacterium avium paratuberculosis [combo 240]. According to a preferred embodiment, the combination vaccine comprises attenuated BVDV (types 1 and/or 2) as described herein and antigen of Escherichia coli, Salmonella spp., preferably Salmonella dublin, Salmonella typhimurium and Salmonella newport, bovine rotavirus and bovine Coronavirus, Cryptosporidium parvum, Adenovirus, Astrovirus, bovine Parvovirus and Mycobacterium avium paratuberculosis [combo 241].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Streptococcus spp., preferably Streptococcus uberus and/or Streptococcus dysgalactiae [combo 242]. According to a preferred embodiment, the combination vaccine comprises attenuated BVDV (types 1 and/or 2) as described herein and antigen of Streptococcus spp., preferably Streptococcus uberus and/or Streptococcus dysgalactiae, [combo 243]. According to a more preferred embodiment, the combination vaccine comprises attenuated BVDV (types 1 and/or 2) as described herein and antigen of Streptococcus spp., preferably Streptococcus uberus and/or Streptococcus dysgalactiae [combo 244].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological, active component effective for the treatment and/or prophylaxis of infections caused by Streptococcus spp., preferably Streptococcus uberus and/or Streptococcus dysgalactiae and/or Staphylococcus aureus [combo 245]. According to a more preferred embodiment, the combination vaccine comprises attenuated BVDV (types 1 and/or 2) as described herein and antigen, of Streptococcus spp., preferably Streptococcus uberus and/or Streptococcus dysgalactiae, and/or Staphylococcus aureus [combo 246].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, and 241], that further comprises an immunological active component effective for the treatment and/or prophylaxis of infections caused by infections caused by Streptococcus spp., preferably Streptococcus uberus and/or Streptococcus dysgalactiae, and/or Staphylococcus aureus [combo 247]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 198, 199, 200, 201, 202, 203, 204 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240 and 241], that further comprises antigen of Streptococcus spp., preferably Streptococcus uberus and/or Streptococcus dysgalactiae, and/or Staphylococcus aureus [combo 248]. According to a further embodiment the present, invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 198, 199, 200, 201, 202, 203, 204 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240 and 241], that further comprises antigen of Streptococcus spp., preferably Streptococcus uberus and/or Streptococcus dysgalactiae, and/or Staphylococcus aureus [combo 249].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Streptococcus spp., preferably Streptococcus uberus and/or Streptococcus dysgalactiae, Staphylococcus aureus, Klebsiella spp. and Mycoplasma spp. [combo 250]. According to a preferred embodiment, the combination vaccine comprises attenuated BVDV (types 1 and/or 2) as described herein and antigen of Streptococcus spp., preferably Streptococcus uberus, Streptococcus dysgalactiae, and/or Streptococcus aureus, Klebsiella spp. and Mycoplasma spp. [combo 251]. According to a more preferred embodiment, the combination vaccine comprises attenuated BVDV (types 1 and/or 2) as described herein and antigen of Streptococcus spp., preferably Streptococcus uberus and/or Streptococcus dysgalactiae, Staphylococcus aureus, Klebsiella spp., Mycoplasma spp. and endotoxin [combo 252].

According to a further embodiment, the present invention relates to a combination vaccine for the treatment and/or prophylaxis of cattle against microbiological infections in cattle, wherein the combination vaccine comprises attenuated BVDV (types 1 and/or 2) as described herein and at least one further immunological active component effective for the treatment and/or prophylaxis of infections caused by Trichophyten and Microsporus, preferably selected from Trichophyton verrucosum, Trichophyton mentagrophytes, Trichophyton equinum, Trichophyton sarkisovii, Microsporum canis, Microsporum canis var. obesum, Microsporum canis var. distortum, and Microsporum gypseum [combo 253]. According to a preferred embodiment, the combination vaccine comprises attenuated BVDV (types 1 and/or 2) as described herein and antigen of Trichophyten, and Microsporus, preferably selected from Trichophyton verrucosum, Trichophyton mentagrophytes, Trichophyton equinum, Trichophyton sarkisovii, Microsporum canis, Microsporum canis var. obesum, Microsporum canis var. distortum, and Microsporum gypseum [combo 254].

According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, and 251], that further comprises an immunological active component effective for the treatment and/or prophylaxis of infections caused by infections caused by Trichophyton and Microsporus, preferably selected from Trichophyton verrucosum, Trichophyton mentagrophytes, Trichophyton equinum, Trichophyton sarkisovii, Microsporum canis, Microsporum canis var. obesum, Microsporum canis var. distortum, and Microsporum gypseum [combo 255]. According to a further embodiment, the present invention relates to a combination vaccine according to any one of [combo 001, 002, 003, 004, 005, 006, 007, 008, 009, 010, 011, 012, 013, 014, 015, 016, 017, 018, 019, 020, 021, 022, 023, 024, 025, 026, 027, 028, 029, 030, 031, 032, 033, 034, 035, 036, 037, 038, 039, 040, 041, 042, 043, 044, 045, 046, 047, 048, 049, 050, 051, 052, 053, 054, 055, 056, 057, 058, 059, 060, 061, 062, 063, 064, 065, 066, 067, 068, 069, 070, 071, 072, 073, 074, 075, 076, 077, 078, 079, 080, 081, 082, 083, 084, 085, 086, 087, 088, 089, 090, 091, 092, 093, 094, 095, 096, 097, 098, 099, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 254, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, and 252], that further comprises antigen of Trichophyten and Microsporus, preferably selected from Trichophyton verrucosum, Trichophyton mentagrophytes, Trichophyton equinum, Trichophyton sarkisovii, Microsporum canis, Microsporum canis var. obesum, Microsporum canis var. distortum, and Microsporum gypseum [combo 256].

According to further embodiment, the source of the combination vaccine is, Alpha 7™, ALPHA-7/MB™, ALPHA-CD™, BAR-VAC® 7, BAR-VAC® 7/SOMNUS, BAR-VAC® 8, BAR-VAC® CD, BAR-VAC® C/DT, BREED-BACK™ FP 10, BREED-BACK™ FP 10 HS, BREED-BACK™ FP 5, BREED-BACK™ FP 5 HS, BREED-BACK-10™, CALIBER® 3, CALIBER® 7, ELITE 4™, ELITE 9™, ELITE 9-HS™ EXPRESS® 10, EXPRESS® 10-HS, EXPRESS® 3, EXPRESS® 3/Lp, EXPRESS 4®, EXPRESS® 5, EXPRESS® 5-HS, EXPRESS® 5-PHM, EXPRESS° I, EXPRESS® I/LP, OCU-GUARD® MB, PULMO-GUARD™ MpB, PULMO-GUARD™ PH-M, PULMO-GUARD™ PH-M/SDT, PULMO-GUARD™ PHM-1, TETGUARD™, VIBRIO-LEPTO-5™ (all of Boehringer Ingelheim, St. Joseph, Mo.); Cobalt™ 7, I-Site™, Lepto 5, Master Guard® Preg 5, Master Guard® 10, Master Guard® 10+ Vibrio, Master Guard® J5, P.H.M. Bac® 1, Prevent 6™, Respromune® 4, Respromune® 4+ Somnumune® (IM, SC), Respromune® 5 I-B-P+BRSV, Respromune® 5+L5, Respromune® 5+L5 Somnus, Respromune® 5+Somnumune, Respromune® 5+VL5, Respromune® 8, Respromune® 9, Respromune® 10, Scour Vac™ 4, Scour Vac™ 9, Scour Vac™ E coli+C, Somnumune®, Titanium™ 3, Titanium™ 4, Titanium™ 4 L5, Titanium™ 5, Titanium™ 5 L5, Titanium® 5+P.H.M. Bac®-1, Titanium™ BRSV 3. Titanium™ IBR, Titanium™ IBR-LP (all of Agri Laboratories Inc., St. Joseph, Mo.); Herd-Vac® 3, Herd-Vac® 3 S, Herd-Vac® 8, Herd-Vac® 9, Surround™ 4, Surround™ 4+HS, Surround™ 8, Surround™ 9, Surround™ 9+HS, Surround™ HS, Surround™ L5, Surround™ V-L5 (all of BioCor, Omaha, Nebr. (Pfizer)); Mycomune® (Biomune Co., Lenexa, Kans.); Bluetongue vaccine, Bovine Virus Diarrhea Vaccine, Campylobacter fetus bacterin-bovine. Essential 1, Essential 2, Essential 2+P, Essential 3, Essential 3+T, Essential 4, Lepto-5, Mannheimia haemolytica-Pasteurella multocida bacterin, Pre-breed 6, Pre-breed 8, Respira-1, Respira-3, Wart Vaccine (all of Colorado Serum Company, Denver, Colo.); Pyramid® 3, Pyramid® 4, Pyramid® 4+Presponse® SQ, Pyramid® 5, Pyramid® 8, Pyramid® 9, Pyramid® IBR, Pyramid® IBR+Lepto, Triangle® 1+Type II BVD, Triangle® 3+VL5, Triangle® 4+HS, Triangle® 4+PH/HS, Triangle® 4+ PH-K, Triangle® 4+Type II BVD, Triangle® 9+HS, Triangle® 9+PH-K, Triangle®+Type II BVD, Trichguard®, Trichguard®+V5L, TriVib 5L® (all of Fort Dodge Animal Health, Overland Park, Kans. (Wyeth)); J-5 Escherichia coli Bacterin, Serpens Species Bacterin; Staphylococcus aureus bacterin-toxoid (all of Hygieia Biological Laboratories, Woodland, Calif.); Endovac-Bovi® with Immuneplus® (Immvac. Inc., Columbia, Mo.); 20/20 Vision® with Spur®, L5 SQ, Neoguard™, MasterGuard® Preg 5, Once PMH®, Once PMH® SQ, Vibralone™-L5, Vision® 7 Somnus with Spur®, Vision® 7 with Spur®, Vision® 8 Somnus with Spur®, Vision® 8 with Spur®, Vision® CD-T with Spur®, Vision® CD with Spur®, Vista™ IBR SQ, Vista™ 3 SQ, Vista™ 5 SQ, Vista™ 5 L5 SQ, Vista™ Once SQ, VL5 SQ, Volar®, (all of Intervet Inc., Millsboro, Del.); Vac®, Reliant® 3, Reliant® 4, Reliant® IBR, Reliant® IBR/BVD, Reliant® IBR/Lepto, Reliant® Plus BVD-K (Dual IBR™), Reliant® Plus (Dual IBR™). Respishield™ 4, Respishield™ 4 L5, Respishield™ HM (all of Merial LTD, Duluth, Ga.); Arsenal® 4.1, Arsenal® IBR, Arsenal® IBR BVD, Bovine Pili Shield™, Bovine Pili Shield™+C, Clostri Shield® 7, Clostri Shield® BCD, Fusogard®, Lepto Shield™ 5, Pinkeye Shield™ XT4, Salmo Shield® T, Salmo Shield® TD, Scour Bos™ 4, Scour Bos™ 9, Somnu Shield™, Trep Shield™ HW, Vib Shield® L5, Vib Shield® Plus, Vib Shield® Plus L5, Vira Shield® 2, Vira Shield® 2+BRSV, Vira Shield® 3, Vira Shield® 3+VL5, Vira Shield® 4, Vira Shield® 4+L5, Vira Shield® 5, Vira Shield® 5+L5, Vira Shield® 5+L5 Somnus, Vira Shield® 5+Somnus, Vira Shield® 5+VL5, Vira Shield® 5+VL5 Somnus, Vira Shield® 6, Vira Shield® 6+Somnus, Wart Shield™ (all of Novartis Animal Health, Basel, Switzerland): Bovi-K® 4, Bovi-Shield™ 3, Bovi-Shield™ 4, Bovi-Shield™ BRSV, Bovi-Shield® FP™ 4+L5, Bovi-Shield® GOLD 3, Bovi-Shield® GOLD 5, Bovi-Shield® GOLD FP™ 5 L5, Bovi-Shield® GOLD FP™ 5 VL5, Bovi-Shield® Gold IBR-BVD, Bovi-Shield® Gold IBR-BVD-BRSV-LP, Bovi-Shield™ IBR, Bovi-Shield™ IBR-BRSV-LP, Bovi-Shield™ IBR-BVD, Bovi-Shield™ IBR-BVD-BRSV-LP, Bovi-Shield™ IBR-PI3-BRSV, Calf-Guard®, CattleMaster® 4, CattleMaster® 4+L5, CattleMaster® 4+VL5, CattleMaster® BVD-K, CattleMaster® Gold FP™ 5, CattleMaster® Gold FP™ 5 L5, Defensor® 3, Fortress® 7, Fortress® 8, Fortress® CD, Leptoferm®-5, One Shot®, One Shot Ultra™ 7, One Shot Ultra™ 8, PregGnard™ FP 9, PregGuard® Gold FP™ 10, Resvac® BRSV/Somubac®, Res Vac® 4/Somubac®, ScourGuard 3® (K), ScourGuard 3® (K)/C, Somubac®, Spirovac®, Spirovac® L5, Spirovac® VL5, Stay Bred™ VL5, TSV-2™, Ultrabac® 7, Ultrabac® 7/Somubac®, Ultrabac® 8, Ultrabac® CD, UltraChoice™ 7, UltraChoice™ 8, UltraChoice™ CD, Upjohn J-5 Bacterin™, Vibrin® (all of Pfizer Inc., New York, N.Y.); Covexin® 8 Vaccine, Electroid® 7 Vaccine, Electroid® D, Guardian™, Jencine® 2, Jencine® 3, Jencine® 4, Nasalgen® IP Vaccine, Piliguard® Pinkeye-1. Trivalent, Piliguard® Pinkeye+7, Piliguard® Pinkeye Triview®, Siteguard® G, Siteguard® MLG Vaccine (all of Schering-Plough Animal Health Corporation, Kenilworth, N.J.): Myco-Bac™ B, Poly-Bac B® 3, Poly-Bac B® Somnus, Super Poly-Bac B® Somnus (ail of Texas Vet Lab, Inc., San Angelo, Tex.), Virabos™-3 with Immunostim®, Virabos™-4+H. somnus with Immunostim®, Virabos™-4 with Immunostim® (all of Bioniche Animal Health, Athens, Ga.), wherein attenuated BVDV, as described herein, is added. Alternatively, when BVDV antigen is present in any of those vaccines, attenuated BVDV, as described herein, is added, or BVDV present any of those vaccines is substituted by attenuated BVDV, as described herein.

### Formulations:

An important aspect of the present invention is the preparation of the combination vaccine(s). The skilled person knows additional components which may be comprised in said composition (see also Remington's Pharmaceutical Sciences. (1990), 18th ed. Mack Publ., Easton). The expert may use known injectable, physiologically acceptable sterile solutions. For preparing a ready-to-use solution for parenteral injection or infusion, aqueous isotonic solutions, such as e.g. saline or corresponding plasma protein solutions are readily available. The pharmaceutical compositions may be present as lyophylisates or dry preparations, which can be reconstituted with a known injectable solution directly before use under sterile conditions, e.g. as a kit of parts.

In addition, the immunogenic and vaccine compositions of the present invention can include one or more veterinary-acceptable carriers. As used herein, “a veterinary-acceptable carrier” includes any and all solvents, dispersion media, coatings, adjuvants, stabilizing agents, diluents, preservatives, antibacterial and antifungal agents, isotonic agents, adsorption delaying agents, and the like.

Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

Adjuvants include, but are not limited to the RIBI adjuvant system (Ribi Inc.), alum, aluminum hydroxide gel, Cholesterol, oil-in water emulsions, water-in-oil emulsions such as, e.g., Freund's complete and incomplete adjuvants, Block co-polymer (CytRx, Atlanta Ga.), SAF-M (Chiron, Emeryville Calif.), CARBOPOL®, AMPHIGENO adjuvant, saponin, Quil A, QS-21 (Cambridge Biotech Inc., Cambridge Mass.), GPI-0100 (Galenica Pharmaceuticals, Inc., Birmingham, Ala.) or other saponin fractions, monophosphoryl lipid A, Avridine lipid-amine adjuvant, heat-labile enterotoxin from E. coli (recombinant or otherwise), cholera toxin, or muramyl dipeptide, among many others.

The immunogenic compositions can further include one or more other immunomodulatory agents such as, e.g., interleukins, interferons, or other cytokines. The immunogenic compositions can also include Gentamicin and Merthiolate. While the amounts and concentrations of adjuvants and additives useful in the context of the present invention can readily be determined by the skilled artisan, the present invention contemplates compositions comprising from about 50 ug to about 2000 ug of adjuvant and preferably about 250 ug/ml dose of the vaccine composition. In another preferred embodiment, the present invention contemplates vaccine compositions comprising from about 1 ug/ml to about 60 ug/ml of antibiotics, and more preferably less than about 30 ug/ml of antibiotics.

For example, in a composition according to the invention, 10⁴ to 10⁶ TCID₅₀ of attenuated BVDV may be solved in 25% (v/v) SGS (Sucrose 75 mg, Gelatine 20 mg, Potassium hydroxide 0.274 mg, L-glutamic acid 0.72 mg, Potassium dihydrogen phosphate 0.536 mg, Dipotassium phosphate 1.254 mg, and 2 ml with water for injection), and 5% (v/v) cell culture medium, and 1 ml with water for injection. This is further mixed with at least one further antigen of a bovine pathogen, as listed above.

According to a further embodiment the combination vaccine is first dehydrated. If the composition is first lyophilized or dehydrated by other methods, then, prior to vaccination, said composition is rehydrated in aqueous (e.g. saline, PBS (phosphate buffered saline)) or non-aqueous solutions (e.g. oil emulsion (mineral oil, or vegetable/metabolizable oil based/single or double emulsion based), aluminum-based, carbomer based adjuvant).

### Dose and Administration:

According to the present invention, an effective amount of a combination vaccine administered to cattle, including pregnant cows and calves nursing pregnant cows, provides effective immunity against microbiological infections caused by BVDV and at least one further pathogen as listed above. Preferred combinations of antigens for the treatment and prophylaxis of microbiological diseases in cattle are listed above.

According to one embodiment, the combination vaccine is administered to calves in two doses at an interval of about 3 to 4 weeks. For example, the first administration is performed when the animal is about 1 to about 3 months of age. The second administration is performed about 1 to about 4 weeks after the first administration of the combination vaccine. According to a further embodiment, revaccination is performed in an interval of 6 to 12 month after administration of the second dose. In a preferred embodiment, the first administration is performed about 5 weeks prior to animal breeding. The second administration is performed about 2 weeks prior to animal breeding. Administration of subsequent vaccine doses is preferably done on a 6 month to an annual basis. In another preferred embodiment, animals vaccinated before the age of about 6 months should be revaccinated after 6 months of age. Administration of subsequent vaccine doses is preferably done on an annual basis.

The amount of combination vaccine that is effective depends on the ingredients of the vaccine and the schedule of administration. Typically, when an inactivated virus or a modified live virus preparation is used in the combination vaccine, an amount of the vaccine containing about 10² to about 10⁹ TCID₅₀ per dose, preferably about 10³ to about 10⁸ TCID₅₀ per dose, more preferably, about 10⁴ to about 10⁸ TCID₅₀ per dose. For example, about 10⁵ to about 10⁸ TCID₅₀ per dose of attenuated BVDV (types 1 and 2) is effective when administered twice to the animal during a period of about 3 to 4 weeks. In general, inactivated antigen is normally used in higher amounts than live modified viruses. Typically, when bacterial antigen is used in the combination vaccine, the vaccine contains an amount of about 10³ to about 10⁹ colony forming units (CFU) per dose, preferably, about 10⁴ to about 10⁸ (CFU) per dose, more preferably about 10⁵ to about 10⁶ (CFU) per dose.

In the event, the combination vaccine comprises live modified IBR, the amount of IBR antigen is preferably in a range of about 10⁵ to 10^{7.5} TCID₅₀ per dose. In the event, the combination vaccine comprises live modified PI3, the amount of PI3 antigen is preferably in a range of about 10⁷ to 10⁹ TCID₅₀ per dose. In the event, the combination vaccine comprises live modified BRSV, the amount of BRSV antigen is preferably in a range of about 10^{4.5} to 10^{6.5} TCID₅₀ per dose. In the event, the combination vaccine comprises killed IBR, the amount of IBR antigen is preferably in a range of about 10^{7.0} to 10^{9.0} TCID₅₀ per dose. In the event, the combination vaccine comprises killed PI3, the amount of PI3 antigen is preferably in a range of about 10^{7.2} to 10^{9.2} TCID₅₀ per dose. In the event, the combination vaccine comprises killed BRSV, the amount of BRSV antigen is preferably in a range of about 10^{5.0} to 10^{7.5} TCID₅₀ per dose. In the event, the combination vaccine comprises killed Leptospira spp, the amount of each Leptospira spp. antigen is preferably in a range of about 10^{7.0} to 10¹⁰ (CFU) per dose. In the event, the combination vaccine comprises killed H. somnus the amount of H. somnus antigen is preferably in a range of about 10^{6.0} to 10⁹ (CFU) per dose.

The composition according to the invention may be applied intradermally, intratracheally, intravaginally, intramuscularly or intranasally, and preferably intramuscularly or intranasally. In an animal body, it can prove advantageous to apply the pharmaceutical compositions as described above via an intravenous or by direct injection into target tissues. For systemic application, the intravenous, intravascular, intramuscular, intranasal, intraarterial, intraperitoneal, oral, or intrathecal routes are preferred. A more local application can be effected subcutaneously, intradermally, intracutaneously, intracardially, intralobally, intramedullarly, intrapulmonarily or directly in or near the tissue to be treated (connective-, bone-, muscle-, nerve-, epithelial tissue). Depending on the desired duration and effectiveness of the treatment, the compositions according to the invention may be administered once or several times, as well as intermittently, for instance on a daily basis for several days, weeks or months and in different dosages.

### Methods for Treatment:

Yet another important embodiment of the invention is a method for the prophylaxis or treatment of diseases caused by BVDV, and further bovine pathogenic microorganism(s), wherein an attenuated BVDV as described herein and further immunological active components effective for the treatment and/or prophylaxis of the infection caused by said further bovine pathogenic microorganism is administered to an animal in need thereof at a suitable dose, as known to the skilled person.

### EXAMPLES

The following examples serve to further illustrate the present invention; but the same should not be construed as limiting the scope of the invention disclosed herein.

### Example 1

### BVDV XIKE-B

### Foetopathogenicity Assessment in Pregnant Heifers

BVDV XIKE-B, an RNase negative mutant of the highly pathogenic BVDV type 2 isolate NewYork'93/C was recovered from the infectious cDNA clone pKANE40B and showed wild type-like (wt-like) growth characteristics in tissue culture. In animal experiments, the mutant virus was found to be considerably attenuated so that it represented a promising candidate for development of a live attenuated vaccine virus (Meyer et al, 2002). To test whether this attenuated virus is still able to cross the placenta and infect the fetus, pregnant heifers were infected with XIKE-B. As a control, wild type BVDV recovered from cDNA clone pKANE40A was used. The respective virus named XIKE-A expresses an active E^{rns} RNase in the infected cell. The study aimed to assess the safety of XIKE-A and XIKE-B in pregnant animals.

### Experimental Design

Ten pregnant heifers were selected from a BVDV negative herd. The following groups of 5 heifers were included in the trial:

| | No. | Inoculation | Virus |
|---|---|---|---|
| Group 1: | 5 | One i.n. administration, | XIKE-A |
| | | 3 ml in each nostril | |
| Group 2: | 5 | One i.n. administration, | XIKE-B |
| | | 3 ml in each nostril | |

Heifers were moved to the experimental facilities 8 days before inoculations. Pregnancy status was confirmed after transport into the experimental facility. Heifers were between days 60 and 90 of gestation on the day of inoculation. Inoculation took place for ail animals at one point of time.

Heifers were monitored for the presence of clinical signs of BVDV infection including abortions during the observation period. Blood samples were collected from the animals for serology, antigen defection and white blood cells were counted. The experiment was terminated 9 weeks after infection. Non-aborted cows were slaughtered, the uterus examined and collected. Fetal organ to samples were collected during routine necropsy and examined for BVDV infection.

The presence of fetal infection was the main evaluation parameter, composed from the number of BVDV-related cow mortalities, the number of BVDV-related abortions and the number of BVDV positive fetuses at termination. In addition to the main parameter, clinical signs characteristic for BVDV infection, viraemia and white blood cell counts in cows, and rectal temperature after challenge were evaluated.

### Animals

Heifers were purchased from a farm free of BVDV. Only animals, which met the following inclusion criteria, were used.

### Inclusion Criteria

free of BVD antibodies; each individual was tested in the serum antibody test prior to transport and at the initiation of the study (at the animal test facility).
Free of BVDV; plasma and/or buffy-coat preparation from each individual was tested by a suitable test.
Clinically healthy at the initiation of the study judged upon physical examination. The health examination of the animals was accomplished in accordance with the current; generally accepted veterinary practice.
Pregnancy confirmed by physical examination before inoculation. Pregnancy was between 60-90 days at the time of inoculation, proven by insemination records.

Test Strain A

| | |
|---|---|
| Description: | XIKE A, live virus BVDV strain |
| Composition: | Experimental material comprising of cell culture |
| | supernatant of low passaged XIKE-A |
| BVD components: | BVDV type II strain: XIKE-A |
| Supplied by: | Dr. Gregor Meyers, “Bundesforschungsanstalt fur |
| | Viruskrankheiten der Tiere” (BFAV), Paul- |
| | Ehrlich-Straβe 28, 72076 Tubingen, Germany |
| Applied BVD virus | Type 1 strain: 10⁵ TCID₅₀/6 ml (TCID = Tissue |
| dose: | Culture Infective Dose) |
| Applied vaccine | 3 ml per nostril |
| volume: | |
| Application route: | Intranasal |
| Preparation of | The inoculum was sent in a pre-diluted frozen form |
| dosage form: | in a 50 ml vial on dry ice and was stored at −70° C. |
| | before inoculation. Immediately before inoculation |
| | of Group 1 heifers, the material was thawed |
| | avoiding local temperatures above 37° C. After no |
| | ice was visible in the fluid, material was gently |
| | stirred and immediately used for inoculation of the |
| | animals. |
| Unused inoculum: | The volume of the unused material was measured |
| | and split on two aliquots before immediate freezing |
| | in dry ice or liquid nitrogen and stored for re- |
| | titration purposes. Virus and contaminated plastic |
| | or glassware were incubated with an appropriate |
| | volume of an 8-10% formaldehyde solution for |
| | at least 24 hours at room temperature before |
| | discarding in order to inactivate viruses. |

Test Strain B

| | |
|---|---|
| Description: | XIKE B, live virus BVDV strain |
| Composition: | Experimental material comprising of cell culture |
| | supernatant of low passaged XIKE-B |
| BVD components: | BVDV type II strain: XIKE-B |
| Supplied by: | Dr. Gregor Meyers, “Bundesforschungsanstalt fur |
| | Viruskrankheiten der Tiere” (BFAV), Paul- |
| | Ehrlich-Straβe 28, 72076 Tubingen, Germany |
| Applied BVD virus | Type 1 strain: 10⁵ TCID₅₀/6 ml (TCID = Tissue |
| dose: | Culture Infective Dose) |
| Applied vaccine | 3 ml per nostril |
| volume: | |
| Application route: | Intranasal |
| Preparation of | The inoculum was sent in a pre-diluted frozen form |
| dosage form: | in a 50 ml vial on dry ice and was stored at −70° C. |
| | before inoculation. Immediately before inoculation |
| | of Group 2 heifers, the material was thawed |
| | avoiding local temperatures above 37° C. After no |
| | ice was visible in the fluid, material was gently |
| | stirred and immediately used for inoculation of the |
| | animals. |
| Unused vaccine: | The volume of the unused material was measured |
| | and split on two aliquots before immediate freezing |
| | in dry ice or liquid nitrogen and stored for re- |
| | titration purposes. Virus and contaminated plastic |
| | or glassware was incubated with an appropriate |
| | volume of an 8-10% formaldehyde solution for |
| | at least 24 hours at room temperature before |
| | discarding in order to inactivate viruses. |

### Pregnancy Control

Pregnancy was confirmed immediately before inoculation.

### Inoculation of Heifers

The date of inoculation is Day 0 of the experiment.

In each nostril, 3 ml of the test material was administered intranasally by syringe without needle. Each time a new sterile syringe was taken. Administration was performed during the aspiration phase in order to minimize loss of fluid via expiration of material.

### Post-Inoculation Observations

### Collection and Examination of Blood Samples

Blood was collected following standard, aseptic procedures (disinfecting the bleeding site). A new sterile syringe and needle was used for each animal.

### Blood Collection to Prepare Serum

At least 10 ml blood was collected from the heifers immediately before inoculation, then weekly after infection and at the termination of the study. Serum was stored at −20° C. until required.

### Blood Collection for Leukocyte Counts and Buffy Coat Preparations

For leukocyte counting, 3 ml blood was transferred immediately after collection to suitable sterile vessels (Venoject, Terumo Europe N.V., Leuven, Belgium), pre-filled with 0.06 ml EDTA (0.235 MOL/L).

For buffy coat preparations, at least 15 ml blood was transferred immediately after collection to suitable sterile vessels, pre-filled with 0.1 ml Heparin solution (Na-heparin for inj., 5 000 IU/ml lot. A7B163A exp. date: November 2000: Gedeon Richter RT, Budapest, Hungary) yielding at least 20 IU Heparin per ml blood in the blood sample. The content was carefully mixed thereafter.

For preparation of buffy coats and leukocyte counting, blood was collected from the heifers
on every day, between Day 0 and Day 14 after infection;
on every second day, between Day 15 and Day 40, or until all animals were negative for virus isolation for three consecutive sampling time points.

### Preparation of Serum

Blood was allowed to clot at room temperature, and separated by centrifugation. Each serum sample was divided into two aliquots of at least 2 ml each. One set of aliquots was assayed for BVDV specific antibodies by ELISA. The rest of the sera was frozen and stored at −20° C. until required.

### Leukocyte Counts

Leukocyte counts were determined with a coulter-counter semi-automated electronic device (Diatron Minicell-16, Messtechnik GmbH, Wien, Austria) with a claimed accuracy of 0.1×10⁹/l, 100/μl. The instrument was used (calibration and leukocyte-counts) according to the manufacturer's recommendations.

### Reparation of Buffy Coats

Heparin blood samples were transported to the laboratory as soon as possible. Buffy coat preparation procedure, following a standard laboratory procedure was performed under aseptic conditions (sterile pipettes, handling, clean bench etc.).

The obtained buffy coats were re-suspended in a small volume (2 ml) of RPMI 1640 and frozen at −70° C. in two aliquots of 0.5 ml. The residual 1 ml bully coats were immediately used for determination of blood cell associated BVDV by co-cultivation in a permissive cell culture.

### BVD Serum Antibody ELISA-Test

Each serum sample was tested for the presence of BVDV-antibodies using a suitable and validated ELISA lest (Svanovir™ BVDV antibody test Cat#10-2200-10). Each test was validated and performed according to the manufacturer's recommendations. Positive samples were diluted according to the log₂ scale to determine BVDV antibody titers.

### BVD Antigen Assay(s)

Each buffy coat sample was assayed for the presence of BVDV by co-cultivation of the freshly prepared buffy-coats with susceptible cells or a cell-line. No freezing was allowed before co-cultivation.

Plasma was collected and provided to Man-Gene from each sample.

### Clinical Observations

### Observation of Heifers

Animals were examined daily from Day 0-42 post inoculation for the presence of clinical symptoms by a sufficiently trained veterinarian.

All clinical signs were recorded and described by their nature, consistence/touch, seventy (mild, medium or severe) location, size of the area affected, and they were scored according to agreed and standard definitions. Special attention was paid to respiratory signs (respiration, its rate; nasal or ocular discharge; conjunctivitis, sneezing, coughing, etc.) and diarrhea,

### Rectal Temperatures

Rectal temperatures were measured daily in each heifer, at the same hour of the day (preferably in the morning) from 5 days prior to the inoculation until 21 days post infection.

Daily measurement of rectal temperature was continued until each animal had rectal temperatures below or equal to 39° C. for at least 3 consecutive days.

### Detection of Interrupted Pregnancy

Pregnancy was confirmed and suspicion for abortion or resorption of the fetus was established by rectal examination. A trained veterinarian examined all animals at inoculation, 1, and 2 months post-inoculation. The examination was carried out according to the generally accepted veterinary practice.

Heifers were examined daily for any sign of abortion until termination of the study (8-12 weeks post-challenge).

### Termination of the Study

The study was terminated by slaughtering the heifers and extracting the fetuses. Fetuses and fetal material were transferred into closed transport containers marked with the number of the cow and to the date/time. Containers were transported to a selected necropsy room.

Necropsy of the heifers was not required. Necropsy was performed on fetuses, findings recorded and a panel of samples collected as described below.

### Post-Mortem Examination

A detailed necropsy of the experimental animals was done in each case of death. Post-mortem examinations were carried out by an experienced veterinary surgeon and the data, were recorded on appropriate data sheets. Further laboratory tests were performed according to the clinical signs and lesions observed. If the diagnosis of the necropsy referred to a disease caused by microbial agent the diagnosis was verified by an appropriate test, specific for the agent. Each tissue sample was collected in at least 2 separate, labeled containers and snap-frozen in liquid nitrogen. Samples were stored at −70° C. until required.

### Aborted Fetuses and Study Termination

At least the following tissue samples were collected from the fetuses:
exudate from the peritoneal cavity or thorax, if present,
mesenteric lymph nodes,
spleen,
thymus,
cerebellum,
kidney,
bone marrow from the sternum,
sample from the placenta, if available.

### Dead or Sacrificed Heifers

At least the following tissue samples were collected:
blood for buffy coat, if available,
blood for serum, if available,
Peyer's patches,
mesenteric lymph nodes,
spleen,
kidney,
uterus, including a sample from the placenta, if available.

### Storage and Transport of Samples

| Samples: | Storage: |
|---|---|
| Serum | −20° C. |
| Buffy coat | −70° C. |
| Virus | −70° C. |
| Tissue from heifers | −70° C. |
| Tissue from fetuses | −70° C. |

Samples were sent for laboratory analysis as required by the sponsor. The choice of samples and the timing of transport were agreed with the study monitor or the project manager. As a matter of general principle, samples coming from aborted material or from new-born calves were investigated as soon as possible,

### Results

### Summary of BVD Related Clinical and Laboratory Data

| | | |
|---|---|---|
| Animal | | |
| No. | Conclusion | BVD |

Group 1

| | | |
|---|---|---|
| 526 | BVD abortion (uterus with placenta post- | NT |
| | mortem) | (no sample found) |
| 598 | BVD abortion (foetus post-mortem) | +(foetus)* |
| 615 | Clinical BVD abortion | −(foetus)* |
| 618 | BVD abortion (foetus post-mortem) | −(foetus)* |
| 626 | Died due to BVD | + (foetus)/+(heifer) |

Group 2

| | | |
|---|---|---|
| 469 | Clinical BVD abortion | −(foetus)* |
| 565 | Expected BVD abortion; non-viable foetus | +(foetus) |
| 588 | Normal | −(foetus) |
| 608 | Normal | +(foetus) |
| 619 | BVD abortion (foetus post-mortem) | − (foetus)* |

NT: not tested
*Foetuses were autolysed at the time of sampling

### Conclusion:

The study aimed to assess the safety of XIKE-A and XIKE-B in pregnant animals. Ten pregnant heifers were selected from a BVDV negative herd. Two groups of 5 heifers were included in the trial: one was inoculated with XIKE-A the other with XIKE-B virus strain. Heifers were between days 60 and 90 of gestation on the day of inoculation. Heifers were monitored for the presence of clinical signs of BVDV infection, including abortions during the observation period. Blood samples were collected from the animals for serology, antigen detection and white blood cells were counted. The experiment was terminated 9 weeks after infection. Non-aborted cows were slaughtered, the uterus examined and collected. Fetal organ samples were collected during routine necropsy and examined for BVDV infection.

The XIKE-B virus proved to be less pathogenic than XIKE-A, nevertheless BVD related abortion and infection of the foetus was observed in the XIKE-B group, too. Therefore it can be concluded that the inactivation of the E^{rns} RNase does not prevent fetal, infection.

### Example 2

### BVDV XIKE-A-NdN

### Foetopathogenicity Assessment in Pregnant Heifers

The N^{pro} gene has been shown to be nonessential for growth of CSFV in tissue culture (Tratschin et al., 1998). Even though a proof for BVDV attenuation in consequence of N^{pro} deletion is still missing, a role of tins protein in the interaction between virus and host seemed to be possible and was actually indicated by recent experiments for CSFV (Mayer et al., 2004, Rüggli et al., 2003). We therefore wanted to investigate, whether the deletion of the major part of the N^{pro} coding sequence leads to a virus that no longer infects the fetus in pregnant heifers. The N^{pro} gene, except for the 5′ terminal 4 codons, was deleted from the full length cDNA clone pKANE40A according to standard procedures. The resulting mutant full length clone was used as template for in vitro transcription and the resulting cRNA was transfected into MDBK cells as described (Meyer et al., 2002). The recovered virus was amplified in tissue culture and then used in the animal experiment described below. BVDV XIKE-B served as a control since it was shown before that it is able to cross the placenta (EXAMPLE 1).

### Objective(s)/Purpose of the Study

The study aims to assess the safety of a live attenuated BVDV with a genomic deletion of most of the N^{pro} coding region in pregnant animals.

Material and Methods applied are described in Example 1

### Study Design

Eight pregnant heifers were assigned at random to two groups. They were treated and observed according to the following schedule:

| | Group 1 | Group 2 |
|---|---|---|
| N | 5 | 3 |
| Treatments | XIKE-A-NdN | XIKE-B/control |

| | |
|---|---|
| Route | Intramuscular |
| Vaccination time | between days 60 and 90 of pregnancy |
| | (day 0 of the study) |
| Observations | Clinical signs |
| Post-vaccination | Serum at days 0, 14, 28, 42 and at termination |
| (in life) | WBC at day 0 and then daily for 14 days |
| | Buffy coat at day 0 and then daily for 14 days |
| Post-mortem | Gross-pathology |
| (day 60) | Organ panel for virus isolation |
| Type of study: | open controlled clinical study |
| Experimental unit: | Individual animal |
| Method of blinding: | Partial blinding. No detailed procedures for blinding |
| | and access to treatment schedule were applied. The |
| | observing veterinarian at the study location and the |
| | pathologist were not be aware of the treatment; they |
| | only received a protocol extract relevant to their |
| | tasks. Vaccination was performed by the |
| | investigator or his assignee. Samples for virus |
| | isolation were coded by the investigator until all |
| | results are available. |

### Results

All heifers were healthy and pregnant at study start. All animals proved to be free of BVDV and BVDV antibodies before the initiation of the study.

### Preparation and Control of the Virus Used for the Infection

Samples were collected throughout, the dilution steps and assayed on the day of preparation, i.e. without freezing by co-cultivation on suitable tissue culture. The results of virus titration are shown in the following table.

| Sample ID | Virus strain | Dilution/description | Log₁₀ titre/ml |
|---|---|---|---|
| VT1a | XIKE-A/NdN | 1:2 (at 4° C.) | 4.4 |
| VT1b | (S) | #2a on ice without opening | 4.0 |
| VT1c | | Return of #2b | 2.8 |
| VT2a | XIKE-B | 1:2.2 (at 4° C.) | 2.3 |
| VT2b | | #3a on ice without opening | 2.8 |
| VT2c | | Return of #3b | Negative |

### Clinical Symptoms of BVDV Infection

The table below gives a summary about the animals that had clinical signs during the observation period.

Clinical Signs and the Days Post Inoculation (DPI) when they were Observed

Group 1 (XIKE-A NdN)

| | |
|---|---|
| Animal | Group 2 (XIKE-B) |
| ID | Animal ID |

| Clinical sign | 1583 | 1438 | 1585 |
|---|---|---|---|
| Loss of appetite | 8 | — | 10 |
| Lachrymation | — | — | — |
| Conjunctivitis | — | — | — |
| Nasal discharge | — | — | — |
| Oral erosion | — | — | — |
| Oral haemorrhage | — | — | — |
| Diarrhoea | — | — | — |
| Coughing | — | 12 | 10-13 |
| Abnormal breathing | — | — | — |
| Elevated respiratory rate | — | — | — |
| Hoof erosion | — | — | — |

Only mild and transient clinical signs were observed in some of the animals in each group. In Group 1, one out of the 5 heifers had loss of appetite on day 8 PI. In Group 2, two out of the 3 animals had clinical signs. Both heifers experienced coughing around day 21 PI that was accompanied with loss of appetite in one of the animals.

### Rectal Temperatures

No abnormal temperature changes were detected before the inoculation of the animals. The few cases of elevated temperatures measured after the inoculation are summarised in the table below.

| Group | Animal ID | Temperature (° C.) | PI day |
|---|---|---|---|
| 1 | 1583 | 39.9 | 8 |
| | 1621 | 39.0 | 5 |
| 2 | 1438 | 39.0 | 2 |
| | 1585 | 40.8 | 9 |

One animal in each group had slightly elevated temperature, and also one animal in each group had fever. Fever was detected on day 8 or 9 PI. Temperature values always returned to normal values on the following day.

### Leukocyte Counts

Some leukopenia was observed in all groups between PI days 3-8. The number of animals with at least 40% reduction in white blood cell count is noted below:

| Group | Number of animals having leukopenia/total |
|---|---|
| 1 | 3/5 (60%) |
| 2 | 1/3 (33%) |

### Serology (BVDV Antibodies)

In compliance with the study protocol, all heifers were free of BVDV antibodies before vaccination. In Group 1 (inoculated with XIKE-A NdN) and Group 2 (inoculated with XIKE-B), complete seroconversion was detected only at study termination (2 months after inoculation).

BVD Virus Isolation from Buffy Coats

No viremia was detected

BVD Virus Isolation from Fetal Tissue Samples

| | Group 1 | Group 2 |
|---|---|---|
| N | 5 | 3 |
| Treatments | XIKE-A-NdN | XIKE-B/control |
| Route | Intramuscular | Intramuscular |
| Number of fetuses | 4 out of 5 foetuses | 2 out of 3 foetuses |
| in which fetal | infected | infected |
| transmission was | | |
| detected: | | |
| Conclusion of the | Fetal transmission | Fetal transmission |
| virus used for | for XIKE-A-NdN | for XIKE-B |
| treatment has the | observed | observed |
| potential to the | | |
| transmitted over | | |
| the placenta: | | |

### Conclusion

The N^{pro} deletion resulted in a considerable attenuation of the BVDV in comparison to lire parental virus XIKE-A that was shown to be highly pathogenic (Meyer et al., 2002), However, the N^{pro} deletion alone is not preventing transmission of a NY93 based virus recombinant to the foetus after inoculation of pregnant cows.

### Example 3

### BVDV XIKE-B-NdN

### Foetopathogenicity Assessment in Pregnant Heifers

To be able to test the potential of a combination of RNase inactivation and N^{pro} deletion with regard to BVDV attenuation and fetal transmission, different BVDV-2 mutants with deletions within the N^{pro} coding region were established based on the infectious cDNA clone pKANE40B, the RNase negative mutant of pKANE40A with a deletion of codon 349. The recovered viruses were analyzed with regard to presence of the desired mutations, the absence of second site mutations in the regions flanking the introduced changes and their growth characteristics in tissue culture. XIKE-B-NdN (V-pK88C), a variant containing a deletion of the complete N^{pro} coding region except for codons 1 to 4 in addition to the RNase inactivating deletion of codon 349 was chosen for an animal experiment since it combined the desired mutations with acceptable growth characteristics. The aim of the study was to assess the safety of a live attenuated BVDV isolate in pregnant animals. Five BVDV-negative, pregnant heifers were inoculated intranasally with an infective dose of 10⁵ TCID₅₀/animal XIKE-B-NdN. Clinical data were recorded daily. Blood samples were collected for white blood cell counting, buffy-coat preparation, and serology. After termination of the study fetal tissues were collected for virus isolation.

material and Methods:

As detailed for example 1:

### Results

No clinical data were observed (data not shown). Leukocyte counts remained virtually unchanged except for a significant decrease by approximately 40% below the baseline value (day 0) in heifer no-1015 on a single day (day 6 p.i.) (data not shown).

### a) Analysis of Buffy Coat Preparations:

Approximately 10⁶ leukocytes were cultured in duplicates with MDBK-cells in 24-well tissue culture plates for 5 days. Samples were freeze-thawed twice. One hundred microliter aliquots of thawed samples were inoculated onto freshly seeded 24-well tissue culture plates and tested for virus by indirect immune-fluorescence staining (mAb Code 4, directed against a conserved epitope in nonstructural protein NS3). No BVDV could be isolated from the buffy coat preparations of animals #921, 1013, 1015, 1055 and 1075, whereas positive controls clearly showed the correct conduction of the test.

### b) Post-Mortem Examination of Fetal Tissues

After termination of the study the following fetal tissues were collected for virus isolation: spleen, kidney, thymus, sternum, cerebellum, placenta, intestine and abdominal fluid. Briefly, tissue suspensions were made in a mortar using sterile sea sand and ice-cold PBS without Ca²⁺ and Mg²⁺. Mortars were rinsed with 1 ml ice-cold PBS without Ca²⁺ and Mg²⁺ and suspensions were centrifuged for 10 minutes at 2000×g (4° C.). The supernatant was first passed through a disposable 0.45 μm filter holder, followed by a second filter passage (0.2 μm pore size). Virus isolation was carried out in duplicate (400 μl fetal tissue suspension or 100 μl fetal abdominal fluid) on a monolayer of MDBK-cells in a 24 wells tissue culture plate (37° C., 7% CO2). Tissue samples were controlled daily for cytopathic effects or bacterial contamination, and after an incubation time of 5 days, plates were frozen and thawed twice. 100 μl of samples were passaged to freshly seeded MDBK-cells. Virus was detected by indirect immuno-fluorescence staining (mAb Code 4). No BVDV could be detected in the tissue samples or fetal abdominal fluid.

### c) Serological Findings

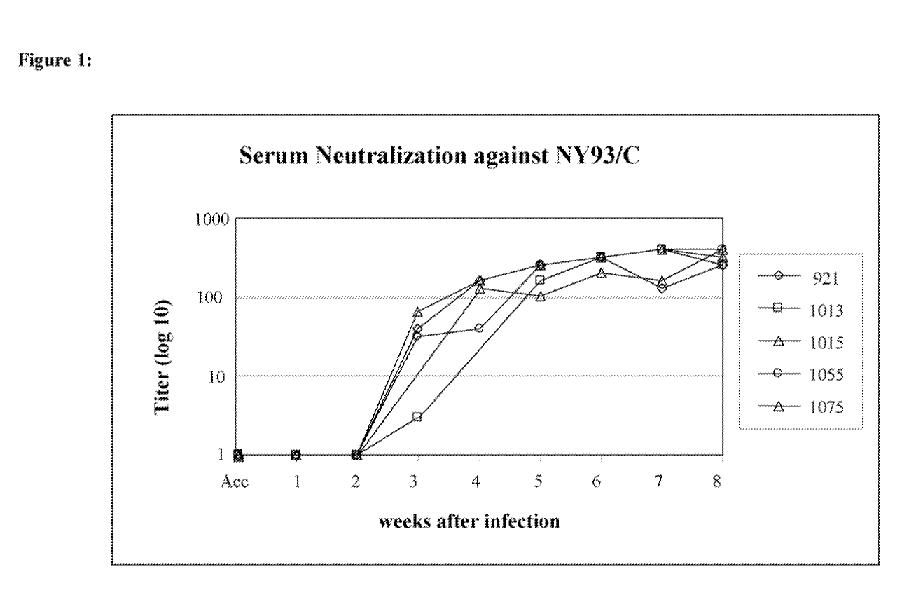

Serum neutralization litres were determined before inoculation, 1 month post-inoculation and at termination of the study. Sera from all animals were tested in triplicate for neutralizing antibodies against NY93/C, and the endpoint dilution was read by indirect immunofluorescence staining. Results were expressed as the endpoint dilution, which neutralized approximately 100 TCID₅₀ and calculated by the method of Kaerber. No definite data could be obtained for day 0, and 1 and 2 weeks post infection as the sera were toxic for MBDK-cells in dilutions up to 1:16 and no neutralization could be detected at higher dilutions. Starting with the third week post vaccination all animals developed neutralizing antibodies against the homologous BVDV-2 virus NY'93/C lasting till the end of the experiment (FIG. 1).

### d) Conclusions

The data obtained during the animal study clearly show that BVDV XIKE-B-NdN represents a highly attenuated virus. In contrast to wild type virus or the single mutants XIKE-B or XIKE-A-NdN that show fetal transmission in pregnant heifers at high rates the double mutant did not cross the placenta. BVDV XIKE-B-NdN as well as similar double mutants are extremely suitable for use in a live attenuated vaccine.

### Efficacy and Crossprotection Study

Two possible problems have to be faced with regard to vaccination with attenuated virus mutants BVDV XIKE-B or BVDV XIKE-B-NdN. First, there is a general problem concerning cross protection between BVDV-1 and BVDV-2. At least vaccination with inactivated BVDV-1 vaccines did not prevent the transmission of BVDV-2 to the foetus in pregnant animals. Since protection against fetal infection represents the major aim of anti BVDV vaccination, such vaccines cannot be regarded to induce a protective immunity on a broad range. The question therefore was, whether vaccination with live attenuated BVDV-2 can prevent virus transmission to the foetus. Second, the reduced growth rates of BVDV XIKE-B-NdN might result in only a low level of protection not able to prevent transplacental infection of the foetus in pregnant heifers. To address these problems, an animal study was started. The animals (2 groups of 10 animals each) were vaccinated either with BVDV XIKE-B or XIKE-B-NdN (intended dosage: 1 ml of supernatant with 10⁵ TCID₅₀ of virus). None of the animals showed significant clinical signs after the vaccination except for one animal of the nonvaccinated control group with mild coughing for one day. Rectal temperature values were below 39° C. except for one animal of the nonvaccinated control group that was 39.1° C. for one day. Buffy coat samples prepared after vaccination were analysed for the presence of virus as described above. The experiments showed that only 5 of the 20 animals contained virus in the blood for 1 or 2 days at 4 to 8 days post infection.

Four weeks after vaccination, insemination of the animals was carried out. Challenge infections were performed 60 to 90 days later using either a BVDV-1 strain (BVDV KE-9, heterologous challenge, animals vaccinated with XIKE-B) or a heterologous BVDV-2 strain (BVDV KE-13, homologous challenge, animals vaccinated with XIKE-B-NdN) (intended dosage: 10⁵ TCID50 in 6 ml). From each group of vaccinated animals 5 pregnant heifers were randomly selected for the challenge infection. Animals vaccinated with BVDV XIKE-B were challenged with the BVDV-1 strain KE-9, whereas heifers vaccinated with BVDV XIKE-B/NdN were challenged with BVDV-2 KE-13. In addition, 2 nonvaccinated control animals were infected with each of the challenge viruses. The vaccinated animals did not show viremia or clinical symptoms upon challenge infection. The challenge was successful as all non-vaccinated controls were BVDV positive. Only mild signs of disease were observed in the control groups. The white blood cell counts were nearly normal (not shown).

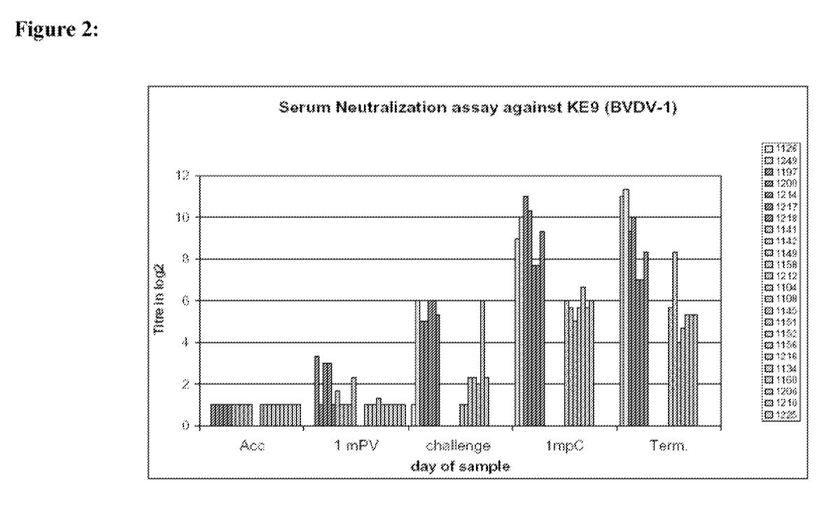

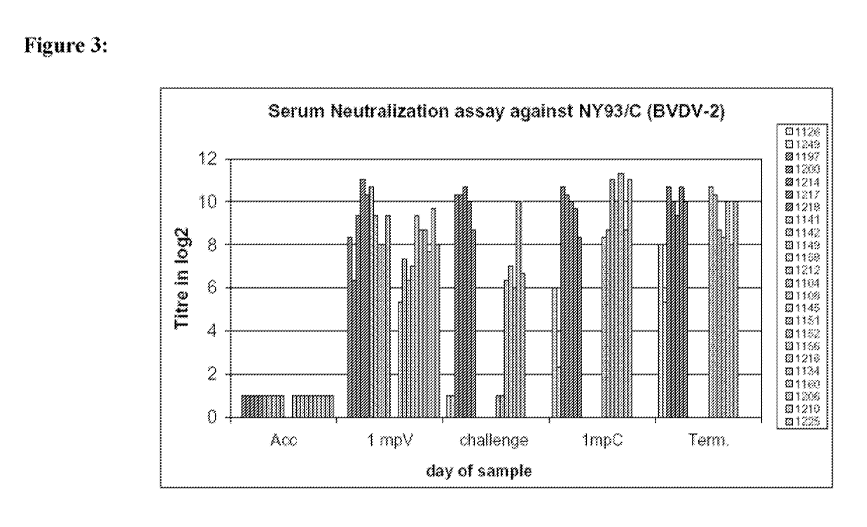

Serum neutralization titers were determined before inoculation, 1 month post-inoculation, before challenge, 1 month after challenge and at termination of the study. Sera from all animals were tested in triplicates for neutralizing antibodies against KE9 and NY93/C (1456Nase), and the endpoint dilution was read by indirect immunofluorescence staining. Results were expressed as the endpoint dilution, which neutralized approximately 100 TCID₅₀ and calculated by the method of Kaerber. At some of the higher antibody litres, the used endpoint dilution was not high enough. Against KE9, only animals vaccinated with XIKE-B developed low antibody titres starting about week 4. At challenge, all animals had antibody titres, which increased considerably starting around week 4 post challenge. XIKE-B vaccinated animals had higher antibody titres then those vaccinated with XIKE-B-NdN vaccinated. All animals developed about the same neutralization litre against NY93/C four weeks post vaccination, with marginally lower titres in XIKE-B-NdN vaccinated animals. After challenge, all animals had high antibody titres. FIG. 2 shows the serum neutralization assay against KE9 (BVDV-1) and FIG. 3 shows the serum neutralization assay against NY93/C (BVDV-2).

Analysis of tissue samples obtained after termination of the study from the foetuses revealed that the material obtained from the vaccinated animals gave negative results whereas transmission had occurred in ail 4 control animals. Thus, it is clear that the established BVDV-2 mutants are well suited as efficient cross protective vaccine viruses.

### Conclusion

The challenge was successful as all non-vaccinated controls were BVDV viraemic and foetuses of all non-vaccinated controls were BVDV positive. Both isolates gave full protection under the present test and assay conditions. Isolate XIKE-B, with the single genetic marker was shown to cross-protect against type 1 BVDV challenge in terms of BVD viraemia and transmission to the foetus after challenge. Isolate XIKE-B-NdN with the double genetic marker was able to fully protect against a heterologue type 2 BVDV challenge strain in terms of BVD viraemia and transmission to the foetus after challenge:

1. Isolate XIKE-B (type 2 isolate) was shown to cross-protect against type 1 BVDV challenge in terms of BVD viraemia and transmission to the foetus after challenge under the present test and assay conditions (n=4).
2. Isolate XIKE-B-NdN (type 2 isolate) fully protected against a heterologues type 2 BVDV challenge strain in terms of BVD viraemia and transmission to the foetus after challenge under the present test and assay conditions (n=5).

### Example 4

### Establishment of N^{pro} Mutants

This Example further analyzes BVDV-2 mutants with N^{pro} deletions. Different mutants with deletions in the N^{pro}-coding region of the genome were established. Initially, only true deletions or a deletion accompanied by a point mutation were introduced.

### A: [N^{pro}]₁-[C-term];

### B: [N^{pro}]₃-[C-term];

### C: [N^{pro}]₄-[C-term];

### D: [N^{pro}]₆-[C-term];

### E: [N^{pro}]₄-[C-term*]

In the formulas [N^{pro}]ₓ represents the number of residues of the aminoterminus of N^{pro} that are left in the mutated polyprotein amino acids, [C-term] is the complete polyprotein except for N^{pro} (starting with the C protein and ending with NS5B), and [C-term*] is the same as [C-term] but with a mutation at position 2 of the C protein (N instead of D). The growth rates of the recovered viruses were considerably lower than those of wild type XIKE-A or the RNase negative mutant XIKE-B. There are two possible explanations for this finding: (i) dependent on the virus strain, sequences of variable length of the N^{pro}-coding region are necessary for efficient translation initiation (Mayers et al., 2001; Tautz et al., 1999) and (ii) the fusion of additional sequences to the aminoterminus of the capsid protein interferes with capsid protein function. To obtain better growing N^{pro} deletion mutants, a second set of mutants was generated with either a bovine ubiquitin gene or a fragment of the bovine LC3-coding sequence replacing the major part of the N^{pro} gene. These constructs allow efficient translation and generate a capsid protein with the correct amino terminus.

### [N^{pro}]₂₂-[PS]-[C-term]

wherein PS is ubiquitin or LC3, C-term is the complete polyprotein except for N^{pro} (starting with the C protein and ending with NS5B).

The growth rates of these mutants were more similar to what was determined for XIKE-A. It even seemed that the two RNase positive viruses according to the formula [N^{pro}]₂₂-[PS]-[C-term] named V-pK87F and V-pK87G showed no significant growth retardation at all, whereas the RNase negative counterpart V-pK88G once again was somewhat hampered in propagation but to a lesser extent than the formerly described mutants.

Further examples of N^{pro} deletion mutants may be:

| | |
|---|---|
| MESDEGSK . . . | (SEQ ID NO 28) |
| MELFSSDEGSK . . . | (SEQ ID NO 29) |
| MELFSNESDEGSK . . . | (SEQ ID NO 30) |
| MELFSNELSDEGSK . . . | (SEQ ID NO 31) |
| MELFSNELLSDEGSK . . . | (SEQ ID NO 32) |
| MELFSNELLYSDEGSK . . . | (SEQ ID NO 33) |
| MELFSNELLYKSDEGSK . . . | (SEQ ID NO 34) |
| MELFSNELLYKTSDEGSK . . . | (SEQ ID NO 35) |

MELFSNELLYKT represents the aminoterminal sequence of N^{pro} of the BVDV isolate New-York93/C.

It may also be possible to use variants of this sequence with one or several mutations. Especially the naturally occurring variations as found in other pestiviruses can be expected to be functional. Therefore, the complete list of the tested or proposed variants with the different parts of the aminoterminal end of N^{pro} can be enlarged by equivalent sets with amino acid exchanges. Below, typical examples of the respective sequences are given for several pestiviruses but the possible variations are not limited to these examples.

| | | |
|---|---|---|
| BVDV New York93/C: | MELFSNELLYKT | |
| BVDV CP13: | [symbol] | (SEQ ID NO 36) |
| BVDV SD1: | [symbol] | (SEQ ID NO 37) |
| CSFV Brescia: | [symbol] | (SEQ ID NO 38) |
| BDV X818: [symbol] | (SEQ ID NO 39) | |

Thus, these variants for example may include: MELI-[PS]₀-[C-term];

MELIS-[PS]₀-[C-term];

MELISN-[PS]₀-[C-term];

MELISNE-[PS]₀-[C-term];

MELISNEL-[PS]₀-[C-term];

MELISNELL-[PS]₀-[C-term];

MELISNELLY-[PS]₀-[C-term];

MELISNELLYK-[PS]₀-[C-term];

MELISNELLYKT-[PS]₀-[C-term];

MELIT-[PS]₀-[C-term];

MELITN-[PS]₀-[C-term];

MELITNE-[PS]₀-[C-term];

MELITNEL-[PS]₀-[C-term];

MELITNELL-[PS]₀-[C-term];

MELITNELLY-[PS]₀-[C-term];

MELITNELLYK-[PS]₀-[C-term];

MELITNELLYKT-[PS]₀-[C-term];

These formulas may also have [PS]₁, i.e. PS may also be one of the PS as described herein. Sequences belonging to the N^{pro} protein are in italics. Amino acid exchanges with regard to the sequence of BVDV NewYork93/C are in bold.

Further examples can be found e.g. by using the GenBank accession numbers given in Becher et al., 2003, Virology 311, 96-104) or by standard sequence data searches.

A further possibility could be the use of a processing signal (PS) inserted between the (residual) N^{pro} sequence and the aminoterminus of the capsid protein. The PS leads to a cleavage that generates a functional capsid protein. The configuration of such constructs could be as follows:

[N^{pro}]₂₂-PS[C-term]

PS: Processing signal. Can either be a target for a protease (e.g. ubiquitin, LC3 as defined herein or a protease or an unstable peptide leading to processing at its own carboxyterminus like e.g. intern (Chong et al. 1998 and references therein) or 3C of picornaviruses, 2A of cardioviruses or aphtoviruses, p15 of rabbit hemorrhagic disease virus or the corresponding protease of other caliciviruses (Proter 1993, and references therein; Meyers et al., 2000 and references therein). When using a PS, a large number of different variants are possible since the PS ensures the generation of the correct amino terminus of the capsid protein C. Thus, when using a PS construct, all kinds of deletions or mutations of the N^{pro} sequence are expected to result in viable mutants as long as the reading frame is not shifted or translation stopped by an in frame stop codon. As an example we established a viable CSFV N^{pro} deletion mutant according to the formula

[N^{pro}]₂₉-PS[C-term]

Especially interesting could be N^{pro} mutations blocking the proteolytic activity of the protein. Rümenapf et. al. (1998) have published the identification of the active site residues of the protease for CSFV Alfort Tübingen. The respective ammo acids (glutamic acid at position 22, histidine at position 49 and cysteine at position 69) are conserved for other pestiviruses. Thus, exchanges of any amino acid expect for serine or threonine for the cysteine at position 69 will result in destruction of the protease activity. Similarly, changing the glutamic acid at position 22 will most likely result in inactivation of the protease unless the new amino acid is aspartic acid. Similarly, most if not all exchanges at position 49 will lead to an inactive protease.

### Example 5

### Preparation of Combination Vaccines According to the Invention

### Vaccine A

### IBR, BVDV Types 1 and 2, BRSV

Attenuated BVDV type 1 and 2 strains, having at least one mutation in the coding sequence for glycoprotein E^{rns} and/or at least another mutation in the coding sequence for N^{pro}, wherein said mutation in the coding sequence for glycoprotein E^{rns} leads to inactivation of RNase activity residing in E^{rns} and/or said mutation in the coding sequence for N^{pro} leads to inactivation of said N^{pro}, are grown in MDBK-cells until a TCID₅₀ of about 10^{5.0} to 10^{8.1} per ml cell culture fluid. A live attenuated strain of IBR is grown in MDBK cells until a TCID₅₀ of about 10^{5.0} to 10^{8.6} per ml cell culture fluid. A live attenuated strain of BRSV is grown in MDBK cells until a TCID₅₀ of about 10^{5.0} to 10^{7.2} per ml cell culture fluid. Each virus containing culture fluid is collected and lyophilized. Equal amounts of the lyphilized antigens are mixed. For reconstitution, an aqueous solution containing 1 to 3%:0.8 ml of Alhydrogel is used. One dose of the combination vaccine contains 4 ml of the reconstituted antigens, A filial dose includes IBR (10^{8.0} to 10^{8.6} TCID₅₀), BVDV-1 (10^{5.0} to 10^{8.1} TCID₅₀), BVDV-2 (10^{5.0} to 10^{8.1} TCID₅₀), and BRSV (10^{5.0} to 10^{7.2} TCID₅₀).

### Vaccine B

### IBR, BVDV types 1 and 2, PI3 BRSV

The preparation of the IBR, BVDV 1 and 2 and BRSV antigens is performed as described for vaccine A. In addition, a live attenuated strain of PI3 is grown in MDBK cells until a TCID₅₀ of about 10^{4.2} to 10^{6.5} per ml cell culture fluid. Afterwards, the PI3 containing culture fluid is harvested and lyophilized. An amount of 10^{4.2} to 10^{6.5} (TCID₅₀) of the lyophilized antigen is mixed with the lyophilized IBR, BVDV types 1 and 2, and BRSV antigens The mixture is then reconstituted in 4 ml as described for Vaccine A. A final dose includes IBR (10^{5.0} to 10^{8.6} TCID₅₀), BVDV-1 (10^{5.0} to 10^{8.1} TCID₅₀), BVDV-2 (10^{5.0} to 10^{8.1} TCID₅₀), BRSV (10^{5.0} to 10^{7.2} TCID₅₀), and PI3 (10^{4.2} to 10^{6.5} TCID₅₀).

### Vaccine C

### BVDV Types 1 and 2, PI3, BRSV, Mannheimia (Pasteurella) haemolytica

BVDV 1 and 2, BRSV and PI3 viruses are grown as described for vaccines A and B. After the culture fluids are harvested, the viruses are inactivated and lyophilized. Mannheima (Pasteurella) haemalytica is grown until 10^{8.0} to 10^{11.0} cells per ml culture. The bacteria are inactivated and the culture fluid is lyophilized or freeze dried. An amount of 10^{8.0} to 10^{11.0} lyophilized or freeze dried bacteria cells are mixed with the lyophilized BVDV types 1 and 2 antigen (each in an amount of 10^{5.0} to 10^{8.1} TCID₅₀), PI3 antigen (10^{7.3} to 10^{8.3} TCID₅₀) and BRSV antigen (10^{5.0} to 10^{7.2} TCID₅₀). The reconstituted suspension (5 ml per dose) further contains 30 to 50 mg Aluminium hydroxide, 0.4 to 0.8 mg Quil A (Saponin), 0.04 to 0.06 mg sodium timerfonate and traces of neomycin. Final antigen amounts per dose are BVDV-1 (10^{5.0} to 10^{8.1} TCID₅₀), BVDV-2 (10^{5.0} to 10^{8.1} TCID₅₀), PI3 (10^{7.3} to 10^{8.3} TCID₅₀) BRSV (10^{5.0} to 10^{7.2} TCID₅₀) and Mannheima (Pasteurella) haemalytica (10^{8.0} to 10^{11.0} cells).

### Vaccine D

### BVDV types 1 and 2, IBR, BRSV, PI3, Leptospira canicola, Leptospira grippo, Leptospira hardjo, Leptospira ponoma, Leptospora borgpetersenii hardjo-bovis

Modified live viruses of BVDV 1 and 2, BRSV, IBR, and PB are grown as described for vaccines A and B. After the culture fluids are harvested, the viruses are lyophilized. Leptospira canicola, Leptospira grippo, Leptospira hardjo, Leptospira ponoma, Leptospora borgpetersenii hardjo-bovis are separately cultivated until reaching 10^{8.0} to 10^{11.0} cells per ml culture. The bacteria cultures are inactivated and the culture fluids are lyophilized or freeze dried. Each of the 10^{8.0} to 10^{11.0} of the lyophilised or freeze dried bacteria cells are reconstituted with the lyophilized modified BVDV types 1 and 2 (each in an amount of 10^{5.0} to 10^{7.0} TCID₅₀), modified live PI3 (10^{7.3} to 10^{8.3} TCID₅₀), modified live BRSV (10^{5.0} to 10^{7.0} TCID₅₀) and modified live IBR (10^{6.1} to 10^{7.7} TCID₅₀). The reconstituted suspension (2 ml per dose) contains traces of neomycin as preservative. Final antigen amounts per dose are BVDV-1 (10^{5.0} to 10^{7.0} TCID₅₀), BVDV-2 (10^{5.0} to 10^{7.0} TCID₅₀), PI3 (10^{7.3} to 10^{8.3} TCID₅₀) BRSV (10^{5.0} to 10^{7.0} TCID₅₀), PI3 (10^{7.3} to 10^{8.3} TCID₅₀), and Leptospira canicola, Leptospira grippo, Leptospira hardjo, Leptospira ponoma, and Leptospora borgpetersenii hardjo-bovis (each 10^{8.0} to 10^{11.0} cells).

### Vaccine E

### BVDV types 1 and 2, IBR, BRSV, PI3, and H. somnus

Modified live viruses of BVDV 1 and 2, BRSV, IBR, and PI3 are grown as described for vaccines A and B. After the culture fluids are harvested, the viruses are lyophilized. H. somnus is cultivated until achieving 10^{7.1} to 10^{9.2} cells per ml culture. The bacteria culture is inactivated and the culture fluid, is lyophilized or freeze dried. 10^{7.1} to 10^{9.2} of the lyophilized or freeze dried bacteria are reconstituted with the lyophilized modified BVDV types 1 and 2 (each in an amount of 10^{5.0} to 10^{7.0} TCID₅₀), modified live PI3 (10^{7.3} to 10^{8.3} TCID₅₀), modified live BRSV (10^{5.0} to 10^{7.0} TCID₅₀) and modified live IBR (10^{6.1} to 10^{7.7} TCID₅₀). The reconstituted suspension (2 ml per dose) contains traces of neomycin as preservative. Final antigen amounts per dose are BVDV-1 (10^{5.0} to 10^{7.0} TCID₅₀), BVDV-2 (10^{5.0} to 10^{7.0} TCID₅₀), PI3 (10^{7.3} to 10^{8.3} TCID₅₀) BRSV (10^{5.0} to 10^{7.0} TCID₅₀), PI3 (10^{7.3} to 10^{8.3} TCID₅₀), and H. somnus (10^{7.1} to 10^{9.2} cells).

### REFERENCES

The teachings and contents of all references (articles, patents, book portions, presentations, and the like) cited herein, including those listed below, are expressly incorporated by reference herein.
Ausubel, F. M. et al. Current Protocols in molecular biology. New York: Greene Publishing Associates and Wiley Interscience. 1994 (updated)
Baker, J. C. 1987. Bovine viral diarrhea virus: a review. J. Am. Vet. Med. Assoc. 190: 1449-1458.
Becher, P., König, M., Paton, D. J., Thiel, H. J., 1995, Further characterization of border disease virus isolates: evidence for the presence of more than, three species within the genus pesivirus. Virology 209 (1), 200-206.
Chong, S., Williams, K. S., Wolkowicz, C., and Xu, M. Q. 1998. Modulation of Protein Splicing of the Saccharomyces cerevisiae Vacuolar Membrane ATPase Intein. J. Biol. Chem. 273: 10567-10577. Donis, R. O., Corapi, W., and Dubovi, E. J. 1988. Neutralizing monoclonal antibodies to bovine viral diarrhea virus bind to the 56K to 58K glycoprotein, J. Gen. Virol. 69: 77-86.
Fuerst T. R. et al. 1986. Eukaryotic transient expression system based on recombinant vaccinia virus that synthesizes bacteriophage T7 RNA polymerase. Proc. Natl. Acad. Set. 83: 8122-8126.
Heinz, F. X., Collett, M. S., Purcell., R. H., Cold, E. A., Howard, C. R., Houghton, M., Moormann, R. J. M., Rice, C. M., and Thiel, H.-J. 2000. Family Flaviviridae. PP 859-878. In: Virus Taxonomy (van Regenmortel., H. H. V., Fauquet, C. M., and Bishop, D. H. L., Eds.). Academic Press, San Diego.
Hulst, M. M., Himes, G., Newbigin, E., Moormann, R. J. M. 1994. Glycoprotein E2 of classical swine fever virus: expression in insect cells and identification as a ribonuclease. Virology 200: 558-565.
Hulst, M. M., F. E. Panoto, A. Hooekmann, H. G. P. van Gennip., and Moormann, R. J. M. 1998. Inactivation of the RNase activity of glycoprotein E^{rns} of classical swine fever virus results in a cytopathogenic virus. J. Virol. 72: 151-157.
Kit, M. and S. Kit. 1991. Sensitive glycoprotein gIII blocking ELISA to distinguish between pseudorabies (Aujesky's disease)-infected and vaccinated pigs. Veterinary Microbiology 28:141-155.
Kunkel, T. A., J. D, Roberts, and R. A, Zakour. 1987, Rapid and efficient site-specific mutagenesis without phenotypic selection. Methods Enzyme). 154:367-392.
König, Matthias, 1994, Virus der klassischen Schweinepest: Untersuchungen zur Pathogenese und zur Induktion einer protektiven Immunantwort. Dissertation, Tier\check{a}rztliche Hochschule Hannover, Germany.
Lindenbach, B. D., and Rice, C. M. 2001. The pestiviruses. In Fields Virology, eds. Knipe, D. M., & Howley, P. M. (Lippincott-Raven, Philadelphia), pp. 991-1042. Mayer, D., Hermann, M. A., and Tratschin, J. D. 2004. Attenuation of classical swine fever virus by deletion of the viral N(pro) gene. Vaccine. 22:317-328.
Meyers, G., Rümenapf, T. and Thiel, H.-J. 1989. Molecular cloning and nucleotide sequence of the genome of hog cholera virus. Virology 171: 555-567.
Meyers, G., Saalmüller, A., and Büttner. M. (1999). Mutations abrogating the RNase activity in glycoprotein e(rns) of the pestivirus classical swine fever virus lead to virus attenuation. J Virol 73: 10224-10235.
Meyers. G., Tautz, N., Becher, P., Thiel, H.-J., & Kümmerer, B. M. 1996b. Recovery of cytopathogenic and noncytopathogenic bovine viral diarrhea viruses from cDNA constructs. J. Virol., 70: 8606-8613.
Meyers, G., Thiel, H.-J., and Rümenapf, T. 1996a. Classical swine fever virus: Recovery of infectious viruses from cDNA constructs and generation of recombinant cytopathogenic swine fever virus. J. Virol. 67:7088-709526.
Meyers, G., Wirblich, C., Thiel. H.-J. and Thumfart, J. O. 2000. Rabbit hemorrhagic disease Virus: genome organization and polyprotein processing of a calicivirus studied after transient expression of cDNA constructs. Virology 276: 349-363.
Moennig, V. and Plagemann, J. 1992. The pestiviruses. Adv. Virus Res. 41: 53-91.
Paton, D. J., Lowings, J. P., Barrett, A. D. 1992. Epitope mapping of the gp53 envelope protein of bovine viral diarrhea virus. Virology 190: 763-772.
Pellerin, C. et. al. Identification of a new group of bovine viral diarrhea virus strains associated with severe outbreaks and high mortalities, Virology 203, 1994:260-268.
Porter, A. G. (1993). Picornavirus nonstructural proteins: emerging roles in virus replication and inhibition of host cell functions. J. Virol. 67, 6917-6921.
Rüggli, N., Tratschin, J. D., Schweizer, M., McCullough, K. C., Hofmann, M. A., Summerfield, A. 2003. Classical swine fever virus interferes with cellular antiviral defense: evidence for a novel function of N(pro). J. Virol. 77:7645-7654.
Rümenapf, T., Stark, R., Heimann, M., and Thiel, H.-J. 1998. N-terminal protease of pestiviruses: identification of putative catalytic residues by site directed mutagenesis. J. Virol. 72: 2544-2547.
Rümenapf, T., Unger, G., Strauss, J. H., and Thiel, H.-J. 1993. Processing of the envelope glycoproteins of pestiviruses. J. Virol. 67: 3288-3294, Schneider, R., G. Unger, R. Stark, E. Schneider-Scherzer, and H.-J. Thiel. 1993. Identification of a structural glycoprotein of an RNA virus as a ribonuclease. Science 261: 1169-1171.
Sambrook, J., Fritsch, E. F. & Maniatis, T., Molecular Cloning: A Laboratory Manual Cold Spring Harbor Laboratory, Cold Spring Harbor, N.Y., 1989
Stark, R., Meyers, G., Rümenapf, T., and Thiel, H.-J. (1993): Processing of pestivirus polyprotein: Cleavage site between autoprotease and nucleocapsid protein of classical swine fever virus. J. Virol., 67, 7088-7095. Thiel, H.-J., Plagemann, G. W., & Moennig, V. 1996. The pestiviruses. In Fields Virology, eds. Fields, B. N., Knipe, D. M., & Howley, P. M. (Lippincott-Raven, Philadelphia), pp. 1059-1073.
Thiel, H.-J., Stark, R., Weiland, E., Rümenapf, T. & Meyers, G. 1991. Hog cholera virus: molecular composition of virions from a pestivirus. J. Virol. 65: 4705-4712.31.
Tratschin, J.-D., Moser, C., Ruggli, N., and Hofmann, M. A., 1998. Classical swine fever virus leader proteinase Npro is not required for viral replication in cell culture. J. Virol. 72, 7681-7684. van Rijn, P. A., van Gennip, H. G., de Meijer, E. J., Moormann, R. J. 1993. Epitope mapping of envelope glycoprotein E1 of hog cholera virus strain Brescia J. Gen. Virol. 74: 2053-2060.
Weiland, E., Thiel, H.-J., Hess, G., and Weiland, F. (1989). Development of monoclonal neutralizing antibodies against bovine viral diarrhea virus after pretreatment of mice with normal bovine cells and cyclophosphamide. J. Virol. Methods 24: 237-244.
Weiland, E., Stark, R., Haas, B., Rümenapf, T., Meyers, G. and Thiel, H.-J. (1990) Pestivirus glycoprotein which induces neutralizing antibodies forms part of a disulfide-linked heterodimer. J. Virology 64, 3563-3569.
Weiland, E., Ahl, R., Stark, R. Weiland, F. and Thiel H.-J. (1992). A second envelope glycoprotein mediates neutralization of a pestivirus, hog cholera virus. J. Virology 66, 3677-3682.
Windisch, J. M., Schneider, R., Stark, R., Weiland, E., Meyers, G., and Thiel, H.-J. 1996. RNase of classical swine fever virus: biochemical characterization and inhibition by virus-neutralizing monoclonal antibodies. J. Virol. 70: 352-358
Wiskerchen, M., Belzer, S. K., and Collet, M. S. 1991. Pestivirus gene expression: the first protein product of the bovine viral diarrhea, virus large open reading frame, p20, possesses proteolytic activity J. Virol. 65:4508-4514.

1. A combination vaccine for the treatment and/or prophylaxis of cattle against microbiological infections, wherein said combination vaccine comprises
a. one or more attenuated BVDV, having at least one mutation in the coding sequence for glycoprotein E^{rns} and/or at least another mutation in the coding sequence for N^{pro}, wherein said mutation in the coding sequence for glycoprotein E^{rns} leads to inactivation of RNase activity residing in E^{rns} and/or said mutation in the coding sequence for N^{pro} leads to inactivation of said N^{pro}; and
b. one or more immunological active component(s) effective for the treatment and/or prophylaxis of microbiological infection in cattle caused by a bovine pathogen other than BVDV.

2. The vaccine according to claim 1, wherein said combination comprises
a. one or more attenuated BVDV, having at least one mutation in the coding sequence for glycoprotein E^{rns}, wherein said mutation in the coding sequence for glycoprotein E^{rns} leads to inactivation of RNase activity residing in E^{rns} and
b. one or more immunological active component(s) effective for the treatment and/or prophylaxis of microbiological infection in cattle caused by a bovine pathogen other than BVDV.

3. The vaccine according to claim 1, wherein said combination comprises
a. one or more attenuated BVDV, having at least one mutation in the coding sequence for N^{pro}, said mutation in the coding sequence for N^{pro} leads to inactivation of said N^{pro}; and
b. one or more immunological active component(s) effective for the treatment and/or prophylaxis of microbiological infection in cattle caused by a bovine pathogen other than BVDV.

4. The vaccine according to claim 1, wherein said combination comprises
a. one or more attenuated BVDV, having at least one mutation in the coding sequence for glycoprotein E^{rns} and at least another mutation in the coding sequence for N^{pro}, wherein said mutation in the coding sequence for glycoprotein E^{rns} leads to inactivation of RNase activity residing in E^{rns} and/or said mutation in the coding sequence for N^{pro} leads to inactivation of said N^{pro}; and
b. one or more immunological active component(s) effective for the treatment and/or prophylaxis of microbiological infection in cattle caused by a bovine pathogen other than BVDV.

5. The vaccine according to claim 1, wherein said combination vaccine comprises attenuated BVDV type 1 and attenuated BVDV type 2, both having at least one mutation in the coding sequence for glycoprotein E^{rns} and at least another mutation in the coding sequence for N^{pro}, wherein said mutation in the coding sequence for glycoprotein E^{rns} leads to inactivation of RNase activity residing in E^{rns} and/or said mutation in the coding sequence for N^{pro} leads to inactivation of said N^{pro}.

6. The combination vaccine according to claim 1, wherein said infection in cattle other than BVDV is caused by at least one pathogen selected from the group consisting of:
Parainfluenza-3 Virus (PI-3), Infectious Bovine Rhinotracheitis virus (IBR), Bovine Respiratory Syncytial Virus (BRSV), Bovine Herpesvirus (BHV), Bovine Rotavirus (BRV), Bovine Enterovirus (BEV), Bovine Coronovirus (BCV), Bovine Rabies (BR), Bovie Parvovirus (PPV), Adenovirus Astrovirus, Mannheimia haemolytica (formerly Pasteurella haemolytica), Pasteurella multocida, Haemophilus somnus (Histophilus ovis and Haemophilus agni), Actinomyces (Corynebacterium), Actinomyces pyogenes, Chlamydia psittaci, Campylobacter fetus venerealis and Campylobacter fetus fetus (formerly C fetus intestinalis), Leptospira interrogans, Leptospira hardjo, Leptospira pomona, and Leptospira grippotyphosa, Leptospira canicola, Leptospira grippotyphosa, Leptospira hardjo (Leptospira, hardjoprajitno and Leptospira hardjo-bovis), Brucella abortus, Brucella suis and Brucella melitensis, Listeria monocytogenes, Chlamydia psittaci, Clostridium chauvoei, Clostridium septicum, Clostridium haemolyticum, Clostridium novyi, Clostridium sordellii, Clostridium perfringens, Clostridium tetani, Moraxella bovis, Klebsiella spp, Klebsiella pneumoniae, Salmonella typhimurium, Salmonella newport, Mycobacterium avium paratuberculosis, Cryptsporidium parvum, Cryptsporidium hominis, Staphylococcus aureus, Streptococcus dysgalactiae, Streptococcus uberus, Mycoplasma spp. Mycoplasma dispar. Mycoplasma bovis, and Ureaplasma spp., Tritrichomonas foetus, Trichophyton verrucosum, Trichophyton mentagrophytes, Trichophyton sarkisovii, Neospora caninum (formerly Toxoplasma gondii), Babesia bigemina and Babesia bovis, and Dictyocaulus viviparous (Lungworm disease).

7. The combination vaccine according to claim 1, wherein said immunological active component is an antigen of one at least one pathogen selected from the group consisting of:
Parainfluenza-3 Virus (PI-3), infectious Bovine Rhinotracheitis virus (IBR), Bovine Respiratory Syncytial Virus (BRSV), Bovine Herpesvirus (BHV), Bovine Rotavirus (BRV) Bovine Enterovirus (BEV), Bovine Coronovirus (BCV), Bovine Rabies (BR), Bovie Parvovirus (PPV), Adenovirus Astrovirus, Mannheimia haemolytica (formerly Pasteurella haemolytica), Pasteurella multocida, Haemophilus somnus (Histophilus ovis and Haemophilus agni), Actinomyces (Corynebacterium), Actinomyces pyogenes, Chlamydia psittaci, Campylobacter fetus venerealis and Campylobacter fetus fetus (formerly C fetus intestinalis), Leptospira interrogans, Leptospira hardjo, Leptospira pomona, and Leptospira grippotyphosa, Leptospira canicola, Leptospira grippotyphosa, Leptospira hardjo (Leptospira hardjoprajitno and Leptospira hardjo-bovis), Brucella abortus, Brucella suis and Brucella melitensis, Listeria monocytogenes, Chlamydia psittaci, Clostridium chauvoei, Clostridium septicum, Clostridium haemolyticum, Clostridium novyi, Clostridium sordellii, Clostridium perfringens, Clostridium tetani, Moraxella bovis, Klebsiella spp., Klebsiella pneumoniae, Salmonella typhimurium, Salmonella newport, Mycobacterium avium paratuberculosis, Cryptsporidium parvum, Cryptsporidium hominis, Staphylococcus aureus, Streptococcus dysgalactiae, Streptococcus uberus, Mycoplasma spp, Mycoplasma dispar, Mycoplasma bovis, and Ureaplasma spp., Tritrichomonas foetus, Trichophyton verrucosum, Trichophyton mentagrophytes, Trichophyton sarkisovii, Neospora caninum (formerly Toxoplasma gondii), Babesia bigemina and Babesia bovis, and Dictyocaulus viviparous (Lungworm disease).


SEQUENCE LISTING (39 sequences)

SEQ ID NO: 1 - DNA, 12332 bp, Artificial Sequence
  Wildtyp BVDV  XIKE-A
gtatacgagattagctaaagaactcgtatatggattggacgtcaacaaatttttaattggcaacgtagggaaccttcccctcagcgaaggccgaaaagaggctagccatgcccttagtaggactagcaaaagtaggggactagcggtagcagtgagttcgttggatggccgaacccctgagtacaggggagtcgtcaatggttcgacactccattagtcgaggagtctcgagatgccatgtggacgagggcatgcccacggcacatcttaacccatgcgggggttgcatgggtgaaagcgctattcgtggcgttatggacacagcctgatagggtgtagcagagacctgctattccgctagtaaaaactctgctgtacatggcacatggagttgttttcaaatgaacttttatacaaaacatataaacaaaaaccagcaggcgtcgtggaacctgtttacgacgtcaacgggcgcccactgtttggagagagcagtgacttgcacccgcagtcaacactaaaactaccacaccaacgaggcagcgccaacatcctgaccaatgctaggtccctaccgcggaaaggtgactgccggagaggtaatgtgtatggaccggtgagtggcatctatatcaaaccaggaccgatctactaccaggattatgtgggccccgtctatcatagagccccactggaactatgtagggaggcaagtatgtgcgaaacaactaggagagttggcagagtgaccggtagtgatgggaaattatatcatatctacatctgcatagatgggtgtatcctcctgaagagggcgactaggaaccaaccagaagtcctgaaatgggtatacaacagattaaattgtcctttatgggtcaccagctgctccgatgaagggagcaagggtgctacaagtaagaagcagcctaagccagataggatagaaaaaggtaagatgaaaatagccccaaaagagacagaaaaagattgcaaaaccagaccccccgacgcgactatagtagtagaaggggttaagtaccaggtgaagaaaaaaggaaaggtaaggggaaaaaatactcaagatgggttatatcacaacaagaataagccccctgaatcaagaaaaaaattggaaaaggcactgctggcttgggccatcttagcagcggtcctgcttcagctggtaacaggagagaatatcacccagtggaacttgatggacaacggcaccgagggaatacagcaagcgatgttcctaagaggggtgaacaggagtctacatggaatttggccagagaaaatttgcaccggagtaccaactcacttagcaacagactatgagcttaaagagatagtggggatgatggacgcgagtgagaagaccaactacacgtgttgcaggttgcaaagacatgagtggaataaacatggttggtgtaactggtttcatatagaaccgtggatatggttgatgaacaaaacccaaaacaacctgacagaagggcaaccgcttagggagtgtgctgtgacttgtaggtatgacaaggaaacagaattgaacatcgtgacacaggctagggacagacctacaactctgacaggttgcaagaaaggcaagaatttctctttcgcaggtgttatactggatgggccctgtaactttaaagtatcggttgaagatgtgctgttcaaggagcacgattgcggcaacatgctgcaagagaccgcgatacagctactcgatggggcaaccaacaccattgagggagcaagggtagggacggccaagttgacaacctggttagggaagcaattagggatccttggtaagaagttggagaacaaaagcaaagcatggtttggtgcacatgcagcaagtccatactgcggagtggagaggaagatcggttacgtatggtatacaaaaaactgcactccagcttgccttccaagaaacactagaataataggccccgggaaatttgataccaacgccgaagatggaaaaatactccatgagatgggggggcacctctcagaatttgtcctattgtccttggtggttctgtctgactttgccccggaaaccgcgagcgtcatctacttggttctacattttgcgatcccgcaaagccacgttgatgtagacacatgcgacaagaaccagctgaatttaacggtagcaaccacagtagcagaggtcataccagggacagtgtggaacctagggaagtatgtctgcataagaccagactggtggccatatgagacgacgacagtcttcgtcatagaggaagcagggcaagtaatcaaattgatgctaagggccatcagagacttaactaggatatggaatgctgccactaccacagctttcttaatctttttagtaaaagcactgaggggacaactaatccaagggctattgtggctgatgctaataacaggagcacagggcttccctgaatgcaaagagggcttccaatatgccatatctaaagacaggaaaatggggttattggggccagagagcttaactacaacatggcacctccccaccaaaaaaatagtggattccatggtgcatgtatggtgtgaaggaaaagacttgaaaatattaaaaatgtgcacaaaggaagagaggtatctagtggctgtgcacgagagagccttatcaaccagtgccgagtttatgcagatcagtgatgggacaataggcccagacgtgatagatatgcctgatgactttgagtttggactctgcccttgtgactcaaaaccagtgataaagggcaaatttaatgccagcttactgaatggaccagctttccagatggtatgcccacaggggtggactggtacaatagaatgcaccctagcgaaccaagacaccttggacacaactgtcattaggacatatagaagaactaccccatttcagcggagaaaatggtgtacctatgaaaaaataataggggaagatatctatgaatgcattctaggtggaaactggacatgcataaccggtgaccatagcaggttgaaagacggacctatcaagaagtgtaagtggtgtggccatgacttcgtcaactcagaggggctaccacactacccaataggcaagtgcatgctcatcaacgagagtgggtacaggtatgtagatgacacctcttgcgataggggtggtgtagccatagttccatctggcaccgtaaagtgtagaataggtaacgtcacggtgcaagttatcgctactaacaatgatctgggacccatgccttgcagcccagctgaagtgatagcaagtgaaggaccagtggaaaagactgcatgcacattcaactattcaaggactctacctaataagtattatgagccaagggaccggtacttccaacaatacatgttaaaaggggagtggcaatattggttcgacctggattctgtagaccaccacaaagactacttctcagagttcataatcatagcagtggtcgccttgttgggtggtaagtacgtactgtggctcttgataacatacacaatactgtctgagcagatggctatgggtgctggagtgaatactgaagagatagtcatgataggcaatttgctgacagacagtgatattgaggttgtggtttatttccttcttctgtacttaatagttaaagaggaactggcgaggaaatggattatactggtataccacatccttgtagccaaccctatgaaaacaattggggtcgtcttactaatgctagggggagtggtgaaggccagcagaatcaatgctgatgaccaaagtgctatggacccatgctttcttctcgtgacaggcgtagtggctgttttgatgatcgctagaagagaacctgccacattaccactgattgtagcattgctagcaataagaacatcaggattcctactgcccgctagcattgatgtaactgtagcagtagtattaattgtacttttgttggctagctacataacagactactttagatataaaaagtggcttcaactcttatttagtctgatagctggtatctttattataaggagcttaaaacatatcaaccagatggaggtaccagaaatatctatgccaagttggagacctctagctctggtccttttctatataacatctacagcaataaccactaattgggacattgacttagcaggcttcctgctgcaatgggcgccagcagtgatcatgatggctaccatgtgggcagactttttgactctgatcatagtcctgcccagttacgagttatctaagctttacttcctaaagaacgtcaggacagacgtggaaaagaactggctcggcaaagtgaaatacagacagatcagttcagtttatgacatctgtgacagtgaggaagcagtgtacctatttccatcaaggcataagagtggaagcaggccagatttcatattaccttttttgaaagccgtgttaataagctgcatcagcagccaatggcaagtggtttacatttcttacctaatactggaaattacatactatatgcacaggaaaatcatagatgaggtgtcaggaggagcaaattttctatcaagactcatagcagccatcatagaattaaattgggccatagatgatgaggaatgtaaaggactgaagaaactgtatctcttgtcagggagagcgaagaatttgatagttaaacataaggtaagaaatgaagccgtccacagatggtttggtgaggaggaaatatacggggcacccaaggtgatcactatcataaaagctagtaccctaagtaaaaacaggcactgcataatctgcacgatctgtgaagggaaagaatggaatggagccaactgcccaaagtgtggaagacaaggaaagcccataacatgtggaatgacactcgcagactttgaggagaaacattacaaaaagatatttataagagaagaatcttcttgtcctgtgccttttgatccttcttgccattgtaattattttcgccacgatgggcctttcaggaaagagtataagggttacgtccaatacacagccagaggacaactctttctgaggaacctaccaattctagcgacgaagatgaagctattaatggtgggaaacctcggcgcagaaattggcgacctggaacatctaggatgggtactgagagggccagccgtgtgcaaaaaaattaccaaccatgagaagtgccacgtaaacatcatggataagctaactgcattttttggaatcatgcctagaggcacgacccctagggcacctgtgaggttccccacagcactactaaaagtgagaagggggctagagacgggatgggcttacacgcaccaaggagggatcagctcggtagaccatgtcacagccggaaaggatttactagtgtgtgacagtatgggcaggaccagggttgtctgtcatagtaacaataagatgactgatgagactgagtatggcatcaagaccgactcagggtgtcccgaaggtgcgaggtgttacgtgctaaacccagaagctgttaacatttctggcacaaaaggagctatggtacacctccagaaaacggggggggagttcacatgtgtcactgcctcagggaccccggctttcttcgatctgaaaaatctaaaaggctggtccgggctaccaatttttgaagcatccagtggcagggtggttggtagggtgaaagtcggcaagaatgaggattccaagcccaccaaactaatgagcggaatccagacagtgtctaagaaccagacagacctagcggacatcgtaaaaaaattgactagtatgaacagaggagagttcaaacagataacattagccactggggcaggaaaaactacggaactgccaaggtccgtcatagaggagatagggaggcacaaaagggtcttagtcctgataccattgagagcagcagcagagtcagtgtatcagtatatgagagtgaagtacccaagtatatctttcaatttgagaataggagatatgaaggaaggtgacatggccactggtatcacctacgcctcatatgggtacttttgtcagcttcctcagcccaaactgagagctgccatggtagagtactcatatatattcttagatgagtaccactgtgctacacccgagcaattagcaataattggaaagatacacaggtttgctgaaaatcttagagtggtagcaatgacagcaaccccagctggaacggtcacaacgactggtcagaaacaccctatagaggagttcatagccccagaggtgatgaaaggtgaagatctaggtagtgaatacttggatattgcagggttgaagataccgactgaagagatgaaaggcaacatgctcgtgttcgcgccaactaggaacatggcagtagaaacagctaagaaattgaaggctaagggatacaactctggatactattacagtggggaaaacccagagaacttgagggtggtaacctcgcaatccccgtatgtggtagtagccaccaatgccatagagtcaggtgtgacattaccagacttagacacagttgtagacactggactaaagtgtgagaagagggtgaggatttcttcaaaaatgcccttcattgtaacaggacttaagagaatggcagtcacaatcggagagcaagcccagcgcaggggtagagtaggaagagtcaagccaggtaggtactataggagtcaagaaacagcttcagggtcaaaagattaccattacgacctactgcaagcccagaggtacggaatagaagatggaattaatgtaacaaagtcattcagggagatgaactatgattggagcctttacgaagaggacagcttgatgataactcaactcgaggtccttaacaacctccttatatcagaagacctgcctgccgcagtgaagaacatcatggcccggaccgatcacccagaacccatacaactggcctataacagttatgaaaaccaaattccagtgctgttcccaaagatcaaaaatggtgaggtgacagacagttatgagaattacacatatctcaatgcaagaaaattaggagaggacgtgccggcatatgtgtacgccacagaggatgaggatctagcagtggatcttctgggtatggattggccggacccaggcaaccaacaggtggtagagacagggagggcattaaaacaagtaactggcttatccacagcagaaaacgccctcttgatagccctattcggctacgtcgggtaccagacactttcaaaaaggcacatacccatgattactgacatctatacacttgaagaccacaggcttgaggacacaacccacctccagtttgccccaaacgctataaggaccgacggcaaggactcagagttgaaggaattagctgtgggagaccttgataaatatgtggacgcactggtagactactccaaacaagggatgaaattcatcaaagtccaagctgaaaaggtcagagactcccagtctacgaaggaaggcttgcaaaccattaaggagtatgtggataagtttatacaatcactaacagagaataaggaggagatcatcaggtatggactatggggagttcacacggcactctacaaaagcttggcagcgagactggggcatgaaacagcttttgcaactttagtggtaaaatggttggcttttgggggcgaaacggtatctgctcacatcaagcaagtagcagttgatctagtagtatattatatcatcaacaaaccatcttttcctggagatacagagacccaacaagaggggaggaagtttgtggctagtctttttatatctgcactagcaacatacacatataaaacctggaattacaacaatctgcaacgggttgtcgaacctgccttagcttacctcccatatgctacaagtgccttgaagttgttcacacccacaagattagagagtgtggtcatactcagttctacaatttacaagacatacctctctataaggaagggtaagagtgacggcttgttaggtacaggcataagtgcagccatggagatcttaaaccaaaacccaatctcagtaggtatatctgtgatgctgggggtaggtgccatcgccgcccataatgcaatagaatctagtgaacagaaaagaactttgctgatgaaggtctttgtaaaaaacttcttagaccaagcagcaacagatgagctagtcaaagagaaccctgaaaaaataatcatggctctatttgaagcagtccagaccataggaaaccccctaagactcatctaccatctgtacggggtgtactataaggggtgggaagcaaaagaactcgcagagaaaactgctggccgcaacttattcacattgatcatgtttgaggcctttgagcttttaggtatggactcagaaggaaagataagaaacttgtcaggcaactacatactggacttaatcttcaacttgcataataaattaaacaaggggctcaaaaaactagtccttgggtgggctcctgcacctttgagctgtgattggacaccaagtgatgagagaataagcctacctcataacaactacttaagggtagaaaccaggtgtccttgtggctatgagatgaaggcaataaaaaatgttgctggtaaattgacaaaagttgaagaaaaggggtccttcctatgcaggaatagattagggagaggacctccaaacttcaaagtaacaaagttctatgatgataacttgatagaagtcaagccagtagctaggctagaaggccaggtggacctctattacaagggagtaacagctaagttagactacaacaatgggaaagtactgttagctaccaacaagtgggaggtggaccacgctttcctgaccagactagtaaagaagcacacagggataggttttaaaggtgcatatttgggtgaccgaccagaccatcaagatcttgtcgatagagattgtgcaactataacgaagaactcagtacagttcctaaaaatgaagaagggttgcgctttcacatatgacctaacaatctctaaccttgtcaggcttattgaactagtccataagaataatttacaagaaagagagatccctaccgtgacagtaactacttggcttgcatattcttttgtcaatgaagacctggggactatcaagcctgtattgggggagaaagtcatcccagaaccccccgaggagttgagtctccaacccaccgtgagactagtcaccactgaaacagcaataaccataacaggggaggctgaagtgatgacgacagggatcacaccagtggtagagatgaaagaagaacctcagctggaccaccagtcaactaccctaaaggtagggttgaaggaaggggaatatccagggccaggagttaaccctaaccatttagcagaggtgatagatgagaaagatgacaggccttttgtcctaatcatcggtaacaaaggttctacctcgaacagagcaagaacggccaagaatatacggctgtacaaaggaaacaacccaagagagatcagggatctgatgagccaaggaagaatattgacggttgctctaaaagagttggacccggaattaaaagaattagtagattacaaggggacctttctcaatagggaagctttagaagccctaagcttaggtaagccaatcaagaggaaaaccacaacagcaatgatcaggaggttaatagagccagaggttgaggaggaactaccagattggttccaagcggaagaacccctatttttggaagcaaaaatacagaatgacttataccacctaattggcagtgtagatagtataaaaagcaaagcaaaggaattaggggccacagataacacaaagatagtgaaggaagttggggctaggacctatacgatgaaattgagcagctggagcacacaagttacaaaaaaacagatgagtctagcccctctctttgaagagctgttattaaagtgccctccatgtagtaaaatttcaaagggacatatggtgtcagcataccaactggctcaaggaaactgggaacccctcgggtgtggggtctatatgggaaccataccagctaggcgtctcaagatccacccttatgaggcttaccttaaactcaaagagctggtggaagttgaatcttcgagggccactgcaaaagaatccatcataagagaacataacacctggatcctgcggaaggtgagacatgaagggaacctaagaaccaaatcaatgatcaaccctgggaaaatatcagatcagctatgcagagatggacacaaaagaaacatatataataagatcataggctcaacaatggcctctgctggtattaggctggagaaactgccagtagtccgagcccaaactgacacaaccagtttccaccaagccataagagaaaaaattgataaaacagaaaacaagcagacccctgaattgcatgaagaactaatgaaggtcttcgactgcttaaagatcccagagctgaaggaatcgtatgatgaagtttcatgggaacaattagaagccgggataaaccgtaagggtgcagcaggctatctagagagcaagaacataggggaagtcctagacacagagaaacacatagtagagcagctgatcaaggatctgaggaaggggaagaagattaggtactatgaaacagccatccccaagaatgagaagagagacgtcagcgacgactgggaagccggagagttcgttgatgaaaagaaaccaagagtaatccagtacccggacgccaaggtgagactggccattacaaaagtgatgtacaaatgggtaaagcaaaaaccagtggtgatacccggctatgaaggtaaaacacctctatttgacatattcaacaaagtgaagaaggaatgggattcattccaggaccccgtagcagtgagctttgacaccaaagcgtgggatacacaagtcaccagtagagacctaatgttgataaaggatatccagaaatattatttcaagagaagtatacacaaatttttagatacaataacagaacacatggtggaggtacctgtcattacagcagacggtgaagtttacataaggaatggtcagaggggtagtggccaacccgacacaagtgctggtaatagtatgttgaatgtcctaaccatgatatatgctttctgtaaaagtacaggcataccttacaggggattcagcagagtggcaagaatccatgtgtgtggtgatgatggctttttgataacagagagaggactgggactgaaattctctgagaagggtatgcagatattacatgaggccgggaagccccagaaaataactgaaggggacaaaatgaaagtggcatacagattcgaggacatagagttttgttcccatactcccgtgccagtcagatgggcagataacaccagtagttacatggcagggaggagcacagccactatactagctaagatggcaaccaggctggattccagcggagagaggggtagcacagcttatgagaaggccgtagccttcagcttccttttgatgtactcatggaatcccgtagttagaaggatctgcttactggtgttgtcacagtttccagaaatatccccatccaaaaacacaatatactactaccaaggggatcccatagctgcgtacagagaagtgatagggaaacagctgtgtgaactgaaaagaacaggatttgagaagctggctggtctgaatttgagtatgaccactctaggcatctggacaaaacatactagtaaaagactaatccaagcctgtgtagaaataggtaagagagaaggtacctggttagttaatgctgacagactgattgcaggaaagactgggaagttttacatcccaagcactggtgtcactctgttgggaaaacactatgaggaaattaacttaaagcaaaaggcggcacaaccgccgatagagggggttgacagatataagttgggccccatagttaatgttatcttgagaaggctgagggtgatgctgatgacagttgccagcggaagctggtgaatccgtccggagcgtcgtgccctcactcaaggtttttaattgtaaatattgtaaatagacagctaagatatttattgtagttggatagtaatgcagtgatagtaaataccccaatttaacactacctccaatgcactaagcactttagctgtgtgaggttaactcgacgtccacggttggactagggaagacctctaacagcccc

SEQ ID NO: 2 - DNA, 11840 bp, Artificial Sequence
  Mutated BVDV XIKE- A-NdN
gtatacgagattagctaaagaactcgtatatggattggacgtcaacaaatttttaattggcaacgtagggaaccttcccctcagcgaaggccgaaaagaggctagccatgcccttagtaggactagcaaaagtaggggactagcggtagcagtgagttcgttggatggccgaacccctgagtacaggggagtcgtcaatggttcgacactccattagtcgaggagtctcgagatgccatgtggacgagggcatgcccacggcacatcttaacccatgcgggggttgcatgggtgaaagcgctattcgtggcgttatggacacagcctgatagggtgtagcagagacctgctattccgctagtaaaaactctgctgtacatggcacatggagttgttttccgatgaagggagcaagggtgctacaagtaagaagcagcctaagccagataggatagaaaaaggtaagatgaaaatagccccaaaagagacagaaaaagattgcaaaaccagaccccccgacgcgactatagtagtagaaggggttaagtaccaggtgaagaaaaaaggaaaggtaaggggaaaaaatactcaagatgggttatatcacaacaagaataagccccctgaatcaagaaaaaaattggaaaaggcactgctggcttgggccatcttagcagcggtcctgcttcagctggtaacaggagagaatatcacccagtggaacttgatggacaacggcaccgagggaatacagcaagcgatgttcctaagaggggtgaacaggagtctacatggaatttggccagagaaaatttgcaccggagtaccaactcacttagcaacagactatgagcttaaagagatagtggggatgatggacgcgagtgagaagaccaactacacgtgttgcaggttgcaaagacatgagtggaataaacatggttggtgtaactggtttcatatagaaccgtggatatggttgatgaacaaaacccaaaacaacctgacagaagggcaaccgcttagggagtgtgctgtgacttgtaggtatgacaaggaaacagaattgaacatcgtgacacaggctagggacagacctacaactctgacaggttgcaagaaaggcaagaatttctctttcgcaggtgttatactggatgggccctgtaactttaaagtatcggttgaagatgtgctgttcaaggagcacgattgcggcaacatgctgcaagagaccgcgatacagctactcgatggggcaaccaacaccattgagggagcaagggtagggacggccaagttgacaacctggttagggaagcaattagggatccttggtaagaagttggagaacaaaagcaaagcatggtttggtgcacatgcagcaagtccatactgcggagtggagaggaagatcggttacgtatggtatacaaaaaactgcactccagcttgccttccaagaaacactagaataataggccccgggaaatttgataccaacgccgaagatggaaaaatactccatgagatgggggggcacctctcagaatttgtcctattgtccttggtggttctgtctgactttgccccggaaaccgcgagcgtcatctacttggttctacattttgcgatcccgcaaagccacgttgatgtagacacatgcgacaagaaccagctgaatttaacggtagcaaccacagtagcagaggtcataccagggacagtgtggaacctagggaagtatgtctgcataagaccagactggtggccatatgagacgacgacagtcttcgtcatagaggaagcagggcaagtaatcaaattgatgctaagggccatcagagacttaactaggatatggaatgctgccactaccacagctttcttaatctttttagtaaaagcactgaggggacaactaatccaagggctattgtggctgatgctaataacaggagcacagggcttccctgaatgcaaagagggcttccaatatgccatatctaaagacaggaaaatggggttattggggccagagagcttaactacaacatggcacctccccaccaaaaaaatagtggattccatggtgcatgtatggtgtgaaggaaaagacttgaaaatattaaaaatgtgcacaaaggaagagaggtatctagtggctgtgcacgagagagccttatcaaccagtgccgagtttatgcagatcagtgatgggacaataggcccagacgtgatagatatgcctgatgactttgagtttggactctgcccttgtgactcaaaaccagtgataaagggcaaatttaatgccagcttactgaatggaccagctttccagatggtatgcccacaggggtggactggtacaatagaatgcaccctagcgaaccaagacaccttggacacaactgtcattaggacatatagaagaactaccccatttcagcggagaaaatggtgtacctatgaaaaaataataggggaagatatctatgaatgcattctaggtggaaactggacatgcataaccggtgaccatagcaggttgaaagacggacctatcaagaagtgtaagtggtgtggccatgacttcgtcaactcagaggggctaccacactacccaataggcaagtgcatgctcatcaacgagagtgggtacaggtatgtagatgacacctcttgcgataggggtggtgtagccatagttccatctggcaccgtaaagtgtagaataggtaacgtcacggtgcaagttatcgctactaacaatgatctgggacccatgccttgcagcccagctgaagtgatagcaagtgaaggaccagtggaaaagactgcatgcacattcaactattcaaggactctacctaataagtattatgagccaagggaccggtacttccaacaatacatgttaaaaggggagtggcaatattggttcgacctggattctgtagaccaccacaaagactacttctcagagttcataatcatagcagtggtcgccttgttgggtggtaagtacgtactgtggctcttgataacatacacaatactgtctgagcagatggctatgggtgctggagtgaatactgaagagatagtcatgataggcaatttgctgacagacagtgatattgaggttgtggtttatttccttcttctgtacttaatagttaaagaggaactggcgaggaaatggattatactggtataccacatccttgtagccaaccctatgaaaacaattggggtcgtcttactaatgctagggggagtggtgaaggccagcagaatcaatgctgatgaccaaagtgctatggacccatgctttcttctcgtgacaggcgtagtggctgttttgatgatcgctagaagagaacctgccacattaccactgattgtagcattgctagcaataagaacatcaggattcctactgcccgctagcattgatgtaactgtagcagtagtattaattgtacttttgttggctagctacataacagactactttagatataaaaagtggcttcaactcttatttagtctgatagctggtatctttattataaggagcttaaaacatatcaaccagatggaggtaccagaaatatctatgccaagttggagacctctagctctggtccttttctatataacatctacagcaataaccactaattgggacattgacttagcaggcttcctgctgcaatgggcgccagcagtgatcatgatggctaccatgtgggcagactttttgactctgatcatagtcctgcccagttacgagttatctaagctttacttcctaaagaacgtcaggacagacgtggaaaagaactggctcggcaaagtgaaatacagacagatcagttcagtttatgacatctgtgacagtgaggaagcagtgtacctatttccatcaaggcataagagtggaagcaggccagatttcatattaccttttttgaaagccgtgttaataagctgcatcagcagccaatggcaagtggtttacatttcttacctaatactggaaattacatactatatgcacaggaaaatcatagatgaggtgtcaggaggagcaaattttctatcaagactcatagcagccatcatagaattaaattgggccatagatgatgaggaatgtaaaggactgaagaaactgtatctcttgtcagggagagcgaagaatttgatagttaaacataaggtaagaaatgaagccgtccacagatggtttggtgaggaggaaatatacggggcacccaaggtgatcactatcataaaagctagtaccctaagtaaaaacaggcactgcataatctgcacgatctgtgaagggaaagaatggaatggagccaactgcccaaagtgtggaagacaaggaaagcccataacatgtggaatgacactcgcagactttgaggagaaacattacaaaaagatatttataagagaagaatcttcttgtcctgtgccttttgatccttcttgccattgtaattattttcgccacgatgggcctttcaggaaagagtataagggttacgtccaatacacagccagaggacaactctttctgaggaacctaccaattctagcgacgaagatgaagctattaatggtgggaaacctcggcgcagaaattggcgacctggaacatctaggatgggtactgagagggccagccgtgtgcaaaaaaattaccaaccatgagaagtgccacgtaaacatcatggataagctaactgcattttttggaatcatgcctagaggcacgacccctagggcacctgtgaggttccccacagcactactaaaagtgagaagggggctagagacgggatgggcttacacgcaccaaggagggatcagctcggtagaccatgtcacagccggaaaggatttactagtgtgtgacagtatgggcaggaccagggttgtctgtcatagtaacaataagatgactgatgagactgagtatggcatcaagaccgactcagggtgtcccgaaggtgcgaggtgttacgtgctaaacccagaagctgttaacatttctggcacaaaaggagctatggtacacctccagaaaacggggggggagttcacatgtgtcactgcctcagggaccccggctttcttcgatctgaaaaatctaaaaggctggtccgggctaccaatttttgaagcatccagtggcagggtggttggtagggtgaaagtcggcaagaatgaggattccaagcccaccaaactaatgagcggaatccagacagtgtctaagaaccagacagacctagcggacatcgtaaaaaaattgactagtatgaacagaggagagttcaaacagataacattagccactggggcaggaaaaactacggaactgccaaggtccgtcatagaggagatagggaggcacaaaagggtcttagtcctgataccattgagagcagcagcagagtcagtgtatcagtatatgagagtgaagtacccaagtatatctttcaatttgagaataggagatatgaaggaaggtgacatggccactggtatcacctacgcctcatatgggtacttttgtcagcttcctcagcccaaactgagagctgccatggtagagtactcatatatattcttagatgagtaccactgtgctacacccgagcaattagcaataattggaaagatacacaggtttgctgaaaatcttagagtggtagcaatgacagcaaccccagctggaacggtcacaacgactggtcagaaacaccctatagaggagttcatagccccagaggtgatgaaaggtgaagatctaggtagtgaatacttggatattgcagggttgaagataccgactgaagagatgaaaggcaacatgctcgtgttcgcgccaactaggaacatggcagtagaaacagctaagaaattgaaggctaagggatacaactctggatactattacagtggggaaaacccagagaacttgagggtggtaacctcgcaatccccgtatgtggtagtagccaccaatgccatagagtcaggtgtgacattaccagacttagacacagttgtagacactggactaaagtgtgagaagagggtgaggatttcttcaaaaatgcccttcattgtaacaggacttaagagaatggcagtcacaatcggagagcaagcccagcgcaggggtagagtaggaagagtcaagccaggtaggtactataggagtcaagaaacagcttcagggtcaaaagattaccattacgacctactgcaagcccagaggtacggaatagaagatggaattaatgtaacaaagtcattcagggagatgaactatgattggagcctttacgaagaggacagcttgatgataactcaactcgaggtccttaacaacctccttatatcagaagacctgcctgccgcagtgaagaacatcatggcccggaccgatcacccagaacccatacaactggcctataacagttatgaaaaccaaattccagtgctgttcccaaagatcaaaaatggtgaggtgacagacagttatgagaattacacatatctcaatgcaagaaaattaggagaggacgtgccggcatatgtgtacgccacagaggatgaggatctagcagtggatcttctgggtatggattggccggacccaggcaaccaacaggtggtagagacagggagggcattaaaacaagtaactggcttatccacagcagaaaacgccctcttgatagccctattcggctacgtcgggtaccagacactttcaaaaaggcacatacccatgattactgacatctatacacttgaagaccacaggcttgaggacacaacccacctccagtttgccccaaacgctataaggaccgacggcaaggactcagagttgaaggaattagctgtgggagaccttgataaatatgtggacgcactggtagactactccaaacaagggatgaaattcatcaaagtccaagctgaaaaggtcagagactcccagtctacgaaggaaggcttgcaaaccattaaggagtatgtggataagtttatacaatcactaacagagaataaggaggagatcatcaggtatggactatggggagttcacacggcactctacaaaagcttggcagcgagactggggcatgaaacagcttttgcaactttagtggtaaaatggttggcttttgggggcgaaacggtatctgctcacatcaagcaagtagcagttgatctagtagtatattatatcatcaacaaaccatcttttcctggagatacagagacccaacaagaggggaggaagtttgtggctagtctttttatatctgcactagcaacatacacatataaaacctggaattacaacaatctgcaacgggttgtcgaacctgccttagcttacctcccatatgctacaagtgccttgaagttgttcacacccacaagattagagagtgtggtcatactcagttctacaatttacaagacatacctctctataaggaagggtaagagtgacggcttgttaggtacaggcataagtgcagccatggagatcttaaaccaaaacccaatctcagtaggtatatctgtgatgctgggggtaggtgccatcgccgcccataatgcaatagaatctagtgaacagaaaagaactttgctgatgaaggtctttgtaaaaaacttcttagaccaagcagcaacagatgagctagtcaaagagaaccctgaaaaaataatcatggctctatttgaagcagtccagaccataggaaaccccctaagactcatctaccatctgtacggggtgtactataaggggtgggaagcaaaagaactcgcagagaaaactgctggccgcaacttattcacattgatcatgtttgaggcctttgagcttttaggtatggactcagaaggaaagataagaaacttgtcaggcaactacatactggacttaatcttcaacttgcataataaattaaacaaggggctcaaaaaactagtccttgggtgggctcctgcacctttgagctgtgattggacaccaagtgatgagagaataagcctacctcataacaactacttaagggtagaaaccaggtgtccttgtggctatgagatgaaggcaataaaaaatgttgctggtaaattgacaaaagttgaagaaaaggggtccttcctatgcaggaatagattagggagaggacctccaaacttcaaagtaacaaagttctatgatgataacttgatagaagtcaagccagtagctaggctagaaggccaggtggacctctattacaagggagtaacagctaagttagactacaacaatgggaaagtactgttagctaccaacaagtgggaggtggaccacgctttcctgaccagactagtaaagaagcacacagggataggttttaaaggtgcatatttgggtgaccgaccagaccatcaagatcttgtcgatagagattgtgcaactataacgaagaactcagtacagttcctaaaaatgaagaagggttgcgctttcacatatgacctaacaatctctaaccttgtcaggcttattgaactagtccataagaataatttacaagaaagagagatccctaccgtgacagtaactacttggcttgcatattcttttgtcaatgaagacctggggactatcaagcctgtattgggggagaaagtcatcccagaaccccccgaggagttgagtctccaacccaccgtgagactagtcaccactgaaacagcaataaccataacaggggaggctgaagtgatgacgacagggatcacaccagtggtagagatgaaagaagaacctcagctggaccaccagtcaactaccctaaaggtagggttgaaggaaggggaatatccagggccaggagttaaccctaaccatttagcagaggtgatagatgagaaagatgacaggccttttgtcctaatcatcggtaacaaaggttctacctcgaacagagcaagaacggccaagaatatacggctgtacaaaggaaacaacccaagagagatcagggatctgatgagccaaggaagaatattgacggttgctctaaaagagttggacccggaattaaaagaattagtagattacaaggggacctttctcaatagggaagctttagaagccctaagcttaggtaagccaatcaagaggaaaaccacaacagcaatgatcaggaggttaatagagccagaggttgaggaggaactaccagattggttccaagcggaagaacccctatttttggaagcaaaaatacagaatgacttataccacctaattggcagtgtagatagtataaaaagcaaagcaaaggaattaggggccacagataacacaaagatagtgaaggaagttggggctaggacctatacgatgaaattgagcagctggagcacacaagttacaaaaaaacagatgagtctagcccctctctttgaagagctgttattaaagtgccctccatgtagtaaaatttcaaagggacatatggtgtcagcataccaactggctcaaggaaactgggaacccctcgggtgtggggtctatatgggaaccataccagctaggcgtctcaagatccacccttatgaggcttaccttaaactcaaagagctggtggaagttgaatcttcgagggccactgcaaaagaatccatcataagagaacataacacctggatcctgcggaaggtgagacatgaagggaacctaagaaccaaatcaatgatcaaccctgggaaaatatcagatcagctatgcagagatggacacaaaagaaacatatataataagatcataggctcaacaatggcctctgctggtattaggctggagaaactgccagtagtccgagcccaaactgacacaaccagtttccaccaagccataagagaaaaaattgataaaacagaaaacaagcagacccctgaattgcatgaagaactaatgaaggtcttcgactgcttaaagatcccagagctgaaggaatcgtatgatgaagtttcatgggaacaattagaagccgggataaaccgtaagggtgcagcaggctatctagagagcaagaacataggggaagtcctagacacagagaaacacatagtagagcagctgatcaaggatctgaggaaggggaagaagattaggtactatgaaacagccatccccaagaatgagaagagagacgtcagcgacgactgggaagccggagagttcgttgatgaaaagaaaccaagagtaatccagtacccggacgccaaggtgagactggccattacaaaagtgatgtacaaatgggtaaagcaaaaaccagtggtgatacccggctatgaaggtaaaacacctctatttgacatattcaacaaagtgaagaaggaatgggattcattccaggaccccgtagcagtgagctttgacaccaaagcgtgggatacacaagtcaccagtagagacctaatgttgataaaggatatccagaaatattatttcaagagaagtatacacaaatttttagatacaataacagaacacatggtggaggtacctgtcattacagcagacggtgaagtttacataaggaatggtcagaggggtagtggccaacccgacacaagtgctggtaatagtatgttgaatgtcctaaccatgatatatgctttctgtaaaagtacaggcataccttacaggggattcagcagagtggcaagaatccatgtgtgtggtgatgatggctttttgataacagagagaggactgggactgaaattctctgagaagggtatgcagatattacatgaggccgggaagccccagaaaataactgaaggggacaaaatgaaagtggcatacagattcgaggacatagagttttgttcccatactcccgtgccagtcagatgggcagataacaccagtagttacatggcagggaggagcacagccactatactagctaagatggcaaccaggctggattccagcggagagaggggtagcacagcttatgagaaggccgtagccttcagcttccttttgatgtactcatggaatcccgtagttagaaggatctgcttactggtgttgtcacagtttccagaaatatccccatccaaaaacacaatatactactaccaaggggatcccatagctgcgtacagagaagtgatagggaaacagctgtgtgaactgaaaagaacaggatttgagaagctggctggtctgaatttgagtatgaccactctaggcatctggacaaaacatactagtaaaagactaatccaagcctgtgtagaaataggtaagagagaaggtacctggttagttaatgctgacagactgattgcaggaaagactgggaagttttacatcccaagcactggtgtcactctgttgggaaaacactatgaggaaattaacttaaagcaaaaggcggcacaaccgccgatagagggggttgacagatataagttgggccccatagttaatgttatcttgagaaggctgagggtgatgctgatgacagttgccagcggaagctggtgaatccgtccggagcgtcgtgccctcactcaaggtttttaattgtaaatattgtaaatagacagctaagatatttattgtagttggatagtaatgcagtgatagtaaataccccaatttaacactacctccaatgcactaagcactttagctgtgtgaggttaactcgacgtccacggttggactagggaagacctctaacagcccc

SEQ ID NO: 3 - DNA, 12329 bp, Artificial Sequence
  Mutated BVDV XIKE-B
gtatacgagattagctaaagaactcgtatatggattggacgtcaacaaatttttaattggcaacgtagggaaccttcccctcagcgaaggccgaaaagaggctagccatgcccttagtaggactagcaaaagtaggggactagcggtagcagtgagttcgttggatggccgaacccctgagtacaggggagtcgtcaatggttcgacactccattagtcgaggagtctcgagatgccatgtggacgagggcatgcccacggcacatcttaacccatgcgggggttgcatgggtgaaagcgctattcgtggcgttatggacacagcctgatagggtgtagcagagacctgctattccgctagtaaaaactctgctgtacatggcacatggagttgttttcaaatgaacttttatacaaaacatataaacaaaaaccagcaggcgtcgtggaacctgtttacgacgtcaacgggcgcccactgtttggagagagcagtgacttgcacccgcagtcaacactaaaactaccacaccaacgaggcagcgccaacatcctgaccaatgctaggtccctaccgcggaaaggtgactgccggagaggtaatgtgtatggaccggtgagtggcatctatatcaaaccaggaccgatctactaccaggattatgtgggccccgtctatcatagagccccactggaactatgtagggaggcaagtatgtgcgaaacaactaggagagttggcagagtgaccggtagtgatgggaaattatatcatatctacatctgcatagatgggtgtatcctcctgaagagggcgactaggaaccaaccagaagtcctgaaatgggtatacaacagattaaattgtcctttatgggtcaccagctgctccgatgaagggagcaagggtgctacaagtaagaagcagcctaagccagataggatagaaaaaggtaagatgaaaatagccccaaaagagacagaaaaagattgcaaaaccagaccccccgacgcgactatagtagtagaaggggttaagtaccaggtgaagaaaaaaggaaaggtaaggggaaaaaatactcaagatgggttatatcacaacaagaataagccccctgaatcaagaaaaaaattggaaaaggcactgctggcttgggccatcttagcagcggtcctgcttcagctggtaacaggagagaatatcacccagtggaacttgatggacaacggcaccgagggaatacagcaagcgatgttcctaagaggggtgaacaggagtctacatggaatttggccagagaaaatttgcaccggagtaccaactcacttagcaacagactatgagcttaaagagatagtggggatgatggacgcgagtgagaagaccaactacacgtgttgcaggttgcaaagacatgagtggaataaaggttggtgtaactggtttcatatagaaccgtggatatggttgatgaacaaaacccaaaacaacctgacagaagggcaaccgcttagggagtgtgctgtgacttgtaggtatgacaaggaaacagaattgaacatcgtgacacaggctagggacagacctacaactctgacaggttgcaagaaaggcaagaatttctctttcgcaggtgttatactggatgggccctgtaactttaaagtatcggttgaagatgtgctgttcaaggagcacgattgcggcaacatgctgcaagagaccgcgatacagctactcgatggggcaaccaacaccattgagggagcaagggtagggacggccaagttgacaacctggttagggaagcaattagggatccttggtaagaagttggagaacaaaagcaaagcatggtttggtgcacatgcagcaagtccatactgcggagtggagaggaagatcggttacgtatggtatacaaaaaactgcactccagcttgccttccaagaaacactagaataataggccccgggaaatttgataccaacgccgaagatggaaaaatactccatgagatgggggggcacctctcagaatttgtcctattgtccttggtggttctgtctgactttgccccggaaaccgcgagcgtcatctacttggttctacattttgcgatcccgcaaagccacgttgatgtagacacatgcgacaagaaccagctgaatttaacggtagcaaccacagtagcagaggtcataccagggacagtgtggaacctagggaagtatgtctgcataagaccagactggtggccatatgagacgacgacagtcttcgtcatagaggaagcagggcaagtaatcaaattgatgctaagggccatcagagacttaactaggatatggaatgctgccactaccacagctttcttaatctttttagtaaaagcactgaggggacaactaatccaagggctattgtggctgatgctaataacaggagcacagggcttccctgaatgcaaagagggcttccaatatgccatatctaaagacaggaaaatggggttattggggccagagagcttaactacaacatggcacctccccaccaaaaaaatagtggattccatggtgcatgtatggtgtgaaggaaaagacttgaaaatattaaaaatgtgcacaaaggaagagaggtatctagtggctgtgcacgagagagccttatcaaccagtgccgagtttatgcagatcagtgatgggacaataggcccagacgtgatagatatgcctgatgactttgagtttggactctgcccttgtgactcaaaaccagtgataaagggcaaatttaatgccagcttactgaatggaccagctttccagatggtatgcccacaggggtggactggtacaatagaatgcaccctagcgaaccaagacaccttggacacaactgtcattaggacatatagaagaactaccccatttcagcggagaaaatggtgtacctatgaaaaaataataggggaagatatctatgaatgcattctaggtggaaactggacatgcataaccggtgaccatagcaggttgaaagacggacctatcaagaagtgtaagtggtgtggccatgacttcgtcaactcagaggggctaccacactacccaataggcaagtgcatgctcatcaacgagagtgggtacaggtatgtagatgacacctcttgcgataggggtggtgtagccatagttccatctggcaccgtaaagtgtagaataggtaacgtcacggtgcaagttatcgctactaacaatgatctgggacccatgccttgcagcccagctgaagtgatagcaagtgaaggaccagtggaaaagactgcatgcacattcaactattcaaggactctacctaataagtattatgagccaagggaccggtacttccaacaatacatgttaaaaggggagtggcaatattggttcgacctggattctgtagaccaccacaaagactacttctcagagttcataatcatagcagtggtcgccttgttgggtggtaagtacgtactgtggctcttgataacatacacaatactgtctgagcagatggctatgggtgctggagtgaatactgaagagatagtcatgataggcaatttgctgacagacagtgatattgaggttgtggtttatttccttcttctgtacttaatagttaaagaggaactggcgaggaaatggattatactggtataccacatccttgtagccaaccctatgaaaacaattggggtcgtcttactaatgctagggggagtggtgaaggccagcagaatcaatgctgatgaccaaagtgctatggacccatgctttcttctcgtgacaggcgtagtggctgttttgatgatcgctagaagagaacctgccacattaccactgattgtagcattgctagcaataagaacatcaggattcctactgcccgctagcattgatgtaactgtagcagtagtattaattgtacttttgttggctagctacataacagactactttagatataaaaagtggcttcaactcttatttagtctgatagctggtatctttattataaggagcttaaaacatatcaaccagatggaggtaccagaaatatctatgccaagttggagacctctagctctggtccttttctatataacatctacagcaataaccactaattgggacattgacttagcaggcttcctgctgcaatgggcgccagcagtgatcatgatggctaccatgtgggcagactttttgactctgatcatagtcctgcccagttacgagttatctaagctttacttcctaaagaacgtcaggacagacgtggaaaagaactggctcggcaaagtgaaatacagacagatcagttcagtttatgacatctgtgacagtgaggaagcagtgtacctatttccatcaaggcataagagtggaagcaggccagatttcatattaccttttttgaaagccgtgttaataagctgcatcagcagccaatggcaagtggtttacatttcttacctaatactggaaattacatactatatgcacaggaaaatcatagatgaggtgtcaggaggagcaaattttctatcaagactcatagcagccatcatagaattaaattgggccatagatgatgaggaatgtaaaggactgaagaaactgtatctcttgtcagggagagcgaagaatttgatagttaaacataaggtaagaaatgaagccgtccacagatggtttggtgaggaggaaatatacggggcacccaaggtgatcactatcataaaagctagtaccctaagtaaaaacaggcactgcataatctgcacgatctgtgaagggaaagaatggaatggagccaactgcccaaagtgtggaagacaaggaaagcccataacatgtggaatgacactcgcagactttgaggagaaacattacaaaaagatatttataagagaagaatcttcttgtcctgtgccttttgatccttcttgccattgtaattattttcgccacgatgggcctttcaggaaagagtataagggttacgtccaatacacagccagaggacaactctttctgaggaacctaccaattctagcgacgaagatgaagctattaatggtgggaaacctcggcgcagaaattggcgacctggaacatctaggatgggtactgagagggccagccgtgtgcaaaaaaattaccaaccatgagaagtgccacgtaaacatcatggataagctaactgcattttttggaatcatgcctagaggcacgacccctagggcacctgtgaggttccccacagcactactaaaagtgagaagggggctagagacgggatgggcttacacgcaccaaggagggatcagctcggtagaccatgtcacagccggaaaggatttactagtgtgtgacagtatgggcaggaccagggttgtctgtcatagtaacaataagatgactgatgagactgagtatggcatcaagaccgactcagggtgtcccgaaggtgcgaggtgttacgtgctaaacccagaagctgttaacatttctggcacaaaaggagctatggtacacctccagaaaacggggggggagttcacatgtgtcactgcctcagggaccccggctttcttcgatctgaaaaatctaaaaggctggtccgggctaccaatttttgaagcatccagtggcagggtggttggtagggtgaaagtcggcaagaatgaggattccaagcccaccaaactaatgagcggaatccagacagtgtctaagaaccagacagacctagcggacatcgtaaaaaaattgactagtatgaacagaggagagttcaaacagataacattagccactggggcaggaaaaactacggaactgccaaggtccgtcatagaggagatagggaggcacaaaagggtcttagtcctgataccattgagagcagcagcagagtcagtgtatcagtatatgagagtgaagtacccaagtatatctttcaatttgagaataggagatatgaaggaaggtgacatggccactggtatcacctacgcctcatatgggtacttttgtcagcttcctcagcccaaactgagagctgccatggtagagtactcatatatattcttagatgagtaccactgtgctacacccgagcaattagcaataattggaaagatacacaggtttgctgaaaatcttagagtggtagcaatgacagcaaccccagctggaacggtcacaacgactggtcagaaacaccctatagaggagttcatagccccagaggtgatgaaaggtgaagatctaggtagtgaatacttggatattgcagggttgaagataccgactgaagagatgaaaggcaacatgctcgtgttcgcgccaactaggaacatggcagtagaaacagctaagaaattgaaggctaagggatacaactctggatactattacagtggggaaaacccagagaacttgagggtggtaacctcgcaatccccgtatgtggtagtagccaccaatgccatagagtcaggtgtgacattaccagacttagacacagttgtagacactggactaaagtgtgagaagagggtgaggatttcttcaaaaatgcccttcattgtaacaggacttaagagaatggcagtcacaatcggagagcaagcccagcgcaggggtagagtaggaagagtcaagccaggtaggtactataggagtcaagaaacagcttcagggtcaaaagattaccattacgacctactgcaagcccagaggtacggaatagaagatggaattaatgtaacaaagtcattcagggagatgaactatgattggagcctttacgaagaggacagcttgatgataactcaactcgaggtccttaacaacctccttatatcagaagacctgcctgccgcagtgaagaacatcatggcccggaccgatcacccagaacccatacaactggcctataacagttatgaaaaccaaattccagtgctgttcccaaagatcaaaaatggtgaggtgacagacagttatgagaattacacatatctcaatgcaagaaaattaggagaggacgtgccggcatatgtgtacgccacagaggatgaggatctagcagtggatcttctgggtatggattggccggacccaggcaaccaacaggtggtagagacagggagggcattaaaacaagtaactggcttatccacagcagaaaacgccctcttgatagccctattcggctacgtcgggtaccagacactttcaaaaaggcacatacccatgattactgacatctatacacttgaagaccacaggcttgaggacacaacccacctccagtttgccccaaacgctataaggaccgacggcaaggactcagagttgaaggaattagctgtgggagaccttgataaatatgtggacgcactggtagactactccaaacaagggatgaaattcatcaaagtccaagctgaaaaggtcagagactcccagtctacgaaggaaggcttgcaaaccattaaggagtatgtggataagtttatacaatcactaacagagaataaggaggagatcatcaggtatggactatggggagttcacacggcactctacaaaagcttggcagcgagactggggcatgaaacagcttttgcaactttagtggtaaaatggttggcttttgggggcgaaacggtatctgctcacatcaagcaagtagcagttgatctagtagtatattatatcatcaacaaaccatcttttcctggagatacagagacccaacaagaggggaggaagtttgtggctagtctttttatatctgcactagcaacatacacatataaaacctggaattacaacaatctgcaacgggttgtcgaacctgccttagcttacctcccatatgctacaagtgccttgaagttgttcacacccacaagattagagagtgtggtcatactcagttctacaatttacaagacatacctctctataaggaagggtaagagtgacggcttgttaggtacaggcataagtgcagccatggagatcttaaaccaaaacccaatctcagtaggtatatctgtgatgctgggggtaggtgccatcgccgcccataatgcaatagaatctagtgaacagaaaagaactttgctgatgaaggtctttgtaaaaaacttcttagaccaagcagcaacagatgagctagtcaaagagaaccctgaaaaaataatcatggctctatttgaagcagtccagaccataggaaaccccctaagactcatctaccatctgtacggggtgtactataaggggtgggaagcaaaagaactcgcagagaaaactgctggccgcaacttattcacattgatcatgtttgaggcctttgagcttttaggtatggactcagaaggaaagataagaaacttgtcaggcaactacatactggacttaatcttcaacttgcataataaattaaacaaggggctcaaaaaactagtccttgggtgggctcctgcacctttgagctgtgattggacaccaagtgatgagagaataagcctacctcataacaactacttaagggtagaaaccaggtgtccttgtggctatgagatgaaggcaataaaaaatgttgctggtaaattgacaaaagttgaagaaaaggggtccttcctatgcaggaatagattagggagaggacctccaaacttcaaagtaacaaagttctatgatgataacttgatagaagtcaagccagtagctaggctagaaggccaggtggacctctattacaagggagtaacagctaagttagactacaacaatgggaaagtactgttagctaccaacaagtgggaggtggaccacgctttcctgaccagactagtaaagaagcacacagggataggttttaaaggtgcatatttgggtgaccgaccagaccatcaagatcttgtcgatagagattgtgcaactataacgaagaactcagtacagttcctaaaaatgaagaagggttgcgctttcacatatgacctaacaatctctaaccttgtcaggcttattgaactagtccataagaataatttacaagaaagagagatccctaccgtgacagtaactacttggcttgcatattcttttgtcaatgaagacctggggactatcaagcctgtattgggggagaaagtcatcccagaaccccccgaggagttgagtctccaacccaccgtgagactagtcaccactgaaacagcaataaccataacaggggaggctgaagtgatgacgacagggatcacaccagtggtagagatgaaagaagaacctcagctggaccaccagtcaactaccctaaaggtagggttgaaggaaggggaatatccagggccaggagttaaccctaaccatttagcagaggtgatagatgagaaagatgacaggccttttgtcctaatcatcggtaacaaaggttctacctcgaacagagcaagaacggccaagaatatacggctgtacaaaggaaacaacccaagagagatcagggatctgatgagccaaggaagaatattgacggttgctctaaaagagttggacccggaattaaaagaattagtagattacaaggggacctttctcaatagggaagctttagaagccctaagcttaggtaagccaatcaagaggaaaaccacaacagcaatgatcaggaggttaatagagccagaggttgaggaggaactaccagattggttccaagcggaagaacccctatttttggaagcaaaaatacagaatgacttataccacctaattggcagtgtagatagtataaaaagcaaagcaaaggaattaggggccacagataacacaaagatagtgaaggaagttggggctaggacctatacgatgaaattgagcagctggagcacacaagttacaaaaaaacagatgagtctagcccctctctttgaagagctgttattaaagtgccctccatgtagtaaaatttcaaagggacatatggtgtcagcataccaactggctcaaggaaactgggaacccctcgggtgtggggtctatatgggaaccataccagctaggcgtctcaagatccacccttatgaggcttaccttaaactcaaagagctggtggaagttgaatcttcgagggccactgcaaaagaatccatcataagagaacataacacctggatcctgcggaaggtgagacatgaagggaacctaagaaccaaatcaatgatcaaccctgggaaaatatcagatcagctatgcagagatggacacaaaagaaacatatataataagatcataggctcaacaatggcctctgctggtattaggctggagaaactgccagtagtccgagcccaaactgacacaaccagtttccaccaagccataagagaaaaaattgataaaacagaaaacaagcagacccctgaattgcatgaagaactaatgaaggtcttcgactgcttaaagatcccagagctgaaggaatcgtatgatgaagtttcatgggaacaattagaagccgggataaaccgtaagggtgcagcaggctatctagagagcaagaacataggggaagtcctagacacagagaaacacatagtagagcagctgatcaaggatctgaggaaggggaagaagattaggtactatgaaacagccatccccaagaatgagaagagagacgtcagcgacgactgggaagccggagagttcgttgatgaaaagaaaccaagagtaatccagtacccggacgccaaggtgagactggccattacaaaagtgatgtacaaatgggtaaagcaaaaaccagtggtgatacccggctatgaaggtaaaacacctctatttgacatattcaacaaagtgaagaaggaatgggattcattccaggaccccgtagcagtgagctttgacaccaaagcgtgggatacacaagtcaccagtagagacctaatgttgataaaggatatccagaaatattatttcaagagaagtatacacaaatttttagatacaataacagaacacatggtggaggtacctgtcattacagcagacggtgaagtttacataaggaatggtcagaggggtagtggccaacccgacacaagtgctggtaatagtatgttgaatgtcctaaccatgatatatgctttctgtaaaagtacaggcataccttacaggggattcagcagagtggcaagaatccatgtgtgtggtgatgatggctttttgataacagagagaggactgggactgaaattctctgagaagggtatgcagatattacatgaggccgggaagccccagaaaataactgaaggggacaaaatgaaagtggcatacagattcgaggacatagagttttgttcccatactcccgtgccagtcagatgggcagataacaccagtagttacatggcagggaggagcacagccactatactagctaagatggcaaccaggctggattccagcggagagaggggtagcacagcttatgagaaggccgtagccttcagcttccttttgatgtactcatggaatcccgtagttagaaggatctgcttactggtgttgtcacagtttccagaaatatccccatccaaaaacacaatatactactaccaaggggatcccatagctgcgtacagagaagtgatagggaaacagctgtgtgaactgaaaagaacaggatttgagaagctggctggtctgaatttgagtatgaccactctaggcatctggacaaaacatactagtaaaagactaatccaagcctgtgtagaaataggtaagagagaaggtacctggttagttaatgctgacagactgattgcaggaaagactgggaagttttacatcccaagcactggtgtcactctgttgggaaaacactatgaggaaattaacttaaagcaaaaggcggcacaaccgccgatagagggggttgacagatataagttgggccccatagttaatgttatcttgagaaggctgagggtgatgctgatgacagttgccagcggaagctggtgaatccgtccggagcgtcgtgccctcactcaaggtttttaattgtaaatattgtaaatagacagctaagatatttattgtagttggatagtaatgcagtgatagtaaataccccaatttaacactacctccaatgcactaagcactttagctgtgtgaggttaactcgacgtccacggttggactagggaagacctctaacagcccc

SEQ ID NO: 4 - DNA, 11837 bp, Artificial Sequence
  Mutated BVDV XIKE-B-NdN
gtatacgagattagctaaagaactcgtatatggattggacgtcaacaaatttttaattggcaacgtagggaaccttcccctcagcgaaggccgaaaagaggctagccatgcccttagtaggactagcaaaagtaggggactagcggtagcagtgagttcgttggatggccgaacccctgagtacaggggagtcgtcaatggttcgacactccattagtcgaggagtctcgagatgccatgtggacgagggcatgcccacggcacatcttaacccatgcgggggttgcatgggtgaaagcgctattcgtggcgttatggacacagcctgatagggtgtagcagagacctgctattccgctagtaaaaactctgctgtacatggcacatggagttgttttccgatgaagggagcaagggtgctacaagtaagaagcagcctaagccagataggatagaaaaaggtaagatgaaaatagccccaaaagagacagaaaaagattgcaaaaccagaccccccgacgcgactatagtagtagaaggggttaagtaccaggtgaagaaaaaaggaaaggtaaggggaaaaaatactcaagatgggttatatcacaacaagaataagccccctgaatcaagaaaaaaattggaaaaggcactgctggcttgggccatcttagcagcggtcctgcttcagctggtaacaggagagaatatcacccagtggaacttgatggacaacggcaccgagggaatacagcaagcgatgttcctaagaggggtgaacaggagtctacatggaatttggccagagaaaatttgcaccggagtaccaactcacttagcaacagactatgagcttaaagagatagtggggatgatggacgcgagtgagaagaccaactacacgtgttgcaggttgcaaagacatgagtggaataaaggttggtgtaactggtttcatatagaaccgtggatatggttgatgaacaaaacccaaaacaacctgacagaagggcaaccgcttagggagtgtgctgtgacttgtaggtatgacaaggaaacagaattgaacatcgtgacacaggctagggacagacctacaactctgacaggttgcaagaaaggcaagaatttctctttcgcaggtgttatactggatgggccctgtaactttaaagtatcggttgaagatgtgctgttcaaggagcacgattgcggcaacatgctgcaagagaccgcgatacagctactcgatggggcaaccaacaccattgagggagcaagggtagggacggccaagttgacaacctggttagggaagcaattagggatccttggtaagaagttggagaacaaaagcaaagcatggtttggtgcacatgcagcaagtccatactgcggagtggagaggaagatcggttacgtatggtatacaaaaaactgcactccagcttgccttccaagaaacactagaataataggccccgggaaatttgataccaacgccgaagatggaaaaatactccatgagatgggggggcacctctcagaatttgtcctattgtccttggtggttctgtctgactttgccccggaaaccgcgagcgtcatctacttggttctacattttgcgatcccgcaaagccacgttgatgtagacacatgcgacaagaaccagctgaatttaacggtagcaaccacagtagcagaggtcataccagggacagtgtggaacctagggaagtatgtctgcataagaccagactggtggccatatgagacgacgacagtcttcgtcatagaggaagcagggcaagtaatcaaattgatgctaagggccatcagagacttaactaggatatggaatgctgccactaccacagctttcttaatctttttagtaaaagcactgaggggacaactaatccaagggctattgtggctgatgctaataacaggagcacagggcttccctgaatgcaaagagggcttccaatatgccatatctaaagacaggaaaatggggttattggggccagagagcttaactacaacatggcacctccccaccaaaaaaatagtggattccatggtgcatgtatggtgtgaaggaaaagacttgaaaatattaaaaatgtgcacaaaggaagagaggtatctagtggctgtgcacgagagagccttatcaaccagtgccgagtttatgcagatcagtgatgggacaataggcccagacgtgatagatatgcctgatgactttgagtttggactctgcccttgtgactcaaaaccagtgataaagggcaaatttaatgccagcttactgaatggaccagctttccagatggtatgcccacaggggtggactggtacaatagaatgcaccctagcgaaccaagacaccttggacacaactgtcattaggacatatagaagaactaccccatttcagcggagaaaatggtgtacctatgaaaaaataataggggaagatatctatgaatgcattctaggtggaaactggacatgcataaccggtgaccatagcaggttgaaagacggacctatcaagaagtgtaagtggtgtggccatgacttcgtcaactcagaggggctaccacactacccaataggcaagtgcatgctcatcaacgagagtgggtacaggtatgtagatgacacctcttgcgataggggtggtgtagccatagttccatctggcaccgtaaagtgtagaataggtaacgtcacggtgcaagttatcgctactaacaatgatctgggacccatgccttgcagcccagctgaagtgatagcaagtgaaggaccagtggaaaagactgcatgcacattcaactattcaaggactctacctaataagtattatgagccaagggaccggtacttccaacaatacatgttaaaaggggagtggcaatattggttcgacctggattctgtagaccaccacaaagactacttctcagagttcataatcatagcagtggtcgccttgttgggtggtaagtacgtactgtggctcttgataacatacacaatactgtctgagcagatggctatgggtgctggagtgaatactgaagagatagtcatgataggcaatttgctgacagacagtgatattgaggttgtggtttatttccttcttctgtacttaatagttaaagaggaactggcgaggaaatggattatactggtataccacatccttgtagccaaccctatgaaaacaattggggtcgtcttactaatgctagggggagtggtgaaggccagcagaatcaatgctgatgaccaaagtgctatggacccatgctttcttctcgtgacaggcgtagtggctgttttgatgatcgctagaagagaacctgccacattaccactgattgtagcattgctagcaataagaacatcaggattcctactgcccgctagcattgatgtaactgtagcagtagtattaattgtacttttgttggctagctacataacagactactttagatataaaaagtggcttcaactcttatttagtctgatagctggtatctttattataaggagcttaaaacatatcaaccagatggaggtaccagaaatatctatgccaagttggagacctctagctctggtccttttctatataacatctacagcaataaccactaattgggacattgacttagcaggcttcctgctgcaatgggcgccagcagtgatcatgatggctaccatgtgggcagactttttgactctgatcatagtcctgcccagttacgagttatctaagctttacttcctaaagaacgtcaggacagacgtggaaaagaactggctcggcaaagtgaaatacagacagatcagttcagtttatgacatctgtgacagtgaggaagcagtgtacctatttccatcaaggcataagagtggaagcaggccagatttcatattaccttttttgaaagccgtgttaataagctgcatcagcagccaatggcaagtggtttacatttcttacctaatactggaaattacatactatatgcacaggaaaatcatagatgaggtgtcaggaggagcaaattttctatcaagactcatagcagccatcatagaattaaattgggccatagatgatgaggaatgtaaaggactgaagaaactgtatctcttgtcagggagagcgaagaatttgatagttaaacataaggtaagaaatgaagccgtccacagatggtttggtgaggaggaaatatacggggcacccaaggtgatcactatcataaaagctagtaccctaagtaaaaacaggcactgcataatctgcacgatctgtgaagggaaagaatggaatggagccaactgcccaaagtgtggaagacaaggaaagcccataacatgtggaatgacactcgcagactttgaggagaaacattacaaaaagatatttataagagaagaatcttcttgtcctgtgccttttgatccttcttgccattgtaattattttcgccacgatgggcctttcaggaaagagtataagggttacgtccaatacacagccagaggacaactctttctgaggaacctaccaattctagcgacgaagatgaagctattaatggtgggaaacctcggcgcagaaattggcgacctggaacatctaggatgggtactgagagggccagccgtgtgcaaaaaaattaccaaccatgagaagtgccacgtaaacatcatggataagctaactgcattttttggaatcatgcctagaggcacgacccctagggcacctgtgaggttccccacagcactactaaaagtgagaagggggctagagacgggatgggcttacacgcaccaaggagggatcagctcggtagaccatgtcacagccggaaaggatttactagtgtgtgacagtatgggcaggaccagggttgtctgtcatagtaacaataagatgactgatgagactgagtatggcatcaagaccgactcagggtgtcccgaaggtgcgaggtgttacgtgctaaacccagaagctgttaacatttctggcacaaaaggagctatggtacacctccagaaaacggggggggagttcacatgtgtcactgcctcagggaccccggctttcttcgatctgaaaaatctaaaaggctggtccgggctaccaatttttgaagcatccagtggcagggtggttggtagggtgaaagtcggcaagaatgaggattccaagcccaccaaactaatgagcggaatccagacagtgtctaagaaccagacagacctagcggacatcgtaaaaaaattgactagtatgaacagaggagagttcaaacagataacattagccactggggcaggaaaaactacggaactgccaaggtccgtcatagaggagatagggaggcacaaaagggtcttagtcctgataccattgagagcagcagcagagtcagtgtatcagtatatgagagtgaagtacccaagtatatctttcaatttgagaataggagatatgaaggaaggtgacatggccactggtatcacctacgcctcatatgggtacttttgtcagcttcctcagcccaaactgagagctgccatggtagagtactcatatatattcttagatgagtaccactgtgctacacccgagcaattagcaataattggaaagatacacaggtttgctgaaaatcttagagtggtagcaatgacagcaaccccagctggaacggtcacaacgactggtcagaaacaccctatagaggagttcatagccccagaggtgatgaaaggtgaagatctaggtagtgaatacttggatattgcagggttgaagataccgactgaagagatgaaaggcaacatgctcgtgttcgcgccaactaggaacatggcagtagaaacagctaagaaattgaaggctaagggatacaactctggatactattacagtggggaaaacccagagaacttgagggtggtaacctcgcaatccccgtatgtggtagtagccaccaatgccatagagtcaggtgtgacattaccagacttagacacagttgtagacactggactaaagtgtgagaagagggtgaggatttcttcaaaaatgcccttcattgtaacaggacttaagagaatggcagtcacaatcggagagcaagcccagcgcaggggtagagtaggaagagtcaagccaggtaggtactataggagtcaagaaacagcttcagggtcaaaagattaccattacgacctactgcaagcccagaggtacggaatagaagatggaattaatgtaacaaagtcattcagggagatgaactatgattggagcctttacgaagaggacagcttgatgataactcaactcgaggtccttaacaacctccttatatcagaagacctgcctgccgcagtgaagaacatcatggcccggaccgatcacccagaacccatacaactggcctataacagttatgaaaaccaaattccagtgctgttcccaaagatcaaaaatggtgaggtgacagacagttatgagaattacacatatctcaatgcaagaaaattaggagaggacgtgccggcatatgtgtacgccacagaggatgaggatctagcagtggatcttctgggtatggattggccggacccaggcaaccaacaggtggtagagacagggagggcattaaaacaagtaactggcttatccacagcagaaaacgccctcttgatagccctattcggctacgtcgggtaccagacactttcaaaaaggcacatacccatgattactgacatctatacacttgaagaccacaggcttgaggacacaacccacctccagtttgccccaaacgctataaggaccgacggcaaggactcagagttgaaggaattagctgtgggagaccttgataaatatgtggacgcactggtagactactccaaacaagggatgaaattcatcaaagtccaagctgaaaaggtcagagactcccagtctacgaaggaaggcttgcaaaccattaaggagtatgtggataagtttatacaatcactaacagagaataaggaggagatcatcaggtatggactatggggagttcacacggcactctacaaaagcttggcagcgagactggggcatgaaacagcttttgcaactttagtggtaaaatggttggcttttgggggcgaaacggtatctgctcacatcaagcaagtagcagttgatctagtagtatattatatcatcaacaaaccatcttttcctggagatacagagacccaacaagaggggaggaagtttgtggctagtctttttatatctgcactagcaacatacacatataaaacctggaattacaacaatctgcaacgggttgtcgaacctgccttagcttacctcccatatgctacaagtgccttgaagttgttcacacccacaagattagagagtgtggtcatactcagttctacaatttacaagacatacctctctataaggaagggtaagagtgacggcttgttaggtacaggcataagtgcagccatggagatcttaaaccaaaacccaatctcagtaggtatatctgtgatgctgggggtaggtgccatcgccgcccataatgcaatagaatctagtgaacagaaaagaactttgctgatgaaggtctttgtaaaaaacttcttagaccaagcagcaacagatgagctagtcaaagagaaccctgaaaaaataatcatggctctatttgaagcagtccagaccataggaaaccccctaagactcatctaccatctgtacggggtgtactataaggggtgggaagcaaaagaactcgcagagaaaactgctggccgcaacttattcacattgatcatgtttgaggcctttgagcttttaggtatggactcagaaggaaagataagaaacttgtcaggcaactacatactggacttaatcttcaacttgcataataaattaaacaaggggctcaaaaaactagtccttgggtgggctcctgcacctttgagctgtgattggacaccaagtgatgagagaataagcctacctcataacaactacttaagggtagaaaccaggtgtccttgtggctatgagatgaaggcaataaaaaatgttgctggtaaattgacaaaagttgaagaaaaggggtccttcctatgcaggaatagattagggagaggacctccaaacttcaaagtaacaaagttctatgatgataacttgatagaagtcaagccagtagctaggctagaaggccaggtggacctctattacaagggagtaacagctaagttagactacaacaatgggaaagtactgttagctaccaacaagtgggaggtggaccacgctttcctgaccagactagtaaagaagcacacagggataggttttaaaggtgcatatttgggtgaccgaccagaccatcaagatcttgtcgatagagattgtgcaactataacgaagaactcagtacagttcctaaaaatgaagaagggttgcgctttcacatatgacctaacaatctctaaccttgtcaggcttattgaactagtccataagaataatttacaagaaagagagatccctaccgtgacagtaactacttggcttgcatattcttttgtcaatgaagacctggggactatcaagcctgtattgggggagaaagtcatcccagaaccccccgaggagttgagtctccaacccaccgtgagactagtcaccactgaaacagcaataaccataacaggggaggctgaagtgatgacgacagggatcacaccagtggtagagatgaaagaagaacctcagctggaccaccagtcaactaccctaaaggtagggttgaaggaaggggaatatccagggccaggagttaaccctaaccatttagcagaggtgatagatgagaaagatgacaggccttttgtcctaatcatcggtaacaaaggttctacctcgaacagagcaagaacggccaagaatatacggctgtacaaaggaaacaacccaagagagatcagggatctgatgagccaaggaagaatattgacggttgctctaaaagagttggacccggaattaaaagaattagtagattacaaggggacctttctcaatagggaagctttagaagccctaagcttaggtaagccaatcaagaggaaaaccacaacagcaatgatcaggaggttaatagagccagaggttgaggaggaactaccagattggttccaagcggaagaacccctatttttggaagcaaaaatacagaatgacttataccacctaattggcagtgtagatagtataaaaagcaaagcaaaggaattaggggccacagataacacaaagatagtgaaggaagttggggctaggacctatacgatgaaattgagcagctggagcacacaagttacaaaaaaacagatgagtctagcccctctctttgaagagctgttattaaagtgccctccatgtagtaaaatttcaaagggacatatggtgtcagcataccaactggctcaaggaaactgggaacccctcgggtgtggggtctatatgggaaccataccagctaggcgtctcaagatccacccttatgaggcttaccttaaactcaaagagctggtggaagttgaatcttcgagggccactgcaaaagaatccatcataagagaacataacacctggatcctgcggaaggtgagacatgaagggaacctaagaaccaaatcaatgatcaaccctgggaaaatatcagatcagctatgcagagatggacacaaaagaaacatatataataagatcataggctcaacaatggcctctgctggtattaggctggagaaactgccagtagtccgagcccaaactgacacaaccagtttccaccaagccataagagaaaaaattgataaaacagaaaacaagcagacccctgaattgcatgaagaactaatgaaggtcttcgactgcttaaagatcccagagctgaaggaatcgtatgatgaagtttcatgggaacaattagaagccgggataaaccgtaagggtgcagcaggctatctagagagcaagaacataggggaagtcctagacacagagaaacacatagtagagcagctgatcaaggatctgaggaaggggaagaagattaggtactatgaaacagccatccccaagaatgagaagagagacgtcagcgacgactgggaagccggagagttcgttgatgaaaagaaaccaagagtaatccagtacccggacgccaaggtgagactggccattacaaaagtgatgtacaaatgggtaaagcaaaaaccagtggtgatacccggctatgaaggtaaaacacctctatttgacatattcaacaaagtgaagaaggaatgggattcattccaggaccccgtagcagtgagctttgacaccaaagcgtgggatacacaagtcaccagtagagacctaatgttgataaaggatatccagaaatattatttcaagagaagtatacacaaatttttagatacaataacagaacacatggtggaggtacctgtcattacagcagacggtgaagtttacataaggaatggtcagaggggtagtggccaacccgacacaagtgctggtaatagtatgttgaatgtcctaaccatgatatatgctttctgtaaaagtacaggcataccttacaggggattcagcagagtggcaagaatccatgtgtgtggtgatgatggctttttgataacagagagaggactgggactgaaattctctgagaagggtatgcagatattacatgaggccgggaagccccagaaaataactgaaggggacaaaatgaaagtggcatacagattcgaggacatagagttttgttcccatactcccgtgccagtcagatgggcagataacaccagtagttacatggcagggaggagcacagccactatactagctaagatggcaaccaggctggattccagcggagagaggggtagcacagcttatgagaaggccgtagccttcagcttccttttgatgtactcatggaatcccgtagttagaaggatctgcttactggtgttgtcacagtttccagaaatatccccatccaaaaacacaatatactactaccaaggggatcccatagctgcgtacagagaagtgatagggaaacagctgtgtgaactgaaaagaacaggatttgagaagctggctggtctgaatttgagtatgaccactctaggcatctggacaaaacatactagtaaaagactaatccaagcctgtgtagaaataggtaagagagaaggtacctggttagttaatgctgacagactgattgcaggaaagactgggaagttttacatcccaagcactggtgtcactctgttgggaaaacactatgaggaaattaacttaaagcaaaaggcggcacaaccgccgatagagggggttgacagatataagttgggccccatagttaatgttatcttgagaaggctgagggtgatgctgatgacagttgccagcggaagctggtgaatccgtccggagcgtcgtgccctcactcaaggtttttaattgtaaatattgtaaatagacagctaagatatttattgtagttggatagtaatgcagtgatagtaaataccccaatttaacactacctccaatgcactaagcactttagctgtgtgaggttaactcgacgtccacggttggactagggaagacctctaacagcccc

SEQ ID NO: 5 - protein, 3913 aa, Artificial Sequence
  Wildtyp BVDV XIKE-A
MELFSNELLYKTYKQKPAGVVEPVYDVNGRPLFGESSDLHPQSTLKLPHQRGSANILTNARSLPRKGDCRRGNVYGPVSGIYIKPGPIYYQDYVGPVYHRAPLELCREASMCETTRRVGRVTGSDGKLYHIYICIDGCILLKRATRNQPEVLKWVYNRLNCPLWVTSCSDEGSKGATSKKQPKPDRIEKGKMKIAPKETEKDCKTRPPDATIVVEGVKYQVKKKGKVRGKNTQDGLYHNKNKPPESRKKLEKALLAWAILAAVLLQLVTGENITQWNLMDNGTEGIQQAMFLRGVNRSLHGIWPEKICTGVPTHLATDYELKEIVGMMDASEKTNYTCCRLQRHEWNKHGWCNWFHIEPWIWLMNKTQNNLTEGQPLRECAVTCRYDKETELNIVTQARDRPTTLTGCKKGKNFSFAGVILDGPCNFKVSVEDVLFKEHDCGNMLQETAIQLLDGATNTIEGARVGTAKLTTWLGKQLGILGKKLENKSKAWFGAHAASPYCGVERKIGYVWYTKNCTPACLPRNTRIIGPGKFDTNAEDGKILHEMGGHLSEFVLLSLVVLSDFAPETASVIYLVLHFAIPQSHVDVDTCDKNQLNLTVATTVAEVIPGTVWNLGKYVCIRPDWWPYETTTVFVIEEAGQVIKLMLRAIRDLTRIWNAATTTAFLIFLVKALRGQLIQGLLWLMLITGAQGFPECKEGFQYAISKDRKMGLLGPESLTTTWHLPTKKIVDSMVHVWCEGKDLKILKMCTKEERYLVAVHERALSTSAEFMQISDGTIGPDVIDMPDDFEFGLCPCDSKPVIKGKFNASLLNGPAFQMVCPQGWTGTIECTLANQDTLDTTVIRTYRRTTPFQRRKWCTYEKIIGEDIYECILGGNWTCITGDHSRLKDGPIKKCKWCGHDFVNSEGLPHYPIGKCMLINESGYRYVDDTSCDRGGVAIVPSGTVKCRIGNVTVQVIATNNDLGPMPCSPAEVIASEGPVEKTACTFNYSRTLPNKYYEPRDRYFQQYMLKGEWQYWFDLDSVDHHKDYFSEFIIIAVVALLGGKYVLWLLITYTILSEQMAMGAGVNTEEIVMIGNLLTDSDIEVVVYFLLLYLIVKEELARKWIILVYHILVANPMKTIGVVLLMLGGVVKASRINADDQSAMDPCFLLVTGVVAVLMIARREPATLPLIVALLAIRTSGFLLPASIDVTVAVVLIVLLLASYITDYFRYKKWLQLLFSLIAGIFIIRSLKHINQMEVPEISMPSWRPLALVLFYITSTAITTNWDIDLAGFLLQWAPAVIMMATMWADFLTLIIVLPSYELSKLYFLKNVRTDVEKNWLGKVKYRQISSVYDICDSEEAVYLFPSRHKSGSRPDFILPFLKAVLISCISSQWQVVYISYLILEITYYMHRKIIDEVSGGANFLSRLIAAIIELNWAIDDEECKGLKKLYLLSGRAKNLIVKHKVRNEAVHRWFGEEEIYGAPKVITIIKASTLSKNRHCIICTICEGKEWNGANCPKCGRQGKPITCGMTLADFEEKHYKKIFIREESSCPVPFDPSCHCNYFRHDGPFRKEYKGYVQYTARGQLFLRNLPILATKMKLLMVGNLGAEIGDLEHLGWVLRGPAVCKKITNHEKCHVNIMDKLTAFFGIMPRGTTPRAPVRFPTALLKVRRGLETGWAYTHQGGISSVDHVTAGKDLLVCDSMGRTRVVCHSNNKMTDETEYGIKTDSGCPEGARCYVLNPEAVNISGTKGAMVHLQKTGGEFTCVTASGTPAFFDLKNLKGWSGLPIFEASSGRVVGRVKVGKNEDSKPTKLMSGIQTVSKNQTDLADIVKKLTSMNRGEFKQITLATGAGKTTELPRSVIEEIGRHKRVLVLIPLRAAAESVYQYMRVKYPSISFNLRIGDMKEGDMATGITYASYGYFCQLPQPKLRAAMVEYSYIFLDEYHCATPEQLAIIGKIHRFAENLRVVAMTATPAGTVTTTGQKHPIEEFIAPEVMKGEDLGSEYLDIAGLKIPTEEMKGNMLVFAPTRNMAVETAKKLKAKGYNSGYYYSGENPENLRVVTSQSPYVVVATNAIESGVTLPDLDTVVDTGLKCEKRVRISSKMPFIVTGLKRMAVTIGEQAQRRGRVGRVKPGRYYRSQETASGSKDYHYDLLQAQRYGIEDGINVTKSFREMNYDWSLYEEDSLMITQLEVLNNLLISEDLPAAVKNIMARTDHPEPIQLAYNSYENQIPVLFPKIKNGEVTDSYENYTYLNARKLGEDVPAYVYATEDEDLAVDLLGMDWPDPGNQQVVETGRALKQVTGLSTAENALLIALFGYVGYQTLSKRHIPMITDIYTLEDHRLEDTTHLQFAPNAIRTDGKDSELKELAVGDLDKYVDALVDYSKQGMKFIKVQAEKVRDSQSTKEGLQTIKEYVDKFIQSLTENKEEIIRYGLWGVHTALYKSLAARLGHETAFATLVVKWLAFGGETVSAHIKQVAVDLVVYYIINKPSFPGDTETQQEGRRFVASLFISALATYTYKTWNYNNLQRVVEPALAYLPYATSALKLFTPTRLESVVILSSTIYKTYLSIRKGKSDGLLGTGISAAMEILNQNPISVGISVMLGVGAIAAHNAIESSEQKRTLLMKVFVKNFLDQAATDELVKENPEKIIMALFEAVQTIGNPLRLIYHLYGVYYKGWEAKELAEKTAGRNLFTLIMFEAFELLGMDSEGKIRNLSGNYILDLIFNLHNKLNKGLKKLVLGWAPAPLSCDWTPSDERISLPHNNYLRVETRCPCGYEMKAIKNVAGKLTKVEEKGSFLCRNRLGRGPPNFKVTKFYDDNLIEVKPVARLEGQVDLYYKGVTAKLDYNNGKVLLATNKWEVDHAFLTRLVKKHTGIGFKGAYLGDRPDHQDLVDRDCATITKNSVQFLKMKKGCAFTYDLTISNLVRLIELVHKNNLQEREIPTVTVTTWLAYSFVNEDLGTIKPVLGEKVIPEPPEELSLQPTVRLVTTETAITITGEAEVMTTGITPVVEMKEEPQLDHQSTTLKVGLKEGEYPGPGVNPNHLAEVIDEKDDRPFVLIIGNKGSTSNRARTAKNIRLYKGNNPREIRDLMSQGRILTVALKELDPELKELVDYKGTFLNREALEALSLGKPIKRKTTTAMIRRLIEPEVEEELPDWFQAEEPLFLEAKIQNDLYHLIGSVDSIKSKAKELGATDNTKIVKEVGARTYTMKLSSWSTQVTKKQMSLAPLFEELLLKCPPCSKISKGHMVSAYQLAQGNWEPLGCGVYMGTIPARRLKIHPYEAYLKLKELVEVESSRATAKESIIREHNTWILRKVRHEGNLRTKSMINPGKISDQLCRDGHKRNIYNKIIGSTMASAGIRLEKLPVVRAQTDTTSFHQAIREKIDKTENKQTPELHEELMKVFDCLKIPELKESYDEVSWEQLEAGINRKGAAGYLESKNIGEVLDTEKHIVEQLIKDLRKGKKIRYYETAIPKNEKRDVSDDWEAGEFVDEKKPRVIQYPDAKVRLAITKVMYKWVKQKPVVIPGYEGKTPLFDIFNKVKKEWDSFQDPVAVSFDTKAWDTQVTSRDLMLIKDIQKYYFKRSIHKFLDTITEHMVEVPVITADGEVYIRNGQRGSGQPDTSAGNSMLNVLTMIYAFCKSTGIPYRGFSRVARIHVCGDDGFLITERGLGLKFSEKGMQILHEAGKPQKITEGDKMKVAYRFEDIEFCSHTPVPVRWADNTSSYMAGRSTATILAKMATRLDSSGERGSTAYEKAVAFSFLLMYSWNPVVRRICLLVLSQFPEISPSKNTIYYYQGDPIAAYREVIGKQLCELKRTGFEKLAGLNLSMTTLGIWTKHTSKRLIQACVEIGKREGTWLVNADRLIAGKTGKFYIPSTGVTLLGKHYEEINLKQKAAQPPIEGVDRYKLGPIVNVILRRLRVMLMTVASGSW

SEQ ID NO: 6 - protein, 3749 aa, Artificial Sequence
  Mutated BVDV XIKE-A-NdN
MELFSDEGSKGATSKKQPKPDRIEKGKMKIAPKETEKDCKTRPPDATIVVEGVKYQVKKKGKVRGKNTQDGLYHNKNKPPESRKKLEKALLAWAILAAVLLQLVTGENITQWNLMDNGTEGIQQAMFLRGVNRSLHGIWPEKICTGVPTHLATDYELKEIVGMMDASEKTNYTCCRLQRHEWNKHGWCNWFHIEPWIWLMNKTQNNLTEGQPLRECAVTCRYDKETELNIVTQARDRPTTLTGCKKGKNFSFAGVILDGPCNFKVSVEDVLFKEHDCGNMLQETAIQLLDGATNTIEGARVGTAKLTTWLGKQLGILGKKLENKSKAWFGAHAASPYCGVERKIGYVWYTKNCTPACLPRNTRIIGPGKFDTNAEDGKILHEMGGHLSEFVLLSLVVLSDFAPETASVIYLVLHFAIPQSHVDVDTCDKNQLNLTVATTVAEVIPGTVWNLGKYVCIRPDWWPYETTTVFVIEEAGQVIKLMLRAIRDLTRIWNAATTTAFLIFLVKALRGQLIQGLLWLMLITGAQGFPECKEGFQYAISKDRKMGLLGPESLTTTWHLPTKKIVDSMVHVWCEGKDLKILKMCTKEERYLVAVHERALSTSAEFMQISDGTIGPDVIDMPDDFEFGLCPCDSKPVIKGKFNASLLNGPAFQMVCPQGWTGTIECTLANQDTLDTTVIRTYRRTTPFQRRKWCTYEKIIGEDIYECILGGNWTCITGDHSRLKDGPIKKCKWCGHDFVNSEGLPHYPIGKCMLINESGYRYVDDTSCDRGGVAIVPSGTVKCRIGNVTVQVIATNNDLGPMPCSPAEVIASEGPVEKTACTFNYSRTLPNKYYEPRDRYFQQYMLKGEWQYWFDLDSVDHHKDYFSEFIIIAVVALLGGKYVLWLLITYTILSEQMAMGAGVNTEEIVMIGNLLTDSDIEVVVYFLLLYLIVKEELARKWIILVYHILVANPMKTIGVVLLMLGGVVKASRINADDQSAMDPCFLLVTGVVAVLMIARREPATLPLIVALLAIRTSGFLLPASIDVTVAVVLIVLLLASYITDYFRYKKWLQLLFSLIAGIFIIRSLKHINQMEVPEISMPSWRPLALVLFYITSTAITTNWDIDLAGFLLQWAPAVIMMATMWADFLTLIIVLPSYELSKLYFLKNVRTDVEKNWLGKVKYRQISSVYDICDSEEAVYLFPSRHKSGSRPDFILPFLKAVLISCISSQWQVVYISYLILEITYYMHRKIIDEVSGGANFLSRLIAAIIELNWAIDDEECKGLKKLYLLSGRAKNLIVKHKVRNEAVHRWFGEEEIYGAPKVITIIKASTLSKNRHCIICTICEGKEWNGANCPKCGRQGKPITCGMTLADFEEKHYKKIFIREESSCPVPFDPSCHCNYFRHDGPFRKEYKGYVQYTARGQLFLRNLPILATKMKLLMVGNLGAEIGDLEHLGWVLRGPAVCKKITNHEKCHVNIMDKLTAFFGIMPRGTTPRAPVRFPTALLKVRRGLETGWAYTHQGGISSVDHVTAGKDLLVCDSMGRTRVVCHSNNKMTDETEYGIKTDSGCPEGARCYVLNPEAVNISGTKGAMVHLQKTGGEFTCVTASGTPAFFDLKNLKGWSGLPIFEASSGRVVGRVKVGKNEDSKPTKLMSGIQTVSKNQTDLADIVKKLTSMNRGEFKQITLATGAGKTTELPRSVIEEIGRHKRVLVLIPLRAAAESVYQYMRVKYPSISFNLRIGDMKEGDMATGITYASYGYFCQLPQPKLRAAMVEYSYIFLDEYHCATPEQLAIIGKIHRFAENLRVVAMTATPAGTVTTTGQKHPIEEFIAPEVMKGEDLGSEYLDIAGLKIPTEEMKGNMLVFAPTRNMAVETAKKLKAKGYNSGYYYSGENPENLRVVTSQSPYVVVATNAIESGVTLPDLDTVVDTGLKCEKRVRISSKMPFIVTGLKRMAVTIGEQAQRRGRVGRVKPGRYYRSQETASGSKDYHYDLLQAQRYGIEDGINVTKSFREMNYDWSLYEEDSLMITQLEVLNNLLISEDLPAAVKNIMARTDHPEPIQLAYNSYENQIPVLFPKIKNGEVTDSYENYTYLNARKLGEDVPAYVYATEDEDLAVDLLGMDWPDPGNQQVVETGRALKQVTGLSTAENALLIALFGYVGYQTLSKRHIPMITDIYTLEDHRLEDTTHLQFAPNAIRTDGKDSELKELAVGDLDKYVDALVDYSKQGMKFIKVQAEKVRDSQSTKEGLQTIKEYVDKFIQSLTENKEEIIRYGLWGVHTALYKSLAARLGHETAFATLVVKWLAFGGETVSAHIKQVAVDLVVYYIINKPSFPGDTETQQEGRRFVASLFISALATYTYKTWNYNNLQRVVEPALAYLPYATSALKLFTPTRLESVVILSSTIYKTYLSIRKGKSDGLLGTGISAAMEILNQNPISVGISVMLGVGAIAAHNAIESSEQKRTLLMKVFVKNFLDQAATDELVKENPEKIIMALFEAVQTIGNPLRLIYHLYGVYYKGWEAKELAEKTAGRNLFTLIMFEAFELLGMDSEGKIRNLSGNYILDLIFNLHNKLNKGLKKLVLGWAPAPLSCDWTPSDERISLPHNNYLRVETRCPCGYEMKAIKNVAGKLTKVEEKGSFLCRNRLGRGPPNFKVTKFYDDNLIEVKPVARLEGQVDLYYKGVTAKLDYNNGKVLLATNKWEVDHAFLTRLVKKHTGIGFKGAYLGDRPDHQDLVDRDCATITKNSVQFLKMKKGCAFTYDLTISNLVRLIELVHKNNLQEREIPTVTVTTWLAYSFVNEDLGTIKPVLGEKVIPEPPEELSLQPTVRLVTTETAITITGEAEVMTTGITPVVEMKEEPQLDHQSTTLKVGLKEGEYPGPGVNPNHLAEVIDEKDDRPFVLIIGNKGSTSNRARTAKNIRLYKGNNPREIRDLMSQGRILTVALKELDPELKELVDYKGTFLNREALEALSLGKPIKRKTTTAMIRRLIEPEVEEELPDWFQAEEPLFLEAKIQNDLYHLIGSVDSIKSKAKELGATDNTKIVKEVGARTYTMKLSSWSTQVTKKQMSLAPLFEELLLKCPPCSKISKGHMVSAYQLAQGNWEPLGCGVYMGTIPARRLKIHPYEAYLKLKELVEVESSRATAKESIIREHNTWILRKVRHEGNLRTKSMINPGKISDQLCRDGHKRNIYNKIIGSTMASAGIRLEKLPVVRAQTDTTSFHQAIREKIDKTENKQTPELHEELMKVFDCLKIPELKESYDEVSWEQLEAGINRKGAAGYLESKNIGEVLDTEKHIVEQLIKDLRKGKKIRYYETAIPKNEKRDVSDDWEAGEFVDEKKPRVIQYPDAKVRLAITKVMYKWVKQKPVVIPGYEGKTPLFDIFNKVKKEWDSFQDPVAVSFDTKAWDTQVTSRDLMLIKDIQKYYFKRSIHKFLDTITEHMVEVPVITADGEVYIRNGQRGSGQPDTSAGNSMLNVLTMIYAFCKSTGIPYRGFSRVARIHVCGDDGFLITERGLGLKFSEKGMQILHEAGKPQKITEGDKMKVAYRFEDIEFCSHTPVPVRWADNTSSYMAGRSTATILAKMATRLDSSGERGSTAYEKAVAFSFLLMYSWNPVVRRICLLVLSQFPEISPSKNTIYYYQGDPIAAYREVIGKQLCELKRTGFEKLAGLNLSMTTLGIWTKHTSKRLIQACVEIGKREGTWLVNADRLIAGKTGKFYIPSTGVTLLGKHYEEINLKQKAAQPPIEGVDRYKLGPIVNVILRRLRVMLMTVASGSW

SEQ ID NO: 7 - protein, 3912 aa, Artificial Sequence
  Mutated BVDV XIKE-B
MELFSNELLYKTYKQKPAGVVEPVYDVNGRPLFGESSDLHPQSTLKLPHQRGSANILTNARSLPRKGDCRRGNVYGPVSGIYIKPGPIYYQDYVGPVYHRAPLELCREASMCETTRRVGRVTGSDGKLYHIYICIDGCILLKRATRNQPEVLKWVYNRLNCPLWVTSCSDEGSKGATSKKQPKPDRIEKGKMKIAPKETEKDCKTRPPDATIVVEGVKYQVKKKGKVRGKNTQDGLYHNKNKPPESRKKLEKALLAWAILAAVLLQLVTGENITQWNLMDNGTEGIQQAMFLRGVNRSLHGIWPEKICTGVPTHLATDYELKEIVGMMDASEKTNYTCCRLQRHEWNKGWCNWFHIEPWIWLMNKTQNNLTEGQPLRECAVTCRYDKETELNIVTQARDRPTTLTGCKKGKNFSFAGVILDGPCNFKVSVEDVLFKEHDCGNMLQETAIQLLDGATNTIEGARVGTAKLTTWLGKQLGILGKKLENKSKAWFGAHAASPYCGVERKIGYVWYTKNCTPACLPRNTRIIGPGKFDTNAEDGKILHEMGGHLSEFVLLSLVVLSDFAPETASVIYLVLHFAIPQSHVDVDTCDKNQLNLTVATTVAEVIPGTVWNLGKYVCIRPDWWPYETTTVFVIEEAGQVIKLMLRAIRDLTRIWNAATTTAFLIFLVKALRGQLIQGLLWLMLITGAQGFPECKEGFQYAISKDRKMGLLGPESLTTTWHLPTKKIVDSMVHVWCEGKDLKILKMCTKEERYLVAVHERALSTSAEFMQISDGTIGPDVIDMPDDFEFGLCPCDSKPVIKGKFNASLLNGPAFQMVCPQGWTGTIECTLANQDTLDTTVIRTYRRTTPFQRRKWCTYEKIIGEDIYECILGGNWTCITGDHSRLKDGPIKKCKWCGHDFVNSEGLPHYPIGKCMLINESGYRYVDDTSCDRGGVAIVPSGTVKCRIGNVTVQVIATNNDLGPMPCSPAEVIASEGPVEKTACTFNYSRTLPNKYYEPRDRYFQQYMLKGEWQYWFDLDSVDHHKDYFSEFIIIAVVALLGGKYVLWLLITYTILSEQMAMGAGVNTEEIVMIGNLLTDSDIEVVVYFLLLYLIVKEELARKWIILVYHILVANPMKTIGVVLLMLGGVVKASRINADDQSAMDPCFLLVTGVVAVLMIARREPATLPLIVALLAIRTSGFLLPASIDVTVAVVLIVLLLASYITDYFRYKKWLQLLFSLIAGIFIIRSLKHINQMEVPEISMPSWRPLALVLFYITSTAITTNWDIDLAGFLLQWAPAVIMMATMWADFLTLIIVLPSYELSKLYFLKNVRTDVEKNWLGKVKYRQISSVYDICDSEEAVYLFPSRHKSGSRPDFILPFLKAVLISCISSQWQVVYISYLILEITYYMHRKIIDEVSGGANFLSRLIAAIIELNWAIDDEECKGLKKLYLLSGRAKNLIVKHKVRNEAVHRWFGEEEIYGAPKVITIIKASTLSKNRHCIICTICEGKEWNGANCPKCGRQGKPITCGMTLADFEEKHYKKIFIREESSCPVPFDPSCHCNYFRHDGPFRKEYKGYVQYTARGQLFLRNLPILATKMKLLMVGNLGAEIGDLEHLGWVLRGPAVCKKITNHEKCHVNIMDKLTAFFGIMPRGTTPRAPVRFPTALLKVRRGLETGWAYTHQGGISSVDHVTAGKDLLVCDSMGRTRVVCHSNNKMTDETEYGIKTDSGCPEGARCYVLNPEAVNISGTKGAMVHLQKTGGEFTCVTASGTPAFFDLKNLKGWSGLPIFEASSGRVVGRVKVGKNEDSKPTKLMSGIQTVSKNQTDLADIVKKLTSMNRGEFKQITLATGAGKTTELPRSVIEEIGRHKRVLVLIPLRAAAESVYQYMRVKYPSISFNLRIGDMKEGDMATGITYASYGYFCQLPQPKLRAAMVEYSYIFLDEYHCATPEQLAIIGKIHRFAENLRVVAMTATPAGTVTTTGQKHPIEEFIAPEVMKGEDLGSEYLDIAGLKIPTEEMKGNMLVFAPTRNMAVETAKKLKAKGYNSGYYYSGENPENLRVVTSQSPYVVVATNAIESGVTLPDLDTVVDTGLKCEKRVRISSKMPFIVTGLKRMAVTIGEQAQRRGRVGRVKPGRYYRSQETASGSKDYHYDLLQAQRYGIEDGINVTKSFREMNYDWSLYEEDSLMITQLEVLNNLLISEDLPAAVKNIMARTDHPEPIQLAYNSYENQIPVLFPKIKNGEVTDSYENYTYLNARKLGEDVPAYVYATEDEDLAVDLLGMDWPDPGNQQVVETGRALKQVTGLSTAENALLIALFGYVGYQTLSKRHIPMITDIYTLEDHRLEDTTHLQFAPNAIRTDGKDSELKELAVGDLDKYVDALVDYSKQGMKFIKVQAEKVRDSQSTKEGLQTIKEYVDKFIQSLTENKEEIIRYGLWGVHTALYKSLAARLGHETAFATLVVKWLAFGGETVSAHIKQVAVDLVVYYIINKPSFPGDTETQQEGRRFVASLFISALATYTYKTWNYNNLQRVVEPALAYLPYATSALKLFTPTRLESVVILSSTIYKTYLSIRKGKSDGLLGTGISAAMEILNQNPISVGISVMLGVGAIAAHNAIESSEQKRTLLMKVFVKNFLDQAATDELVKENPEKIIMALFEAVQTIGNPLRLIYHLYGVYYKGWEAKELAEKTAGRNLFTLIMFEAFELLGMDSEGKIRNLSGNYILDLIFNLHNKLNKGLKKLVLGWAPAPLSCDWTPSDERISLPHNNYLRVETRCPCGYEMKAIKNVAGKLTKVEEKGSFLCRNRLGRGPPNFKVTKFYDDNLIEVKPVARLEGQVDLYYKGVTAKLDYNNGKVLLATNKWEVDHAFLTRLVKKHTGIGFKGAYLGDRPDHQDLVDRDCATITKNSVQFLKMKKGCAFTYDLTISNLVRLIELVHKNNLQEREIPTVTVTTWLAYSFVNEDLGTIKPVLGEKVIPEPPEELSLQPTVRLVTTETAITITGEAEVMTTGITPVVEMKEEPQLDHQSTTLKVGLKEGEYPGPGVNPNHLAEVIDEKDDRPFVLIIGNKGSTSNRARTAKNIRLYKGNNPREIRDLMSQGRILTVALKELDPELKELVDYKGTFLNREALEALSLGKPIKRKTTTAMIRRLIEPEVEEELPDWFQAEEPLFLEAKIQNDLYHLIGSVDSIKSKAKELGATDNTKIVKEVGARTYTMKLSSWSTQVTKKQMSLAPLFEELLLKCPPCSKISKGHMVSAYQLAQGNWEPLGCGVYMGTIPARRLKIHPYEAYLKLKELVEVESSRATAKESIIREHNTWILRKVRHEGNLRTKSMINPGKISDQLCRDGHKRNIYNKIIGSTMASAGIRLEKLPVVRAQTDTTSFHQAIREKIDKTENKQTPELHEELMKVFDCLKIPELKESYDEVSWEQLEAGINRKGAAGYLESKNIGEVLDTEKHIVEQLIKDLRKGKKIRYYETAIPKNEKRDVSDDWEAGEFVDEKKPRVIQYPDAKVRLAITKVMYKWVKQKPVVIPGYEGKTPLFDIFNKVKKEWDSFQDPVAVSFDTKAWDTQVTSRDLMLIKDIQKYYFKRSIHKFLDTITEHMVEVPVITADGEVYIRNGQRGSGQPDTSAGNSMLNVLTMIYAFCKSTGIPYRGFSRVARIHVCGDDGFLITERGLGLKFSEKGMQILHEAGKPQKITEGDKMKVAYRFEDIEFCSHTPVPVRWADNTSSYMAGRSTATILAKMATRLDSSGERGSTAYEKAVAFSFLLMYSWNPVVRRICLLVLSQFPEISPSKNTIYYYQGDPIAAYREVIGKQLCELKRTGFEKLAGLNLSMTTLGIWTKHTSKRLIQACVEIGKREGTWLVNADRLIAGKTGKFYIPSTGVTLLGKHYEEINLKQKAAQPPIEGVDRYKLGPIVNVILRRLRVMLMTVASGSW

SEQ ID NO: 8 - protein, 3748 aa, Artificial Sequence
  Mutated BVDV XIKE-B-NdN
MELFSDEGSKGATSKKQPKPDRIEKGKMKIAPKETEKDCKTRPPDATIVVEGVKYQVKKKGKVRGKNTQDGLYHNKNKPPESRKKLEKALLAWAILAAVLLQLVTGENITQWNLMDNGTEGIQQAMFLRGVNRSLHGIWPEKICTGVPTHLATDYELKEIVGMMDASEKTNYTCCRLQRHEWNKGWCNWFHIEPWIWLMNKTQNNLTEGQPLRECAVTCRYDKETELNIVTQARDRPTTLTGCKKGKNFSFAGVILDGPCNFKVSVEDVLFKEHDCGNMLQETAIQLLDGATNTIEGARVGTAKLTTWLGKQLGILGKKLENKSKAWFGAHAASPYCGVERKIGYVWYTKNCTPACLPRNTRIIGPGKFDTNAEDGKILHEMGGHLSEFVLLSLVVLSDFAPETASVIYLVLHFAIPQSHVDVDTCDKNQLNLTVATTVAEVIPGTVWNLGKYVCIRPDWWPYETTTVFVIEEAGQVIKLMLRAIRDLTRIWNAATTTAFLIFLVKALRGQLIQGLLWLMLITGAQGFPECKEGFQYAISKDRKMGLLGPESLTTTWHLPTKKIVDSMVHVWCEGKDLKILKMCTKEERYLVAVHERALSTSAEFMQISDGTIGPDVIDMPDDFEFGLCPCDSKPVIKGKFNASLLNGPAFQMVCPQGWTGTIECTLANQDTLDTTVIRTYRRTTPFQRRKWCTYEKIIGEDIYECILGGNWTCITGDHSRLKDGPIKKCKWCGHDFVNSEGLPHYPIGKCMLINESGYRYVDDTSCDRGGVAIVPSGTVKCRIGNVTVQVIATNNDLGPMPCSPAEVIASEGPVEKTACTFNYSRTLPNKYYEPRDRYFQQYMLKGEWQYWFDLDSVDHHKDYFSEFIIIAVVALLGGKYVLWLLITYTILSEQMAMGAGVNTEEIVMIGNLLTDSDIEVVVYFLLLYLIVKEELARKWIILVYHILVANPMKTIGVVLLMLGGVVKASRINADDQSAMDPCFLLVTGVVAVLMIARREPATLPLIVALLAIRTSGFLLPASIDVTVAVVLIVLLLASYITDYFRYKKWLQLLFSLIAGIFIIRSLKHINQMEVPEISMPSWRPLALVLFYITSTAITTNWDIDLAGFLLQWAPAVIMMATMWADFLTLIIVLPSYELSKLYFLKNVRTDVEKNWLGKVKYRQISSVYDICDSEEAVYLFPSRHKSGSRPDFILPFLKAVLISCISSQWQVVYISYLILEITYYMHRKIIDEVSGGANFLSRLIAAIIELNWAIDDEECKGLKKLYLLSGRAKNLIVKHKVRNEAVHRWFGEEEIYGAPKVITIIKASTLSKNRHCIICTICEGKEWNGANCPKCGRQGKPITCGMTLADFEEKHYKKIFIREESSCPVPFDPSCHCNYFRHDGPFRKEYKGYVQYTARGQLFLRNLPILATKMKLLMVGNLGAEIGDLEHLGWVLRGPAVCKKITNHEKCHVNIMDKLTAFFGIMPRGTTPRAPVRFPTALLKVRRGLETGWAYTHQGGISSVDHVTAGKDLLVCDSMGRTRVVCHSNNKMTDETEYGIKTDSGCPEGARCYVLNPEAVNISGTKGAMVHLQKTGGEFTCVTASGTPAFFDLKNLKGWSGLPIFEASSGRVVGRVKVGKNEDSKPTKLMSGIQTVSKNQTDLADIVKKLTSMNRGEFKQITLATGAGKTTELPRSVIEEIGRHKRVLVLIPLRAAAESVYQYMRVKYPSISFNLRIGDMKEGDMATGITYASYGYFCQLPQPKLRAAMVEYSYIFLDEYHCATPEQLAIIGKIHRFAENLRVVAMTATPAGTVTTTGQKHPIEEFIAPEVMKGEDLGSEYLDIAGLKIPTEEMKGNMLVFAPTRNMAVETAKKLKAKGYNSGYYYSGENPENLRVVTSQSPYVVVATNAIESGVTLPDLDTVVDTGLKCEKRVRISSKMPFIVTGLKRMAVTIGEQAQRRGRVGRVKPGRYYRSQETASGSKDYHYDLLQAQRYGIEDGINVTKSFREMNYDWSLYEEDSLMITQLEVLNNLLISEDLPAAVKNIMARTDHPEPIQLAYNSYENQIPVLFPKIKNGEVTDSYENYTYLNARKLGEDVPAYVYATEDEDLAVDLLGMDWPDPGNQQVVETGRALKQVTGLSTAENALLIALFGYVGYQTLSKRHIPMITDIYTLEDHRLEDTTHLQFAPNAIRTDGKDSELKELAVGDLDKYVDALVDYSKQGMKFIKVQAEKVRDSQSTKEGLQTIKEYVDKFIQSLTENKEEIIRYGLWGVHTALYKSLAARLGHETAFATLVVKWLAFGGETVSAHIKQVAVDLVVYYIINKPSFPGDTETQQEGRRFVASLFISALATYTYKTWNYNNLQRVVEPALAYLPYATSALKLFTPTRLESVVILSSTIYKTYLSIRKGKSDGLLGTGISAAMEILNQNPISVGISVMLGVGAIAAHNAIESSEQKRTLLMKVFVKNFLDQAATDELVKENPEKIIMALFEAVQTIGNPLRLIYHLYGVYYKGWEAKELAEKTAGRNLFTLIMFEAFELLGMDSEGKIRNLSGNYILDLIFNLHNKLNKGLKKLVLGWAPAPLSCDWTPSDERISLPHNNYLRVETRCPCGYEMKAIKNVAGKLTKVEEKGSFLCRNRLGRGPPNFKVTKFYDDNLIEVKPVARLEGQVDLYYKGVTAKLDYNNGKVLLATNKWEVDHAFLTRLVKKHTGIGFKGAYLGDRPDHQDLVDRDCATITKNSVQFLKMKKGCAFTYDLTISNLVRLIELVHKNNLQEREIPTVTVTTWLAYSFVNEDLGTIKPVLGEKVIPEPPEELSLQPTVRLVTTETAITITGEAEVMTTGITPVVEMKEEPQLDHQSTTLKVGLKEGEYPGPGVNPNHLAEVIDEKDDRPFVLIIGNKGSTSNRARTAKNIRLYKGNNPREIRDLMSQGRILTVALKELDPELKELVDYKGTFLNREALEALSLGKPIKRKTTTAMIRRLIEPEVEEELPDWFQAEEPLFLEAKIQNDLYHLIGSVDSIKSKAKELGATDNTKIVKEVGARTYTMKLSSWSTQVTKKQMSLAPLFEELLLKCPPCSKISKGHMVSAYQLAQGNWEPLGCGVYMGTIPARRLKIHPYEAYLKLKELVEVESSRATAKESIIREHNTWILRKVRHEGNLRTKSMINPGKISDQLCRDGHKRNIYNKIIGSTMASAGIRLEKLPVVRAQTDTTSFHQAIREKIDKTENKQTPELHEELMKVFDCLKIPELKESYDEVSWEQLEAGINRKGAAGYLESKNIGEVLDTEKHIVEQLIKDLRKGKKIRYYETAIPKNEKRDVSDDWEAGEFVDEKKPRVIQYPDAKVRLAITKVMYKWVKQKPVVIPGYEGKTPLFDIFNKVKKEWDSFQDPVAVSFDTKAWDTQVTSRDLMLIKDIQKYYFKRSIHKFLDTITEHMVEVPVITADGEVYIRNGQRGSGQPDTSAGNSMLNVLTMIYAFCKSTGIPYRGFSRVARIHVCGDDGFLITERGLGLKFSEKGMQILHEAGKPQKITEGDKMKVAYRFEDIEFCSHTPVPVRWADNTSSYMAGRSTATILAKMATRLDSSGERGSTAYEKAVAFSFLLMYSWNPVVRRICLLVLSQFPEISPSKNTIYYYQGDPIAAYREVIGKQLCELKRTGFEKLAGLNLSMTTLGIWTKHTSKRLIQACVEIGKREGTWLVNADRLIAGKTGKFYIPSTGVTLLGKHYEEINLKQKAAQPPIEGVDRYKLGPIVNVILRRLRVMLMTVASGSW

SEQ ID NO: 9 - protein, 3913 aa, Artificial Sequence
  XIKE-C BVDV-Sequence
MELFSNELLYKTYKQKPAGVVEPVYDVNGRPLFGESSDLHPQSTLKLPHQRGSANILTNARSLPRKGDCRRGNVYGPVSGIYIKPGPIYYQDYVGPVYHRAPLELCREASMCETTRRVGRVTGSDGKLYHIYICIDGCILLKRATRNQPEVLKWVYNRLNCPLWVTSCSDEGSKGATSKKQPKPDRIEKGKMKIAPKETEKDCKTRPPDATIVVEGVKYQVKKKGKVRGKNTQDGLYHNKNKPPESRKKLEKALLAWAILAAVLLQLVTGENITQWNLMDNGTEGIQQAMFLRGVNRSLLGIWPEKICTGVPTHLATDYELKEIVGMMDASEKTNYTCCRLQRHEWNKHGWCNWFHIEPWIWLMNKTQNNLTEGQPLRECAVTCRYDKETELNIVTQARDRPTTLTGCKKGKNFSFAGVILDGPCNFKVSVEDVLFKEHDCGNMLQETAIQLLDGATNTIEGARVGTAKLTTWLGKQLGILGKKLENKSKAWFGAHAASPYCGVERKIGYVWYTKNCTPACLPRNTRIIGPGKFDTNAEDGKILHEMGGHLSEFVLLSLVVLSDFAPETASVIYLVLHFAIPQSHVDVDTCDKNQLNLTVATTVAEVIPGTVWNLGKYVCIRPDWWPYETTTVFVIEEAGQVIKLMLRAIRDLTRIWNAATTTAFLIFLVKALRGQLIQGLLWLMLITGAQGFPECKEGFQYAISKDRKMGLLGPESLTTTWHLPTKKIVDSMVHVWCEGKDLKILKMCTKEERYLVAVHERALSTSAEFMQISDGTIGPDVIDMPDDFEFGLCPCDSKPVIKGKFNASLLNGPAFQMVCPQGWTGTIECTLANQDTLDTTVIRTYRRTTPFQRRKWCTYEKIIGEDIYECILGGNWTCITGDHSRLKDGPIKKCKWCGHDFVNSEGLPHYPIGKCMLINESGYRYVDDTSCDRGGVAIVPSGTVKCRIGNVTVQVIATNNDLGPMPCSPAEVIASEGPVEKTACTFNYSRTLPNKYYEPRDRYFQQYMLKGEWQYWFDLDSVDHHKDYFSEFIIIAVVALLGGKYVLWLLITYTILSEQMAMGAGVNTEEIVMIGNLLTDSDIEVVVYFLLLYLIVKEELARKWIILVYHILVANPMKTIGVVLLMLGGVVKASRINADDQSAMDPCFLLVTGVVAVLMIARREPATLPLIVALLAIRTSGFLLPASIDVTVAVVLIVLLLASYITDYFRYKKWLQLLFSLIAGIFIIRSLKHINQMEVPEISMPSWRPLALVLFYITSTAITTNWDIDLAGFLLQWAPAVIMMATMWADFLTLIIVLPSYELSKLYFLKNVRTDVEKNWLGKVKYRQISSVYDICDSEEAVYLFPSRHKSGSRPDFILPFLKAVLISCISSQWQVVYISYLILEITYYMHRKIIDEVSGGANFLSRLIAAIIELNWAIDDEECKGLKKLYLLSGRAKNLIVKHKVRNEAVHRWFGEEEIYGAPKVITIIKASTLSKNRHCIICTICEGKEWNGANCPKCGRQGKPITCGMTLADFEEKHYKKIFIREESSCPVPFDPSCHCNYFRHDGPFRKEYKGYVQYTARGQLFLRNLPILATKMKLLMVGNLGAEIGDLEHLGWVLRGPAVCKKITNHEKCHVNIMDKLTAFFGIMPRGTTPRAPVRFPTALLKVRRGLETGWAYTHQGGISSVDHVTAGKDLLVCDSMGRTRVVCHSNNKMTDETEYGIKTDSGCPEGARCYVLNPEAVNISGTKGAMVHLQKTGGEFTCVTASGTPAFFDLKNLKGWSGLPIFEASSGRVVGRVKVGKNEDSKPTKLMSGIQTVSKNQTDLADIVKKLTSMNRGEFKQITLATGAGKTTELPRSVIEEIGRHKRVLVLIPLRAAAESVYQYMRVKYPSISFNLRIGDMKEGDMATGITYASYGYFCQLPQPKLRAAMVEYSYIFLDEYHCATPEQLAIIGKIHRFAENLRVVAMTATPAGTVTTTGQKHPIEEFIAPEVMKGEDLGSEYLDIAGLKIPTEEMKGNMLVFAPTRNMAVETAKKLKAKGYNSGYYYSGENPENLRVVTSQSPYVVVATNAIESGVTLPDLDTVVDTGLKCEKRVRISSKMPFIVTGLKRMAVTIGEQAQRRGRVGRVKPGRYYRSQETASGSKDYHYDLLQAQRYGIEDGINVTKSFREMNYDWSLYEEDSLMITQLEVLNNLLISEDLPAAVKNIMARTDHPEPIQLAYNSYENQIPVLFPKIKNGEVTDSYENYTYLNARKLGEDVPAYVYATEDEDLAVDLLGMDWPDPGNQQVVETGRALKQVTGLSTAENALLIALFGYVGYQTLSKRHIPMITDIYTLEDHRLEDTTHLQFAPNAIRTDGKDSELKELAVGDLDKYVDALVDYSKQGMKFIKVQAEKVRDSQSTKEGLQTIKEYVDKFIQSLTENKEEIIRYGLWGVHTALYKSLAARLGHETAFATLVVKWLAFGGETVSAHIKQVAVDLVVYYIINKPSFPGDTETQQEGRRFVASLFISALATYTYKTWNYNNLQRVVEPALAYLPYATSALKLFTPTRLESVVILSSTIYKTYLSIRKGKSDGLLGTGISAAMEILNQNPISVGISVMLGVGAIAAHNAIESSEQKRTLLMKVFVKNFLDQAATDELVKENPEKIIMALFEAVQTIGNPLRLIYHLYGVYYKGWEAKELAEKTAGRNLFTLIMFEAFELLGMDSEGKIRNLSGNYILDLIFNLHNKLNKGLKKLVLGWAPAPLSCDWTPSDERISLPHNNYLRVETRCPCGYEMKAIKNVAGKLTKVEEKGSFLCRNRLGRGPPNFKVTKFYDDNLIEVKPVARLEGQVDLYYKGVTAKLDYNNGKVLLATNKWEVDHAFLTRLVKKHTGIGFKGAYLGDRPDHQDLVDRDCATITKNSVQFLKMKKGCAFTYDLTISNLVRLIELVHKNNLQEREIPTVTVTTWLAYSFVNEDLGTIKPVLGEKVIPEPPEELSLQPTVRLVTTETAITITGEAEVMTTGITPVVEMKEEPQLDHQSTTLKVGLKEGEYPGPGVNPNHLAEVIDEKDDRPFVLIIGNKGSTSNRARTAKNIRLYKGNNPREIRDLMSQGRILTVALKELDPELKELVDYKGTFLNREALEALSLGKPIKRKTTTAMIRRLIEPEVEEELPDWFQAEEPLFLEAKIQNDLYHLIGSVDSIKSKAKELGATDNTKIVKEVGARTYTMKLSSWSTQVTKKQMSLAPLFEELLLKCPPCSKISKGHMVSAYQLAQGNWEPLGCGVYMGTIPARRLKIHPYEAYLKLKELVEVESSRATAKESIIREHNTWILRKVRHEGNLRTKSMINPGKISDQLCRDGHKRNIYNKIIGSTMASAGIRLEKLPVVRAQTDTTSFHQAIREKIDKTENKQTPELHEELMKVFDCLKIPELKESYDEVSWEQLEAGINRKGAAGYLESKNIGEVLDTEKHIVEQLIKDLRKGKKIRYYETAIPKNEKRDVSDDWEAGEFVDEKKPRVIQYPDAKVRLAITKVMYKWVKQKPVVIPGYEGKTPLFDIFNKVKKEWDSFQDPVAVSFDTKAWDTQVTSRDLMLIKDIQKYYFKRSIHKFLDTITEHMVEVPVITADGEVYIRNGQRGSGQPDTSAGNSMLNVLTMIYAFCKSTGIPYRGFSRVARIHVCGDDGFLITERGLGLKFSEKGMQILHEAGKPQKITEGDKMKVAYRFEDIEFCSHTPVPVRWADNTSSYMAGRSTATILAKMATRLDSSGERGSTAYEKAVAFSFLLMYSWNPVVRRICLLVLSQFPEISPSKNTIYYYQGDPIAAYREVIGKQLCELKRTGFEKLAGLNLSMTTLGIWTKHTSKRLIQACVEIGKREGTWLVNADRLIAGKTGKFYIPSTGVTLLGKHYEEINLKQKAAQPPIEGVDRYKLGPIVNVILRRLRVMLMTVASGSW

SEQ ID NO: 10 - DNA, 12332 bp, Artificial Sequence
  XIKE-C BVDV-Sequence
gtatacgagattagctaaagaactcgtatatggattggacgtcaacaaatttttaattggcaacgtagggaaccttcccctcagcgaaggccgaaaagaggctagccatgcccttagtaggactagcaaaagtaggggactagcggtagcagtgagttcgttggatggccgaacccctgagtacaggggagtcgtcaatggttcgacactccattagtcgaggagtctcgagatgccatgtggacgagggcatgcccacggcacatcttaacccatgcgggggttgcatgggtgaaagcgctattcgtggcgttatggacacagcctgatagggtgtagcagagacctgctattccgctagtaaaaactctgctgtacatggcacatggagttgttttcaaatgaacttttatacaaaacatataaacaaaaaccagcaggcgtcgtggaacctgtttacgacgtcaacgggcgcccactgtttggagagagcagtgacttgcacccgcagtcaacactaaaactaccacaccaacgaggcagcgccaacatcctgaccaatgctaggtccctaccgcggaaaggtgactgccggagaggtaatgtgtatggaccggtgagtggcatctatatcaaaccaggaccgatctactaccaggattatgtgggccccgtctatcatagagccccactggaactatgtagggaggcaagtatgtgcgaaacaactaggagagttggcagagtgaccggtagtgatgggaaattatatcatatctacatctgcatagatgggtgtatcctcctgaagagggcgactaggaaccaaccagaagtcctgaaatgggtatacaacagattaaattgtcctttatgggtcaccagctgctccgatgaagggagcaagggtgctacaagtaagaagcagcctaagccagataggatagaaaaaggtaagatgaaaatagccccaaaagagacagaaaaagattgcaaaaccagaccccccgacgcgactatagtagtagaaggggttaagtaccaggtgaagaaaaaaggaaaggtaaggggaaaaaatactcaagatgggttatatcacaacaagaataagccccctgaatcaagaaaaaaattggaaaaggcactgctggcttgggccatcttagcagcggtcctgcttcagctggtaacaggagagaatatcacccagtggaacttgatggacaacggcaccgagggaatacagcaagcgatgttcctaagaggggtgaacaggagtctattaggaatttggccagagaaaatttgcaccggagtaccaactcacttagcaacagactatgagcttaaagagatagtggggatgatggacgcgagtgagaagaccaactacacgtgttgcaggttgcaaagacatgagtggaataaacatggttggtgtaactggtttcatatagaaccgtggatatggttgatgaacaaaacccaaaacaacctgacagaagggcaaccgcttagggagtgtgctgtgacttgtaggtatgacaaggaaacagaattgaacatcgtgacacaggctagggacagacctacaactctgacaggttgcaagaaaggcaagaatttctctttcgcaggtgttatactggatgggccctgtaactttaaagtatcggttgaagatgtgctgttcaaggagcacgattgcggcaacatgctgcaagagaccgcgatacagctactcgatggggcaaccaacaccattgagggagcaagggtagggacggccaagttgacaacctggttagggaagcaattagggatccttggtaagaagttggagaacaaaagcaaagcatggtttggtgcacatgcagcaagtccatactgcggagtggagaggaagatcggttacgtatggtatacaaaaaactgcactccagcttgccttccaagaaacactagaataataggccccgggaaatttgataccaacgccgaagatggaaaaatactccatgagatgggggggcacctctcagaatttgtcctattgtccttggtggttctgtctgactttgccccggaaaccgcgagcgtcatctacttggttctacattttgcgatcccgcaaagccacgttgatgtagacacatgcgacaagaaccagctgaatttaacggtagcaaccacagtagcagaggtcataccagggacagtgtggaacctagggaagtatgtctgcataagaccagactggtggccatatgagacgacgacagtcttcgtcatagaggaagcagggcaagtaatcaaattgatgctaagggccatcagagacttaactaggatatggaatgctgccactaccacagctttcttaatctttttagtaaaagcactgaggggacaactaatccaagggctattgtggctgatgctaataacaggagcacagggcttccctgaatgcaaagagggcttccaatatgccatatctaaagacaggaaaatggggttattggggccagagagcttaactacaacatggcacctccccaccaaaaaaatagtggattccatggtgcatgtatggtgtgaaggaaaagacttgaaaatattaaaaatgtgcacaaaggaagagaggtatctagtggctgtgcacgagagagccttatcaaccagtgccgagtttatgcagatcagtgatgggacaataggcccagacgtgatagatatgcctgatgactttgagtttggactctgcccttgtgactcaaaaccagtgataaagggcaaatttaatgccagcttactgaatggaccagctttccagatggtatgcccacaggggtggactggtacaatagaatgcaccctagcgaaccaagacaccttggacacaactgtcattaggacatatagaagaactaccccatttcagcggagaaaatggtgtacctatgaaaaaataataggggaagatatctatgaatgcattctaggtggaaactggacatgcataaccggtgaccatagcaggttgaaagacggacctatcaagaagtgtaagtggtgtggccatgacttcgtcaactcagaggggctaccacactacccaataggcaagtgcatgctcatcaacgagagtgggtacaggtatgtagatgacacctcttgcgataggggtggtgtagccatagttccatctggcaccgtaaagtgtagaataggtaacgtcacggtgcaagttatcgctactaacaatgatctgggacccatgccttgcagcccagctgaagtgatagcaagtgaaggaccagtggaaaagactgcatgcacattcaactattcaaggactctacctaataagtattatgagccaagggaccggtacttccaacaatacatgttaaaaggggagtggcaatattggttcgacctggattctgtagaccaccacaaagactacttctcagagttcataatcatagcagtggtcgccttgttgggtggtaagtacgtactgtggctcttgataacatacacaatactgtctgagcagatggctatgggtgctggagtgaatactgaagagatagtcatgataggcaatttgctgacagacagtgatattgaggttgtggtttatttccttcttctgtacttaatagttaaagaggaactggcgaggaaatggattatactggtataccacatccttgtagccaaccctatgaaaacaattggggtcgtcttactaatgctagggggagtggtgaaggccagcagaatcaatgctgatgaccaaagtgctatggacccatgctttcttctcgtgacaggcgtagtggctgttttgatgatcgctagaagagaacctgccacattaccactgattgtagcattgctagcaataagaacatcaggattcctactgcccgctagcattgatgtaactgtagcagtagtattaattgtacttttgttggctagctacataacagactactttagatataaaaagtggcttcaactcttatttagtctgatagctggtatctttattataaggagcttaaaacatatcaaccagatggaggtaccagaaatatctatgccaagttggagacctctagctctggtccttttctatataacatctacagcaataaccactaattgggacattgacttagcaggcttcctgctgcaatgggcgccagcagtgatcatgatggctaccatgtgggcagactttttgactctgatcatagtcctgcccagttacgagttatctaagctttacttcctaaagaacgtcaggacagacgtggaaaagaactggctcggcaaagtgaaatacagacagatcagttcagtttatgacatctgtgacagtgaggaagcagtgtacctatttccatcaaggcataagagtggaagcaggccagatttcatattaccttttttgaaagccgtgttaataagctgcatcagcagccaatggcaagtggtttacatttcttacctaatactggaaattacatactatatgcacaggaaaatcatagatgaggtgtcaggaggagcaaattttctatcaagactcatagcagccatcatagaattaaattgggccatagatgatgaggaatgtaaaggactgaagaaactgtatctcttgtcagggagagcgaagaatttgatagttaaacataaggtaagaaatgaagccgtccacagatggtttggtgaggaggaaatatacggggcacccaaggtgatcactatcataaaagctagtaccctaagtaaaaacaggcactgcataatctgcacgatctgtgaagggaaagaatggaatggagccaactgcccaaagtgtggaagacaaggaaagcccataacatgtggaatgacactcgcagactttgaggagaaacattacaaaaagatatttataagagaagaatcttcttgtcctgtgccttttgatccttcttgccattgtaattattttcgccacgatgggcctttcaggaaagagtataagggttacgtccaatacacagccagaggacaactctttctgaggaacctaccaattctagcgacgaagatgaagctattaatggtgggaaacctcggcgcagaaattggcgacctggaacatctaggatgggtactgagagggccagccgtgtgcaaaaaaattaccaaccatgagaagtgccacgtaaacatcatggataagctaactgcattttttggaatcatgcctagaggcacgacccctagggcacctgtgaggttccccacagcactactaaaagtgagaagggggctagagacgggatgggcttacacgcaccaaggagggatcagctcggtagaccatgtcacagccggaaaggatttactagtgtgtgacagtatgggcaggaccagggttgtctgtcatagtaacaataagatgactgatgagactgagtatggcatcaagaccgactcagggtgtcccgaaggtgcgaggtgttacgtgctaaacccagaagctgttaacatttctggcacaaaaggagctatggtacacctccagaaaacggggggggagttcacatgtgtcactgcctcagggaccccggctttcttcgatctgaaaaatctaaaaggctggtccgggctaccaatttttgaagcatccagtggcagggtggttggtagggtgaaagtcggcaagaatgaggattccaagcccaccaaactaatgagcggaatccagacagtgtctaagaaccagacagacctagcggacatcgtaaaaaaattgactagtatgaacagaggagagttcaaacagataacattagccactggggcaggaaaaactacggaactgccaaggtccgtcatagaggagatagggaggcacaaaagggtcttagtcctgataccattgagagcagcagcagagtcagtgtatcagtatatgagagtgaagtacccaagtatatctttcaatttgagaataggagatatgaaggaaggtgacatggccactggtatcacctacgcctcatatgggtacttttgtcagcttcctcagcccaaactgagagctgccatggtagagtactcatatatattcttagatgagtaccactgtgctacacccgagcaattagcaataattggaaagatacacaggtttgctgaaaatcttagagtggtagcaatgacagcaaccccagctggaacggtcacaacgactggtcagaaacaccctatagaggagttcatagccccagaggtgatgaaaggtgaagatctaggtagtgaatacttggatattgcagggttgaagataccgactgaagagatgaaaggcaacatgctcgtgttcgcgccaactaggaacatggcagtagaaacagctaagaaattgaaggctaagggatacaactctggatactattacagtggggaaaacccagagaacttgagggtggtaacctcgcaatccccgtatgtggtagtagccaccaatgccatagagtcaggtgtgacattaccagacttagacacagttgtagacactggactaaagtgtgagaagagggtgaggatttcttcaaaaatgcccttcattgtaacaggacttaagagaatggcagtcacaatcggagagcaagcccagcgcaggggtagagtaggaagagtcaagccaggtaggtactataggagtcaagaaacagcttcagggtcaaaagattaccattacgacctactgcaagcccagaggtacggaatagaagatggaattaatgtaacaaagtcattcagggagatgaactatgattggagcctttacgaagaggacagcttgatgataactcaactcgaggtccttaacaacctccttatatcagaagacctgcctgccgcagtgaagaacatcatggcccggaccgatcacccagaacccatacaactggcctataacagttatgaaaaccaaattccagtgctgttcccaaagatcaaaaatggtgaggtgacagacagttatgagaattacacatatctcaatgcaagaaaattaggagaggacgtgccggcatatgtgtacgccacagaggatgaggatctagcagtggatcttctgggtatggattggccggacccaggcaaccaacaggtggtagagacagggagggcattaaaacaagtaactggcttatccacagcagaaaacgccctcttgatagccctattcggctacgtcgggtaccagacactttcaaaaaggcacatacccatgattactgacatctatacacttgaagaccacaggcttgaggacacaacccacctccagtttgccccaaacgctataaggaccgacggcaaggactcagagttgaaggaattagctgtgggagaccttgataaatatgtggacgcactggtagactactccaaacaagggatgaaattcatcaaagtccaagctgaaaaggtcagagactcccagtctacgaaggaaggcttgcaaaccattaaggagtatgtggataagtttatacaatcactaacagagaataaggaggagatcatcaggtatggactatggggagttcacacggcactctacaaaagcttggcagcgagactggggcatgaaacagcttttgcaactttagtggtaaaatggttggcttttgggggcgaaacggtatctgctcacatcaagcaagtagcagttgatctagtagtatattatatcatcaacaaaccatcttttcctggagatacagagacccaacaagaggggaggaagtttgtggctagtctttttatatctgcactagcaacatacacatataaaacctggaattacaacaatctgcaacgggttgtcgaacctgccttagcttacctcccatatgctacaagtgccttgaagttgttcacacccacaagattagagagtgtggtcatactcagttctacaatttacaagacatacctctctataaggaagggtaagagtgacggcttgttaggtacaggcataagtgcagccatggagatcttaaaccaaaacccaatctcagtaggtatatctgtgatgctgggggtaggtgccatcgccgcccataatgcaatagaatctagtgaacagaaaagaactttgctgatgaaggtctttgtaaaaaacttcttagaccaagcagcaacagatgagctagtcaaagagaaccctgaaaaaataatcatggctctatttgaagcagtccagaccataggaaaccccctaagactcatctaccatctgtacggggtgtactataaggggtgggaagcaaaagaactcgcagagaaaactgctggccgcaacttattcacattgatcatgtttgaggcctttgagcttttaggtatggactcagaaggaaagataagaaacttgtcaggcaactacatactggacttaatcttcaacttgcataataaattaaacaaggggctcaaaaaactagtccttgggtgggctcctgcacctttgagctgtgattggacaccaagtgatgagagaataagcctacctcataacaactacttaagggtagaaaccaggtgtccttgtggctatgagatgaaggcaataaaaaatgttgctggtaaattgacaaaagttgaagaaaaggggtccttcctatgcaggaatagattagggagaggacctccaaacttcaaagtaacaaagttctatgatgataacttgatagaagtcaagccagtagctaggctagaaggccaggtggacctctattacaagggagtaacagctaagttagactacaacaatgggaaagtactgttagctaccaacaagtgggaggtggaccacgctttcctgaccagactagtaaagaagcacacagggataggttttaaaggtgcatatttgggtgaccgaccagaccatcaagatcttgtcgatagagattgtgcaactataacgaagaactcagtacagttcctaaaaatgaagaagggttgcgctttcacatatgacctaacaatctctaaccttgtcaggcttattgaactagtccataagaataatttacaagaaagagagatccctaccgtgacagtaactacttggcttgcatattcttttgtcaatgaagacctggggactatcaagcctgtattgggggagaaagtcatcccagaaccccccgaggagttgagtctccaacccaccgtgagactagtcaccactgaaacagcaataaccataacaggggaggctgaagtgatgacgacagggatcacaccagtggtagagatgaaagaagaacctcagctggaccaccagtcaactaccctaaaggtagggttgaaggaaggggaatatccagggccaggagttaaccctaaccatttagcagaggtgatagatgagaaagatgacaggccttttgtcctaatcatcggtaacaaaggttctacctcgaacagagcaagaacggccaagaatatacggctgtacaaaggaaacaacccaagagagatcagggatctgatgagccaaggaagaatattgacggttgctctaaaagagttggacccggaattaaaagaattagtagattacaaggggacctttctcaatagggaagctttagaagccctaagcttaggtaagccaatcaagaggaaaaccacaacagcaatgatcaggaggttaatagagccagaggttgaggaggaactaccagattggttccaagcggaagaacccctatttttggaagcaaaaatacagaatgacttataccacctaattggcagtgtagatagtataaaaagcaaagcaaaggaattaggggccacagataacacaaagatagtgaaggaagttggggctaggacctatacgatgaaattgagcagctggagcacacaagttacaaaaaaacagatgagtctagcccctctctttgaagagctgttattaaagtgccctccatgtagtaaaatttcaaagggacatatggtgtcagcataccaactggctcaaggaaactgggaacccctcgggtgtggggtctatatgggaaccataccagctaggcgtctcaagatccacccttatgaggcttaccttaaactcaaagagctggtggaagttgaatcttcgagggccactgcaaaagaatccatcataagagaacataacacctggatcctgcggaaggtgagacatgaagggaacctaagaaccaaatcaatgatcaaccctgggaaaatatcagatcagctatgcagagatggacacaaaagaaacatatataataagatcataggctcaacaatggcctctgctggtattaggctggagaaactgccagtagtccgagcccaaactgacacaaccagtttccaccaagccataagagaaaaaattgataaaacagaaaacaagcagacccctgaattgcatgaagaactaatgaaggtcttcgactgcttaaagatcccagagctgaaggaatcgtatgatgaagtttcatgggaacaattagaagccgggataaaccgtaagggtgcagcaggctatctagagagcaagaacataggggaagtcctagacacagagaaacacatagtagagcagctgatcaaggatctgaggaaggggaagaagattaggtactatgaaacagccatccccaagaatgagaagagagacgtcagcgacgactgggaagccggagagttcgttgatgaaaagaaaccaagagtaatccagtacccggacgccaaggtgagactggccattacaaaagtgatgtacaaatgggtaaagcaaaaaccagtggtgatacccggctatgaaggtaaaacacctctatttgacatattcaacaaagtgaagaaggaatgggattcattccaggaccccgtagcagtgagctttgacaccaaagcgtgggatacacaagtcaccagtagagacctaatgttgataaaggatatccagaaatattatttcaagagaagtatacacaaatttttagatacaataacagaacacatggtggaggtacctgtcattacagcagacggtgaagtttacataaggaatggtcagaggggtagtggccaacccgacacaagtgctggtaatagtatgttgaatgtcctaaccatgatatatgctttctgtaaaagtacaggcataccttacaggggattcagcagagtggcaagaatccatgtgtgtggtgatgatggctttttgataacagagagaggactgggactgaaattctctgagaagggtatgcagatattacatgaggccgggaagccccagaaaataactgaaggggacaaaatgaaagtggcatacagattcgaggacatagagttttgttcccatactcccgtgccagtcagatgggcagataacaccagtagttacatggcagggaggagcacagccactatactagctaagatggcaaccaggctggattccagcggagagaggggtagcacagcttatgagaaggccgtagccttcagcttccttttgatgtactcatggaatcccgtagttagaaggatctgcttactggtgttgtcacagtttccagaaatatccccatccaaaaacacaatatactactaccaaggggatcccatagctgcgtacagagaagtgatagggaaacagctgtgtgaactgaaaagaacaggatttgagaagctggctggtctgaatttgagtatgaccactctaggcatctggacaaaacatactagtaaaagactaatccaagcctgtgtagaaataggtaagagagaaggtacctggttagttaatgctgacagactgattgcaggaaagactgggaagttttacatcccaagcactggtgtcactctgttgggaaaacactatgaggaaattaacttaaagcaaaaggcggcacaaccgccgatagagggggttgacagatataagttgggccccatagttaatgttatcttgagaaggctgagggtgatgctgatgacagttgccagcggaagctggtgaatccgtccggagcgtcgtgccctcactcaaggtttttaattgtaaatattgtaaatagacagctaagatatttattgtagttggatagtaatgcagtgatagtaaataccccaatttaacactacctccaatgcactaagcactttagctgtgtgaggttaactcgacgtccacggttggactagggaagacctctaacagcccc

SEQ ID NO: 11 - DNA, 11840 bp, Artificial Sequence
  XIKE-C-NdN BVDV-Sequence
gtatacgagattagctaaagaactcgtatatggattggacgtcaacaaatttttaattggcaacgtagggaaccttcccctcagcgaaggccgaaaagaggctagccatgcccttagtaggactagcaaaagtaggggactagcggtagcagtgagttcgttggatggccgaacccctgagtacaggggagtcgtcaatggttcgacactccattagtcgaggagtctcgagatgccatgtggacgagggcatgcccacggcacatcttaacccatgcgggggttgcatgggtgaaagcgctattcgtggcgttatggacacagcctgatagggtgtagcagagacctgctattccgctagtaaaaactctgctgtacatggcacatggagttgttttccgatgaagggagcaagggtgctacaagtaagaagcagcctaagccagataggatagaaaaaggtaagatgaaaatagccccaaaagagacagaaaaagattgcaaaaccagaccccccgacgcgactatagtagtagaaggggttaagtaccaggtgaagaaaaaaggaaaggtaaggggaaaaaatactcaagatgggttatatcacaacaagaataagccccctgaatcaagaaaaaaattggaaaaggcactgctggcttgggccatcttagcagcggtcctgcttcagctggtaacaggagagaatatcacccagtggaacttgatggacaacggcaccgagggaatacagcaagcgatgttcctaagaggggtgaacaggagtctattaggaatttggccagagaaaatttgcaccggagtaccaactcacttagcaacagactatgagcttaaagagatagtggggatgatggacgcgagtgagaagaccaactacacgtgttgcaggttgcaaagacatgagtggaataaacatggttggtgtaactggtttcatatagaaccgtggatatggttgatgaacaaaacccaaaacaacctgacagaagggcaaccgcttagggagtgtgctgtgacttgtaggtatgacaaggaaacagaattgaacatcgtgacacaggctagggacagacctacaactctgacaggttgcaagaaaggcaagaatttctctttcgcaggtgttatactggatgggccctgtaactttaaagtatcggttgaagatgtgctgttcaaggagcacgattgcggcaacatgctgcaagagaccgcgatacagctactcgatggggcaaccaacaccattgagggagcaagggtagggacggccaagttgacaacctggttagggaagcaattagggatccttggtaagaagttggagaacaaaagcaaagcatggtttggtgcacatgcagcaagtccatactgcggagtggagaggaagatcggttacgtatggtatacaaaaaactgcactccagcttgccttccaagaaacactagaataataggccccgggaaatttgataccaacgccgaagatggaaaaatactccatgagatgggggggcacctctcagaatttgtcctattgtccttggtggttctgtctgactttgccccggaaaccgcgagcgtcatctacttggttctacattttgcgatcccgcaaagccacgttgatgtagacacatgcgacaagaaccagctgaatttaacggtagcaaccacagtagcagaggtcataccagggacagtgtggaacctagggaagtatgtctgcataagaccagactggtggccatatgagacgacgacagtcttcgtcatagaggaagcagggcaagtaatcaaattgatgctaagggccatcagagacttaactaggatatggaatgctgccactaccacagctttcttaatctttttagtaaaagcactgaggggacaactaatccaagggctattgtggctgatgctaataacaggagcacagggcttccctgaatgcaaagagggcttccaatatgccatatctaaagacaggaaaatggggttattggggccagagagcttaactacaacatggcacctccccaccaaaaaaatagtggattccatggtgcatgtatggtgtgaaggaaaagacttgaaaatattaaaaatgtgcacaaaggaagagaggtatctagtggctgtgcacgagagagccttatcaaccagtgccgagtttatgcagatcagtgatgggacaataggcccagacgtgatagatatgcctgatgactttgagtttggactctgcccttgtgactcaaaaccagtgataaagggcaaatttaatgccagcttactgaatggaccagctttccagatggtatgcccacaggggtggactggtacaatagaatgcaccctagcgaaccaagacaccttggacacaactgtcattaggacatatagaagaactaccccatttcagcggagaaaatggtgtacctatgaaaaaataataggggaagatatctatgaatgcattctaggtggaaactggacatgcataaccggtgaccatagcaggttgaaagacggacctatcaagaagtgtaagtggtgtggccatgacttcgtcaactcagaggggctaccacactacccaataggcaagtgcatgctcatcaacgagagtgggtacaggtatgtagatgacacctcttgcgataggggtggtgtagccatagttccatctggcaccgtaaagtgtagaataggtaacgtcacggtgcaagttatcgctactaacaatgatctgggacccatgccttgcagcccagctgaagtgatagcaagtgaaggaccagtggaaaagactgcatgcacattcaactattcaaggactctacctaataagtattatgagccaagggaccggtacttccaacaatacatgttaaaaggggagtggcaatattggttcgacctggattctgtagaccaccacaaagactacttctcagagttcataatcatagcagtggtcgccttgttgggtggtaagtacgtactgtggctcttgataacatacacaatactgtctgagcagatggctatgggtgctggagtgaatactgaagagatagtcatgataggcaatttgctgacagacagtgatattgaggttgtggtttatttccttcttctgtacttaatagttaaagaggaactggcgaggaaatggattatactggtataccacatccttgtagccaaccctatgaaaacaattggggtcgtcttactaatgctagggggagtggtgaaggccagcagaatcaatgctgatgaccaaagtgctatggacccatgctttcttctcgtgacaggcgtagtggctgttttgatgatcgctagaagagaacctgccacattaccactgattgtagcattgctagcaataagaacatcaggattcctactgcccgctagcattgatgtaactgtagcagtagtattaattgtacttttgttggctagctacataacagactactttagatataaaaagtggcttcaactcttatttagtctgatagctggtatctttattataaggagcttaaaacatatcaaccagatggaggtaccagaaatatctatgccaagttggagacctctagctctggtccttttctatataacatctacagcaataaccactaattgggacattgacttagcaggcttcctgctgcaatgggcgccagcagtgatcatgatggctaccatgtgggcagactttttgactctgatcatagtcctgcccagttacgagttatctaagctttacttcctaaagaacgtcaggacagacgtggaaaagaactggctcggcaaagtgaaatacagacagatcagttcagtttatgacatctgtgacagtgaggaagcagtgtacctatttccatcaaggcataagagtggaagcaggccagatttcatattaccttttttgaaagccgtgttaataagctgcatcagcagccaatggcaagtggtttacatttcttacctaatactggaaattacatactatatgcacaggaaaatcatagatgaggtgtcaggaggagcaaattttctatcaagactcatagcagccatcatagaattaaattgggccatagatgatgaggaatgtaaaggactgaagaaactgtatctcttgtcagggagagcgaagaatttgatagttaaacataaggtaagaaatgaagccgtccacagatggtttggtgaggaggaaatatacggggcacccaaggtgatcactatcataaaagctagtaccctaagtaaaaacaggcactgcataatctgcacgatctgtgaagggaaagaatggaatggagccaactgcccaaagtgtggaagacaaggaaagcccataacatgtggaatgacactcgcagactttgaggagaaacattacaaaaagatatttataagagaagaatcttcttgtcctgtgccttttgatccttcttgccattgtaattattttcgccacgatgggcctttcaggaaagagtataagggttacgtccaatacacagccagaggacaactctttctgaggaacctaccaattctagcgacgaagatgaagctattaatggtgggaaacctcggcgcagaaattggcgacctggaacatctaggatgggtactgagagggccagccgtgtgcaaaaaaattaccaaccatgagaagtgccacgtaaacatcatggataagctaactgcattttttggaatcatgcctagaggcacgacccctagggcacctgtgaggttccccacagcactactaaaagtgagaagggggctagagacgggatgggcttacacgcaccaaggagggatcagctcggtagaccatgtcacagccggaaaggatttactagtgtgtgacagtatgggcaggaccagggttgtctgtcatagtaacaataagatgactgatgagactgagtatggcatcaagaccgactcagggtgtcccgaaggtgcgaggtgttacgtgctaaacccagaagctgttaacatttctggcacaaaaggagctatggtacacctccagaaaacggggggggagttcacatgtgtcactgcctcagggaccccggctttcttcgatctgaaaaatctaaaaggctggtccgggctaccaatttttgaagcatccagtggcagggtggttggtagggtgaaagtcggcaagaatgaggattccaagcccaccaaactaatgagcggaatccagacagtgtctaagaaccagacagacctagcggacatcgtaaaaaaattgactagtatgaacagaggagagttcaaacagataacattagccactggggcaggaaaaactacggaactgccaaggtccgtcatagaggagatagggaggcacaaaagggtcttagtcctgataccattgagagcagcagcagagtcagtgtatcagtatatgagagtgaagtacccaagtatatctttcaatttgagaataggagatatgaaggaaggtgacatggccactggtatcacctacgcctcatatgggtacttttgtcagcttcctcagcccaaactgagagctgccatggtagagtactcatatatattcttagatgagtaccactgtgctacacccgagcaattagcaataattggaaagatacacaggtttgctgaaaatcttagagtggtagcaatgacagcaaccccagctggaacggtcacaacgactggtcagaaacaccctatagaggagttcatagccccagaggtgatgaaaggtgaagatctaggtagtgaatacttggatattgcagggttgaagataccgactgaagagatgaaaggcaacatgctcgtgttcgcgccaactaggaacatggcagtagaaacagctaagaaattgaaggctaagggatacaactctggatactattacagtggggaaaacccagagaacttgagggtggtaacctcgcaatccccgtatgtggtagtagccaccaatgccatagagtcaggtgtgacattaccagacttagacacagttgtagacactggactaaagtgtgagaagagggtgaggatttcttcaaaaatgcccttcattgtaacaggacttaagagaatggcagtcacaatcggagagcaagcccagcgcaggggtagagtaggaagagtcaagccaggtaggtactataggagtcaagaaacagcttcagggtcaaaagattaccattacgacctactgcaagcccagaggtacggaatagaagatggaattaatgtaacaaagtcattcagggagatgaactatgattggagcctttacgaagaggacagcttgatgataactcaactcgaggtccttaacaacctccttatatcagaagacctgcctgccgcagtgaagaacatcatggcccggaccgatcacccagaacccatacaactggcctataacagttatgaaaaccaaattccagtgctgttcccaaagatcaaaaatggtgaggtgacagacagttatgagaattacacatatctcaatgcaagaaaattaggagaggacgtgccggcatatgtgtacgccacagaggatgaggatctagcagtggatcttctgggtatggattggccggacccaggcaaccaacaggtggtagagacagggagggcattaaaacaagtaactggcttatccacagcagaaaacgccctcttgatagccctattcggctacgtcgggtaccagacactttcaaaaaggcacatacccatgattactgacatctatacacttgaagaccacaggcttgaggacacaacccacctccagtttgccccaaacgctataaggaccgacggcaaggactcagagttgaaggaattagctgtgggagaccttgataaatatgtggacgcactggtagactactccaaacaagggatgaaattcatcaaagtccaagctgaaaaggtcagagactcccagtctacgaaggaaggcttgcaaaccattaaggagtatgtggataagtttatacaatcactaacagagaataaggaggagatcatcaggtatggactatggggagttcacacggcactctacaaaagcttggcagcgagactggggcatgaaacagcttttgcaactttagtggtaaaatggttggcttttgggggcgaaacggtatctgctcacatcaagcaagtagcagttgatctagtagtatattatatcatcaacaaaccatcttttcctggagatacagagacccaacaagaggggaggaagtttgtggctagtctttttatatctgcactagcaacatacacatataaaacctggaattacaacaatctgcaacgggttgtcgaacctgccttagcttacctcccatatgctacaagtgccttgaagttgttcacacccacaagattagagagtgtggtcatactcagttctacaatttacaagacatacctctctataaggaagggtaagagtgacggcttgttaggtacaggcataagtgcagccatggagatcttaaaccaaaacccaatctcagtaggtatatctgtgatgctgggggtaggtgccatcgccgcccataatgcaatagaatctagtgaacagaaaagaactttgctgatgaaggtctttgtaaaaaacttcttagaccaagcagcaacagatgagctagtcaaagagaaccctgaaaaaataatcatggctctatttgaagcagtccagaccataggaaaccccctaagactcatctaccatctgtacggggtgtactataaggggtgggaagcaaaagaactcgcagagaaaactgctggccgcaacttattcacattgatcatgtttgaggcctttgagcttttaggtatggactcagaaggaaagataagaaacttgtcaggcaactacatactggacttaatcttcaacttgcataataaattaaacaaggggctcaaaaaactagtccttgggtgggctcctgcacctttgagctgtgattggacaccaagtgatgagagaataagcctacctcataacaactacttaagggtagaaaccaggtgtccttgtggctatgagatgaaggcaataaaaaatgttgctggtaaattgacaaaagttgaagaaaaggggtccttcctatgcaggaatagattagggagaggacctccaaacttcaaagtaacaaagttctatgatgataacttgatagaagtcaagccagtagctaggctagaaggccaggtggacctctattacaagggagtaacagctaagttagactacaacaatgggaaagtactgttagctaccaacaagtgggaggtggaccacgctttcctgaccagactagtaaagaagcacacagggataggttttaaaggtgcatatttgggtgaccgaccagaccatcaagatcttgtcgatagagattgtgcaactataacgaagaactcagtacagttcctaaaaatgaagaagggttgcgctttcacatatgacctaacaatctctaaccttgtcaggcttattgaactagtccataagaataatttacaagaaagagagatccctaccgtgacagtaactacttggcttgcatattcttttgtcaatgaagacctggggactatcaagcctgtattgggggagaaagtcatcccagaaccccccgaggagttgagtctccaacccaccgtgagactagtcaccactgaaacagcaataaccataacaggggaggctgaagtgatgacgacagggatcacaccagtggtagagatgaaagaagaacctcagctggaccaccagtcaactaccctaaaggtagggttgaaggaaggggaatatccagggccaggagttaaccctaaccatttagcagaggtgatagatgagaaagatgacaggccttttgtcctaatcatcggtaacaaaggttctacctcgaacagagcaagaacggccaagaatatacggctgtacaaaggaaacaacccaagagagatcagggatctgatgagccaaggaagaatattgacggttgctctaaaagagttggacccggaattaaaagaattagtagattacaaggggacctttctcaatagggaagctttagaagccctaagcttaggtaagccaatcaagaggaaaaccacaacagcaatgatcaggaggttaatagagccagaggttgaggaggaactaccagattggttccaagcggaagaacccctatttttggaagcaaaaatacagaatgacttataccacctaattggcagtgtagatagtataaaaagcaaagcaaaggaattaggggccacagataacacaaagatagtgaaggaagttggggctaggacctatacgatgaaattgagcagctggagcacacaagttacaaaaaaacagatgagtctagcccctctctttgaagagctgttattaaagtgccctccatgtagtaaaatttcaaagggacatatggtgtcagcataccaactggctcaaggaaactgggaacccctcgggtgtggggtctatatgggaaccataccagctaggcgtctcaagatccacccttatgaggcttaccttaaactcaaagagctggtggaagttgaatcttcgagggccactgcaaaagaatccatcataagagaacataacacctggatcctgcggaaggtgagacatgaagggaacctaagaaccaaatcaatgatcaaccctgggaaaatatcagatcagctatgcagagatggacacaaaagaaacatatataataagatcataggctcaacaatggcctctgctggtattaggctggagaaactgccagtagtccgagcccaaactgacacaaccagtttccaccaagccataagagaaaaaattgataaaacagaaaacaagcagacccctgaattgcatgaagaactaatgaaggtcttcgactgcttaaagatcccagagctgaaggaatcgtatgatgaagtttcatgggaacaattagaagccgggataaaccgtaagggtgcagcaggctatctagagagcaagaacataggggaagtcctagacacagagaaacacatagtagagcagctgatcaaggatctgaggaaggggaagaagattaggtactatgaaacagccatccccaagaatgagaagagagacgtcagcgacgactgggaagccggagagttcgttgatgaaaagaaaccaagagtaatccagtacccggacgccaaggtgagactggccattacaaaagtgatgtacaaatgggtaaagcaaaaaccagtggtgatacccggctatgaaggtaaaacacctctatttgacatattcaacaaagtgaagaaggaatgggattcattccaggaccccgtagcagtgagctttgacaccaaagcgtgggatacacaagtcaccagtagagacctaatgttgataaaggatatccagaaatattatttcaagagaagtatacacaaatttttagatacaataacagaacacatggtggaggtacctgtcattacagcagacggtgaagtttacataaggaatggtcagaggggtagtggccaacccgacacaagtgctggtaatagtatgttgaatgtcctaaccatgatatatgctttctgtaaaagtacaggcataccttacaggggattcagcagagtggcaagaatccatgtgtgtggtgatgatggctttttgataacagagagaggactgggactgaaattctctgagaagggtatgcagatattacatgaggccgggaagccccagaaaataactgaaggggacaaaatgaaagtggcatacagattcgaggacatagagttttgttcccatactcccgtgccagtcagatgggcagataacaccagtagttacatggcagggaggagcacagccactatactagctaagatggcaaccaggctggattccagcggagagaggggtagcacagcttatgagaaggccgtagccttcagcttccttttgatgtactcatggaatcccgtagttagaaggatctgcttactggtgttgtcacagtttccagaaatatccccatccaaaaacacaatatactactaccaaggggatcccatagctgcgtacagagaagtgatagggaaacagctgtgtgaactgaaaagaacaggatttgagaagctggctggtctgaatttgagtatgaccactctaggcatctggacaaaacatactagtaaaagactaatccaagcctgtgtagaaataggtaagagagaaggtacctggttagttaatgctgacagactgattgcaggaaagactgggaagttttacatcccaagcactggtgtcactctgttgggaaaacactatgaggaaattaacttaaagcaaaaggcggcacaaccgccgatagagggggttgacagatataagttgggccccatagttaatgttatcttgagaaggctgagggtgatgctgatgacagttgccagcggaagctggtgaatccgtccggagcgtcgtgccctcactcaaggtttttaattgtaaatattgtaaatagacagctaagatatttattgtagttggatagtaatgcagtgatagtaaataccccaatttaacactacctccaatgcactaagcactttagctgtgtgaggttaactcgacgtccacggttggactagggaagacctctaacagcccc

SEQ ID NO: 12 - protein, 3749 aa, Artificial Sequence
  XIKE-C-NdN
MELFSDEGSKGATSKKQPKPDRIEKGKMKIAPKETEKDCKTRPPDATIVVEGVKYQVKKKGKVRGKNTQDGLYHNKNKPPESRKKLEKALLAWAILAAVLLQLVTGENITQWNLMDNGTEGIQQAMFLRGVNRSLLGIWPEKICTGVPTHLATDYELKEIVGMMDASEKTNYTCCRLQRHEWNKHGWCNWFHIEPWIWLMNKTQNNLTEGQPLRECAVTCRYDKETELNIVTQARDRPTTLTGCKKGKNFSFAGVILDGPCNFKVSVEDVLFKEHDCGNMLQETAIQLLDGATNTIEGARVGTAKLTTWLGKQLGILGKKLENKSKAWFGAHAASPYCGVERKIGYVWYTKNCTPACLPRNTRIIGPGKFDTNAEDGKILHEMGGHLSEFVLLSLVVLSDFAPETASVIYLVLHFAIPQSHVDVDTCDKNQLNLTVATTVAEVIPGTVWNLGKYVCIRPDWWPYETTTVFVIEEAGQVIKLMLRAIRDLTRIWNAATTTAFLIFLVKALRGQLIQGLLWLMLITGAQGFPECKEGFQYAISKDRKMGLLGPESLTTTWHLPTKKIVDSMVHVWCEGKDLKILKMCTKEERYLVAVHERALSTSAEFMQISDGTIGPDVIDMPDDFEFGLCPCDSKPVIKGKFNASLLNGPAFQMVCPQGWTGTIECTLANQDTLDTTVIRTYRRTTPFQRRKWCTYEKIIGEDIYECILGGNWTCITGDHSRLKDGPIKKCKWCGHDFVNSEGLPHYPIGKCMLINESGYRYVDDTSCDRGGVAIVPSGTVKCRIGNVTVQVIATNNDLGPMPCSPAEVIASEGPVEKTACTFNYSRTLPNKYYEPRDRYFQQYMLKGEWQYWFDLDSVDHHKDYFSEFIIIAVVALLGGKYVLWLLITYTILSEQMAMGAGVNTEEIVMIGNLLTDSDIEVVVYFLLLYLIVKEELARKWIILVYHILVANPMKTIGVVLLMLGGVVKASRINADDQSAMDPCFLLVTGVVAVLMIARREPATLPLIVALLAIRTSGFLLPASIDVTVAVVLIVLLLASYITDYFRYKKWLQLLFSLIAGIFIIRSLKHINQMEVPEISMPSWRPLALVLFYITSTAITTNWDIDLAGFLLQWAPAVIMMATMWADFLTLIIVLPSYELSKLYFLKNVRTDVEKNWLGKVKYRQISSVYDICDSEEAVYLFPSRHKSGSRPDFILPFLKAVLISCISSQWQVVYISYLILEITYYMHRKIIDEVSGGANFLSRLIAAIIELNWAIDDEECKGLKKLYLLSGRAKNLIVKHKVRNEAVHRWFGEEEIYGAPKVITIIKASTLSKNRHCIICTICEGKEWNGANCPKCGRQGKPITCGMTLADFEEKHYKKIFIREESSCPVPFDPSCHCNYFRHDGPFRKEYKGYVQYTARGQLFLRNLPILATKMKLLMVGNLGAEIGDLEHLGWVLRGPAVCKKITNHEKCHVNIMDKLTAFFGIMPRGTTPRAPVRFPTALLKVRRGLETGWAYTHQGGISSVDHVTAGKDLLVCDSMGRTRVVCHSNNKMTDETEYGIKTDSGCPEGARCYVLNPEAVNISGTKGAMVHLQKTGGEFTCVTASGTPAFFDLKNLKGWSGLPIFEASSGRVVGRVKVGKNEDSKPTKLMSGIQTVSKNQTDLADIVKKLTSMNRGEFKQITLATGAGKTTELPRSVIEEIGRHKRVLVLIPLRAAAESVYQYMRVKYPSISFNLRIGDMKEGDMATGITYASYGYFCQLPQPKLRAAMVEYSYIFLDEYHCATPEQLAIIGKIHRFAENLRVVAMTATPAGTVTTTGQKHPIEEFIAPEVMKGEDLGSEYLDIAGLKIPTEEMKGNMLVFAPTRNMAVETAKKLKAKGYNSGYYYSGENPENLRVVTSQSPYVVVATNAIESGVTLPDLDTVVDTGLKCEKRVRISSKMPFIVTGLKRMAVTIGEQAQRRGRVGRVKPGRYYRSQETASGSKDYHYDLLQAQRYGIEDGINVTKSFREMNYDWSLYEEDSLMITQLEVLNNLLISEDLPAAVKNIMARTDHPEPIQLAYNSYENQIPVLFPKIKNGEVTDSYENYTYLNARKLGEDVPAYVYATEDEDLAVDLLGMDWPDPGNQQVVETGRALKQVTGLSTAENALLIALFGYVGYQTLSKRHIPMITDIYTLEDHRLEDTTHLQFAPNAIRTDGKDSELKELAVGDLDKYVDALVDYSKQGMKFIKVQAEKVRDSQSTKEGLQTIKEYVDKFIQSLTENKEEIIRYGLWGVHTALYKSLAARLGHETAFATLVVKWLAFGGETVSAHIKQVAVDLVVYYIINKPSFPGDTETQQEGRRFVASLFISALATYTYKTWNYNNLQRVVEPALAYLPYATSALKLFTPTRLESVVILSSTIYKTYLSIRKGKSDGLLGTGISAAMEILNQNPISVGISVMLGVGAIAAHNAIESSEQKRTLLMKVFVKNFLDQAATDELVKENPEKIIMALFEAVQTIGNPLRLIYHLYGVYYKGWEAKELAEKTAGRNLFTLIMFEAFELLGMDSEGKIRNLSGNYILDLIFNLHNKLNKGLKKLVLGWAPAPLSCDWTPSDERISLPHNNYLRVETRCPCGYEMKAIKNVAGKLTKVEEKGSFLCRNRLGRGPPNFKVTKFYDDNLIEVKPVARLEGQVDLYYKGVTAKLDYNNGKVLLATNKWEVDHAFLTRLVKKHTGIGFKGAYLGDRPDHQDLVDRDCATITKNSVQFLKMKKGCAFTYDLTISNLVRLIELVHKNNLQEREIPTVTVTTWLAYSFVNEDLGTIKPVLGEKVIPEPPEELSLQPTVRLVTTETAITITGEAEVMTTGITPVVEMKEEPQLDHQSTTLKVGLKEGEYPGPGVNPNHLAEVIDEKDDRPFVLIIGNKGSTSNRARTAKNIRLYKGNNPREIRDLMSQGRILTVALKELDPELKELVDYKGTFLNREALEALSLGKPIKRKTTTAMIRRLIEPEVEEELPDWFQAEEPLFLEAKIQNDLYHLIGSVDSIKSKAKELGATDNTKIVKEVGARTYTMKLSSWSTQVTKKQMSLAPLFEELLLKCPPCSKISKGHMVSAYQLAQGNWEPLGCGVYMGTIPARRLKIHPYEAYLKLKELVEVESSRATAKESIIREHNTWILRKVRHEGNLRTKSMINPGKISDQLCRDGHKRNIYNKIIGSTMASAGIRLEKLPVVRAQTDTTSFHQAIREKIDKTENKQTPELHEELMKVFDCLKIPELKESYDEVSWEQLEAGINRKGAAGYLESKNIGEVLDTEKHIVEQLIKDLRKGKKIRYYETAIPKNEKRDVSDDWEAGEFVDEKKPRVIQYPDAKVRLAITKVMYKWVKQKPVVIPGYEGKTPLFDIFNKVKKEWDSFQDPVAVSFDTKAWDTQVTSRDLMLIKDIQKYYFKRSIHKFLDTITEHMVEVPVITADGEVYIRNGQRGSGQPDTSAGNSMLNVLTMIYAFCKSTGIPYRGFSRVARIHVCGDDGFLITERGLGLKFSEKGMQILHEAGKPQKITEGDKMKVAYRFEDIEFCSHTPVPVRWADNTSSYMAGRSTATILAKMATRLDSSGERGSTAYEKAVAFSFLLMYSWNPVVRRICLLVLSQFPEISPSKNTIYYYQGDPIAAYREVIGKQLCELKRTGFEKLAGLNLSMTTLGIWTKHTSKRLIQACVEIGKREGTWLVNADRLIAGKTGKFYIPSTGVTLLGKHYEEINLKQKAAQPPIEGVDRYKLGPIVNVILRRLRVMLMTVASGSW

SEQ ID NO: 13 - protein, 13 aa, Artificial Sequence
  BVDV
SLHGIWFEKICTG

SEQ ID NO: 14 - protein, 20 aa, Artificial Sequence
  BVDV
LQRHEWNKHGWCNWFHIEPW

SEQ ID NO: 15 - protein, 20 aa, Artificial Sequence
  BVDV
LQRHEWNKHGWCNWYNIEPW

SEQ ID NO: 16 - protein, 11 aa, Artificial Sequence
  BVDV
SLHGIWPEKIC

SEQ ID NO: 17 - protein, 12 aa, Artificial Sequence
  BVDV
RHEWNKHGWCNW

SEQ ID NO: 18 - protein, 4 aa, Artificial Sequence
  BVDV
MELF

SEQ ID NO: 19 - protein, 6 aa, Artificial Sequence
  BVDV
MELFSN

SEQ ID NO: 20 - protein, 7 aa, Artificial Sequence
  BVDV
MELFSNE

SEQ ID NO: 21 - protein, 8 aa, Artificial Sequence
  BVDV
MELFSNEL

SEQ ID NO: 22 - protein, 9 aa, Artificial Sequence
  BVDV
MELFSNELL

SEQ ID NO: 23 - protein, 10 aa, Artificial Sequence
  BVDV
MELFSNELLY

SEQ ID NO: 24 - protein, 11 aa, Artificial Sequence
  BVDV
MELFSNELLYK

SEQ ID NO: 25 - protein, 12 aa, Artificial Sequence
  BVDV
MELFSNELLYKT

SEQ ID NO: 26 - protein, 6 aa, Artificial Sequence
  BVDV
SNEGSK

SEQ ID NO: 27 - protein, 6 aa, Artificial Sequence
  BVDV
SDEGSK

SEQ ID NO: 28 - protein, 8 aa, Artificial Sequence
  BVDV
MESDQGSK

SEQ ID NO: 29 - protein, 11 aa, Artificial Sequence
  BVDV
MELFSSDQGSK

SEQ ID NO: 30 - protein, 13 aa, Artificial Sequence
  BVDV
MELFSNESDQGSK

SEQ ID NO: 31 - protein, 14 aa, Artificial Sequence
  BVDV
MELFSNELSDQGSK

SEQ ID NO: 32 - protein, 15 aa, Artificial Sequence
  BVDV
MELFSNELLSDQGSK

SEQ ID NO: 33 - protein, 16 aa, Artificial Sequence
  BVDV
MELFSNELLYSDQGSK

SEQ ID NO: 34 - protein, 17 aa, Artificial Sequence
  BVDV
MELFSNELLYKSDQGSK

SEQ ID NO: 35 - protein, 18 aa, Artificial Sequence
  BVDV
MELFSNELLYKTSDQGSK

SEQ ID NO: 36 - protein, 12 aa, Artificial Sequence
  BVDV
MELISNELLYKT

SEQ ID NO: 37 - protein, 12 aa, Artificial Sequence
  BVDV
MELITNELLYKT

SEQ ID NO: 38 - protein, 12 aa, Artificial Sequence
  BVDV
MELNHFELLYKT

SEQ ID NO: 39 - protein, 12 aa, Artificial Sequence
  BVDV
MELNKFELLYKT